(19) United States
(12) Patent Application Publication
(10) Pub. No.: US 2024/0327519 A1
(43) Pub. Date: Oct. 3, 2024

(54) METHODS AND COMPOSITIONS FOR TREATING CANCER

(71) Applicants: Genentech, Inc., South San Francisco, CA (US); Hoffmann-La Roche Inc., Little Falls, NJ (US)

(72) Inventors: Henry KAO, Basel (CH); Christoph MARKERT, Penzberg (DE); Christine MCINTYRE, Welwyn Garden City (GB); Raymond D. MENG, Mountain View, CA (US); Merlind MUECKE, Basel (CH); Volker TEICHGRAEBER, Basel (CH); Laura CODARRI DEAK, Schlieren (CH)

(21) Appl. No.: 18/424,297

(22) Filed: Jan. 26, 2024

Related U.S. Application Data
(63) Continuation of application No. PCT/US22/74279, filed on Jul. 28, 2022.
(60) Provisional application No. 63/226,714, filed on Jul. 28, 2021.

(51) Int. Cl. C07K 16/28; A61K 39/00; C07K 16/22

(52) U.S. Cl. CPC C07K 16/2818; C07K 16/22; C07K 16/2803; A61K 2039/505; A61K 2039/507; A61K 2039/545; C07K 2317/24; C07K 2317/31; C07K 2317/52; C07K 2317/522; C07K 2317/55; C07K 2317/56; C07K 2317/90

(57) ABSTRACT

This invention relates to methods and compositions for use in treating cancer in a subject by administering to the subject a bispecific antibody targeting programmed cell death protein 1 (PD-1) and lymphocyte activation gene-3 (LAG3).

### SEQUENCE LISTING

The instant application contains a Sequence Listing which has been submitted electronically in XML format and is hereby incorporated by reference in its entirety. Said XML copy, created on Nov. 21, 2023, is named 50474-304003_Sequence_Listing_11_21_23 and is 68,787 bytes in size.

### FIELD OF THE INVENTION

This invention relates to methods and compositions for use in treating cancer in a subject by administering to the subject a bispecific antibody targeting programmed cell death protein 1 (PD-1) and lymphocyte activation gene-3 (LAG3) (PD1-LAG3), optionally with an anti-TIGIT antagonist antibody (e.g., tiragolumab) or a VEGF antagonist (e.g., bevacizumab).

### BACKGROUND OF THE INVENTION

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Cancer care thus represents a significant and ever-increasing societal burden.

There is a particularly pressing need for therapeutic approaches for treatment of cancers that are common and difficult to treat.

Melanoma is a malignant tumor of melanocytes. This potentially deadly form of skin cancer is one of the fastest-growing malignancies. More than 300,000 people worldwide are currently diagnosed with melanoma each year, and 57,000 people die of the disease. Most people with advanced melanoma have a poor prognosis. Patients with lymph-node involvement (Stage III) have a high risk of local and distant relapse after surgery, and the 5-year survival rate is 32%-93% in this patient group. Few patients have metastatic disease (Stage IV) at presentation, but some develop metastases after their initial definitive treatment. Immunotherapy and targeted therapies have improved the outcomes of those patients, and the 5-year survival rate is around 50%. Melanoma continues to be a serious health issue, with a high medical need and a steadily increasing incidence over the past 30 years. Hence, there remains a significant need for novel therapeutic approaches in this population.

Liver cancer is the fifth most common cancer and the second most frequent cause of cancer-related death globally, with 854,000 new cases and 810,000 deaths per year. Hepatocellular carcinoma (HCC) is the most prevalent form of primary liver cancer and represents approximately 90% of all primary hepatic malignancies. Less prevalent primary liver cancers include intrahepatic cholangiocarcinoma (iCCA), angiosarcoma, and hepatoblastoma. Upon diagnosis, most patients with primary liver cancer present with advanced disease, a stage when treatment with curative therapies is not recommended. The WHO estimates that more than 1 million people will die from liver cancer in 2030, highlighting a significant global public health issue.

Thus, there is an unmet need in the field for the development of efficacious immunotherapies and methods of dosing the same for the treatment of cancer, including melanoma and liver cancer.

### SUMMARY OF THE INVENTION

In one aspect, the disclosure provides a method for treating a subject having a cancer, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3, wherein the method comprises administering to the subject the bispecific antibody at a fixed dose of 600 mg every three weeks.

In some aspects, the cancer is a solid tumor.

In some aspects, the cancer is locally advanced or metastatic.

In some aspects, the cancer is a skin cancer, a liver cancer, a lung cancer, a renal cancer, a bladder cancer, a breast cancer or an esophageal cancer. In some aspects, the skin cancer is a melanoma. In some aspects, the skin cancer is a previously untreated unresectable or metastatic melanoma. In some aspects, the melanoma is (a) a Stage III melanoma with measurable lymph node metastases; (b) an unresectable Stage III melanoma; or (c) a Stage IV melanoma, optionally wherein the melanoma is not a mucosal melanoma or a uveal melanoma. In some aspects, the liver cancer is hepatocellular carcinoma (HCC). In some aspects, the lung cancer is a non-small cell lung cancer (NSCLC). In some aspects, the renal cancer is renal cell carcinoma (RCC). In some aspects, the bladder cancer is metastatic urothelial carcinoma (mUC). In some aspects, the breast cancer is a triple-negative breast cancer (TNBC). In some aspects, the esophageal cancer is esophageal squamous cell carcinoma (ESCC).

In another aspect, the disclosure provides a method for treating a subject having a melanoma, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3, wherein the method comprises administering to the subject the bispecific antibody at a fixed dose of 600 mg every three weeks, and wherein the melanoma is (a) an unresectable Stage III melanoma; or (b) a Stage IV melanoma. In some aspects, the subject does not have ocular melanoma.

In another aspect, the disclosure provides a method for treating a subject having a liver cancer, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3, wherein the method comprises administering to the subject the bispecific antibody at a fixed dose of 600 mg every three weeks.

In some aspects, the liver cancer is hepatocellular carcinoma (HCC). In some aspects, the HCC is locally advanced, metastatic, and/or unresectable.

In some aspects, the subject has not previously received a systemic anti-cancer therapy.

In some aspects, the length of each of the one or more dosing cycles is 21 days. In some aspects, the method comprises administering to the subject the bispecific antibody on Day 1 of each of the one or more dosing cycles.

In some aspects, the method comprises administering to the subject the bispecific antibody intravenously.

In some aspects, the method further comprises administering to the subject bevacizumab at a dose of about 15 mg/kg every three weeks. In some aspects, the length of each of the one or more dosing cycles is 21 days and the method comprises administering to the subject the bevacizumab on Day 1 of each of the one or more dosing cycles. In some aspects, the bevacizumab is administered intravenously.

In some aspects, the subject has not previously been treated for metastatic or unresectable disease.

In some aspects, the subject has not previously been treated with an anti-cancer therapy comprising an immunomodulatory agent.

In some aspects, the subject has not previously been treated with an anti-LAG3 therapy.

In some aspects, the bispecific antibody targeting PD-1 and LAG3 comprises a first antigen-binding domain that specifically binds to PD-1 comprising a VH domain comprising (i) an HVR-H1 sequence comprising the amino acid sequence of SEQ ID NO: 25; (ii) an HVR-H2 sequence comprising the amino acid sequence GGR; and (iii) an HVR-H3 sequence comprising an amino acid sequence of SEQ ID NO: 26; and a VL domain comprising (i) an HVR-L1 sequence comprising the amino acid sequence of SEQ ID NO: 27; (ii) an HVR-L2 sequence comprising the amino acid sequence RSS; and (iii) an HVR-L3 sequence comprising the amino acid sequence of SEQ ID NO: 28.

In some aspects, the bispecific antibody targeting PD-1 and LAG3 comprises a second antigen-binding domain that specifically binds to LAG3 comprising a VH domain comprising (i) an HVR-H1 sequence comprising the amino acid sequence of SEQ ID NO: 31; (ii) an HVR-H2 sequence comprising the amino acid sequence of SEQ ID NO: 32; and (iii) an HVR-H3 sequence comprising an amino acid sequence of SEQ ID NO: 33; and a VL domain comprising (i) an HVR-L1 sequence comprising the amino acid sequence of SEQ ID NO: 34; (ii) an HVR-L2 sequence comprising the amino acid sequence of SEQ ID NO: 35; and (iii) an HVR-L3 sequence comprising the amino acid sequence of SEQ ID NO: 36.

In some aspects, the first antigen-binding domain comprises a VH domain comprising the amino acid sequence of SEQ ID NO: 29 and a VL domain comprising the amino acid sequence of SEQ ID NO: 30, and the second antigen-binding domain comprises a VH domain comprising the amino acid sequence of SEQ ID NO: 37 and a VL domain comprising the amino acid sequence of SEQ ID NO: 38.

In some aspects, the bispecific antibody is a full-length antibody.

In some aspects, the bispecific antibody targeting PD-1 and LAG3 comprises a Fc domain that is an IgG, optionally wherein the IgG Fc domain is an IgG1 Fc domain or an IgG4 Fc domain. In some aspects, the Fc domain comprises one or more amino acid substitutions that reduce binding to an Fc receptor, optionally wherein the Fc receptor is an Fcγ receptor.

In some aspects, the bispecific antibody targeting PD-1 and LAG3 comprises (a) an Fc domain of human IgG1 subclass with the amino acid mutations L234A, L235A, and P329G (numbering according to Kabat EU index); and/or (b) an Fc domain comprising a modification promoting the association of the first and second subunit of the Fc domain.

In some aspects, the first subunit of the Fc domain comprises the amino acid substitutions S354C and T366W (numbering according to Kabat EU index) and the second subunit of the Fc domain comprises the amino acid substitutions Y349C, T366S, and Y407V (numbering according to Kabat EU index).

In some aspects, the bispecific antibody targeting PD-1 and LAG3 comprises an Fc domain, a first Fab fragment comprising the first antigen-binding domain, and a second Fab fragment comprising the second antigen-binding domain. In some aspects, in one of the Fab fragments of the bispecific antibody targeting PD-1 and LAG3 the variable domains VL and VH are replaced by each other so that the VH domain is part of the light chain and the VL domain is part of the heavy chain, optionally wherein in the first Fab fragment the variable domains VL and VH are replaced by each other. In some aspects, in the constant domain CL of one of the Fab fragments the amino acid at position 124 is substituted independently by lysine (K), arginine (R), or histidine (H) (numbering according to Kabat EU Index), and in the constant domain CH1 the amino acids at positions 147 and 213 are substituted independently by glutamic acid (E) or aspartic acid (D) (numbering according to Kabat EU index), optionally wherein in the constant domain CL of the second Fab fragment the amino acid at position 124 is substituted independently by lysine (K), arginine (R), or histidine (H) (numbering according to Kabat EU Index), and in the constant domain CH1 the amino acids at positions 147 and 213 are substituted independently by glutamic acid (E) or aspartic acid (D) (numbering according to Kabat EU index).

In some aspects, the bispecific antibody comprises a first heavy chain comprising an amino acid sequence with at least 95% sequence identity to the sequence of SEQ ID NO: 39, a first light chain comprising an amino acid sequence with at least 95% sequence identity to the sequence of SEQ ID NO: 40, a second heavy chain comprising an amino acid sequence with at least 95% sequence identity to the sequence of SEQ ID NO: 41, and a second light chain comprising an amino acid sequence with at least 95% sequence identity to the sequence of SEQ ID NO: 42. In some aspects, the bispecific antibody comprises a first heavy chain comprising the amino acid sequence of SEQ ID NO: 39, a first light chain comprising the amino acid sequence of SEQ ID NO: 40, a second heavy chain comprising the amino acid sequence of SEQ ID NO: 41, and a second light chain comprising the amino acid sequence of SEQ ID NO: 42.

In some aspects, the bispecific antibody achieves at least 90% LAG3 receptor occupancy (RO) in the tumor.

In some aspects, the subject is a human.

In another aspect, the disclosure provides a bispecific antibody targeting PD-1 and LAG3 for use in a method of treating a subject having a cancer, wherein the bispecific antibody comprises a first antigen-binding domain that specifically binds to PD-1 and a second antigen-binding domain that specifically binds to LAG3, and wherein the method comprises administering to the subject the bispecific antibody at a fixed dose of 600 mg every three weeks.

In some aspects, the cancer is a solid tumor.

In some aspects, the cancer is locally advanced or metastatic.

In some aspects, the cancer is a skin cancer, a liver cancer, a lung cancer, a renal cancer, a bladder cancer, a breast cancer or an esophageal cancer. In some aspects, the skin cancer is a melanoma. In some aspects, the skin cancer is a previously untreated unresectable or metastatic melanoma. In some aspects, the melanoma is (a) a Stage III melanoma with measurable lymph node metastases; (b) an unresectable Stage III melanoma; or (c) a Stage IV melanoma, optionally wherein the melanoma is not a mucosal melanoma or a uveal melanoma. In some aspects, the liver cancer is hepatocellular carcinoma (HCC). In some aspects, the lung cancer is a non-small cell lung cancer (NSCLC). In some aspects, the renal cancer is renal cell carcinoma (RCC). In some aspects, the bladder cancer is metastatic urothelial carcinoma (mUC). In some aspects, the breast cancer is a triple-negative breast cancer (TNBC). In some aspects, the esophageal cancer is esophageal squamous cell carcinoma (ESCC).

In another aspect, the disclosure provides a bispecific antibody targeting PD-1 and LAG3 for use in a method of treating a subject having a melanoma, wherein the bispecific antibody comprises a first antigen-binding domain that specifically binds to PD-1 and a second antigen-binding domain that specifically binds to LAG3, wherein the method comprises administering to the subject the bispecific antibody at a fixed dose of 600 mg every three weeks, and wherein the melanoma is (a) an unresectable Stage III melanoma; or (b) a Stage IV melanoma. In some aspects, the patient does not have ocular melanoma.

In another aspect, the disclosure provides a bispecific antibody targeting PD-1 and LAG3 for use in a method for treating a subject having a liver cancer, wherein the bispecific antibody comprises a first antigen-binding domain that specifically binds to PD-1 and a second antigen-binding domain that specifically binds to LAG3, and wherein the method comprises administering to the subject the bispecific antibody at a fixed dose of 600 mg every three weeks. In some aspects, the liver cancer is HCC. In some aspects, the HCC is locally advanced, metastatic, and/or unresectable.

In some aspects, the subject has not previously received a systemic anti-cancer therapy.

In some aspects, the length of each of the one or more dosing cycles is 21 days. In some aspects, the method comprises administering to the subject the bispecific antibody on Day 1 of each of the one or more dosing cycles.

In some aspects, the method comprises administering to the subject the bispecific antibody intravenously.

In some aspects, the method further comprises administering to the subject bevacizumab at a dose of about 15 mg/kg every three weeks. In some aspects, the length of each of the one or more dosing cycles is 21 days and the method comprises administering to the subject the bevacizumab on Day 1 of each of the one or more dosing cycles. In some aspects, the bevacizumab is administered intravenously.

In some aspects, the subject has not previously been treated for metastatic or unresectable disease.

In some aspects, the subject has not previously been treated with an anti-cancer therapy comprising an immunomodulatory agent.

In some aspects, the subject has not previously been treated with an anti-LAG3 therapy.

In some aspects, the bispecific antibody targeting PD-1 and LAG3 comprises a first antigen-binding domain that specifically binds to PD-1 comprising a VH domain comprising (i) an HVR-H1 sequence comprising the amino acid sequence of SEQ ID NO: 25; (ii) an HVR-H2 sequence comprising the amino acid sequence GGR; and (iii) an HVR-H3 sequence comprising an amino acid sequence of SEQ ID NO: 26; and a VL domain comprising (i) an HVR-L1 sequence comprising the amino acid sequence of SEQ ID NO: 27; (ii) an HVR-L2 sequence comprising the amino acid sequence RSS; and (iii) an HVR-L3 sequence comprising the amino acid sequence of SEQ ID NO: 28.

In some aspects, the bispecific antibody targeting PD-1 and LAG3 comprises a second antigen-binding domain that specifically binds to LAG3 comprising a VH domain comprising (i) an HVR-H1 sequence comprising the amino acid sequence of SEQ ID NO: 31; (ii) an HVR-H2 sequence comprising the amino acid sequence of SEQ ID NO: 32; and (iii) an HVR-H3 sequence comprising an amino acid sequence of SEQ ID NO: 33; and a VL domain comprising (i) an HVR-L1 sequence comprising the amino acid sequence of SEQ ID NO: 34; (ii) an HVR-L2 sequence comprising the amino acid sequence of SEQ ID NO: 35; and (iii) an HVR-L3 sequence comprising the amino acid sequence of SEQ ID NO: 36.

In some aspects, the first antigen-binding domain comprises a VH domain comprising the amino acid sequence of SEQ ID NO: 29 and a VL domain comprising the amino acid sequence of SEQ ID NO: 30, and the second antigen-binding domain comprises a VH domain comprising the amino acid sequence of SEQ ID NO: 37 and a VL domain comprising the amino acid sequence of SEQ ID NO: 38.

In some aspects, the bispecific antibody is a full-length antibody.

In some aspects, the bispecific antibody targeting PD-1 and LAG3 comprises a Fc domain that is an IgG, optionally wherein the IgG Fc domain is an IgG1 Fc domain or an IgG4 Fc domain. In some aspects, the Fc domain comprises one or more amino acid substitutions that reduce binding to an Fc receptor, optionally wherein the Fc receptor is an Fcγ receptor.

In some aspects, the bispecific antibody targeting PD-1 and LAG3 comprises (a) an Fc domain of human IgG1 subclass with the amino acid mutations L234A, L235A, and P329G (numbering according to Kabat EU index); and/or (b) an Fc domain comprising a modification promoting the association of the first and second subunit of the Fc domain.

In some aspects, the first subunit of the Fc domain comprises the amino acid substitutions S354C and T366W (numbering according to Kabat EU index) and the second subunit of the Fc domain comprises the amino acid substitutions Y349C, T366S, and Y407V (numbering according to Kabat EU index).

In some aspects, the bispecific antibody targeting PD-1 and LAG3 comprises an Fc domain, a first Fab fragment comprising the first antigen-binding domain, and a second Fab fragment comprising the second antigen-binding domain. In some aspects, in one of the Fab fragments of the bispecific antibody targeting PD-1 and LAG3 the variable domains VL and VH are replaced by each other so that the VH domain is part of the light chain and the VL domain is part of the heavy chain, optionally wherein in the first Fab fragment the variable domains VL and VH are replaced by each other. In some aspects, in the constant domain CL of one of the Fab fragments the amino acid at position 124 is substituted independently by lysine (K), arginine (R), or histidine (H) (numbering according to Kabat EU Index), and in the constant domain CH1 the amino acids at positions 147 and 213 are substituted independently by glutamic acid (E) or aspartic acid (D) (numbering according to Kabat EU index), optionally wherein in the constant domain CL of the second Fab fragment the amino acid at position 124 is substituted independently by lysine (K), arginine (R), or histidine (H) (numbering according to Kabat EU Index), and in the constant domain CH1 the amino acids at positions 147 and 213 are substituted independently by glutamic acid (E) or aspartic acid (D) (numbering according to Kabat EU index).

In some aspects, the bispecific antibody comprises a first heavy chain comprising an amino acid sequence with at least 95% sequence identity to the sequence of SEQ ID NO: 39, a first light chain comprising an amino acid sequence with at least 95% sequence identity to the sequence of SEQ ID NO: 40, a second heavy chain comprising an amino acid sequence with at least 95% sequence identity to the sequence of SEQ ID NO: 41, and a second light chain comprising an amino acid sequence with at least 95% sequence identity to the sequence of SEQ ID NO: 42. In some aspects, the bispecific antibody comprises a first heavy chain comprising the amino acid sequence of SEQ ID NO: 39, a first light chain comprising the amino acid sequence of SEQ ID NO: 40, a second heavy chain comprising the amino acid sequence of SEQ ID NO: 41, and a second light chain comprising the amino acid sequence of SEQ ID NO: 42.

In some aspects, the bispecific antibody achieves at least 90% LAG3 receptor occupancy (RO) in the tumor.

In some aspects, the subject is a human.

In another aspect, the disclosure provides use of a bispecific antibody targeting PD-1 and LAG3 in the manufacture of a medicament for treating a subject having a cancer, wherein the bispecific antibody comprises a first antigen-binding domain that specifically binds to PD-1 and a second antigen-binding domain that specifically binds to LAG3, and wherein the bispecific antibody is to be administered to the subject at a fixed dose of 600 mg every three weeks.

In some aspects, the cancer is a solid tumor.

In some aspects, the cancer is locally advanced or metastatic.

In some aspects, the cancer is a skin cancer, a liver cancer, a lung cancer, a renal cancer, a bladder cancer, a breast cancer or an esophageal cancer. In some aspects, the skin cancer is a melanoma. In some aspects, the skin cancer is a previously untreated unresectable or metastatic melanoma. In some aspects, the melanoma is (a) a Stage III melanoma with measurable lymph node metastases; (b) an unresectable Stage III melanoma; or (c) a Stage IV melanoma, optionally wherein the melanoma is not a mucosal melanoma or a uveal melanoma. In some aspects, the liver cancer is hepatocellular carcinoma (HCC). In some aspects, the lung cancer is a non-small cell lung cancer (NSCLC). In some aspects, the renal cancer is renal cell carcinoma (RCC). In some aspects, the bladder cancer is metastatic urothelial carcinoma (mUC). In some aspects, the breast cancer is a triple-negative breast cancer (TNBC). In some aspects, the esophageal cancer is esophageal squamous cell carcinoma (ESCC).

In another aspect, the disclosure provides use of a bispecific antibody targeting PD-1 and LAG3 in the manufacture of a medicament for treating a subject having a melanoma, wherein the bispecific antibody comprises a first antigen-binding domain that specifically binds to PD-1 and a second antigen-binding domain that specifically binds to LAG3, wherein the bispecific antibody is to be administered to the subject at a fixed dose of 600 mg every three weeks, and wherein the melanoma is (a) an unresectable Stage III melanoma; or (b) a Stage IV melanoma. In some aspects, the subject does not have ocular melanoma.

In another aspect, the disclosure provides use of a bispecific antibody targeting PD-1 and LAG3 in the manufacture of a medicament for treating a subject having a liver cancer, wherein the bispecific antibody comprises a first antigen-binding domain that specifically binds to PD-1 and a second antigen-binding domain that specifically binds to LAG3, wherein the bispecific antibody is to be administered to the subject at a fixed dose of 600 mg every three weeks. In some aspects, the liver cancer is HCC. In some aspects, the HCC is locally advanced, metastatic, and/or unresectable.

In some aspects, the subject has not previously received a systemic anti-cancer therapy.

In some aspects, the length of each of the one or more dosing cycles is 21 days. In some aspects, the bispecific antibody is to be administered to the subject on Day 1 of each of the one or more dosing cycles.

In some aspects, the bispecific antibody is to be administered to the subject intravenously.

In some aspects, bevacizumab is to be administered to the subject at a dose of about 15 mg/kg every three weeks. In some aspects, the length of each of the one or more dosing cycles is 21 days and the bevacizumab is to be administered to the subject on Day 1 of each of the one or more dosing cycles. In some aspects, the bevacizumab is administered intravenously.

In some aspects, the subject has not previously been treated for metastatic or unresectable disease.

In some aspects, the subject has not previously been treated with an anti-cancer therapy comprising an immunomodulatory agent.

In some aspects, the subject has not previously been treated with an anti-LAG3 therapy.

In some aspects, the bispecific antibody targeting PD-1 and LAG3 comprises a first antigen-binding domain that specifically binds to PD-1 comprising a VH domain comprising (i) an HVR-H1 sequence comprising the amino acid sequence of SEQ ID NO: 25; (ii) an HVR-H2 sequence comprising the amino acid sequence GGR; and (iii) an HVR-H3 sequence comprising an amino acid sequence of SEQ ID NO: 26; and a VL domain comprising (i) an HVR-L1 sequence comprising the amino acid sequence of SEQ ID NO: 27; (ii) an HVR-L2 sequence comprising the amino acid sequence RSS; and (iii) an HVR-L3 sequence comprising the amino acid sequence of SEQ ID NO: 28.

In some aspects, the bispecific antibody targeting PD-1 and LAG3 comprises a second antigen-binding domain that specifically binds to LAG3 comprising a VH domain comprising (i) an HVR-H1 sequence comprising the amino acid sequence of SEQ ID NO: 31; (ii) an HVR-H2 sequence comprising the amino acid sequence of SEQ ID NO: 32; and (iii) an HVR-H3 sequence comprising an amino acid sequence of SEQ ID NO: 33; and a VL domain comprising (i) an HVR-L1 sequence comprising the amino acid sequence of SEQ ID NO: 34; (ii) an HVR-L2 sequence comprising the amino acid sequence of SEQ ID NO: 35; and (iii) an HVR-L3 sequence comprising the amino acid sequence of SEQ ID NO: 36.

In some aspects, the first antigen-binding domain comprises a VH domain comprising the amino acid sequence of SEQ ID NO: 29 and a VL domain comprising the amino acid sequence of SEQ ID NO: 30, and the second antigen-binding domain comprises a VH domain comprising the amino acid sequence of SEQ ID NO: 37 and a VL domain comprising the amino acid sequence of SEQ ID NO: 38.

In some aspects, the bispecific antibody is a full-length antibody.

In some aspects, the bispecific antibody targeting PD-1 and LAG3 comprises a Fc domain that is an IgG, optionally wherein the IgG Fc domain is an IgG1 Fc domain or an IgG4 Fc domain. In some aspects, the Fc domain comprises one or more amino acid substitutions that reduce binding to an Fc receptor, optionally wherein the Fc receptor is an Fcγ receptor.

In some aspects, the bispecific antibody targeting PD-1 and LAG3 comprises (a) an Fc domain of human IgG1 subclass with the amino acid mutations L234A, L235A, and P329G (numbering according to Kabat EU index); and/or (b) an Fc domain comprising a modification promoting the association of the first and second subunit of the Fc domain.

In some aspects, the first subunit of the Fc domain comprises the amino acid substitutions S354C and T366W (numbering according to Kabat EU index) and the second subunit of the Fc domain comprises the amino acid substitutions Y349C, T366S, and Y407V (numbering according to Kabat EU index).

In some aspects, the bispecific antibody targeting PD-1 and LAG3 comprises an Fc domain, a first Fab fragment comprising the first antigen-binding domain, and a second Fab fragment comprising the second antigen-binding domain. In some aspects, in one of the Fab fragments of the bispecific antibody targeting PD-1 and LAG3 the variable domains VL and VH are replaced by each other so that the VH domain is part of the light chain and the VL domain is part of the heavy chain, optionally wherein in the first Fab fragment the variable domains VL and VH are replaced by each other. In some aspects, in the constant domain CL of one of the Fab fragments the amino acid at position 124 is substituted independently by lysine (K), arginine (R), or histidine (H) (numbering according to Kabat EU Index), and in the constant domain CH1 the amino acids at positions 147 and 213 are substituted independently by glutamic acid (E) or aspartic acid (D) (numbering according to Kabat EU index), optionally wherein in the constant domain CL of the second Fab fragment the amino acid at position 124 is substituted independently by lysine (K), arginine (R), or histidine (H) (numbering according to Kabat EU Index), and in the constant domain CH1 the amino acids at positions 147 and 213 are substituted independently by glutamic acid (E) or aspartic acid (D) (numbering according to Kabat EU index).

In some aspects, the bispecific antibody comprises a first heavy chain comprising an amino acid sequence with at least 95% sequence identity to the sequence of SEQ ID NO: 39, a first light chain comprising an amino acid sequence with at least 95% sequence identity to the sequence of SEQ ID NO: 40, a second heavy chain comprising an amino acid sequence with at least 95% sequence identity to the sequence of SEQ ID NO: 41, and a second light chain comprising an amino acid sequence with at least 95% sequence identity to the sequence of SEQ ID NO: 42. In some aspects, the bispecific antibody comprises a first heavy chain comprising the amino acid sequence of SEQ ID NO: 39, a first light chain comprising the amino acid sequence of SEQ ID NO: 40, a second heavy chain comprising the amino acid sequence of SEQ ID NO: 41, and a second light chain comprising the amino acid sequence of SEQ ID NO: 42.

In some aspects, the bispecific antibody achieves at least 90% LAG3 receptor occupancy (RO) in the tumor.

In some aspects, the subject is a human.

### BRIEF DESCRIPTION OF THE DRAWINGS

FIG. 1 is a flow chart showing the study design of the phase Ib/II clinical trial in patients with melanoma. Atezo=atezolizumab; CIT=cancer immunotherapy; CLND=completion lymph node dissection; Ipi=ipilimumab; Nivo=nivolumab; R=randomization; Tira=tiragolumab.

FIG. 2 is a schematic diagram of the study schema showing an overview of the study schedule and activities in Cohort 1 of the phase Ib/II clinical trial in patients with melanoma. CLND=completion lymph node dissection; Comp.=completion; CT=computed tomography; Discon.=discontinuation; M=month; R=randomization; Q3M=every 3 months; SFU=survival follow-up; Tx=treatment; W=week.

FIG. 3 is a schematic diagram showing a minimal physiologically-based pharmacokinetics (mPBPK) model for pembrolizumab. Jt=pembrolizumab flow into tumor compartment; kint=pembrolizumab-PD-1 complex internalization rate constant; koff=dissociation rate constant; kon=association rate constant; kdeg=degradation rate of PD-1; Ksyn=synthesis rate of PD-1; Lt=lymph flow from tumor compartment; L1=lymph flow from tight tissue compartment; L2=lymph flow from leaky tissue compartment; mAb=pembrolizumab concentration; Qt=tumor plasma flow rate; RC=pembrolizumab-PD-1 complex concentration; Rmax=total PD-1 concentration; Vleaky=volume of tissue compartment with leaky vascular structure; Vlymph=volume of lymph compartment; Vp=volume of plasma compartment; Vtight=volume of tissue compartment with tight vascular structure; Vtumor=volume of tumor compartment; σtight=vascular reflection coefficient of tight tissue; σleaky=vascular reflection coefficient of leaky tissue; σLy=lymph reflection coefficient.

FIG. 4 is a schematic diagram showing the additional LAG3 receptor added to the mPBPK model for PD1-LAG3.

FIG. 5 is a table showing an overview of adverse events in safety evaluable patients in the dose escalation (Part A1, Q2W) portion of the NP41300 study.

FIG. 6 is a table showing an overview of adverse events in safety evaluable patients in the dose escalation (Part A1, Q2W) portion of the NP41300 study.

FIG. 7 is a set of box plots showing predicted C_{trough} after the first and third administrations PD1-LAG3 at a dose of 600 mg or 1200 mg in a Q2W (every 2 weeks) or Q3W (every 3 weeks) dosing regimen. The lower and upper hinges correspond to the first and third quartiles (the 25th and 75th percentiles). The upper whisker extends from the hinge to the largest value no further than 1.5*IQR from the hinge (where IQR is the inter-quartile range, or distance between the first and third quartiles). The lower whisker extends from the hinge to the smallest value at most 1.5*IQR of the hinge. Data beyond the end of the whiskers are called “outlying” points and are plotted individually. Simulations were performed using a population pharmacokinetic model (nonlinear mixed effect modeling approach). For each dosing regimen, 500 individuals were simulated with a set of covariates bootstrapped (with replacement) from the original analysis dataset.

FIG. 8 is a graph showing simulated PD1 and LAG3 engagement over a range of RO7247669 doses administered Q3W after 3 cycles.

FIG. 9 is a flow chart showing the study design of the BP43963 trial in patients with melanoma.

FIG. 10 is a flow chart showing the study design of the GO42216 clinical trial. CIT=cancer immunotherapy; HCC=hepatocellular carcinoma; R=randomization.

FIG. 11 is a flow chart showing the detailed study design of the GO42216 clinical trial. Bev=bevacizumab; HCC=hepatocellular carcinoma; Q2W=every 2 weeks; Q3W=every 3 weeks; R=randomization.

### DETAILED DESCRIPTION OF THE INVENTION

The present invention provides therapeutic methods and compositions for treatment of cancer. Compositions, uses, and kits involving such combinations and/or dosing regimens are also provided herein.

### 1. Definitions

The following abbreviations are used herein:

| | |
|---|---|
| ASCT | autologous stem cell therapy |
| CAR | chimeric antigen receptor |
| CAS | Chemical Abstracts Service |
| CDR | complementarity determining region |
| CR | complete response |
| CRC | colorectal cancer |
| DNA | deoxyribonucleic acid |
| DCR | disease control rate |
| DOR | duration of response |
| Fab | fragment antigen-binding |
| Fc | fragment crystallizable |
| FFPE | formalin-fixed and paraffin-embedded |
| FR | framework |
| HCC | hepatocellular carcinoma |
| HVR | hypervariable region |
| IHC | immunohistochemistry |
| IV | intravenous |
| MSI | microsatellite instability |
| NHL | non-Hodgkin's lymphoma |
| ORR | overall response rate/objective |
| | response rate |
| OS | overall survival |
| PD-1 | programmed death 1 |
| PD-L1 | programmed death ligand 1 |
| PD-L2 | programmed death ligand 2 |
| PFS | progression-free survival |
| PR | partial response |
| Q2W | once every 2 weeks |
| Q3W | once every 3 weeks |
| Q4W | once every 4 weeks |
| RNA | ribonucleic acid |
| SC | subcutaneous |
| SLD | sum of the longest diameters |

The term “about” as used herein refers to the usual error range for the respective value readily known to the skilled person in this technical field. Reference to “about” a value or parameter herein includes (and describes) aspects that are directed to that value or parameter per se. For example, description referring to “about X” includes description of “X.”

The term “TIGIT” or “T-cell immunoreceptor with Ig and ITIM domains” as used herein refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term encompasses “full-length,” unprocessed TIGIT (e.g., full-length human TIGIT having the amino acid sequence of SEQ ID NO: 20), as well as any form of TIGIT that results from processing in the cell (e.g., processed human TIGIT without a signal sequence, having the amino acid sequence of SEQ ID NO: 21). The term also encompasses naturally occurring variants of TIGIT, e.g., splice variants or allelic variants. The amino acid sequence of an exemplary human TIGIT may be found under UniProt Accession Number Q495A1.

As used herein, “tiragolumab” is a fully human IgG1/kappa MAb-derived in Open Monoclonal Technology (OMT) rats that binds TIGIT and comprises the heavy chain sequence of SEQ ID NO: 23 and the light chain sequence of SEQ ID NO: 24. Tiragolumab comprises two N-linked glycosylation sites (N306) in the Fc domain. Tiragolumab is also described in WHO Drug Information (International Nonproprietary Names for Pharmaceutical Substances), Proposed INN: List 117, Vol. 31, No. 2, published Jul. 7, 2017 (see page 343).

The term “anti-TIGIT antagonist antibody” refers to an antibody or an antigen-binding fragment or variant thereof that is capable of binding TIGIT with sufficient affinity such that it substantially or completely inhibits the biological activity of TIGIT. For example, an anti-TIGIT antagonist antibody may block signaling through PVR, PVRL2, and/or PVRL3 so as to restore a functional response by T-cells (e.g., proliferation, cytokine production, target cell killing) from a dysfunctional state to antigen stimulation. For example, an anti-TIGIT antagonist antibody may block signaling through PVR without impacting PVR-CD226 interaction. It will be understood by one of ordinary skill in the art that in some instances, an anti-TIGIT antagonist antibody may antagonize one TIGIT activity without affecting another TIGIT activity. For example, an anti-TIGIT antagonist antibody for use in certain of the methods or uses described herein is an anti-TIGIT antagonist antibody that antagonizes TIGIT activity in response to one of PVR interaction, PVRL3 interaction, or PVRL2 interaction, e.g., without affecting or minimally affecting any of the other TIGIT interactions. In one aspect, the extent of binding of an anti-TIGIT antagonist antibody to an unrelated, non-TIGIT protein is less than about 10% of the binding of the antibody to TIGIT as measured, e.g., by a radioimmunoassay (RIA). In certain aspects, an anti-TIGIT antagonist antibody that binds to TIGIT has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, an anti-TIGIT antagonist antibody binds to an epitope of TIGIT that is conserved among TIGIT from different species or an epitope on TIGIT that allows for cross-species reactivity. In some aspects, the anti-TIGIT binding antibody has intact Fc-mediated effector function (e.g., tiragolumab, vibostolimab, etigilimab, EOS084448, or TJ-T6). In some aspects, the anti-TIGIT binding antibody has enhanced Fc-mediated effector function (e.g., SGN-TGT). In other aspects, the anti-TIGIT binding antibody lacks Fc-mediated effector function (e.g., domvanalimab, BMS-986207, ASP8374, or COM902). In some aspects, the anti-TIGIT binding antibody is an IgG1 class antibody (e.g., tiragolumab, vibostolimab, domvanalimab, BMS-986207, etigilimab, BGB-A1217, SGN-TGT, EOS084448 (EOS-448), TJ-T6, or AB308). In other aspects, the anti-TIGIT binding antibody is an IgG4 class antibody (e.g., ASP8374 or COM902). In one aspect, the anti-TIGIT antagonist antibody is tiragolumab.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The terms “programmed death ligand 1” and “PD-L1” refer herein to native sequence human PD-L1 polypeptide. Native sequence PD-L1 polypeptides are provided under Uniprot Accession No. Q9NZQ7. For example, the native sequence PD-L1 may have the amino acid sequence as set forth in Uniprot Accession No. Q9NZQ7-1 (isoform 1) (SEQ ID NO: 22). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

For the purposes herein, “atezolizumab” is an Fc-engineered, humanized, non-glycosylated IgG1 kappa immunoglobulin that binds PD-L1 and comprises the heavy chain sequence of SEQ ID NO: 62 and the light chain sequence of SEQ ID NO: 63. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

The term “cancer” refers to a disease caused by an uncontrolled division of abnormal cells in a part of the body. In one instance, the cancer is skin cancer (e.g., melanoma, basal cell carcinoma (BCC), squamous cell carcinoma, cutaneous T-cell lymphoma, dermatofibrosarcoma protuberans (DFSP), Merkel cell carcinoma, or sebaceous carcinoma). In another instance, the cancer is a liver cancer (e.g., hepatocellular carcinoma (HCC), e.g., locally advanced or metastatic HCC and/or unresectable HCC). Cancers include solid tumor cancers and non-solid tumor cancers and locally advanced or metastatic cancers (e.g., locally advanced or metastatic tumors). Examples of cancer include but are not limited to, carcinoma, lymphoma, blastoma, sarcoma, and leukemia or lymphoid malignancies. More particular examples of such cancers include, but are not limited to urothelial carcinoma (UC), including locally advanced and metastatic UC (mUC), bladder cancer (e.g., muscle invasive bladder cancer (MIBC) and non-muscle invasive bladder cancer (NMIBC), e.g., BCG-refractory NMIBC), MIBC urothelial bladder cancer (UBC); kidney or renal cancer (e.g., renal cell carcinoma (RCC)); cancer of the urinary tract; lung cancer, such as small cell lung cancer (SCLC), which includes extensive stage SCLC (ES-SCLC); non-small cell lung cancer (NSCLC), which includes squamous NSCLC or non-squamous NSCLC, including locally advanced unresectable NSCLC (e.g., Stage IIIB NSCLC), or recurrent or metastatic NSCLC (e.g., Stage IV NSCLC), adenocarcinoma of the lung, or squamous cell cancer (e.g., epithelial squamous cell cancer (e.g., squamous carcinoma of the lung); pancreatic cancer (e.g., pancreatic ductal adenocarcinoma (PDAC), e.g., metastatic PDAC)); head and neck cancer (e.g., SCCHN, e.g., recurrent/metastatic PD-L1-positive SCCHN, and head and neck squamous cell cancer (HNSCC); ovarian cancer (OC); esophageal cancer; cancer of the peritoneum; hepatocellular cancer; gastric cancer (GC) (e.g., gastroesophageal junction (GEJ) cancer) or stomach cancer, including gastrointestinal cancer and gastrointestinal stromal cancer; glioblastoma; cancer of the urinary tract; hepatoma; breast cancer (e.g., HER2+ breast cancer and triple-negative breast cancer (TNBC (e.g., early TNBC (eTNBC)), which are estrogen receptors (ER−), progesterone receptors (PgR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer (e.g., pancreatic ductal adenocarcinoma (PDAC)); glioblastoma; cervical cancer (e.g., a Stage IVB, metastatic, recurrent, or persistent cervical cancer, e.g., a metastatic and/or recurrent PD-L1-positive cervical carcinoma); ovarian cancer; hepatoma; colon cancer; rectal cancer; colorectal cancer (CRC; e.g., CRC with microsatellite-stable (MSS) and microsatellite instability (MSI) low (MSI-Low)); endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL)); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

In some instances, the cancer (e.g., the skin cancer (e.g., melanoma) or liver cancer (e.g., HCC)) is a tumor having a tumor microenvironment comprising LAG3-expressing CD8+ T cells.

In some instances, the cancer may be unresectable (e.g., unresectable locally advanced or metastatic cancer).

Further examples of cancer include, but are not limited to, carcinoma, lymphoma, blastoma, sarcoma, and leukemia or lymphoid malignancies. More particular examples of cancers include, but are not limited to, esophageal cancer (e.g., squamous cell carcinoma (e.g., esophageal squamous-cell carcinoma (ESCC)), adenocarcinoma (e.g., esophageal adenocarcinoma (EAC)), or esophageal cancers having neuroendocrine histopathology (e.g., esophageal neuroendocrine carcinoma (ENEC)). Additional examples include metastatic esophageal cancer (e.g., metastatic ESCC, metastatic EAC, or metastatic ENEC). In one instance, the cancer is a colorectal cancer (CRC). As used herein, the term “colorectal cancer,” “CRC,” “colon cancer,” or “bowel cancer” refers to a cancer that develops from the large intestine, e.g., the colon or rectum (e.g., colorectal adenomacarcinoma). Other examples of cancer include, but are not limited to, hematologic cancers, such as mature B cell cancers, excluding Hodgkin's lymphoma, but including non-Hodgkin's lymphoma (NHL), such as diffuse large B cell lymphoma (DLBCL), which may be relapsed or refractory DLBCL or a Richter's transformation. Other specific examples of cancer also include germinal-center B cell-like (GCB) diffuse large B cell lymphoma (DLBCL), activated B cell-like (ABC) DLBCL, follicular lymphoma (FL), transformed FL, mantle cell lymphoma (MCL), acute myeloid leukemia (AML), chronic lymphoid leukemia (CLL), marginal zone lymphoma (MZL), transformed MZL, high grade B-cell lymphoma, primary mediastinal (thymic) large B cell lymphoma (PMLBCL), small lymphocytic leukemia (SLL), lymphoplasmacytic lymphoma (LL), transformed LL, Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, heavy chain diseases, α heavy chain disease, γ heavy chain disease, μ heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle center lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of cancer include, but are not limited to, carcinoma, lymphoma, blastoma, sarcoma, and leukemia or lymphoid malignancies, including B cell lymphomas. More particular examples of such cancers include, but are not limited to, multiple myeloma (MM); low-grade/follicular NHL; small lymphocytic (SL) NHL; intermediate-grade/follicular NHL; intermediate-grade diffuse NHL; high-grade immunoblastic NHL; high-grade lymphoblastic NHL; high-grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD).

The terms “B cell proliferative disorder” or “B cell malignancy” refer to disorders that are associated with some degree of abnormal B cell proliferation and include, for example, lymphomas, leukemias, myelomas, and myelodysplastic syndromes. In some instances, the B cell proliferative disorder is a lymphoma, such as non-Hodgkin's lymphoma (NHL), including, for example, follicular lymphoma (FL) (e.g., a relapsed and/or refractory FL or transformed FL), diffuse large B cell lymphoma (DLBCL) (e.g., a relapsed or refractory DLBCL or a Richter's transformation), MCL, high grade B-cell lymphoma, or PMLBCL). In another embodiment, the B cell proliferative disorder is a leukemia, such as chronic lymphocytic leukemia (CLL). In one embodiment, the B-cell proliferative disorder is a relapsed and/or refractory FL.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

A “tumor cell,” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

As used herein, “treating” comprises effective cancer treatment with an effective amount of a therapeutic agent (e.g., a bispecific antibody targeting PD-1 and LAG3)). Treating herein includes, inter alia, adjuvant therapy, neoadjuvant therapy, non-metastatic cancer therapy (e.g., locally advanced cancer therapy), and metastatic cancer therapy. The treatment may be first-line treatment (e.g., the patient may be previously untreated or not have received prior systemic therapy), or second line or later treatment.

Herein, an “effective amount” refers to the amount of a therapeutic agent (e.g., a a bispecific antibody targeting PD-1 and LAG3 or a combination of therapeutic agents (e.g., a bispecific antibody targeting PD-1 and LAG3 and an anti-TIGIT antagonist antibody, tiragolumab or a VEGF antagonist, e.g., an anti-VEGF antibody (e.g., bevacizumab)), that achieves a therapeutic result. In some examples, the effective amount of a therapeutic agent or a combination of therapeutic agents is the amount of the agent or of the combination of agents that achieves a clinical endpoint of improved pathologic response rate (PRR), improved overall response rate (ORR), improved disease control rate (DCR), a complete response (CR), a pathological complete response (pCR), a partial response (PR), improved survival (e.g., disease-free survival (DFS), and/or progression-free survival (PFS) and/or overall survival (OS)), and/or improved duration of response (DOR).

As used herein, “complete response” and “CR” refers to disappearance of all target lesions.

As used herein, “partial response” and “PR” refers to at least a 30% decrease in the sum of the longest diameters (SLD) of target lesions, taking as reference the baseline SLD prior to treatment.

As used here, “progressive disease” and “PD” refers to at least a 20% increase in the SLD of target lesions, taking as reference the smallest sum on study (nadir), including baseline. The appearance of one or more new lesions may also be considered PD.

As used herein, “stable disease” and “SD” refers to neither sufficient shrinkage to qualify for PR nor sufficient increase to qualify for PD, taking as reference the smallest sum.

As used herein, “disease control rate” and “DCR” refer to the percentage of patients with advanced or metastatic cancer who have achieved CR, PR, and stable disease (SD). For example, DCR may be defined as the proportion of patients with SD for ≥12 weeks or a CR or PR, as determined by the investigator according to RECIST v1.1.

As used herein, “overall response rate,” “objective response rate,” and “ORR” refer interchangeably to the sum of CR rate and PR rate. For example, objective response may be defined as a CR or PR per Response Evaluation Criteria in Solid Tumors (RECIST) v.1.1, as determined by investigator assessment and confirmed by repeat assessment≥4 weeks after initial documentation. In another example, ORR may be defined as the proportion of patients with CR or PR on two consecutive occasions≥4 weeks apart, as determined by the investigator according to RECIST v1.1.

As used herein, “pathologic response rate” and “pRR” refer interchangeably to the proportion of patients with pathologic complete response (pCR, e.g., a complete absence of viable tumor in the treated tumor bed), pathologic near complete response (pnCR, e.g., <10% of of the treated tumor bed is occupied by viable tumor cells), and pathologic partial response (pPR, e.g., <50% of the treated tumor bed is occupied by viable tumor cells), e.g., at the time of surgery.

As used herein, “progression-free survival” and “PFS” refer to the length of time during and after treatment during which the cancer does not get worse. PFS may include the amount of time patients have experienced a CR or a PR, as well as the amount of time patients have experienced stable disease. For example, PFS may be defined as the time from the first study treatment to the first occurrence of progression or death from any cause, whichever occurs first, per RECIST v.1.1 as determined by the investigator. In another example, PFS may be defined as the time from study enrollment to the first occurrence of progression or death from any cause, whichever occurs first, per RECIST v.1.1 as determined by the investigator.

As used herein, “overall survival” and “OS” refer to the length of time from either the date of diagnosis or the start of treatment for a disease (e.g., cancer) that the patient is still alive. For example, OS may be defined as the time from first study treatment to death from any cause.

As used herein, the term “duration of response” and “DOR” refer to a length of time from documentation of a tumor response until disease progression or death from any cause, whichever occurs first. For example, DOR may be defined as the time from the first occurrence of a documented objective response to the time of the first documented disease progression or death from any cause, whichever occurs first, per RECIST v1.1 as determined by the investigator.

As used herein, the term “chemotherapeutic agent” refers to a compound useful in the treatment of cancer. Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy) quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl) methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino) phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis (2-methoxyethoxy) quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

The term “patient” or “subject” refers to a human patient or subject. For example, the patient or subject may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

A “naked antibody” refers to an antibody that is not conjugated to a heterologous moiety (e.g., a cytotoxic moiety) or radiolabel. The naked antibody may be present in a pharmaceutical composition.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).
   Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “monospecific” antibody as used herein denotes an antibody that has one or more binding sites each of which bind to the same epitope of the same antigen. The term “bispecific” antibody as used herein means that the antibody is able to specifically bind to at least two distinct antigens, for example two binding sites each formed by a pair of an antibody heavy chain variable domain (VH) and an antibody light chain variable domain (VL) binding to different antigens or to different epitopes on the same antigen. Such a bispecific antibody is an 1+1 format. Other bispecific antibody formats are 2+1 formats (comprising two binding sites for a first antigen or epitope and one binding site for a second antigen or epitope) or 2+2 formats (comprising two binding sites for a first antigen or epitope and two binding sites for a second antigen or epitope). Typically, a bispecific antibody comprises two antigen binding sites, each of which is specific for a different antigen.

As used herein, a “PD-L1-positive tumor cell fraction” is the percentage of viable tumor cells showing partial or complete membrane staining (exclusive of cytoplasmic staining) at any intensity relative to all viable tumor cells present in a sample, following staining of the sample in the context of an immunohistochemical (IHC) assay, e.g., an IHC assay staining for PD-L1 using the antibody SP142, SP263, 22C3, or 28-8. Accordingly, a PD-L1-positive tumor cell fraction may be calculated using the PD-L1 IHC SP142 (Ventana) assay, for example, by the formula PD-L1-positive tumor cell fraction=(number of PD-L1-positive tumor cells)/(total number of PD-L1-positive and PD-L1 negative tumor cells), wherein PD-L1 cytoplasmic staining of tumor cells and all non-tumor cells (e.g., tumor-infiltrating immune cells, normal cells, necrotic cells, and debris) are excluded from evaluation and scoring. It will be appreciated that any given diagnostic PD-L1 antibody may correspond with a particular IHC assay protocol and/or scoring terminology that can be used to derive a PD-L1-positive tumor cell fraction. For example, a PD-L1-positive tumor cell fraction can be derived from a tumor cell sample stained with SP263, 22C3, SP142, or 28-8 using OPTIVIEW® detection on Benchmark ULTRA, EnVision Flex on AutostainerLink 48, OPTIVIEW® detection and amplification on Benchmark ULTRA, or EnVision Flex on AutostainerLink 48, respectively.

As used herein, the “Ventana SP142 IHC assay” is conducted according to the Ventana PD-L1 (SP142) Assay package insert (Tucson, AZ: Ventana Medical Systems, Inc.), which is incorporated herein by reference in its entirety.

As used herein, the “Ventana SP263 IHC assay” is conducted according to the Ventana PD-L1 (SP263) Assay package insert (Tucson, AZ: Ventana Medical Systems, Inc.), which is incorporated herein by reference in its entirety.

As used herein, the “pharmDx 22C3 IHC assay” is conducted according to the PD-L1 IHC 22C3 pharmDx package insert (Carpinteria, CA: Dako, Agilent Pathology Solutions), which is incorporated herein by reference in its entirety.

As used herein, the “pharmDx 28-8 IHC assay” is conducted according to the PD-L1 IHC 28-8 pharmDx package insert (Carpinteria, CA: Dako, Agilent Pathology Solutions), which is incorporated herein by reference in its entirety.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a bispecific antibody targeting programmed cell death protein 1 (PD-1) and lymphocyte activation gene-3 (LAG3) and an anti-TIGIT antagonist antibody (e.g., tiragolumab) or a VEGF antagonist, e.g., an anti-VEGF antibody (e.g., bevacizumab). As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3-week cycle.

As used herein, the term “adverse event” or “AE” refers to any unfavorable and unintended sign (including an abnormal laboratory finding), symptom, or disease temporally associated with the use of a medical treatment or procedure that may or may not be considered related to the medical treatment or procedure. Adverse events may be classified by “grade,” as defined by the National Cancer Institute Common Terminology Criteria for Adverse Events v4.0 or v5.0 (NIH CTCAE). In some aspects, the AE is a low-grade AE, e.g., a Grade 1 or Grade 2 AE. Grade 1 includes AEs that are asymptomatic or have mild symptoms. Grade 2 includes AEs that are moderate and limit age-appropriate instrumental activities of daily living (e.g., preparing meals, shopping for groceries or clothes) and that indicate local or noninvasive intervention. In other instances, the AE is a high-grade AE, e.g., a Grade 3, Grade 4, or Grade 5 AE. In some instances, the AE is a Grade 3 or a Grade 4 AE. Grade 3 includes AEs that are severe or medically significant, but not immediately life-threatening, and that indicate hospitalization or prolongation of hospitalization. Grade 4 includes AEs that have life-threatening consequences and indicate urgent intervention. Grade 5 includes AEs that result in or relate to death.

As used herein, the term “treatment-related AE” refers to an AE that is judged by an investigator to have occurred as a result of a treatment, e.g., a PD-1 axis binding antagonist therapy (e.g., atezolizumab therapy) and/or an anti-TIGIT antagonist antibody therapy (e.g., tiragolumab therapy).

The term “valent” as used within the current application denotes the presence of a specified number of binding domains in an antigen binding molecule. As such, the terms “bivalent,” “tetravalent,” and “hexavalent” denote the presence of two binding domains, four binding domains, and six binding domains, respectively, in an antigen binding molecule. The bispecific antibodies according to the invention are at least “bivalent” and may be “trivalent” or “multivalent” (e.g., “tetravalent” or “hexavalent”). In a particular aspect, the antibodies of the present invention have two or more binding sites and are bispecific. That is, the antibodies may be bispecific even in cases where there are more than two binding sites (i.e., that the antibody is trivalent or multivalent).

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F (ab′) 2; diabodies, triabodies, tetrabodies, cross-Fab fragments; linear antibodies; single-chain antibody molecules (e.g., scFv); multispecific antibodies formed from antibody fragments and single domain antibodies. For a review of certain antibody fragments, see Hudson et al., Nat Med 9, 129-134 (2003). For a review of scFv fragments, see e.g., Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F (ab′) 2 fragments comprising salvage receptor binding epitope residues and having increased in vivo half-life, see U.S. Pat. No. 5,869,046. Diabodies are antibody fragments with two antigen-binding domains that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g., U.S. Pat. No. 6,248,516 B1). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full-length antibodies. Antibody fragments can be made by various techniques, including but not limited to proteolytic digestion of an intact antibody as well as production by recombinant host cells (e.g., E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments wherein the cysteine residue(s) of the constant domains bear a free thiol group. Pepsin treatment yields an F (ab′) 2 fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e., the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g., position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding domain which binds specifically to an antigen and wherein said linker is a polypeptide of at least 30 amino acids. In addition, these x-scFab molecules might be further stabilized by generation of interchain disulfide bonds via insertion of cysteine residues (e.g., position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g., described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full-length antibodies.

A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the B-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can be engineered to include up to 25 amino acids without affecting the overall fold of the microprotein. For further details of engineered knottin domains, see WO2008098796.

The term “a bispecific antibody comprising a first antigen-binding domain that specifically binds to PD-1 and a second antigen-binding domain that specifically binds to LAG3,” “a bispecific antibody that specifically binds PD-1 and LAG3,” “bispecific antigen binding molecule specific for PD-1 and LAG3” or an “anti-PD-1/anti-LAG3 antibody” are used interchangeably herein and refer to a bispecific antibody that is capable of binding PD-1 and LAG3 with sufficient affinity such that the antibody is useful as a diagnostic and/or therapeutic agent in targeting PD-1 and LAG3.

The term “PD-1,” also known as Programmed cell death protein 1, is a type I membrane protein of 288 amino acids that was first described in 1992 (Ishida et al., EMBO J., 11 (1992), 3887-3895). PD-1 is a member of the extended CD28/CTLA-4 family of T cell regulators and has two ligands, PD-L1 (B7-H1, CD274) and PD-L2 (B7-DC, CD273). The protein's structure includes an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates TCR signals. This is consistent with binding of SHP-1 and SHP-2 phosphatases to the cytoplasmic tail of PD-1 upon ligand binding. While PD-1 is not expressed on naïve T cells, it is upregulated following T cell receptor (TCR)-mediated activation and is observed on both activated and exhausted T cells (Agata et al., Int. Immunology 8 (1996), 765-772). These exhausted T-cells have a dysfunctional phenotype and are unable to respond appropriately. Although PD-1 has a relatively wide expression pattern its most important role is likely as a coinhibitory receptor on T cells (Chinai et al, Trends in Pharmacological Sciences 36 (2015), 587-595). Current therapeutic approaches thus focus on blocking the interaction of PD-1 with its ligands to enhance T cell response. The terms “Programmed Death 1,” “Programmed Cell Death 1,” “Protein PD-1,” “PD-1,” PD1,” “PDCD1,” “hPD-1” and “hPD-1” can be used interchangeably, and include variants, isoforms, species homologs of human PD-1, and analogs having at least one common epitope with PD-1. The amino acid sequence of human PD-1 is shown in UniProt (www.uniprot.org) accession no. Q15116 (SEQ ID NO: 55).

The terms “LAG3” or “Lag-3” or “Lymphocyte activation gene-3” or “CD223” as used herein refer to any native LAG3 from any vertebrate source, including mammals such as primates (e.g., humans) and rodents (e.g., mice and rats), unless otherwise indicated. The term encompasses “full-length,” unprocessed LAG3 as well as any form of LAG3 resulting from processing in the cell. The term also encompasses naturally occurring variants of LAG3, e.g., splice variants or allelic variants. In one preferred embodiment the term “LAG3” refers to human LAG3. The amino acid sequence of an exemplary processed (without signal sequences) LAG3 is shown in SEQ ID NO: 56. The amino acid sequence of an exemplary Extracellular Domain (ECD) LAG3 is shown in SEQ ID NO: 57.

The terms “anti-LAG3 antibody” and “an antibody that binds to LAG3” refer to an antibody that is capable of binding LAG3 with sufficient affinity such that the antibody is useful as a diagnostic and/or therapeutic agent in targeting LAG3. In one aspect, the extent of binding of an anti-LAG3 antibody to an unrelated, non-LAG3 protein is less than about 10% of the binding of the antibody to LAG3 as measured, e.g., by a radioimmunoassay (RIA). In certain embodiments, an antibody that binds to LAG3 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, an anti-LAG3 antibody binds to an epitope of LAG3 that is conserved among LAG3 from different species. In one preferred embodiment, an “anti-LAG3 antibody,” “an antibody that specifically binds to human LAG3,” and “an antibody that binds to human LAG3” refers to an antibody specifically binding to the human LAG3 antigen or its Extracellular Domain (ECD) with a binding affinity of a K_{D}-value of 1.0×10⁻⁸ mol/l or lower, in one embodiment of a K_{D}-value of 1.0×10⁻⁹ mol/l or lower, in one embodiment of a K_{D}-value of 1.0×10⁻⁹ mol/l to 1.0×10⁻¹³ mol/l. In this context the binding affinity is determined with a standard binding assay, such as surface plasmon resonance technique (BIAcore®, GE-Healthcare Uppsala, Sweden) e.g., using the LAG3 extracellular domain. The term “anti-LAG3 antibody” also encompasses bispecific antibodies that are capable of binding LAG3 and a second antigen.

The “knob-into-hole” technology is described e.g., in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). The protuberance and cavity can be made by altering the nucleic acid encoding the polypeptides, e.g., by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO: 46) GGGGSGGGGS (SEQ ID NO: 47), SGGGGSGGGG (SEQ ID NO: 48) and GGGGSGGGGSGGGG (SEQ ID NO: 49), but also include the sequences GSPGSSSSGS (SEQ ID NO: 50), (G4S) 3 (SEQ ID NO: 51), (G4S) 4 (SEQ ID NO: 52), GSGSGSGS (SEQ ID NO: 53), GSGSGNGS (SEQ ID NO: 54), GGSGSGSG (SEQ ID NO: 55), GGSGSG (SEQ ID NO: 56), GGSG (SEQ ID NO: 57), GGSGNGSG (SEQ ID NO: 58), GGNGSGSG (SEQ ID NO: 59) and GGNGSG (SEQ ID NO: 60). Peptide linkers of particular interest are (G4S) (SEQ ID NO: 46), (G₄S)₂ or GGGGGGGGS (SEQ ID NO: 47), (G₄S)₃ (SEQ ID NO: 51) and (G₄S)₄ (SEQ ID NO: 53), more particularly (G₄S)₂ (SEQ ID NO: 47) or GGGGSGGGGS (SEQ ID NO: 47).

By “fused to” or “connected to” is meant that the components (e.g., an antigen-binding domain and a Fc domain) are linked by peptide bonds, either directly or via one or more peptide linkers.

A “VEGF antagonist” or “VEGF-specific antagonist” refers to a molecule capable of binding to VEGF, reducing VEGF expression levels, or neutralizing, blocking, inhibiting, abrogating, reducing, or interfering with VEGF biological activities, including, but not limited to, VEGF binding to one or more VEGF receptors, VEGF signaling, and VEGF mediated angiogenesis and endothelial cell survival or proliferation. For example, a molecule capable of neutralizing, blocking, inhibiting, abrogating, reducing, or interfering with VEGF biological activities can exert its effects by binding to one or more VEGF receptor (VEGFR) (e.g., VEGFR1, VEGFR2, VEGFR3, membrane-bound VEGF receptor (mbVEGFR), or soluble VEGF receptor (sVEGFR)). Such antagonists are also referred to herein as “VEGFR inhibitors.” Included as VEGF-specific antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF-specific antagonists also include antagonist variants of VEGF polypeptides, antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers. VEGF antagonists also include polypeptides that bind to VEGFR, anti-VEGFR antibodies (e.g., bevacizumab), and antigen-binding fragments thereof, and derivatives which bind to VEGFR thereby blocking, inhibiting, abrogating, reducing, or interfering with VEGF biological activities (e.g., VEGF signaling), or fusions proteins. VEGF-specific antagonists also include nonpeptide small molecules that bind to VEGF or VEGFR and are capable of blocking, inhibiting, abrogating, reducing, or interfering with VEGF biological activities. Thus, the term “VEGF biological activities” specifically includes VEGF-mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF. In some embodiments, the VEGF inhibited by the VEGF-specific antagonist is VEGF (8-109), VEGF (1-109), or VEGF₁₆₅.

As used herein VEGF antagonists can include, but are not limited to, anti-VEGFR2 antibodies and related molecules (e.g., ramucirumab, tanibirumab, aflibercept), anti-VEGFR1 antibodies and related molecules (e.g., icrucumab, aflibercept (VEGF Trap-Eye; EYLEA®), and ziv-aflibercept (VEGF Trap; ZALTRAP®)), bispecific VEGF antibodies (e.g., MP-0250, vanucizumab (VEGF-ANG2), and bispecific antibodies disclosed in US 2001/0236388), bispecific antibodies including combinations of two of anti-VEGF, anti-VEGFR1, and anti-VEGFR2 arms, anti-VEGFA antibodies (e.g., bevacizumab, sevacizumab), anti-VEGFB antibodies, anti-VEGFC antibodies (e.g., VGX-100), anti-VEGFD antibodies, and nonpeptide small molecule VEGF antagonists (e.g., pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, and tivozanib). In some examples, the VEGF antagonist may be a tyrosine kinase inhibitor, including a receptor tyrosine kinase inhibitors (e.g., a multi-targeted receptor tyrosine kinase inhibitor such as sunitinib or axitinib).

An “anti-VEGF antibody” is an antibody that binds to VEGF with sufficient affinity and specificity. In certain embodiments, the antibody will have a sufficiently high binding affinity for VEGF, for example, the antibody may bind hVEGF with a K_{D} value of between 100 nM and 1 pM. Antibody affinities may be determined, e.g., by a surface plasmon resonance-based assay (such as the BIAcore® assay as described in PCT Application Publication No. WO2005/012359); enzyme-linked immunoabsorbent assay (ELISA); and competition assays (e.g., radioimmunoassays (RIAs)).

In certain embodiments, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PIGF, PDGF, or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (Cancer Res. 57:4593-4599, 1997), including, but not limited to, the antibody known as bevacizumab (BV; AVASTIN®).

The anti-VEGF antibody “bevacizumab (BV),” also known as “rhuMAb VEGF” or “AVASTIN®,” is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879, issued Feb. 26, 2005, the entire disclosure of which is expressly incorporated herein by reference.

### II. Therapeutic Methods and Compositions for Cancer

### A. Methods Comprising a Bispecific Antibody Targeting PD-1 and LAG3

In one aspect, the disclosure provides a method for treating a subject having a cancer, the method comprising administering to the subject one or more dosing cycles of a bispecific antibody targeting programmed cell death protein 1 (PD-1) and lymphocyte activation gene 3 (LAG3) comprising a first antigen-binding domain that specifically binds to PD-1 and a second antigen-binding domain that specifically binds to LAG3, wherein the method comprises administering to the subject the bispecific antibody at a fixed dose of about 600 mg (e.g., a fixed dose of 600 mg) every three weeks.

Exemplary bispecific antibodies targeting PD-1 and LAG3 are provided in Section VIII, below. A particular example for a bispecific antibody targeting PD-1 and LAG3 is PD1-LAG3 as defined herein.

Clinical proof-of-concept for the dual inhibition of PD-1 and LAG3 was recently provided by the combination of relatlimab and nivolumab in patients with previously untreated metastatic or unresectable melanoma (Tawbi et al., N Engl J Med, 386 (1): 24-34, 2022). By targeting both PD-1 and LAG-3 on dysfunctional tumor-specific T lymphocytes, bispecific antibodies targeting PD-1 and LAG3 aim to restore an effective anti-tumor immune-response and to provide even more survival benefit to cancer patients than the currently available checkpoint inhibitors. By preferentially targeting PD-1/LAG-3 co-expressing dysfunctional T cells and potentially reduced targeting of LAG-3 expressing Tregs in the tumor microenvironment, bispecific antibodies targeting PD-1 and LAG3 might avoid reinvigorating Treg mediated immunosuppressive effects while restoring the anti-tumor immune response.

In some aspects, the length of each of the one or more dosing cycles is 21 days. In some aspects, the method comprises administering (e.g., intravenously administering) to the subject the bispecific antibody on Day 1 of each of the one or more dosing cycles.

The cancer may be a solid tumor (e.g., a solid tumor having a tumor microenvironment comprising LAG3-expressing CD8+ T cells). For example, in some aspects, the cancer may be a skin cancer (e.g., a melanoma), a liver cancer (e.g., a hepatocellular carcinoma (HCC)), a lung cancer (e.g., a non-small cell lung cancer (NSCLC)), a kidney cancer, a renal cancer (e.g., a renal cell carcinoma (RCC)), a bladder cancer (e.g., a metastatic urothelial carcinoma (mUC)), a breast cancer (e.g., a triple-negative breast cancer (TNBC)), an esophageal cancer (e.g., esophageal squamous cell carcinoma (ESCC)), a pancreatic cancer, a cervical cancer, a head and neck cancer, a gastric cancer, a colorectal cancer, or an ovarian cancer. In some aspects, the cancer is a skin cancer (e.g., a melanoma), a liver cancer (e.g., a HCC), a lung cancer (e.g., a NSCLC), a kidney cancer, a renal cancer (e.g., a RCC), a bladder cancer (e.g., a mUC), a breast cancer (e.g., a TNBC), or an esophageal cancer (e.g., ESCC). The cancer may be locally advanced or metastatic.

In aspects in which the skin cancer is a melanoma, the melanoma may be, e.g., a previously untreated unresectable or metastatic melanoma (e.g., a histologically confirmed unresectable or metastatic melanoma per the American Joint Committee on Cancer (AJCC) staging system (unresectable Stage III or Stage IV). In some aspects, the melanoma is (a) a Stage III melanoma with measurable lymph node metastases; (b) an unresectable Stage III melanoma; or (c) a Stage IV melanoma. In some aspects, the melanoma is not a mucosal melanoma or a uveal melanoma.

In some aspects, the subject has not previously received a systemic anti-cancer therapy.

In some aspects, the subject has not previously been treated with an anti-cancer therapy comprising an immunomodulatory agent, e.g., has not been treated with an anti-cancer agent comprising a checkpoint inhibitor (CPI), e.g., has not been treated with an anti-programmed death-ligand 1 (PD-L1)/PD-1 agent or has not been treated with an anti-cytotoxic T lymphocyte-associated antigen (CTLA-4)) agent. In other aspects, the subject previously has been treated with an immunomodulatory agent (e.g., a CPI) as an adjuvant or neoadjuvant therapy.

In some aspects, the subject has not previously been treated for metastatic or unresectable disease.

In some aspects, the subject has not previously been treated with an anti-LAG3 therapy.

In some aspects, the bispecific antibody achieves at least 90% LAG3 receptor occupancy (RO) in the tumor, e.g., achieves at least 90%, 91%, 92%, 93%, 94%, 95%, 96%, 97%, 98%, 99%, or 100% RO in the tumor.

In some aspects, the subject is a human.

### B. Methods Comprising a Bispecific Antibody Targeting PD-1 and LAG3 and Bevacizumab

In some aspects, the method further comprises administering (e.g., intravenously administering) to the subject a VEGF antagonist, e.g., an anti-VEGF antibody (e.g., bevacizumab). Exemplary VEGF antagonists are provided in Section IX, below.

In some aspects, the VEGF antagonist is administered before the bispecific antibody targeting PD-1 and LAG3. In other aspects, the VEGF antagonist is administered after the bispecific antibody targeting PD-1 and LAG3. In still further aspects, the VEGF antagonist and the bispecific antibody targeting PD-1 and LAG3 are administered simultaneously.

In some aspects, the method comprises administering to the subject a VEGF antagonist (e.g., bevacizumab) at a dose of about 15 mg/kg (e.g., a dose of 15 mg/kg) every three weeks.

In some aspects, the length of each of the one or more dosing cycles is 21 days and the method comprises administering to the subject the VEGF antagonist (e.g., bevacizumab) on Day 1 of each of the one or more dosing cycles.

Accordingly, in one aspect, the disclosure provides a method for treating a subject having a cancer, the method comprising administering to the subject one or more dosing cycles of (1) a bispecific antibody targeting PD-1 and LAG3 comprising a first antigen-binding domain that specifically binds to PD-1 and a second antigen-binding domain that specifically binds to LAG3 (e.g., a bispecific antibody targeting PD-1 and LAG3 provided in Section VIII, below) and (2) a VEGF antagonist (e.g., bevacizumab), wherein the method comprises administering to the subject the bispecific antibody at a fixed dose of 600 mg every three weeks and the VEGF antagonist at a dose of 15 mg/kg every three weeks.

In other aspects, the method further comprises administering to the subject a VEGF antagonist (e.g., bevacizumab) at a dose of about 10 mg/kg (e.g., a dose of 10 mg/kg) every two weeks. In some aspects, the length of each of the one or more dosing cycles is 28 days and the method comprises administering to the subject the VEGF antagonist (e.g., bevacizumab) on Days 1 and 15 of each of the one or more dosing cycles.

In another aspect, the disclosure provides a bispecific antibody targeting PD-1 and LAG3 for use in a method of treating a subject having a cancer (e.g., a bispecific antibody targeting PD-1 and LAG3 for use in any of the above methods), wherein the bispecific antibody comprises a first antigen-binding domain that specifically binds to PD-1 and a second antigen-binding domain that specifically binds to LAG3, and wherein the method comprises administering to the subject the bispecific antibody at a fixed dose of 600 mg every three weeks. A particular example for a bispecific antibody targeting PD-1 and LAG3 is PD1-LAG3 as defined herein.

In another aspect, the disclosure provides use of a bispecific antibody targeting PD-1 and LAG3 in the manufacture of a medicament for treating a subject having a cancer, wherein the bispecific antibody comprises a first antigen-binding domain that specifically binds to PD-1 and a second antigen-binding domain that specifically binds to LAG3, and wherein the bispecific antibody is to be administered to the subject at a fixed dose of 600 mg every three weeks.

### C. Methods Comprising a Bispecific Antibody Targeting PD-1 and LAG3 and an Anti-TIGIT Antagonist Antibody

In some aspects, the method further comprises administering (e.g., intravenously administering) to the subject an anti-TIGIT antagonist antibody (e.g., tiragolumab).

Exemplary anti-TIGIT antagonist antibodies are provided in Section VII, below.

In some aspects, the anti-TIGIT antagonist antibody is administered before the bispecific antibody targeting PD-1 and LAG3. In other aspects, the anti-TIGIT antagonist antibody is administered after the bispecific antibody targeting PD-1 and LAG3. In still further aspects, the anti-TIGIT antagonist antibody and the bispecific antibody targeting PD-1 and LAG3 are administered simultaneously.

In some aspects, the method comprises administering to the subject an anti-TIGIT antagonist antibody (e.g., tiragolumab) at a fixed dose of about 600 mg (e.g., a fixed dose of 600 mg) every three weeks.

In some aspects, the length of each of the one or more dosing cycles is 21 days and the method comprises administering to the subject the anti-TIGIT antagonist antibody on about Day 1 (e.g., on Day 1) of each of the one or more dosing cycles.

Accordingly, in one aspect, the disclosure provides a method for treating a subject having a cancer, the method comprising administering to the subject one or more dosing cycles of (1) a bispecific antibody targeting PD-1 and LAG3 comprising a first antigen-binding domain that specifically binds to PD-1 and a second antigen-binding domain that specifically binds to LAG3 (e.g., a bispecific antibody targeting PD-1 and LAG3 provided in Section VIII, below; in particular PD1-LAG3 as defined herein) and (2) an anti-TIGIT antagonist antibody (e.g., tiragolumab), wherein the method comprises administering to the subject the bispecific antibody at a fixed dose of 600 mg every three weeks and the anti-TIGIT antagonist antibody at a fixed dose of 600 mg every three weeks.

### III. Therapeutic Methods and Compositions for Liver Cancer

### A. Methods Comprising a Bispecific Antibody Targeting PD-1 and LAG3

In one aspect, the disclosure provides a method for treating a subject having a liver cancer, the method comprising administering to the subject one or more dosing cycles of a bispecific antibody targeting programmed cell death protein 1 (PD-1) and lymphocyte activation gene 3 (LAG3) comprising a first antigen-binding domain that specifically binds to PD-1 and a second antigen-binding domain that specifically binds to LAG3, wherein the method comprises administering to the subject the bispecific antibody at a fixed dose of about 600 mg (e.g., a fixed dose of 600 mg) every three weeks.

Exemplary bispecific antibodies targeting PD-1 and LAG3 are provided in Section VIII, below. A particular example for a bispecific antibody targeting PD-1 and LAG3 is PD1-LAG3 as defined herein.

In another aspect, the disclosure provides a method for treating a subject having a liver cancer, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3, wherein the method comprises administering to the subject the bispecific antibody at a fixed dose of 1200 mg every three weeks.

In some aspects, the length of each of the one or more dosing cycles is 21 days. In some aspects, the method comprises administering (e.g., intravenously administering) to the subject the bispecific antibody on Day 1 of each of the one or more dosing cycles.

In still another aspect, the disclosure provides a method for treating a subject having a liver cancer, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3, wherein the method comprises administering to the subject the bispecific antibody at a fixed dose of 2100 mg every two weeks.

In some aspects, the length of each of the one or more dosing cycles is 28 days. In some aspects, the method comprises administering (e.g., intravenously administering) to the subject the bispecific antibody on Days 1 and 15 of each of the one or more dosing cycles.

In some aspects, the liver cancer is a hepatocellular carcinoma (HCC). In some aspects, the liver cancer (e.g., HCC) has a tumor microenvironment comprising LAG3-expressing CD8+ T cells. The HCC may be, e.g., locally advanced, metastatic, and/or unresectable.

In some aspects, the subject has not previously received a systemic anti-cancer therapy.

In some aspects, the subject has not previously been treated with an anti-cancer therapy comprising an immunomodulatory agent, e.g., has not been treated with an anti-cancer agent comprising a checkpoint inhibitor (CPI), e.g., has not been treated with an anti-programmed death-ligand 1 (PD-L1)/PD-1 agent or has not been treated with an anti-cytotoxic T lymphocyte-associated antigen (CTLA-4)) agent. In other aspects, the subject previously has been treated with an immunomodulatory agent (e.g., a CPI) as an adjuvant or neoadjuvant therapy.

In some aspects, the subject has not previously been treated for metastatic or unresectable disease.

In some aspects, the subject has not previously been treated with an anti-LAG3 therapy.

In some aspects, the bispecific antibody achieves at least 90% LAG3 receptor occupancy (RO) in the tumor, e.g., achieves at least 90%, 91%, 92%, 93%, 94%, 95%, 96%, 97%, 98%, 99%, or 100% RO in the tumor.

In some aspects, the subject is a human.

### B. Methods Comprising a Bispecific Antibody Targeting PD-1 and LAG3 and Bevacizumab

In some aspects, the method further comprises administering (e.g., intravenously administering) to the subject a VEGF antagonist, e.g., an anti-VEGF antibody (e.g., bevacizumab). Exemplary VEGF antagonists are provided in Section IX, below.

In some aspects, the anti-TIGIT antagonist antibody is administered before the bispecific antibody targeting PD-1 and LAG3. In other aspects, the anti-TIGIT antagonist antibody is administered after the bispecific antibody targeting PD-1 and LAG3. In still further aspects, the anti-TIGIT antagonist antibody and the bispecific antibody targeting PD-1 and LAG3 are administered simultaneously.

In some aspects, the method comprises administering to the subject a VEGF antagonist (e.g., bevacizumab) at a dose of about 15 mg/kg (e.g., a dose of 15 mg/kg) every three weeks.

In some aspects, the length of each of the one or more dosing cycles is 21 days and the method comprises administering to the subject the VEGF antagonist (e.g., bevacizumab) on Day 1 of each of the one or more dosing cycles.

Accordingly, in one aspect, the disclosure provides a method for treating a subject having a liver cancer, the method comprising administering to the subject one or more dosing cycles of (1) a bispecific antibody targeting PD-1 and LAG3 comprising a first antigen-binding domain that specifically binds to PD-1 and a second antigen-binding domain that specifically binds to LAG3 (e.g., a bispecific antibody targeting PD-1 and LAG3 provided in Section VIII, below and in particular PD1-LAG3) and (2) a VEGF antagonist, wherein the method comprises administering to the subject the bispecific antibody at a fixed dose of 600 mg every three weeks and the VEGF antagonist at a dose of 15 mg/kg every three weeks.

In another aspect, the disclosure provides a method for treating a subject having a liver cancer, the method comprising administering to the subject one or more dosing cycles of (1) a bispecific antibody targeting PD-1 and LAG3 comprising a first antigen-binding domain that specifically binds to PD-1 and a second antigen-binding domain that specifically binds to LAG3 (e.g., a bispecific antibody targeting PD-1 and LAG3 provided in Section VIII, below) and (2) a VEGF antagonist, wherein the method comprises administering to the subject the bispecific antibody at a fixed dose of 1200 mg every three weeks and the VEGF antagonist at a dose of 15 mg/kg every three weeks.

In other aspects, the method further comprises administering to the subject a VEGF antagonist (e.g., bevacizumab) at a dose of about 10 mg/kg (e.g., a dose of 10 mg/kg) every two weeks. In some aspects, the length of each of the one or more dosing cycles is 28 days and the method comprises administering to the subject the VEGF antagonist (e.g., bevacizumab) on Days 1 and 15 of each of the one or more dosing cycles.

Accordingly, in another aspect, the disclosure provides a method for treating a subject having a liver cancer, the method comprising administering to the subject one or more dosing cycles of (1) a bispecific antibody targeting PD-1 and LAG3 comprising a first antigen-binding domain that specifically binds to PD-1 and a second antigen-binding domain that specifically binds to LAG3 (e.g., a bispecific antibody targeting PD-1 and LAG3 provided in Section VIII, below) and (2) a VEGF antagonist (e.g., bevacizumab), wherein the method comprises administering to the subject the bispecific antibody at a fixed dose of 2100 mg every two weeks and the VEGF antagonist at a dose of 10 mg/kg every two weeks.

### IV. Therapeutic Methods and Compositions for Melanoma

### A. Methods Comprising an Anti-TIGIT Antagonist Antibody and a Bispecific Antibody Targeting PD-1 and LAG3

In one aspect, the disclosure provides a method for treating a subject having a melanoma, the method comprising administering to the subject an anti-TIGIT antagonist antibody and a bispecific antibody targeting PD-1 and LAG3 comprising a first antigen-binding domain that specifically binds to programmed cell death protein 1 (PD-1) and a second antigen-binding domain that specifically binds to lymphocyte activation gene 3 (LAG3). In some embodiments, the anti-TIGIT antagonist antibody and the bispecific antibody are administered to the subject in a dosing regimen that comprises one or more dosing cycles.

In some aspects, the method comprises administering to the subject (a) the anti-TIGIT antagonist antibody at a fixed dose of about 600 mg (e.g., a fixed dose of 600 mg) every three weeks; and (b) the bispecific antibody at a fixed dose of about 2100 mg (e.g., a fixed dose of 2100 mg) every three weeks.

In particular aspects, the method comprises administering to the subject (a) the anti-TIGIT antagonist antibody at a fixed dose of about 600 mg (e.g., a fixed dose of 600 mg) every three weeks; and (b) the bispecific antibody at a fixed dose of about 600 mg (e.g., a fixed dose of 600 mg) every three weeks.

In another aspect, the disclosure provides a method for treating a subject having a melanoma, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3 (e.g., a bispecific antibody targeting PD-1 and LAG3 provided in Section VIII, below, and in particular PD1-LAG3), wherein the method comprises administering to the subject the bispecific antibody at a fixed dose of 600 mg every three weeks, and wherein the melanoma is: (a) an unresectable Stage III melanoma; or (b) a Stage IV melanoma (e.g., a histologically confirmed unresectable or metastatic melanoma per the American Joint Committee on Cancer (AJCC) staging system (unresectable Stage III or Stage IV).

In some aspects, the length of each of the one or more dosing cycles is 21 days. In some aspects, the method comprises administering to the subject the anti-TIGIT antagonist antibody and the bispecific antibody on about Day 1 (e.g., on Day 1) of each of the one or more dosing cycles.

In some aspects, the method comprises administering to the subject the bispecific antibody before the anti-TIGIT antagonist antibody. In other aspects, the method comprises administering to the subject the anti-TIGIT antagonist antibody before the bispecific antibody.

In some aspects, the method comprises administering to the subject the bispecific antibody and the anti-TIGIT antagonist antibody intravenously.

i. Neoadjuvant Therapy

In some aspects, the one or more dosing cycles are administered as a neoadjuvant therapy.

In some aspects, the anti-TIGIT antagonist antibody and the bispecific antibody targeting PD-1 and LAG3 are administered as a neoadjuvant therapy.

In some aspects, the melanoma is a Stage III melanoma with measurable lymph node metastases.

In some aspects, the subject has not had in-transit metastases within six months prior to the initiation of treatment.

In some aspects, the subject has not previously been treated with a cancer immunotherapy.

In some aspects, the melanoma is not a mucosal melanoma or a uveal melanoma.

In some aspects, a first dosing cycle is initiated prior to a surgery.

In some aspects, at least one dosing cycle or (e.g., one, two, three, four, or more than four dosing cycles) or at least two dosing cycles (e.g., two, three, four, or more than four dosing cycles) are completed prior to the surgery. In some aspects, two dosing cycles are completed prior to the surgery.

In some aspects, the surgery is performed within about one week after the last dosing cycle.

In some aspects, the surgery is a completion lymph node dissection (CLND).

In some aspects, the treating results in an increase in pathologic response rate (pRR) as compared to a reference pRR. In some aspects, the reference pRR is a pRR of a population of subjects who have received a control therapy. In some aspects, the control therapy is a therapy comprising an anti-TIGIT antagonist antibody and not comprising a bispecific antibody targeting PD-1 and LAG3; a therapy comprising a bispecific antibody targeting PD-1 and LAG3 and not comprising an anti-TIGIT antagonist antibody; or a therapy comprising ipilimumab and nivolumab.

In some aspects, the treating results in an increase in event-free survival (EFS) as compared to a reference EFS; an increase in recurrence-free survival (RFS) as compared to a reference RFS; an increase in overall survival (OS) as compared to a reference OS; and/or an increase in overall response rate (ORR) as compared to a reference ORR. In some aspects, the reference EFS, RFS, OS, or ORR is one of a population of subjects who have received a control therapy. In some aspects, the control therapy is a therapy comprising an anti-TIGIT antagonist antibody and not comprising a bispecific antibody targeting PD-1 and LAG3; a therapy comprising a bispecific antibody targeting PD-1 and LAG3 and not comprising an anti-TIGIT antagonist antibody; or a therapy comprising ipilimumab and nivolumab.

ii. Treatment of Stage IV Melanoma

In some aspects, the melanoma is a Stage IV melanoma.

In some aspects, (a) the subject has received no more than two prior lines of systemic treatment; or (b) the melanoma is a BRAF-mutant melanoma and the subject has received no more than three prior lines of systemic treatment.

In some aspects, the treating results in an increase in overall response rate (ORR) as compared to a reference ORR. In some aspects, the reference ORR is an ORR of a population of subjects who have received (a) a treatment comprising a bispecific antibody targeting PD-1 and LAG3 and not comprising an anti-TIGIT antagonist antibody; and/or (b) a treatment comprising an anti-TIGIT antagonist antibody and not comprising a bispecific antibody targeting PD-1 and LAG3.

In some aspects, the treating results in an increase in progression-free survival (PFS) as compared to a reference PFS; an increase in duration of response (DOR) as compared to a reference DOR; an increase in OS as compared to a reference OS; an increase in disease control rate (DCR, e.g., stable disease for 12 or more weeks, a complete response (CR), or a partial response (PR)) as compared to a reference DCR. In some aspects, the reference PFS, OS, DOR, or DCR is one of a population of subjects who have received a control therapy. In some aspects, the control therapy is a therapy comprising an anti-TIGIT antagonist antibody and not comprising a bispecific antibody targeting PD-1 and LAG3; a therapy comprising a bispecific antibody targeting PD-1 and LAG3 and not comprising an anti-TIGIT antagonist antibody; or a therapy comprising ipilimumab and nivolumab.

In some aspects, the subject is a human.

### B. Methods Comprising a Bispecific Antibody Targeting PD-1 and LAG3

In another aspect, the disclosure features a method for treating a subject having a melanoma, the method comprising administering to the subject a bispecific antibody targeting PD-1 and LAG3 comprising a first antigen-binding domain that specifically binds to PD-1 and a second antigen-binding domain that specifically binds to LAG3. In some embodiments, the bispecific antibody is administered to the subject in a dosing regimen that comprises one or more dosing cycles. In some embodiments, the one or more dosing cycles are administered as a neoadjuvant therapy.

In some aspects, the method comprises administering to the subject the bispecific antibody at a fixed dose about 2100 mg (e.g., a fixed dose of 2100 mg) every three weeks.

In particular aspects, the method comprises administering to the subject the bispecific antibody at a fixed dose about 600 mg (e.g., a fixed dose of 600 mg) every three weeks.

In some aspects, the length of each of the one or more dosing cycles is 21 days. In some aspects, the method comprises administering to the subject the bispecific antibody on about Day 1 (e.g., on Day 1) of each of the one or more dosing cycles.

In some aspects, the method comprises administering to the subject the bispecific antibody intravenously.

In some aspects, the melanoma is a Stage III melanoma with measurable lymph node metastases.

In some aspects, the subject has not had in-transit metastases within six months prior to the initiation of treatment.

In some aspects, the subject has not previously been treated with a cancer immunotherapy.

In some aspects, the melanoma is not a mucosal melanoma or a uveal melanoma.

In some aspects, a first dosing cycle is initiated prior to a surgery.

In some aspects, at least one dosing cycle or (e.g., one, two, three, four, or more than four dosing cycles) or at least two dosing cycles (e.g., two, three, four, or more than four dosing cycles) are completed prior to the surgery. In some aspects, two dosing cycles are completed prior to the surgery.

In some aspects, the surgery is performed within about one week after the last dosing cycle.

In some aspects, the surgery is a completion lymph node dissection (CLND).

In some aspects, the treating results in an increase in pathologic response rate (pRR) as compared to a reference pRR. In some aspects, the reference pRR is a pRR of a population of subjects who have received a control therapy. In some aspects, the control therapy is a therapy comprising ipilimumab and nivolumab.

In some aspects, the treating results in an increase in event-free survival (EFS) as compared to a reference EFS; an increase in recurrence-free survival (RFS) as compared to a reference RFS; an increase in overall survival (OS) as compared to a reference OS; and/or an increase in overall response rate (ORR) as compared to a reference ORR. In some aspects, the reference EFS, RFS, OS, or ORR is one of a population of subjects who have received a control therapy. In some aspects, the control therapy is a therapy comprising ipilimumab and nivolumab.

In some aspects, the subject is a human.

### C. Methods Comprising an Anti-TIGIT Antagonist Antibody and a PD-1 Axis Binding Antagonist

In another aspect, the disclosure features a method for treating a subject having a melanoma, the method comprising administering to the subject one or more dosing cycles of an anti-TIGIT antagonist antibody and a PD-1 axis binding antagonist, wherein the one or more dosing cycles are administered as a neoadjuvant therapy. In another aspect, the disclosure features a method for treating a subject having a melanoma, the method comprising administering to the subject an anti-TIGIT antagonist antibody and a PD-1 axis binding antagonist, wherein the anti-TIGIT antagonist antibody and the PD-1 axis binding antagonist are administered as a neoadjuvant therapy.

In some aspects, the method comprises administering to the subject (a) the anti-TIGIT antagonist antibody at a fixed dose of about 600 mg (e.g., a fixed dose of 600 mg) every three weeks; and (b) the PD-1 axis binding antagonist at a fixed dose of about 600 mg (e.g., a fixed dose of 600 mg) every three weeks.

In some aspects, the length of each of the one or more dosing cycles is 21 days.

In some aspects, the method comprises administering to the subject the anti-TIGIT antagonist antibody and the PD-1 axis binding antagonist on about Day 1 (e.g., on Day 1) each of the one or more dosing cycles.

In some aspects, the method comprises administering to the subject the PD-1 axis binding antagonist before the anti-TIGIT antagonist antibody. In other aspects, the method comprises administering to the subject the anti-TIGIT antagonist antibody before the PD-1 axis binding antagonist.

In some aspects, the method comprises administering to the subject the PD-1 axis binding antagonist and the anti-TIGIT antagonist antibody intravenously.

In some aspects, the melanoma is a Stage III melanoma with measurable lymph node metastases.

In some aspects, the subject has not had in-transit metastases within six months prior to the initiation of treatment.

In some aspects, the subject has not previously been treated with a cancer immunotherapy.

In some aspects, the melanoma is not a mucosal melanoma or a uveal melanoma.

In some aspects, a first dosing cycle is initiated prior to a surgery.

In some aspects, at least one dosing cycle or (e.g., one, two, three, four, or more than four dosing cycles) or at least two dosing cycles (e.g., two, three, four, or more than four dosing cycles) are completed prior to the surgery. In some aspects, two dosing cycles are completed prior to the surgery.

In some aspects, the surgery is performed within about one week after the last dosing cycle.

In some aspects, the surgery is a completion lymph node dissection (CLND).

In some aspects, the treating results in an increase in pathologic response rate (pRR) as compared to a reference pRR. In some aspects, the reference pRR is a pRR of a population of subjects who have received a control therapy. In some aspects, the control therapy is a therapy comprising an anti-TIGIT antagonist antibody and not comprising a PD-1 axis binding antagonist; a therapy comprising a PD-1 axis binding antagonist and not comprising an anti-TIGIT antagonist antibody; or a therapy comprising ipilimumab and nivolumab.

In some aspects, the treating results in an increase in event-free survival (EFS) as compared to a reference EFS; an increase in recurrence-free survival (RFS) as compared to a reference RFS; an increase in overall survival (OS) as compared to a reference OS; and/or an increase in overall response rate (ORR) as compared to a reference ORR. In some aspects, the reference EFS, RFS, OS, or ORR is one of a population of subjects who have received a control therapy. In some aspects, the control therapy is a therapy comprising an anti-TIGIT antagonist antibody and not comprising a PD-1 axis binding antagonist; a therapy comprising a PD-1 axis binding antagonist and not comprising an anti-TIGIT antagonist antibody; or a therapy comprising ipilimumab and nivolumab.

In some aspects, the subject is a human.

### D. Agents for Use in Methods of Treating Melanoma

i. Bispecific Antibodies Targeting PD-1 and LAG3

Further examples of bispecific antibodies targeting PD-1 and LAG3, and dosing regimens for the same, are provided in Section VIII, below. A particular example for a bispecific antibody targeting PD-1 and LAG3 is PD1-LAG3 as defined herein.

ii. Anti-TIGIT Antagonist Antibodies

Exemplary anti-TIGIT antagonist antibodies, and dosing regimens for the same, are provided in Section VII, below.

iii. PD-1 Axis Binding Antagonists

Exemplary PD-1 axis binding antagonists, and dosing regimens for the same, are provided in Section X, below.

### V. Assessment of PD-L1 Expression

The expression of PD-L1 may be assessed in a subject treated according to any of the methods and compositions for use described herein. The methods and compositions for use may include determining the expression level of PD-L1 in a biological sample (e.g., a tumor sample) obtained from the subject having a cancer (e.g., esophageal cancer (e.g., metastatic esophageal cancer)). In other examples, the expression level of PD-L1 in a biological sample (e.g., a tumor sample) obtained from the subject has been determined prior to initiation of treatment or after initiation of treatment. PD-L1 expression may be determined using any suitable approach. For example, PD-L1 expression may be determined as described in U.S. patent application Ser. Nos. 15/787,988 and 15/790,680. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the subject has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the subject has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some aspects, the esophageal cancer of a subject treated according to any of the methods provided herein has a PD-L1-positive tumor cell (TC) fraction or tumor-infiltrating immune cell (IC) fraction of <5%. In some aspects, the esophageal cancer has a PD-L1-positive TC fraction of <1%. In other aspects, the esophageal cancer of a subject treated according to any of the methods provided herein has a PD-L1-positive TC fraction or IC fraction of ≥5%. In some aspects, PD-L1 is detected using a Ventana SP142 IHC assay, a Ventana SP263 IHC assay, a pharmDx 22C3 IHC assay, or a pharmDx 28-8 IHC assay.

In some examples, tumor samples may be scored for PD-L1 positivity in tumor-infiltrating immune cells and/or in tumor cells according to the criteria for diagnostic assessment shown in Table 2 and/or Table 3, respectively.

TABLE 2
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells | |
| covering <1% of tumor area occupied by tumor | |
| cells, associated intratumoral stroma, and | |
| contiguous peri-tumoral desmoplastic stroma | |
| Presence of discernible PD-L1 staining of any | IC1 |
| intensity in tumor-infiltrating immune cells | |
| covering ≥1% to <5% of tumor area occupied | |
| by tumor cells, associated intratumoral stroma, | |
| and contiguous peri-tumoral desmoplastic stroma | |
| Presence of discernible PD-L1 staining of any | IC2 |
| intensity in tumor-infiltrating immune cells | |
| covering ≥5% to <10% of tumor area occupied | |
| by tumor cells, associated intratumoral stroma, | |
| and contiguous peri-tumoral desmoplastic stroma | |
| Presence of discernible PD-L1 staining of any | IC3 |
| intensity in tumor-infiltrating immune cells | |
| covering ≥10% of tumor area occupied | |
| by tumor cells, associated intratumoral stroma, | |
| and contiguous peri-tumoral desmoplastic stroma | |

TABLE 3
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

### VI. Assessment of TIGIT Expression

The expression level of TIGIT may be assessed in a subject having a cancer (e.g., esophageal cancer (e.g., metastatic esophageal cancer)) who has been treated according to any of the methods, uses, and compositions for use described herein. The methods, uses, and compositions for use may include determining the expression level of TIGIT in a biological sample (e.g., a tumor sample) obtained from the subject. In other examples, the expression level of TIGIT in a biological sample (e.g., a tumor sample) obtained from the subject has been determined prior to initiation of treatment or after initiation of treatment. TIGIT expression may be determined using any suitable approach. Any suitable tumor sample may be used, e.g., a formalin-fixed and paraffin-embedded (FFPE) tumor sample, an archival tumor sample, a fresh tumor sample, or a frozen tumor sample.

For example, TIGIT expression may be determined in terms of the percentage of a tumor sample comprised by tumor-infiltrating immune cells expressing a detectable expression level of TIGIT, as the percentage of tumor-infiltrating immune cells in a tumor sample expressing a detectable expression level of TIGIT, and/or as the percentage of tumor cells in a tumor sample expressing a detectable expression level of TIGIT. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-TIGIT antagonist antibody. Any suitable anti-TIGIT antagonist antibody may be used. In some examples, the anti-TIGIT antagonist antibody is 10A7 (WO 2009/126688A3; U.S. Pat. No. 9,499,596).

### VII. Anti-TIGIT Antagonist Antibodies

The invention provides anti-TIGIT antagonist antibodies useful for treating cancer in a subject (e.g., a human) having a cancer.

In some instances, the anti-TIGIT antagonist antibody is tiragolumab (CAS Registry Number: 1918185-84-8). Tiragolumab (Genentech) is also known as MTIG7192A.

In certain instances, the anti-TIGIT antagonist antibody includes at least one, two, three, four, five, or six HVRs selected from: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and/or (f) an HVR-L3 comprising the amino acid sequence of QQYYSTPFT (SEQ ID NO: 6), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 1-6.

In some instances, anti-TIGIT antagonist antibodies may include (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4); (e) an HVR-L2 comprising the amino acid sequence of WASTRES (SEQ ID NO: 5); and (f) an HVR-L3 comprising the amino acid sequence of QQYYSTPFT (SEQ ID NO: 6). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, EVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSS (SEQ ID NO: 17) or an amino acid sequence having at least 90% sequence identity (e.g., at least 91%, 92%, 93%, 94%, 95%, 96%, 97%, 98%, or 99% sequence identity) to, or the sequence of, QVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSS (SEQ ID NO: 18); and/or a VL domain comprising an amino acid sequence having at least 90% sequence identity (e.g., at least 91%, 92%, 93%, 94%, 95%, 96%, 97%, 98%, or 99% sequence identity) to, or the sequence of, DIVMTQSPDSLAVSLGERATINCKSSQTVLYSSNNKKYLAWYQQKPGQPPNLLIYWASTRESGVPDRFSG SGSGTDFTLTISSLQAEDVAVYYCQQYYSTPFTFGPGTKVEIK (SEQ ID NO: 19). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 17 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising the amino acid sequence of SEQ ID NO: 17 and a VL domain comprising the amino acid sequence of SEQ ID NO: 19. In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 18 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising the amino acid sequence of SEQ ID NO: 18 and a VL domain comprising the amino acid sequence of SEQ ID NO: 19.

In some instances, the anti-TIGIT antagonist antibody includes a heavy chain and a light chain sequence, wherein: (a) the heavy chain comprises the amino acid sequence: EVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSSASTKGPSVFPLAP SSKSTSGGTAALGCLVKDYFPEPVTVSWNSGALTSGVHTFPAVLQSSGLYSLSSVVTVPSSSLGTQTYIC NVNHKPSNTKVDKKVEPKSCDKTHTCPPCPAPELLGGPSVFLFPPKPKDTLMISRTPEVTCVVVDVSHED PEVKFNWYVDGVEVHNAKTKPREEQYNSTYRVVSVLTVLHQDWLNGKEYKCKVSNKALPAPIEKTISKAK GQPREPQVYTLPPSREEMTKNQVSLTCLVKGFYPSDIAVEWESNGQPENNYKTTPPVLDSDGSFFLYSK LTVDKSRWQQGNVFSCSVMHEALHNHYTQKSLSLSPGK (SEQ ID NO: 23); and (b) the light chain comprises the amino acid sequence: DIVMTQSPDSLAVSLGERATINCKSSQTVLYSSNNKKYLAWYQQKPGQPPNLLIYWASTRESGVPDRFSG SGSGTDFTLTISSLQAEDVAVYYCQQYYSTPFTFGPGTKVEIKRTVAAPSVFIFPPSDEQLKSGTASVVCLL NNFYPREAKVQWKVDNALQSGNSQESVTEQDSKDSTYSLSSTLTLSKADYEKHKVYACEVTHQGLSSPV TKSFNRGEC (SEQ ID NO: 24).

In some instances, the anti-TIGIT antagonist antibody further comprises at least one, two, three, or four of the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and/or an FR-L4 comprising the amino acid sequence of FGPGTKVEIK (SEQ ID NO: 10), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 7-20. In some instances, for example, the antibody further comprises an FR-L1 comprising the amino acid sequence of DIVMTQSPDSLAVSLGERATINC (SEQ ID NO: 7); an FR-L2 comprising the amino acid sequence of WYQQKPGOPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10).

In some instances, the anti-TIGIT antagonist antibody further comprises at least one, two, three, or four of the following heavy chain variable region FRs: an FR-H1 comprising the amino acid sequence of XIVQLQQSGPGLVKPSQTLSLTCAISGDSVS (SEQ ID NO: 11), wherein X1 is E or Q; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 11-14. The anti-TIGIT antagonist antibody may further include, for example, at least one, two, three, or four of the following heavy chain variable region FRs: an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLOLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-15. In some instances, the anti-TIGIT antagonist antibody includes an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14). In another instance, for example, the anti-TIGIT antagonist antibody may further include at least one, two, three, or four of the following heavy chain variable region FRs: an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-14 and 16. In some instances, the anti-TIGIT antagonist antibody includes an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14).

In another aspect, an anti-TIGIT antagonist antibody is provided, wherein the antibody comprises a VH as in any of the instances provided above, and a VL as in any of the instances provided above, wherein one or both of the variable domain sequences include post-translational modifications.

In some instances, any one of the anti-TIGIT antagonist antibodies described above is capable of binding to rabbit TIGIT, in addition to human TIGIT. In some instances, any one of the anti-TIGIT antagonist antibodies described above is capable of binding to both human TIGIT and cynomolgus monkey (cyno) TIGIT. In some instances, any one of the anti-TIGIT antagonist antibodies described above is capable of binding to human TIGIT, cyno TIGIT, and rabbit TIGIT. In some instances, any one of the anti-TIGIT antagonist antibodies described above is capable of binding to human TIGIT, cyno TIGIT, and rabbit TIGIT, but not murine TIGIT.

In some instances, the anti-TIGIT antagonist antibody binds human TIGIT with a K_{D} of about 10 nM or lower and cyno TIGIT with a K_{D} of about 10 nM or lower (e.g., binds human TIGIT with a K_{D} of about 0.1 nM to about 1 nM and cyno TIGIT with a K_{D} of about 0.5 nM to about 1 nM, e.g., binds human TIGIT with a K_{D} of about 0.1 nM or lower and cyno TIGIT with a K_{D} of about 0.5 nM or lower).

In some instances, the anti-TIGIT antagonist antibody specifically binds TIGIT and inhibits or blocks TIGIT interaction with poliovirus receptor (PVR) (e.g., the antagonist antibody inhibits intracellular signaling mediated by TIGIT binding to PVR). In some instances, the antagonist antibody inhibits or blocks binding of human TIGIT to human PVR with an IC50 value of 10 nM or lower (e.g., 1 nM to about 10 nM). In some instances, the anti-TIGIT antagonist antibody specifically binds TIGIT and inhibits or blocks TIGIT interaction with PVR, without impacting PVR-CD226 interaction. In some instances, the antagonist antibody inhibits or blocks binding of cyno TIGIT to cyno PVR with an IC50 value of 50 nM or lower (e.g., 1 nM to about 50 nM, e.g., 1 nM to about 5 nM). In some instances, the anti-TIGIT antagonist antibody inhibits and/or blocks the interaction of CD226 with TIGIT. In some instances, the anti-TIGIT antagonist antibody inhibits and/or blocks the ability of TIGIT to disrupt CD226 homodimerization.

In some instances, the methods or uses described herein may include using or administering an isolated anti-TIGIT antagonist antibody that competes for binding to TIGIT with any of the anti-TIGIT antagonist antibodies described above. For example, the method may include administering an isolated anti-TIGIT antagonist antibody that competes for binding to TIGIT with an anti-TIGIT antagonist antibody having the following six HVRs: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and (f) an HVR-L3 comprising the amino acid sequence of QQYYSTPFT (SEQ ID NO: 6). The methods described herein may also include administering an isolated anti-TIGIT antagonist antibody that binds to the same epitope as an anti-TIGIT antagonist antibody described above.

In some aspects, the anti-TIGIT antagonist antibody exhibits Fc-mediated effector function, e.g., participates in antibody-dependent cellular cytotoxicity (ADCC). In some aspects, the anti-TIGIT antagonist antibody is an antibody having intact Fc-mediated effector function (e.g., tiragolumab, vibostolimab, etigilimab, EOS084448, or TJ-T6) or enhanced effector function (e.g., SGN-TGT).

In other aspects, the anti-TIGIT antagonist antibody is an antibody that lacks Fc-mediated effector function (e.g., domvanalimab, BMS-986207, ASP8374, or COM902).

In some aspects, the anti-TIGIT antagonist antibody is an IgG class antibody. In some aspects, the anti-TIGIT antagonist antibody is an IgG1 class antibody, e.g., tiragolumab, vibostolimab, domvanalimab, BMS-986207, etigilimab, BGB-A1217, SGN-TGT, EOS084448 (EOS-448), TJ-T6, or AB308. In some aspects, the antibody is a human monoclonal full-length IgG1 class antibody comprising an Fc region.

In some aspects, the anti-TIGIT antagonist antibody is a human, monoclonal full-length IgG1 subclass antibody comprising a human IgG1 Fc region, a heavy chain variable region (VH) comprising the amino acid sequence of SEQ ID NO: 17, and a light chain variable region (VL) comprising the amino acid sequence of SEQ ID NO: 19.

In other aspects, the anti-TIGIT antagonist antibody is an IgG4 class antibody, e.g., ASP8374 or COM902.

The anti-TIGIT antagonist antibodies (e.g., tiragolumab) useful in this invention, including compositions containing such antibodies, may be used in combination with a PD-1 axis binding antagonist (e.g., PD-L1 binding antagonists (e.g., anti-PD-L1 antagonist antibodies, e.g., atezolizumab), PD-1 binding antagonists (e.g., anti-PD-1 antagonist antibodies, e.g., pembrolizumab), and PD-L2 binding antagonists (e.g., anti-PD-L2 antagonist antibodies)).

In some embodiments, the anti-TIGIT antagonist antibody functions to inhibit TIGIT signaling. In some embodiments, the anti-TIGIT antagonist antibody inhibits the binding of TIGIT to its binding partners. Exemplary TIGIT binding partners include CD155 (PVR), CD112 (PVRL2 or Nectin-2), and CD113 (PVRL3 or Nectin-3). In some embodiments, the anti-TIGIT antagonist antibody is capable of inhibiting binding between TIGIT and CD155. In some embodiments, the anti-TIGIT antagonist antibody may inhibit binding between TIGIT and CD112. In some embodiments, the anti-TIGIT antagonist antibody inhibits binding between TIGIT and CD113. In some embodiments, the anti-TIGIT antagonist antibody inhibits TIGIT-mediated cellular signaling in immune cells. In some embodiments, the anti-TIGIT antagonist antibody inhibits TIGIT by depleting regulatory T cells (e.g., when engaging a FcγR).

In some embodiments, the anti-TIGIT antibody is a monoclonal antibody. In some embodiments, the anti-TIGIT antibody is an antibody fragment selected from the group consisting of Fab, Fab′-SH, Fv, scFv, and (Fab′) 2 fragments. In some embodiments, the anti-TIGIT antibody is a humanized antibody. In some embodiments, the anti-TIGIT antibody is a human antibody. In some embodiments, the anti-TIGIT antibody described herein binds to human TIGIT. In some embodiments, the anti-TIGIT antibody is an Fc fusion protein.

In some embodiments, the anti-TIGIT antibody is selected from the group consisting of tiragolumab (MTIG7192A, RG6058 or RO7092284), vibostolimab (MK-7684), ASP8374 (PTZ-201), EOS884448 (EOS-448), SEA-TGT (SGN-TGT)), BGB-A1217, BMS-986207 (ONO-4686), COM902 (CGEN-15137), IBI939, domvanalimab (AB154), M6223, AB308, AB154, TJ-T6, MG1131, NB6253, HLX301, HLX53, SL-9258 (TIGIT-Fc-LIGHT), STW264, and YBL-012. In some embodiments, the anti-TIGIT antibody is selected from the group consisting of tiragolumab (MTIG7192A, RG6058 or RO7092284), vibostolimab (MK-7684), ASP8374 (PTZ-201), EOS-448, and SEA-TGT (SGN-TGT). The anti-TIGIT antibody may be tiragolumab (MTIG7192A, RG6058 or RO7092284).

In some embodiments, the anti-TIGIT antibody comprises at least one, two, three, four, five, or six complementarity determining regions (CDRs) of any of the anti-TIGIT antibodies disclosed herein. In some embodiments, the anti-TIGIT antibody comprises the six CDRs of any of the anti-TIGIT antibodies disclosed herein. In some embodiments, the anti-TIGIT antibody comprises the six CDRs of any one of the antibodies selected from the group consisting of tiragolumab, ASP8374 (PTZ-201), BGB-A1217, BMS-986207 (ONO-4686), COM902 (CGEN-15137), M6223, IBI939, EOS884448 (EOS-448), domvanalimab (AB154), vibostolimab (MK-7684), and SEA-TGT (SGN-TGT).

In some embodiments, the anti-TIGIT antibody comprises a heavy chain and a light chain, wherein the heavy chain comprises a heavy chain variable region (VH) sequence of any one of the anti-TIGIT antibodies disclosed herein and the light chain comprises a light chain variable region (VL) of the same antibody. In some embodiments, the anti-TIGIT antibody comprises the VH and VL of an anti-TIGIT antibody selected from the group consisting of tiragolumab, ASP8374 (PTZ-201), BGB-A1217, BMS-986207 (ONO-4686), COM902 (CGEN-15137), M6223, IBI939, EOS884448 (EOS-448), domvanalimab (AB154), vibostolimab (MK-7684), and SEA-TGT (SGN-TGT).

In some embodiments, the anti-TIGIT antibody comprises the heavy chain and the light chain of any of the anti-TIGIT antibodies disclosed herein. In some embodiments, the anti-TIGIT antibody comprises the heavy chain and the light chain of an anti-TIGIT antibody selected from the group consisting of tiragolumab, ASP8374 (PTZ-201), BGB-A1217, BMS-986207 (ONO-4686), COM902 (CGEN-15137), M6223, IBI939, EOS884448 (EOS-448), domvanalimab (AB154), vibostolimab (MK-7684), and SEA-TGT (SGN-TGT).

### VIII. Bispecific Antibodies Targeting PD-1 and LAG3

A. Exemplary Bispecific Antibodies that Bind to PD-1 and LAG3

In one aspect, the invention provides a bispecific antibody comprising a first antigen-binding domain that specifically binds to PD-1 and a second antigen-binding domain that specifically binds to LAG3, wherein said first antigen-binding domain specifically binding to PD-1 comprises a VH domain comprising:
(i) HVR-H1 comprising the amino acid sequence of GFSFSSY (SEQ ID NO: 25),
(ii) HVR-H2 comprising the amino acid sequence GGR, and
(iii) HVR-H3 comprising an amino acid sequence of TGRVYFALD (SEQ ID NO: 26); and
a VL domain comprising
(i) HVR-L1 comprising the amino acid sequence of SESVDTSDNSF (SEQ ID NO: 27);
(ii) HVR-L2 comprising the amino acid sequence RSS, and
(iii) HVR-L3 comprising the amino acid sequence of NYDVPW (SEQ ID NO: 28).

In one aspect, the bispecific antibody comprises a Fc domain that is an IgG, particularly an IgG1 Fc domain or an IgG4 Fc domain and wherein the Fc domain has reduced or even abolished effector function. In particular, the Fc domain comprises one or more amino acid substitution that reduces binding to an Fc receptor, in particular towards Fcγ receptor.

In a further aspect, provided is a bispecific antibody comprising a first antigen-binding domain that specifically binds to PD-1 and a second antigen-binding domain that specifically binds to LAG3, wherein the bispecific antibody comprises a Fc domain that is an IgG, particularly an IgG1 Fc domain or an IgG4 Fc domain and wherein the Fc domain comprises one or more amino acid substitution that reduces binding to an Fc receptor, in particular towards Fcγ receptor.

In another aspect, provided is a bispecific antibody comprising a first antigen-binding domain that specifically binds to PD-1 and a second antigen-binding domain that specifically binds to LAG3, wherein the second antigen-binding domain that specifically binds to LAG3 comprises a VH domain comprising:
(i) HVR-H1 comprising the amino acid sequence of DYTMN (SEQ ID NO: 31),
(ii) HVR-H2 comprising the amino acid sequence of VISWDGGGTYYTDSVKG (SEQ ID NO: 32), and
(iii) HVR-H3 comprising an amino acid sequence of GLTDTTLYGSDY (SEQ ID NO: 33); and
a VL domain comprising
(i) HVR-L1 comprising the amino acid sequence of RASQSISSYLN (SEQ ID NO:34),
(ii) HVR-L2 comprising the amino acid sequence of AASTLOS (SEQ ID NO:35), and
(iii) HVR-L3 comprising the amino acid sequence of QQTYSSPLT (SEQ ID NO:36).

In a further aspect, provided is a bispecific antibody comprising a first antigen-binding domain that specifically binds to PD-1 and a second antigen-binding domain that specifically binds to LAG3, wherein the first antigen-binding domain specifically binding to PD-1 comprises a VH domain comprising the amino acid sequence of EVQLLESGGGLVQPGGSLRLSCAASGFSFSSYTMSWVRQAPGKGLEWVATISGGGRDIYYPDSV KGRFTISRDNSKNTLYLQMNSLRAEDTAVYYCVLLTGRVYFALDSWGQGTLVTVSS (SEQ ID NO: 29) and a VL domain comprising the amino acid sequence of DIVMTQSPDSLAVSLGERATINCKASESVDTSDNSFIHWYQQKPGQSPKLLIYRSSTLESGVPDRF SGSGSGTDFTLTISSLQAEDVAVYYCQQNYDVPWTFGQGTKVEIK (SEQ ID NO: 30).

In another aspect, provided is a bispecific antibody comprising a first antigen-binding domain that specifically binds to PD-1 and a second antigen-binding domain that specifically binds to LAG3, wherein the second antigen-binding domain specifically binding to LAG3 comprises
a VH domain comprising the amino acid sequence of EVQLLESGGGLVQPGGSLRL SCAASGFIFDDYTMNWVRQAPGKGLEWVAVISWDGGGTYYTDSVKGRFTISRDDFKNTLY LQMNSLRAEDTAVYYCAKGLTDTTLYGSDYWGQGTLVTVSS (SEQ ID NO: 37) and a VL domain
comprising the amino acid sequence of DIQMTQSPSSLSASVGDRVTITCRASQSISSYLNWYQQKPGKAPKLLIYAASTLQSGVPSRFSGSG SGTDFTLTISSLOPEDFATYYCQQ TYSSPLTFGGGTKVEIK (SEQ ID NO: 38).

In one aspect, the bispecific antibody targeting PD-1 and LAG3 comprises a first antigen-binding domain that specifically binds to PD-1 and a second antigen-binding domain that specifically binds to LAG3, wherein the first antigen-binding domain specifically binding to PD-1 comprises a VH domain having at least 90% identity to (e.g., having 90%, 91%, 92%, 93%, 94%, 95%, 96%, 97%, 98%, 99%, or more than 99% identity to) the amino acid sequence of SEQ ID NO: 29 and a VL domain having at least 90% identity to (e.g., having 90%, 91%, 92%, 93%, 94%, 95%, 96%, 97%, 98%, 99%, or more than 99% identity to) the amino acid sequence of SEQ ID NO: 30. In one aspect, the first antigen-binding domain specifically binding to PD-1 comprises a VH domain comprising the amino acid sequence of SEQ ID NO: 29 and a VL domain comprising the amino acid sequence of SEQ ID NO: 30.

In one aspect, the bispecific antibody targeting PD-1 and LAG3 comprises a first antigen-binding domain that specifically binds to PD-1 and a second antigen-binding domain that specifically binds to LAG3, wherein the second antigen-binding domain specifically binding to LAG3 comprises a VH domain having at least 90% identity to (e.g., having 90%, 91%, 92%, 93%, 94%, 95%, 96%, 97%, 98%, 99%, or more than 99% identity to) the amino acid sequence of SEQ ID NO: 37 and a VL domain having at least 90% identity to (e.g., having 90%, 91%, 92%, 93%, 94%, 95%, 96%, 97%, 98%, 99%, or more than 99% identity to) the amino acid sequence of SEQ ID NO: 38. In one aspect, the second antigen-binding domain specifically binding to LAG3 comprises a VH domain comprising the amino acid sequence of SEQ ID NO: 37 and a VL domain comprising the amino acid sequence of SEQ ID NO: 38.

In one aspect, the bispecific antibody targeting PD-1 and LAG3 comprises a first antigen-binding domain that specifically binds to PD-1 and a second antigen-binding domain that specifically binds to LAG3, wherein the first antigen-binding domain specifically binding to PD-1 comprises a VH domain having at least 90% identity to (e.g., having 90%, 91%, 92%, 93%, 94%, 95%, 96%, 97%, 98%, 99%, or more than 99% identity to) the amino acid sequence of SEQ ID NO: 29 and a VL domain having at least 90% identity to (e.g., having 90%, 91%, 92%, 93%, 94%, 95%, 96%, 97%, 98%, 99%, or more than 99% identity to) the amino acid sequence of SEQ ID NO: 30 and the second antigen-binding domain specifically binding to LAG3 comprises a VH domain having at least 90% identity to (e.g., having 90%, 91%, 92%, 93%, 94%, 95%, 96%, 97%, 98%, 99%, or more than 99% identity to) the amino acid sequence of SEQ ID NO: 37 and a VL domain having at least 90% identity to (e.g., having 90%, 91%, 92%, 93%, 94%, 95%, 96%, 97%, 98%, 99%, or more than 99% identity to) the amino acid sequence of SEQ ID NO: 38.

In another aspect, provided is a bispecific antibody comprising a first antigen-binding domain that specifically binds to PD-1 and a second antigen-binding domain that specifically binds to LAG3, wherein
the first antigen-binding domain specifically binding to PD-1 comprises a VH domain comprising the amino acid sequence of SEQ ID NO: 29 and a VL domain comprising the amino acid sequence of SEQ ID NO: 30,
and the second antigen-binding domain specifically binding to LAG3 comprises a VH domain comprising the amino acid sequence of SEQ ID NO: 37 and a VL domain comprising the amino acid sequence of SEQ ID NO: 38.

In a further aspect, the bispecific antibody comprising a first antigen-binding domain that specifically binds to PD-1 and a second antigen-binding domain that specifically binds to LAG3 is a human, humanized or chimeric antibody. In particular, it is a humanized or chimeric antibody.

In one aspect, the bispecific antibody comprising a first antigen-binding domain that specifically binds to PD-1 and a second antigen-binding domain that specifically binds to LAG3 is bivalent. This means that the bispecific antibody comprises one antigen-binding domain that specifically binds to PD-1 and one antigen-binding domain that specifically binds to LAG3 (1+1 format).

In one aspect, provided is a bispecific antibody comprising a first antigen-binding domain that specifically binds to PD-1 and a second antigen-binding domain that specifically binds to LAG3, wherein the bispecific antibody comprises an Fc domain, a first Fab fragment comprising the antigen-binding domain that specifically binds to PD-1 and a second Fab fragment comprising the antigen-binding domain that specifically binds to LAG3. In a particular aspect, in one of the Fab fragments the variable domains VL and VH are replaced by each other so that the VH domain is part of the light chain and the VL domain is part of the heavy chain. In a particular aspect, in the first Fab fragment comprising the antigen-binding domain that specifically binds to PD-1 the variable domains VL and VH are replaced by each other.

In a particular aspect, provided is a bispecific antibody comprising a first antigen-binding domain that specifically binds to PD-1 and a second antigen-binding domain that specifically binds to LAG3, wherein the bispecific antibody comprises a first heavy chain comprising an amino acid sequence with at least 95% sequence identity to the sequence of SEQ ID NO: 39, a first light chain comprising an amino acid sequence with at least 95% sequence identity to the sequence of SEQ ID NO: 40, a second heavy chain comprising an amino acid sequence with at least 95% sequence identity to the sequence of SEQ ID NO: 41, and a second light chain comprising an amino acid sequence with at least 95% sequence identity to the sequence of SEQ ID NO:42. For example, in one aspect, the bispecific antibody comprises a first heavy chain comprising the amino acid sequence of SEQ ID NO: 39, a first light chain comprising the amino acid sequence of SEQ ID NO: 40, a second heavy chain comprising the amino acid sequence of SEQ ID NO: 41, and a second light chain comprising the amino acid sequence of SEQ ID NO:42 (PD1-LAG3).

In a further aspect, provided is a bispecific antibody comprising a first antigen-binding domain that specifically binds to PD-1 and a second antigen-binding domain that specifically binds to LAG3, wherein the bispecific antibody comprises an Fc domain, a first Fab fragment comprising the antigen-binding domain that specifically binds to PD-1 and a second Fab fragment comprising the antigen-binding domain that specifically binds to LAG3 that is fused to the C-terminus of the Fc domain. Particularly, the Fab fragment comprising the antigen-binding domain that specifically binds to LAG3 is fused to the C-terminus of the Fc domain via its VH domain (trans 1+1 format).

In one aspect, the bispecific antibody comprises a first heavy chain comprising an amino acid sequence with at least 95% sequence identity to the sequence of SEQ ID NO: 39, a first light chain comprising an amino acid sequence with at least 95% sequence identity to the sequence of SEQ ID NO: 40, a second heavy chain comprising an amino acid sequence with at least 95% sequence identity to the sequence of SEQ ID NO: 61, and a second light chain comprising an amino acid sequence with at least 95% sequence identity to the sequence of SEQ ID NO: 42. More particularly, the bispecific antibody may comprise a first heavy chain comprising an amino acid sequence of SEQ ID NO: 39, a first light chain comprising an amino acid sequence of SEQ ID NO: 40, a second heavy chain comprising an amino acid sequence of SEQ ID NO: 61, and a second light chain comprising an amino acid sequence of SEQ ID NO: 42.

i. Fc Domain Modifications Reducing Fc Receptor Binding and/or Effector Function

In certain aspects, provided is a bispecific antibody comprising a first antigen-binding domain that specifically binds to PD-1 and a second antigen-binding domain that specifically binds to LAG3, wherein the bispecific antibody comprises a Fc domain comprising one or more amino acid modifications that reduce binding to an Fc receptor, in particular towards Fcγ receptor, and reduce or abolish effector function.

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

The following section describes preferred aspects of the bispecific antigen binding molecules of the invention comprising Fc domain modifications reducing Fc receptor binding and/or effector function. In one aspect, the invention relates to a bispecific antibody comprising a first antigen-binding domain that specifically binds to PD-1 and a second antigen-binding domain that specifically binds to LAG3, wherein the Fc domain comprises one or more amino acid substitution that reduces binding to an Fc receptor, in particular towards Fcγ receptor. In particular, the Fc domain is of human IgG1 subclass with the amino acid mutations L234A, L235A and P329G (numbering according to Kabat EU index).

The Fc domain confers favorable pharmacokinetic properties to the bispecific antibodies of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular embodiments the Fc domain of the the bispecific antibodies of the invention exhibits reduced binding affinity to an Fc receptor and/or reduced effector function, as compared to a native IgG Fc domain, in particular an IgG1 Fc domain or an IgG4 Fc domain. More particularly, the Fc domain is an IgG1 FC domain.

In one such aspect the Fc domain (or the bispecific antigen binding molecule of the invention comprising said Fc domain) exhibits less than 50%, preferably less than 20%, more preferably less than 10% and most preferably less than 5% of the binding affinity to an Fc receptor, as compared to a native IgG1 Fc domain (or the bispecific antigen binding molecule of the invention comprising a native IgG1 Fc domain), and/or less than 50%, preferably less than 20%, more preferably less than 10% and most preferably less than 5% of the effector function, as compared to a native IgG1 Fc domain (or the bispecific antigen binding molecule of the invention comprising a native IgG1 Fc domain). In one aspect, the Fc domain (or the bispecific antigen binding molecule of the invention comprising said Fc domain) does not substantially bind to an Fc receptor and/or induce effector function. In a particular aspect the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. In one aspect, the Fc receptor is an inhibitory Fc receptor. In a specific aspect, the Fc receptor is an inhibitory human Fcγ receptor, more specifically human FcγRIIB. In one aspect the effector function is one or more of CDC, ADCC, ADCP, and cytokine secretion. In a particular aspect, the effector function is ADCC. In one aspect, the Fc domain domain exhibits substantially similar binding affinity to neonatal Fc receptor (FcRn), as compared to a native IgG1 Fc domain. Substantially similar binding to FcRn is achieved when the Fc domain (or the the bispecific antigen binding molecule of the invention comprising said Fc domain) exhibits greater than about 70%, particularly greater than about 80%, more particularly greater than about 90% of the binding affinity of a native IgG1 Fc domain (or the the bispecific antigen binding molecule of the invention comprising a native IgG1 Fc domain) to FcRn.

In a particular aspect, the Fc domain is engineered to have reduced binding affinity to an Fc receptor and/or reduced effector function, as compared to a non-engineered Fc domain. In a particular aspect, the Fc domain of the bispecific antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In one aspect, the amino acid mutation reduces the binding affinity of the Fc domain to an Fc receptor. In another aspect, the amino acid mutation reduces the binding affinity of the Fc domain to an Fc receptor by at least 2-fold, at least 5-fold, or at least 10-fold. In one aspect, the bispecific antigen binding molecule of the invention comprising an engineered Fc domain exhibits less than 20%, particularly less than 10%, more particularly less than 5% of the binding affinity to an Fc receptor as compared to bispecific antibodies of the invention comprising a non-engineered Fc domain. In a particular aspect, the Fc receptor is an Fcγ receptor. In other aspects, the Fc receptor is a human Fc receptor. In one aspect, the Fc receptor Is an inhibitory Fc receptor. In a specific aspect, the Fc receptor is an inhibitory human Fcγ receptor, more specifically human FcγRIIB. In some aspects the Fc receptor is an activating Fc receptor. In a specific aspect, the Fc receptor is an activating human Fcγ receptor, more specifically human FcγRIIIa, FcγRI or FcγRIIa, most specifically human FcγRIIIa. Preferably, binding to each of these receptors is reduced. In some aspects, binding affinity to a complement component, specifically binding affinity to C1q, is also reduced. In one aspect, binding affinity to neonatal Fc receptor (FcRn) is not reduced. Substantially similar binding to FcRn, i.e. preservation of the binding affinity of the Fc domain to said receptor, is achieved when the Fc domain (or the bispecific antigen binding molecule of the invention comprising said Fc domain) exhibits greater than about 70% of the binding affinity of a non-engineered form of the Fc domain (or the bispecific antigen binding molecule of the invention comprising said non-engineered form of the Fc domain) to FcRn. The Fc domain, or the the bispecific antigen binding molecule of the invention comprising said Fc domain, may exhibit greater than about 80% and even greater than about 90% of such affinity. In certain embodiments the Fc domain of the bispecific antigen binding molecule of the invention is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described. (e.g. U.S. Pat. No. 6,737,056; WO 2004/056312, and Shields, R. L. et al., J. Biol. Chem. 276 (2001) 6591-6604).

In one aspect of the invention, the Fc domain comprises an amino acid substitution at a position of E233, L234, L235, N297, P331 and P329. In some aspects, the Fc domain comprises the amino acid substitutions L234A and L235A (“LALA”). In one such embodiment, the Fc domain is an IgG1 Fc domain, particularly a human IgG1 Fc domain. In one aspect, the Fc domain comprises an amino acid substitution at position P329. In a more specific aspect, the amino acid substitution is P329A or P329G, particularly P329G. In one embodiment the Fc domain comprises an amino acid substitution at position P329 and a further amino acid substitution selected from the group consisting of E233P, L234A, L235A, L235E, N297A, N297D or P331S. In more particular embodiments the Fc domain comprises the amino acid mutations L234A, L235A and P329G (“P329G LALA”). The “P329G LALA” combination of amino acid substitutions almost completely abolishes Fcγ receptor binding of a human IgG1 Fc domain, as described in PCT Patent Application No. WO 2012/130831 A1. Said document also describes methods of preparing such mutant Fc domains and methods for determining its properties such as Fc receptor binding or effector functions. Such antibody is an IgG1 with mutations L234A and L235A or with mutations L234A, L235A and P329G (numbering according to EU index of Kabat et al., Sequences of Proteins of Immunological Interest, 5th Ed. Public Health Service, National Institutes of Health, Bethesda, M D, 1991).

In one aspect, the bispecific antibody of the invention comprises (all positions according to EU index of Kabat) (i) a homodimeric Fc-region of the human IgG1 subclass optionally with the mutations P329G, L234A and L235A, or (ii) a homodimeric Fc-region of the human IgG4 subclass optionally with the mutations P329G, S228P and L235E, or (iii) a homodimeric Fc-region of the human IgG1 subclass optionally with the mutations P329G, L234A, L235A, 1253A, H310A, and H435A, or optionally with the mutations P329G, L234A, L235A, H310A, H433A, and Y436A, or (iv) a heterodimeric Fc-region wherein one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C, or (v) a heterodimeric Fc-region of the human IgG1 subclass wherein both Fc-region polypeptides comprise the mutations P329G, L234A and L235A and one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C.

In one aspect, the Fc domain is an IgG4 Fc domain. In a more specific embodiment, the Fc domain is an IgG4 Fc domain comprising an amino acid substitution at position S228 (Kabat numbering), particularly the amino acid substitution S228P. In a more specific embodiment, the Fc domain is an IgG4 Fc domain comprising amino acid substitutions L235E and S228P and P329G. This amino acid substitution reduces in vivo Fab arm exchange of IgG4 antibodies (see Stubenrauch et al., Drug Metabolism and Disposition 38, 84-91 (2010)). Thus, in one aspect, provided is a bispecific antibody, comprising (all positions according to EU index of Kabat) a heterodimeric Fc-region of the human IgG4 subclass wherein both Fc-region polypeptides comprise the mutations P329G, S228P and L235E and one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C.

Antibodies with increased half lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan, A. R. and Winter, G., Nature 322 (1988) 738-740; U.S. Pat. Nos. 5,648,260; 5,624,821; and WO 94/29351 concerning other examples of Fc region variants.

Binding to Fc receptors can be easily determined, e.g., by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor. Effector function of an Fc domain, or bispecific antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and Cyto Tox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc Natl Acad Sci USA 95, 652-656 (1998).

The following section describes preferred aspects of the bispecific antibodies of the invention comprising Fc domain modifications reducing Fc receptor binding and/or effector function. In one aspect, the invention relates to the bispecific comprising a first antigen-binding domain that specifically binds PD-1 and a second antigen-binding domain that specifically binds to LAG3, wherein the Fc domain comprises one or more amino acid substitution that reduces the binding affinity of the antibody to an Fc receptor, in particular towards Fcγ receptor. In another aspect, the invention relates to the bispecific antibody comprising a first antigen-binding domain that specifically binds to PD-1 and a second antigen-binding domain that specifically binds to LAG3, wherein the Fc domain comprises one or more amino acid substitution that reduces effector function. In particular aspect, the Fc domain is of human IgG1 subclass with the amino acid mutations L234A, L235A and P329G (numbering according to Kabat EU index).

### ii Fc Domain Modifications Promoting Heterodimerization

The bispecific antigen binding molecules of the invention comprise different antigen-binding domains, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific antibodies of the invention in recombinant production, it will thus be advantageous to introduce in the Fc domain of the bispecific antigen binding molecules of the invention a modification promoting the association of the desired polypeptides.

Accordingly, in particular aspects the invention relates to a bispecific antibody comprising a first antigen-binding domain that specifically binds to PD-1 and a second antigen-binding domain that specifically binds to LAG3, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect said modification is a so-called “knob-into-hole” modification, comprising a “knob” modification in one of the two subunits of the Fc domain and a “hole” modification in the other one of the two subunits of the Fc domain. Thus, the invention relates to a bispecific antibody comprising a first antigen-binding domain that specifically binds to PD-1 and a second antigen-binding site that specifically binds to LAG3, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g., in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in one aspect, in the CH3 domain of the first subunit of the Fc domain of the bispecific antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g., by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues leads to the formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

But also other knobs-in-holes technologies as described by EP 1 870 459, can be used alternatively or additionally. In one embodiment the multispecific antibody comprises the mutations R409D and K370E in the CH3 domain of the “knobs chain” and the mutations D399K and E357K in the CH3 domain of the “hole-chain” (numbering according to Kabat EU index).

In one aspect, the bispecific antibody comprises a T366W mutation in the CH3 domain of the “knobs chain” and the mutations T366S, L368A and Y407V in the CH3 domain of the “hole chain” and additionally the mutations R409D and K370E in the CH3 domain of the “knobs chain” and the mutations D399K and E357K in the CH3 domain of the “hole chain” (numbering according to the Kabat EU index).

In one aspect, the bispecific antibody comprises the mutations Y349C and T366W in one of the two CH3 domains and the mutations S354C, T366S, L368A and Y407V in the other of the two CH3 domains, or the multispecific antibody comprises the mutations Y349C and T366W in one of the two CH3 domains and the mutations S354C, T366S, L368A and Y407V in the other of the two CH3 domains and additionally the mutations R409D and K370E in the CH3 domain of the “knobs chain” and the mutations D399K and E357K in the CH3 domain of the “hole chain” (numbering according to the Kabat EU index).

In an alternative aspect, a modification promoting association of the first and the second subunit of the Fc domain comprises a modification mediating electrostatic steering effects, e.g., as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

Apart from the “knob-into-hole technology” other techniques for modifying the CH3 domains of the heavy chains of a multispecific antibody to enforce heterodimerization are known in the art. These technologies, especially the ones described in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291 are contemplated herein as alternatives to the “knob-into-hole technology” in combination with a bispecific antibody.

In one aspect, in the bispecific antibody the approach described in EP 1870459 is used to support heterodimerization of the first heavy chain and the second heavy chain of the multispecific antibody. This approach is based on the introduction of charged amino acids with opposite charges at specific amino acid positions in the CH3/CH3-domain-interface between both, the first and the second heavy chain.

Accordingly, in this aspect in the tertiary structure of the multispecific antibody the CH3 domain of the first heavy chain and the CH3 domain of the second heavy chain form an interface that is located between the respective antibody CH3 domains, wherein the respective amino acid sequences of the CH3 domain of the first heavy chain and the amino acid sequence of the CH3 domain of the second heavy chain each comprise a set of amino acids that is located within said interface in the tertiary structure of the antibody, wherein from the set of amino acids that is located in the interface in the CH3 domain of one heavy chain a first amino acid is substituted by a positively charged amino acid and from the set of amino acids that is located in the interface in the CH3 domain of the other heavy chain a second amino acid is substituted by a negatively charged amino acid. The bispecific antibody according to this aspect is herein also referred to as “CH3(+/−)-engineered bispecific antibody” (wherein the abbreviation “+/−” stands for the oppositely charged amino acids that were introduced in the respective CH3 domains).

In one aspect, in the CH3(+/−)-engineered bispecific antibody the positively charged amino acid is selected from K, R and H, and the negatively charged amino acid is selected from E or D.

In one aspect, in the CH3(+/−)-engineered bispecific antibody the positively charged amino acid is selected from K and R, and the negatively charged amino acid is selected from E or D.

In one aspect, in the CH3(+/−)-engineered bispecific antibody the positively charged amino acid is K, and the negatively charged amino acid is E.

In one aspect, in the CH3(+/−)-engineered bispecific antibody in the CH3 domain of one heavy chain the amino acid R at position 409 is substituted by D and the amino acid K at position is substituted by E, and in the CH3 domain of the other heavy chain the amino acid D at position 399 is substituted by K and the amino acid E at position 357 is substituted by K (numbering according to Kabat EU index).

In one aspect, the approach described in WO 2013/157953 is used to support heterodimerization of the first heavy chain and the second heavy chain of the multispecific antibody. In one embodiment in the CH3 domain of one heavy chain the amino acid T at position 366 is substituted by K, and in the CH3 domain of the other heavy chain the amino acid L at position 351 is substituted by D (numbering according to Kabat EU index). In another embodiment in the CH3 domain of one heavy chain the amino acid T at position 366 is substituted by K and the amino acid L at position 351 is substituted by K, and in the CH3 domain of the other heavy chain the amino acid L at position 351 is substituted by D (numbering according to Kabat EU index).

In another aspect, in the CH3 domain of one heavy chain the amino acid T at position 366 is substituted by K and the amino acid L at position 351 is substituted by K, and in the CH3 domain of the other heavy chain the amino acid L at position 351 is substituted by D (numbering according to Kabat EU index). Additionally at least one of the following substitutions is comprised in the CH3 domain of the other heavy chain: the amino acid Y at position 349 is substituted by E, the amino acid Y at position 349 is substituted by D and the amino acid L at position 368 is substituted by E (numbering according to Kabat EU index). In one embodiment the amino acid L at position 368 is substituted by E (numbering according to Kabat EU index).

In one aspect, the approach described in WO 2012/058768 is used to support heterodimerization of the first heavy chain and the second heavy chain of the multispecific antibody. In one aspect, in the CH3 domain of one heavy chain the amino acid L at position 351 is substituted by Y and the amino acid Y at position 407 is substituted by A, and in the CH3 domain of the other heavy chain the amino acid T at position 366 is substituted by A and the amino acid K at position 409 is substituted by F (numbering according to Kabat EU index). In another embodiment, in addition to the aforementioned substitutions, in the CH3 domain of the other heavy chain at least one of the amino acids at positions 411 (originally T), 399 (originally D), 400 (originally S), 405 (originally F), 390 (originally N) and 392 (originally K) is substituted (numbering according to Kabat EU index). Preferred substitutions are:
substituting the amino acid T at position 411 by an amino acid selected from N, R, Q, K, D, E and W (numbering according to Kabat EU index),
substituting the amino acid D at position 399 by an amino acid selected from R, W, Y, and K (numbering according to Kabat EU index),
substituting the amino acid S at position 400 by an amino acid selected from E, D, R and K (numbering according to Kabat EU index),
substituting the amino acid F at position 405 by an amino acid selected from I, M, T, S, V and W (numbering according to Kabat EU index;
substituting the amino acid N at position 390 by an amino acid selected from R, K and D (numbering according to Kabat EU index; and
substituting the amino acid K at position 392 by an amino acid selected from V, M, R, L, F and E (numbering according to Kabat EU index).

In another aspect, the bispecific antibody is engineered according to WO 2012/058768), i.e. in the CH3 domain of one heavy chain the amino acid L at position 351 is substituted by Y and the amino acid Y at position 407 is substituted by A, and in the CH3 domain of the other heavy chain the amino acid T at position 366 is substituted by V and the amino acid K at position 409 is substituted by F (numbering according to Kabat EU index). In another embodiment of the multispecific antibody, in the CH3 domain of one heavy chain the amino acid Y at position 407 is substituted by A, and in the CH3 domain of the other heavy chain the amino acid T at position 366 is substituted by A and the amino acid K at position 409 is substituted by F (numbering according to Kabat EU index). In the last aforementioned embodiment, in the CH3 domain of the other heavy chain the amino acid K at position 392 is substituted by E, the amino acid T at position 411 is substituted by E, the amino acid D at position 399 is substituted by R and the amino acid S at position 400 is substituted by R (numbering according to Kabat EU index).

In one aspect, the approach described in WO 2011/143545 is used to support heterodimerization of the first heavy chain and the second heavy chain of the multispecific antibody. In one aspect, amino acid modifications in the CH3 domains of both heavy chains are introduced at positions 368 and/or 409 (numbering according to Kabat EU index).

In one aspect, the approach described in WO 2011/090762 is used to support heterodimerization of the first heavy chain and the second heavy chain of the bispecific antibody. WO 2011/090762 relates to amino acid modifications according to the “knob-into-hole” (KiH) technology. In one embodiment in the CH3 domain of one heavy chain the amino acid T at position 366 is substituted by W, and in the CH3 domain of the other heavy chain the amino acid Y at position 407 is substituted by A (numbering according to Kabat EU index). In another embodiment in the CH3 domain of one heavy chain the amino acid T at position 366 is substituted by Y, and in the CH3 domain of the other heavy chain the amino acid Y at position 407 is substituted by T (numbering according to Kabat EU index).

In one aspect, the approach described in WO 2009/089004 is used to support heterodimerization of the first heavy chain and the second heavy chain of the bispecific antibody. In one embodiment in the CH3 domain of one heavy chain the amino acid K or N at position 392 is substituted by a negatively charged amino acid (in one embodiment by E or D, in one preferred embodiment by D), and in the CH3 domain of the other heavy chain the amino acid D at position 399 the amino acid E or D at position 356 or the amino acid E at position 357 is substituted by a positively charged amino acid (in one embodiment K or R, in one preferred embodiment by K, in one preferred embodiment the amino acids at positions 399 or 356 are substituted by K) (numbering according to Kabat EU index). In one further embodiment, in addition to the aforementioned substitutions, in the CH3 domain of the one heavy chain the amino acid K or R at position 409 is substituted by a negatively charged amino acid (in one embodiment by E or D, in one preferred embodiment by D) (numbering according to Kabat EU index). In one even further aspect, in addition to or alternatively to the aforementioned substitutions, in the CH3 domain of the one heavy chain the amino acid K at position 439 and/or the amino acid K at position 370 is substituted independently from each other by a negatively charged amino acid (in one embodiment by E or D, in one preferred embodiment by D) (numbering according to Kabat EU index).

In one aspect, the approach described in WO 2007/147901 is used to support heterodimerization of the first heavy chain and the second heavy chain of the multispecific antibody. In one embodiment in the CH3 domain of one heavy chain the amino acid K at position 253 is substituted by E, the amino acid D at position 282 is substituted by K and the amino acid K at position 322 is substituted by D, and in the CH3 domain of the other heavy chain the amino acid D at position 239 is substituted by K, the amino acid E at position 240 is substituted by K and the amino acid K at position 292 is substituted by D (numbering according to Kabat EU index).

The C-terminus of the heavy chain of the bispecific antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG.

In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one embodiment of all aspects as reported herein, a bispecific antibody comprising a heavy chain including a C-terminal CH3 domain, as specified herein, comprises a C-terminal glycine residue (G446, numbering according to Kabat EU index).

iii. Modifications in the Fab Domains

In one aspect, the invention relates to a bispecific antibody comprising a first Fab fragment that specifically binds to PD-1 and a second Fab fragment that specifically binds to LAG3, wherein in one of the Fab fragments either the variable domains VH and VL or the constant domains CH1 and CL are exchanged. The bispecific antibodies are prepared according to the Crossmab technology.

Multispecific antibodies with a domain replacement/exchange in one binding arm (CrossMabVH-VL or CrossMabCH-CL) are described in detail in WO2009/080252, WO2009/080253 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In a particular aspect, the invention relates to a bispecific antibody comprising a first Fab fragment that specifically binds to PD-1 and a second Fab fragment that specifically binds to LAG3, wherein in one of the Fab fragments the variable domains VL and VH are replaced by each other so that the VH domain is part of the light chain and the VL domain is part of the heavy chain. In a particular aspect, the bispecific antibody is one, wherein in the first Fab fragment comprising the antigen-binding domain that specifically binds to PD-1 the variable domains VL and VH are replaced by each other.

In another aspect, and to further improve correct pairing, the bispecific antibody comprising a first Fab fragment that specifically binds to PD-1 and a second Fab fragment that specifically binds to LAG3, can contain different charged amino acid substitutions (so-called “charged residues”). These modifications are introduced in the crossed or non-crossed CH1 and CL domains. Such modifications are described e.g., in WO2015/150447, WO2016/020309 and PCT/EP2016/073408.

In a particular aspect, the invention is concerned with a bispecific antibody comprising a first Fab fragment that specifically binds to PD-1 and a second Fab fragment that specifically binds to LAG3, wherein in one of the Fab fragments in the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index). In a particular aspect, the bispecific antibody is one, wherein in the second Fab fragment comprising the antigen-binding domain that specifically binds to TIM3 the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index).

In a particular aspect, the invention relates to a bispecific antibody comprising a first Fab fragment that specifically binds to PD-1 and a second Fab fragment that specifically binds to LAG3, wherein in one of CL domains the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E). In a particular aspect, the bispecific antibody is one, wherein in the second Fab fragment comprising the antigen-binding domain that specifically binds to LAG3 the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E).

In a further aspect, the bispecific antibody is a bivalent antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other.

The antibody under a) does not contain a modification as reported under b) and the heavy chain and the light chain under a) are isolated chains.

In the antibody under b) within the light chain the variable light chain domain VL is replaced by the variable heavy chain domain VH of said antibody, and within the heavy chain the variable heavy chain domain VH is replaced by the variable light chain domain VL of said antibody.

In one aspect, (i) in the constant domain CL of the first light chain under a) the amino acid at position 124 (numbering according to Kabat) is substituted by a positively charged amino acid, and wherein in the constant domain CH1 of the first heavy chain under a) the amino acid at position 147 or the amino acid at position 213 (numbering according to Kabat EU index) is substituted by a negatively charged amino acid, or (ii) in the constant domain CL of the second light chain under b) the amino acid at position 124 (numbering according to Kabat) is substituted by a positively charged amino acid, and wherein in the constant domain CH1 of the second heavy chain under b) the amino acid at position 147 or the amino acid at position 213 (numbering according to Kabat EU index) is substituted by a negatively charged amino acid.

In another aspect, (i) in the constant domain CL of the first light chain under a) the amino acid at position 124 is substituted independently by lysine (K), arginine (R) or histidine (H) (numbering according to Kabat) (in one preferred embodiment independently by lysine (K) or arginine (R)), and wherein in the constant domain CH1 of the first heavy chain under a) the amino acid at position 147 or the amino acid at position 213 is substituted independently by glutamic acid (E) or aspartic acid (D) (numbering according to Kabat EU index), or (ii) in the constant domain CL of the second light chain under b) the amino acid at position 124 is substituted independently by lysine (K), arginine (R) or histidine (H) (numbering according to Kabat) (in one preferred embodiment independently by lysine (K) or arginine (R)), and wherein in the constant domain CH1 of the second heavy chain under b) the amino acid at position 147 or the amino acid at position 213 is substituted independently by glutamic acid (E) or aspartic acid (D) (numbering according to Kabat EU index).

In one aspect, in the constant domain CL of the second heavy chain the amino acids at position 124 and 123 are substituted by K (numbering according to Kabat EU index).

In one aspect, in the constant domain CL of the second heavy chain the amino acid at position 123 is substituted by R and the amino acid as position 124 is substituted by K (numbering according to Kabat EU index).

In one aspect, in the constant domain CH1 of the second light chain the amino acids at position 147 and 213 are substituted by E (numbering according to EU index of Kabat).

In one aspect, in the constant domain CL of the first light chain the amino acids at position 124 and 123 are substituted by K, and in the constant domain CH1 of the first heavy chain the amino acids at position 147 and 213 are substituted by E (numbering according to Kabat EU index).

In one aspect, in the constant domain CL of the first light chain the amino acid at position 123 is substituted by R and the amino acid at position 124 is substituted by K, and in the constant domain CH1 of the first heavy chain the amino acids at position 147 and 213 are both substituted by E (numbering according to Kabat EU index).

In one aspect, in the constant domain CL of the second heavy chain the amino acids at position 124 and 123 are substituted by K, and wherein in the constant domain CH1 of the second light chain the amino acids at position 147 and 213 are substituted by E, and in the variable domain VL of the first light chain the amino acid at position 38 is substituted by K, in the variable domain VH of the first heavy chain the amino acid at position 39 is substituted by E, in the variable domain VL of the second heavy chain the amino acid at position 38 is substituted by K, and in the variable domain VH of the second light chain the amino acid at position 39 is substituted by E (numbering according to Kabat EU index).

In one aspect, the bispecific antibody is a bivalent antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other, and wherein the constant domains CL and CH1 of the second light chain and the second heavy chain are replaced by each other.

The antibody under a) does not contain a modification as reported under b) and the heavy chain and the light chain und a) are isolated chains. In the antibody under b) within the light chain the variable light chain domain VL is replaced by the variable heavy chain domain VH of said antibody, and the constant light chain domain CL is replaced by the constant heavy chain domain CH1 of said antibody; and within the heavy chain the variable heavy chain domain VH is replaced by the variable light chain domain VL of said antibody, and the constant heavy chain domain CH1 is replaced by the constant light chain domain CL of said antibody.

In one aspect, the bispecific antibody is a bivalent antibody comprising
a) a first light chain and a first heavy chain of an antibody specifically binding to a first antigen, and
b) a second light chain and a second heavy chain of an antibody specifically binding to a second antigen, wherein the constant domains CL and CH1 of the second light chain and the second heavy chain are replaced by each other.

The antibody under a) does not contain a modification as reported under b) and the heavy chain and the light chain under a) are isolated chains. In the antibody under b) within the light chain the constant light chain domain CL is replaced by the constant heavy chain domain CH1 of said antibody; and within the heavy chain the constant heavy chain domain CH1 is replaced by the constant light chain domain CL of said antibody.

In one aspect, the bispecific antibody is a bispecific antibody comprising
a) a full-length antibody specifically binding to a first antigen and consisting of two antibody heavy chains and two antibody light chains, and
b) one, two, three or four single chain Fab fragments specifically binding to a second antigen, wherein said single chain Fab fragments under b) are fused to said full-length antibody under a) via a peptide linker at the C- or N-terminus of the heavy or light chain of said full length antibody.

In one aspect, one or two identical single chain Fab fragments binding to a second antigen are fused to the full-length antibody via a peptide linker at the C terminus of the heavy or light chains of said full-length antibody.

In one aspect, one or two identical single chain Fab (scFab) fragments binding to a second antigen are fused to the full-length antibody via a peptide linker at the C terminus of the heavy chains of said full-length antibody.

In one aspect, one or two identical single chain Fab (scFab) fragments binding to a second antigen are fused to the full-length antibody via a peptide linker at the C terminus of the light chains of said full-length antibody.

In one aspect, two identical single chain Fab (scFab) fragments binding to a second antigen are fused to the full-length antibody via a peptide linker at the C-terminus of each heavy or light chain of said full-length antibody.

In one aspect, two identical single chain Fab (scFab) fragments binding to a second antigen are fused to the full-length antibody via a peptide linker at the C-terminus of each heavy chain of said full-length antibody.

In one aspect, two identical single chain Fab (scFab) fragments binding to a second antigen are fused to the full-length antibody via a peptide linker at the C-terminus of each light chain of said full-length antibody.

In one aspect, the bispecific antibody is a trivalent antibody comprising
a) a full-length antibody specifically binding to a first antigen and consisting of two antibody heavy chains and two antibody light chains,
b) a first polypeptide consisting of
   ba) an antibody heavy chain variable domain (VH), or
   bb) an antibody heavy chain variable domain (VH) and an antibody constant domain 1 (CH1),
wherein said first polypeptide is fused with the N-terminus of its VH domain via a peptidic linker to the C-terminus of one of the two heavy chains of said full-length antibody,
c) a second polypeptide consisting of
   ca) an antibody light chain variable domain (VL), or
   cb) an antibody light chain variable domain (VL) and an antibody light chain constant domain (CL),
wherein said second polypeptide is fused with the N-terminus of the VL domain via a peptide linker to the C-terminus of the other of the two heavy chains of said full-length antibody, and
wherein the antibody heavy chain variable domain (VH) of the first polypeptide and the antibody light chain variable domain (VL) of the second polypeptide together form an antigen-binding domain specifically binding to a second antigen.

In one aspect, the antibody heavy chain variable domain (VH) of the polypeptide under b) and the antibody light chain variable domain (VL) of the polypeptide under c) are linked and stabilized via an interchain disulfide bridge by introduction of a disulfide bond between the following positions:
(i) heavy chain variable domain position 44 to light chain variable domain position 100, or
(ii) heavy chain variable domain position 105 to light chain variable domain position 43, or
(iii) heavy chain variable domain position 101 to light chain variable domain position 100 (numbering always according to Kabat EU index).

Techniques to introduce unnatural disulfide bridges for stabilization are described, e.g., in WO 94/029350, Rajagopal, V., et al., Prot. Eng. (1997) 1453-1459; Kobayashi, H., et al., Nucl. Med. Biol. 25 (1998) 387-393; and Schmidt, M., et al., Oncogene 18 (1999) 1711-1721. In one embodiment the optional disulfide bond between the variable domains of the polypeptides under b) and c) is between heavy chain variable domain position 44 and light chain variable domain position 100. In one embodiment the optional disulfide bond between the variable domains of the polypeptides under b) and c) is between heavy chain variable domain position 105 and light chain variable domain position 43 (numbering always according to Kabat). In one embodiment a trivalent, bispecific antibody without said optional disulfide stabilization between the variable domains VH and VL of the single chain Fab fragments is preferred.

In one aspect, the bispecific antibody is a trispecific or tetraspecific antibody, comprising
a) a first light chain and a first heavy chain of a full-length antibody which specifically binds to a first antigen, and
b) a second (modified) light chain and a second (modified) heavy chain of a full-length antibody which specifically binds to a second antigen, wherein the variable domains VL and VH are replaced by each other, and/or wherein the constant domains CL and CH1 are replaced by each other, and
c) wherein one to four antigen-binding domains which specifically bind to one or two further antigens (i.e., to a third and/or fourth antigen) are fused via a peptide linker to the C- or N-terminus of the light chains or heavy chains of a) and/or b).

The antibody under a) does not contain a modification as reported under b) and the heavy chain and the light chain und a) are isolated chains.

In one aspect, the trispecific or tetraspecific antibody comprises under c) one or two antigen-binding domains which specifically bind to one or two further antigens.

In one aspect, the antigen-binding domains are selected from the group of a scFv fragment and a scFab fragment.

In one aspect, the antigen-binding domains are scFv fragments.

In one aspect, the antigen-binding domains are scFab fragments.

In one aspect, the antigen-binding domains are fused to the C-terminus of the heavy chains of a) and/or b).

In one aspect, the trispecific or tetraspecific antibody comprises under c) one or two antigen-binding domains which specifically bind to one further antigen.

In one aspect, the trispecific or tetraspecific antibody comprises under c) two identical antigen-binding domains which specifically bind to a third antigen. In one preferred embodiment such two identical antigen-binding domains are fused both via the same peptidic linker to the C-terminus of the heavy chains of a) and b). In one preferred embodiment the two identical antigen-binding domains are either a scFv fragment or a scFab fragment.

In one aspect, the trispecific or tetraspecific antibody comprises under c) two antigen-binding domains which specifically bind to a third and a fourth antigen. In one embodiment said two antigen-binding domains are fused both via the same peptide connector to the C-terminus of the heavy chains of a) and b). In one preferred embodiment said two antigen-binding domains are either a scFv fragment or a scFab fragment.

In one aspect, the bispecific antibody is a bispecific, tetravalent antibody comprising
a) two light chains and two heavy chains of an antibody, which specifically bind to a first antigen (and comprise two Fab fragments),
b) two additional Fab fragments of an antibody, which specifically bind to a second antigen, wherein said additional Fab fragments are fused both via a peptidic linker either to the C- or N-termini of the heavy chains of a), and
wherein in the Fab fragments the following modifications were performed
(i) in both Fab fragments of a), or in both Fab fragments of b), the variable domains VL and VH are replaced by each other, and/or the constant domains CL and CH1 are replaced by each other, or
(ii) in both Fab fragments of a) the variable domains VL and VH are replaced by each other, and the constant domains CL and CH1 are replaced by each other, and in both Fab fragments of b) the variable domains VL and VH are replaced by each other, or the constant domains CL and CH1 are replaced by each other, or
(iii) in both Fab fragments of a) the variable domains VL and VH are replaced by each other, or the constant domains CL and CH1 are replaced by each other, and in both Fab fragments of b) the variable domains VL and VH are replaced by each other, and the constant domains CL and CH1 are replaced by each other, or
(iv) in both Fab fragments of a) the variable domains VL and VH are replaced by each other, and in both Fab fragments of b) the constant domains CL and CH1 are replaced by each other, or
(v) in both Fab fragments of a) the constant domains CL and CH1 are replaced by each other, and in both Fab fragments of b) the variable domains VL and VH are replaced by each other.

In one aspect, said additional Fab fragments are fused both via a peptidic linker either to the C-termini of the heavy chains of a), or to the N-termini of the heavy chains of a).

In one aspect, said additional Fab fragments are fused both via a peptidic linker to the C-termini of the heavy chains of a).

In one aspect, said additional Fab fragments are fused both via a peptide linker to the N-termini of the heavy chains of a).

In one aspect, in the Fab fragments the following modifications are performed: in both Fab fragments of a), or in both Fab fragments of b), the variable domains VL and VH are replaced by each other, and/or the constant domains CL and CH1 are replaced by each other.

In one aspect, the bispecific antibody is a tetravalent antibody comprising:
a) a (modified) heavy chain of a first antibody, which specifically binds to a first antigen and comprises a first VH-CH1 domain pair, wherein to the C terminus of said heavy chain the N-terminus of a second VH-CH1 domain pair of said first antibody is fused via a peptide linker,
b) two light chains of said first antibody of a),
c) a (modified) heavy chain of a second antibody, which specifically binds to a second antigen and comprises a first VH-CL domain pair, wherein to the C-terminus of said heavy chain the N-terminus of a second VH-CL domain pair of said second antibody is fused via a peptide linker, and
d) two (modified) light chains of said second antibody of c), each comprising a CL-CH1 domain pair.

In one aspect, the bispecific antibody comprises
a) the heavy chain and the light chain of a first full-length antibody that specifically binds to a first antigen, and
b) the heavy chain and the light chain of a second full-length antibody that specifically binds to a second antigen, wherein the N-terminus of the heavy chain is connected to the C-terminus of the light chain via a peptide linker.

The antibody under a) does not contain a modification as reported under b) and the heavy chain and the light chain are isolated chains.

In one aspect, the bispecific antibody comprises
a) a full-length antibody specifically binding to a first antigen and consisting of two antibody heavy chains and two antibody light chains, and
b) an Fv fragment specifically binding to a second antigen comprising a VH2 domain and a VL2 domain, wherein both domains are connected to each other via a disulfide bridge, wherein only either the VH2 domain or the VL2 domain is fused via a peptide linker to the heavy or light chain of the full-length antibody specifically binding to a first antigen.

In the bispecific antibody the heavy chains and the light chains under a) are isolated chains.

In one aspect, the other of the VH2 domain or the VL2 domain is not fused via a peptide linker to the heavy or light chain of the full-length antibody specifically binding to a first antigen.

In all aspects as reported herein the first light chain comprises a VL domain and a CL domain and the first heavy chain comprises a VH domain, a CH1 domain, a hinge region, a CH2 domain and a CH3 domain.

In one aspect, the bispecific antibody is a trivalent antibody comprising
a) two Fab fragments that specifically binds to a first antigen,
b) one CrossFab fragment that specifically binds to a second antigen in which the CH1 and the CL domain are exchanged for each other,
c) one Fc-region comprising a first Fc-region heavy chain and a second Fc region heavy chain,
wherein the C-terminus of CH1 domains of the two Fab fragments are connected to the N-terminus of the heavy chain Fc-region polypeptides, and wherein the C-terminus of the CL domain of the CrossFab fragment is connected to the N-terminus of the VH domain of one of the Fab fragments.

In one aspect, the bispecific antibody is a trivalent antibody comprising
a) two Fab fragments that specifically binds to a first antigen,
b) one CrossFab fragment that specifically binds to a second antigen in which the CH1 and the CL domain are exchanged for each other,
c) one Fc-region comprising a first Fc-region heavy chain and a second Fc region heavy chain,
wherein the C-terminus of CH1 domain of the first Fab fragment is connected to the N-terminus of one of the heavy chain Fc-region polypeptides and the C-terminus of the CL-domain of the CrossFab fragment is connected to the N-terminus of the other heavy chain Fc-region polypeptide, and wherein the C-terminus of the CH1 domain of the second Fab fragment is connected to the N-terminus of the VH domain of the first Fab fragment or to the N-terminus of the VH domain of the CrossFab fragment.

In one aspect, the bispecific antibody comprises
a) a full-length antibody specifically binding to a first antigen and consisting of two antibody heavy chains and two antibody light chains, and
b) a Fab fragment specifically binding to a second antigen comprising a VH2 domain and a VL2 domain comprising a heavy chain fragment and a light chain fragment, wherein within the light chain fragment the variable light chain domain VL2 is replaced by the variable heavy chain domain VH2 of said antibody, and within the heavy chain fragment the variable heavy chain domain VH2 is replaced by the variable light chain domain VL2 of said antibody
wherein the heavy chain Fab fragment is inserted between the CH1 domain of one of the heavy chains of the full-length antibody and the respective Fc-region of the full-length antibody, and the N-terminus of the light chain Fab fragment is conjugated to the C-terminus of the light chain of the full-length antibody that is paired with the heavy chain of the full-length antibody into which the heavy chain Fab fragment has been inserted.

In one aspect, the bispecific antibody comprises
a) a full-length antibody specifically binding to a first antigen and consisting of two antibody heavy chains and two antibody light chains, and
b) a Fab fragment specifically binding to a second antigen comprising a VH2 domain and a VL2 domain comprising a heavy chain fragment and a light chain fragment, wherein within the light chain fragment the variable light chain domain VL2 is replaced by the variable heavy chain domain VH2 of said antibody, and within the heavy chain fragment the variable heavy chain domain VH2 is replaced by the variable light chain domain VL2 of said antibody and wherein the C-terminus of the heavy chain fragment of the Fab fragment is conjugated to the N-terminus of one of the heavy chains of the full-length antibody and the C-terminus of the light chain fragment of the Fab fragment is conjugated to the N-terminus of the light chain of the full-length antibody that pairs with the heavy chain of the full-length antibody to which the heavy chain fragment of the Fab fragment is conjugated.
   B. Dosing of Bispecific Antibodies that Bind to PD-1 and LAG3

For the prevention or treatment of disease, the appropriate dosage of a bispecific antibodies comprising a first antigen-binding domain that specifically binds to PD-1 and a second antigen-binding domain that specifically binds to LAG3 of the invention (when used alone or in combination with one or more other additional therapeutic agents) will depend on the type of disease to be treated, the route of administration, the body weight of the subject, the type of fusion protein, the severity and course of the disease, whether the bispecific antibody is administered for preventive or therapeutic purposes, previous or concurrent therapeutic interventions, the subject's clinical history and response to the fusion protein, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The bispecific antibody comprising a first antigen-binding domain that specifically binds to PD-1 and a second antigen-binding domain that specifically binds to LAG3 as defined herein is suitably administered to the subject at one time or over a series of treatments. Depending on the type and severity of the disease, about 1 μg/kg to 15 mg/kg (e.g., 0.1 mg/kg-10 mg/kg) of the bispecific antibody can be an initial candidate dosage for administration to the subject, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the bispecific antibody would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 5 mg/kg body weight to about 100 mg/kg body weight, about 5 μg/kg body weight to about 500 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the subject. Such doses may be administered intermittently, e.g. every week or every three weeks (e.g. such that the subject receives from about two to about twenty, or e.g. about six doses of the fusion protein). An initial higher loading dose, followed by one or more lower doses may be administered. However, other dosage regimens may be useful. The progress of this therapy is easily monitored by conventional techniques and assays.

In one particular aspect, the bispecific antibody targeting PD-1 and LAG3 is administered to the subject at a fixed dose of about 600 mg every three weeks (Q3W), e.g., at a fixed dose of 600 mg Q3W.
In another aspect, the bispecific antibody targeting PD-1 and LAG3 is administered to the subject at a fixed dose of about 1200 mg every three weeks, e.g., at a fixed dose of 1200 mg Q3W.
In another aspect, the bispecific antibody targeting PD-1 and LAG3 is administered to the subject at a fixed dose of about 2100 mg every two weeks (Q2W), e.g., at a fixed dose of 2100 mg Q2W.

### IX. VEGF Antagonists

VEGF antagonists include any molecule capable of binding VEGF, reducing VEGF expression levels, or neutralizing, blocking, inhibiting, abrogating, reducing, or interfering with VEGF biological activities. An exemplary human VEGF is shown under UniProtKB/Swiss-Prot Accession No. P15692, Gene ID (NCBI): 7422.

In some instances, the VEGF antagonist is an anti-VEGF antibody. In some embodiments, the anti-VEGF antibody is bevacizumab, also known as “rhuMab VEGF” or “AVASTIN®.” Bevacizumab is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879, issued Feb. 26, 2005, the entire disclosure of which is expressly incorporated herein by reference.

Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al. (Journal of Immunological Methods 288:149-164, 2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89.

In other instances, the VEGF antagonist is an anti-VEGFR2 antibody or related molecule (e.g., ramucirumab, tanibirumab, aflibercept); an anti-VEGFR1 antibody or related molecules (e.g., icrucumab, aflibercept (VEGF Trap-Eye; EYLEA®), or ziv-aflibercept (VEGF Trap; ZALTRAP®)); a bispecific VEGF antibody (e.g., MP-0250, vanucizumab (VEGF-ANG2), or bispecific antibodies disclosed in US 2001/0236388); a bispecific antibody including a combination of two of anti-VEGF, anti-VEGFR1, and anti-VEGFR2 arms; an anti-VEGFA antibody (e.g., bevacizumab, sevacizumab); an anti-VEGFB antibody; an anti-VEGFC antibody (e.g., VGX-100), an anti-VEGFD antibody; or a nonpeptide small molecule VEGF antagonist (e.g., pazopanib, axitinib, vandetanib, stivarga, cabozantinib, lenvatinib, nintedanib, orantinib, telatinib, dovitinib, cediranib, motesanib, sulfatinib, apatinib, foretinib, famitinib, or tivozanib). In some examples, the VEGF antagonist may be a tyrosine kinase inhibitor, including a receptor tyrosine kinase inhibitors (e.g., a multi-targeted receptor tyrosine kinase inhibitor such as sunitinib or axitinib).

### X. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used for treating a subject having a cancer.

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′) 2 fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, K_{D}036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:
(a) an HVR-H1, HVR-H2, and HVR-H3 sequence of GFTFSDSWIH (SEQ ID NO: 64), AWISPYGGSTYYADSVKG (SEQ ID NO: 65) and RHWPGGFDY (SEQ ID NO: 66), respectively, and
(b) an HVR-L1, HVR-L2, and HVR-L3 sequence of RASQDVSTAVA (SEQ ID NO: 67), SASFLYS (SEQ ID NO: 68) and QQYLYHPAT (SEQ ID NO: 69), respectively.

In one embodiment, the anti-PD-L1 antibody comprises:
(a) a heavy chain variable region (VH) comprising the amino acid sequence: EVOLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 70), and
(b) the light chain variable region (VL) comprising the amino acid sequence: DIQMTQSPSSLSASVGDRVTITCRASQDVSTAVAWYQQKPGKAPKLLIYSASFLYSGVPSRFSGSGSGTD FTLTISSLOPEDFATYYCQQYLYHPATFGQGTKVEIKR (SEQ ID NO: 71).

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:

(a) the heavy chain amino acid sequence: EVOLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSSASTKGPSVFPLAPSSKSTS GGTAALGCLVKDYFPEPVTVSWNSGALTSGVHTFPAVLQSSGLYSLSSVVTVPSSSLGTQTYICNVNHKP SNTKVDKKVEPKSCDKTHTCPPCPAPELLGGPSVFLFPPKPKDTLMISRTPEVTCVVVDVSHEDPEVKFN WYVDGVEVHNAKTKPREEQYASTYRVVSVLTVLHQDWLNGKEYKCKVSNKALPAPIEKTISKAKGQPRE PQVYTLPPSREEMTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 62), and

(b) the light chain amino acid sequence: DIQMTQSPSSLSASVGDRVTITCRASQDVSTAVAWYQQKPGKAPKLLIYSASFLYSGVPSRFSGSGSGTD FTLTISSLQPEDFATYYCQQYLYHPATFGQGTKVEIKRTVAAPSVFIFPPSDEQLKSGTASVVCLLNNFYPR EAKVQWKVDNALQSGNSQESVTEQDSKDSTYSLSSTLTLSKADYEKHKVYACEVTHQGLSSPVTKSFNR GEC (SEQ ID NO: 63).

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen-binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

### B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DClg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances, the anti-PD-1 antibody is a monoclonal antibody. In some instances, the anti-PD-1 antibody is an antibody fragment selected from the group consisting of Fab, Fab′, Fab′-SH, Fv, scFv, and (Fab′) 2 fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

### C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′) 2 fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

### XI. Pharmaceutical Compositions and Formulations

Also provided herein are pharmaceutical compositions and formulations comprising a bispecific antibody targeting PD-1 and LAG3 and, optionally, a pharmaceutically acceptable carrier. The disclosure also provides: (i) pharmaceutical compositions and formulations comprising a bispecific antibody targeting PD-1 and LAG3 and an anti-VEGF antibody (e.g., bevacizumab), and optionally, a pharmaceutically acceptable carrier; and (ii) pharmaceutical compositions and formulations comprising an anti-TIGIT antagonist antibody and a bispecific antibody targeting PD-1 and LAG3 and optionally, a pharmaceutically acceptable carrier. Pharmaceutical compositions and formulations of a bispecific antibody targeting PD-1 and LAG3 and/or other agents described herein (e.g., dexamethasone) can be prepared by mixing the agent or agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. In some embodiments, mosunetuzumab is formulated for administration subcutaneously. In some embodiments, mosunetuzumab is formulated for administration intravenously.

Pharmaceutical compositions and formulations as described herein can be prepared by mixing the active ingredients (e.g., a bispecific antibody targeting PD-1 and LAG3, an anti-TIGIT antagonist antibody, and/or an anti-VEGF antibody) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions.

An exemplary tiragolumab formulation comprises a histidine solution containing polysorbate 20, sucrose, L-methionine, and WFI. Tiragolumab may be provided in a 15-mL vial containing 10 ml of tiragolumab drug product at an approximate concentration of tiragolumab antibody of 60 mg/mL.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

### XII. Articles of Manufacture or Kits

### A. Kits Comprising a Bispecific Antibody Targeting PD-1 and LAG3 and an Anti-TIGIT Antagonist Antibody

In another aspect, provided herein is an article of manufacture or a kit comprising a bispecific antibody targeting PD-1 and LAG3 and an anti-TIGIT antagonist antibody (e.g., tiragolumab). In some instances, the article of manufacture or kit further comprises a package insert comprising instructions for using the anti-TIGIT antagonist antibody in combination with the bispecific antibody targeting PD-1 and LAG3 to treat or delay progression of cancer in a subject. Any of the bispecific antibody targeting PD-1 and LAG3 and/or anti-TIGIT antagonist antibodies described herein may be included in the article of manufacture or kits.

In another embodiment of the invention, a kit is provided comprising a bispecific antibody targeting PD-1 and LAG3 for use in combination with an anti-TIGIT antagonist antibody for treating a subject having a cancer according to any of the methods described herein. In some instances, the kit further comprises the anti-TIGIT antagonist antibody. In some instances, the article of manufacture or kit further comprises a package insert comprising instructions for using the bispecific antibody targeting PD-1 and LAG3 in combination with the anti-TIGIT antagonist antibody (e.g., tiragolumab) to treat or delay progression of a cancer in a subject.

In some instances, the bispecific antibody targeting PD-1 and LAG3 and the anti-TIGIT antagonist antibody are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags and syringes.

Any of the bispecific antibodies targeting PD-1 and LAG3 and/or anti-TIGIT antagonist antibodies described herein may be included in the article of manufacture or kits. Any of the articles of manufacture or kits may include instructions to administer a bispecific antibody targeting PD-1 and LAG3 and/or an anti-TIGIT antagonist antibody to a subject in accordance with any of the methods described herein, e.g., any of the methods set forth in Section III above.

### B. Kits Comprising a Bispecific Antibody Targeting PD-1 and LAG3 and an Anti-VEGF Antibody

In another aspect, provided herein is an article of manufacture or a kit comprising a bispecific antibody targeting PD-1 and LAG3 and an anti-VEGF antibody (e.g., bevacizumab). In some instances, the article of manufacture or kit further comprises a package insert comprising instructions for using the anti-VEGF antibody in combination with the bispecific antibody targeting PD-1 and LAG3 to treat or delay progression of cancer in a subject. Any of the bispecific antibody targeting PD-1 and LAG3 and/or anti-VEGF antibodies described herein may be included in the article of manufacture or kits.

In another embodiment of the invention, a kit is provided comprising a bispecific antibody targeting PD-1 and LAG3 for use in combination with an anti-VEGF antibody for treating a subject having a cancer according to any of the methods described herein. In some instances, the kit further comprises the anti-VEGF antibody. In some instances, the article of manufacture or kit further comprises a package insert comprising instructions for using the bispecific antibody targeting PD-1 and LAG3 in combination with the anti-VEGF antibody to treat or delay progression of a cancer in a subject.

In some instances, the bispecific antibody targeting PD-1 and LAG3 and the anti-VEGF antibody are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags and syringes.

Any of the bispecific antibodies targeting PD-1 and LAG3 and/or anti-VEGF antibodies described herein may be included in the article of manufacture or kits. Any of the articles of manufacture or kits may include instructions to administer a bispecific antibody targeting PD-1 and LAG3 and/or an anti-VEGF antibody to a subject in accordance with any of the methods described herein, e.g., any of the methods set forth in Section III above.

### EXAMPLES

### Example 1: A Phase Ib/II, Open-Label, Multicenter, Randomized Umbrella Study Evaluating the Efficacy and Safety of Multiple Treatment Combinations in Patients with Melanoma

Melanoma is a potentially deadly form of skin cancer and is one of the fastest-growing malignancies (Algazi et al. Cancer Manag Res. 2:197-211, 2010; Finn et al. BMC Med. 10:23, 2012). More than 300,000 people worldwide are currently diagnosed with melanoma each year, and 57,000 people die of the disease. The clinical outcomes of patients with melanoma are highly dependent on the stage at presentation. Most people who present with more advanced melanoma have a poor prognosis (Finn et al. BMC Med. 10:23, 2012). Patients with lymph-node involvement (Stage III) have a high risk of local and distant relapse after surgery, and the 5-year survival rate is 32%-93% in this patient group (Gershenwald et al. C A Cancer J Clin. 67:472-492, 2017). Few patients have metastatic disease (Stage IV) at presentation, but some develop metastases after their initial definitive treatment. Immunotherapy and targeted therapies have improved the outcomes of those patients, and the 5-year survival rate is around 50% (Larkin et al. N Engl J Med. 373:23-34, 2015; Wolchok et al. N Engl J Med. 377:1345-1356, 2017; Larkin et al. N Engl J Med. 381:1535-1546, 2019; Robert et al. Lancet Oncol. 20:1239-1251, 2019; Long et al. J Clin Oncol. 38 (Suppl 15): 10013, 2020). Despite recent therapeutic advances, melanoma continues to be a serious health issue, with a high medical need and a steadily increasing incidence over the past 30 years (Bataille. Expert Rev Dermatol. 4:533-539, 2009).

BO43328 is a Phase Ib/II, open-label, multicenter, randomized, umbrella study in patients with resectable Stage III (Cohort 1) or Stage IV (Cohort 2) melanoma. The study is designed with the flexibility to open new treatment arms as new treatments become available, close existing treatment arms that demonstrate minimal clinical activity or unacceptable toxicity, modify the patient population (e.g., with regard to prior anti-cancer treatment or biomarker status), or introduce additional cohorts of patients with other types of melanoma.

### A. Overview of Study Design

This study evaluates the efficacy, safety, and pharmacokinetics of treatment combinations in cancer immunotherapy (CIT)-naive patients with resectable Stage III melanoma (Cohort 1) and in patients with Stage IV melanoma (Cohort 2). Specific objectives and corresponding endpoints for the study are outlined below for Cohort 1 (see Table 5) and Cohort 2 (see Table 6).

TABLE 5
Objectives and Corresponding Endpoints for Cohort 1

| | Corresponding Endpoints |
|---|---|
| Primary Efficacy Objective | |
| To evaluate the efficacy | pRR, (defined as the proportion of |
| of treatment | patients with pCR, pnCR, and pPR) at |
| | time of surgery, as determined by |
| | independent pathologic review. |
| Secondary Efficacy | |
| Objective | |
| To evaluate the efficacy | pRR (defined as the proportion of |
| of treatment | patients with pCR, pnCR, and pPR) |
| | at time of surgery, as determined |
| | by local pathologic assessment. |
| | EFS, defined as the time from |
| | randomization to any of the following |
| | events (whichever occurs first): |
| | Disease progression that precludes |
| | surgery, as assessed by the |
| | investigator according to RECIST |
| | v1.1; local, regional or distant |
| | disease recurrence; or death from |
| | any cause. |
| | RFS, defined as the time from surgery |
| | to the first documented recurrence |
| | of disease or death from any cause. |
| | OS, defined as the time from |
| | randomization to death from any |
| | cause. |
| | ORR, defined as the proportion of |
| | patients with a CR or PR as |
| | determined by the investigator |
| | according to RECIST v1.1, prior to |
| | surgery. |
| | Responses are assessed and determined |
| | according to RECIST v1.1 but are not |
| | required to be confirmed by later |
| | imaging studies. |
| Exploratory Efficacy | |
| Objective | |
| To evaluate the efficacy | Landmark EFS, defined as the time |
| of treatment | from randomization to any of the |
| | following events (whichever occurs |
| | first): Disease progression that |
| | precludes surgery, as assessed by |
| | the investigator according to RECIST |
| | v1.1; local, regional or distant |
| | disease recurrence; or death from |
| | any cause at specific timepoints |
| | (1, 2, 3, and 5 years). |
| | Landmark RFS, defined as the time |
| | from surgery to the first documented |
| | recurrence of disease or death from |
| | any cause at specific timepoints |
| | (1, 2, 3, and 5 years). |
| | Landmark OS, defined as the time |
| | from randomization to death from |
| | any cause at specific timepoints |
| | (1, 2, 3, and 5 years). |
| Safety Objective | |
| To evaluate the safety | Incidence, nature, and severity of |
| of treatment | adverse events and laboratory |
| | abnormalities, with severity |
| | determined according to NCI |
| | CTCAE v5.0. |
| | CRS severity is also determined |
| | according to the ASTCT CRS |
| | Consensus Grading Scale. |
| | Incidence and nature of immune- |
| | related adverse events Grade ≥3 |
| | during the first 12 weeks. |
| | Rate and duration of delayed |
| | surgery due to treatment-related |
| | adverse events. |
| | Surgical complication rates according |
| | to Clavien-Dindo surgical classification |
| | after CLND. |
| Exploratory Pharmacokinetic | |
| Objectives | |
| To characterize the PK | Plasma or serum concentrations of |
| profile of drugs that are | each drug (as appropriate) at |
| administered as part of | specified timepoints |
| treatment | |
| To evaluate potential | Relationship between plasma or serum |
| relationships between drug | concentration or PK parameters for |
| exposure and the efficacy | each drug (as appropriate, on the |
| and safety of treatment | basis of available data) and efficacy |
| | endpoints. |
| | Relationship between plasma or serum |
| | concentration or PK parameters for |
| | each drug (as appropriate, on the |
| | basis of available data) and safety |
| | endpoints. |
| Exploratory Immunogenicity | |
| Objectives | |
| To evaluate the immune | For drugs for which ADA formation is |
| response to drugs that are | measured: Presence of ADAs during the |
| administered | study relative to the presence of |
| | ADAs at baseline. |
| To evaluate potential | For drugs for which ADA formation is |
| effects of ADAs | measured: Relationship between ADA |
| | status and efficacy, safety, or |
| | PK endpoints. |
| Exploratory Biomarker | |
| Objective | |
| To identify biomarkers | Relationship between biomarkers in |
| biology that are predictive | blood and tumor tissue and efficacy, |
| of response to study | safety, PK, immunogenicity, or |
| treatment (i.e., predictive | other biomarker endpoints. |
| biomarkers), are associated | |
| with progression to a more | |
| severe disease state (i.e., | |
| prognostic biomarkers), are | |
| associated with resistance | |
| to study treatment, are | |
| associated with susceptibility | |
| to developing adverse events | |
| (i.e., safety biomarkers), | |
| can provide evidence of study | |
| treatment activity (i.e., | |
| pharmacodynamic biomarkers), | |
| or can increase the knowledge | |
| and understanding of disease | |
| biology. | |

ADA = anti-drug antibody; ASTCT = American Society for Transplantation and Cellular Therapy; CLND = completion lymph node dissection; CR = complete response; CRS = cytokine-release syndrome; EFS = event-free survival; NCI CTCAE v5.0 = National Cancer Institute Common Terminology Criteria for Adverse Events, Version 5.0; ORR = objective response rate; OS = overall survival; pCR = pathologic complete response; PK = pharmacokinetic; pnCR = pathologic near complete response; pPR = pathologic partial response; PR = partial response; pRR = pathologic response rate; RECIST v1.1 = Response Evaluation Criteria in Solid Tumors, Version 1.1; RFS = relapse-free survival.

TABLE 6
Objectives and Corresponding Endpoints for Cohort 2

| | Corresponding Endpoint |
|---|---|
| Primary Efficacy Objective | |
| To evaluate the efficacy | ORR, defined as the proportion of |
| of treatment | patients with a CR or PR on two |
| | consecutive occasions ≥4 weeks |
| | apart, as determined by the |
| | investigator according to |
| | RECIST v1.1. |
| Secondary Efficacy | |
| Objective | |
| To evaluate the efficacy | PFS after randomization/enrollment, |
| of treatment | defined as the time from |
| | randomization/enrollment to the |
| | first occurrence of disease |
| | progression or death from any cause |
| | (whichever occurs first), as |
| | determined by the investigator |
| | according to RECIST v1.1. |
| | OS after randomization/enrollment, |
| | defined as the time from |
| | randomization/enrollment to death |
| | from any cause. |
| | OS at specific timepoints (e.g., |
| | 6 months). |
| | DOR, defined as the time from the |
| | first occurrence of a documented |
| | objective response to disease |
| | progression or death from any cause |
| | (whichever occurs first), as |
| | determined by the investigator |
| | according to RECIST v1.1. |
| | Disease control, defined as stable |
| | disease for ≥12 weeks or a CR or |
| | PR, as determined by the investigator |
| | according to RECIST v1.1. |
| Exploratory Efficacy | |
| Objective | |
| To evaluate the efficacy | ORR, PFS, DOR, and disease control |
| of treatment | as determined by the investigator |
| | according to iRECIST. |
| Safety Objective | Corresponding Endpoint |
| To evaluate the safety | Incidence, nature, and severity of |
| of treatment | adverse events and laboratory |
| | abnormalities, with severity |
| | determined according to NCI |
| | CTCAE v5.0. |
| | CRS severity will also be determined |
| | according to the ASTCT CRS |
| | Consensus Grading Scale. |
| Exploratory | |
| Pharmacokinetic Objective | |
| To characterize the PK | Plasma or serum concentration of |
| profile of drugs that are | each drug (as appropriate) at |
| administered as part of | specified timepoints. |
| treatment | |
| To evaluate potential | Relationship between plasma or |
| relationships between drug | serum concentration or PK parameters |
| exposure and the efficacy | for each drug (as appropriate on |
| and safety of treatment. | the basis of available data) and |
| | efficacy endpoints. |
| | Relationship between plasma or |
| | serum concentration or PK parameters |
| | for each drug (as appropriate on |
| | the basis of available data) and |
| | safety endpoints. |
| Exploratory | |
| Immunogenicity Objectives | |
| To evaluate the immune | For drugs for which ADA formation is |
| response to drugs that | measured: Presence of ADAs during |
| are administered. | the study relative to the presence of |
| | ADAs at baseline. |
| To evaluate potential effects | For drugs for which ADA formation is |
| of ADAs. | measured: Relationship between ADA |
| | status and efficacy, safety, or PK |
| | endpoints. |
| Exploratory Biomarker | |
| Objective | |
| To identify biomarkers that | Relationship between biomarkers in |
| are predictive of response | blood and tumor tissue and efficacy, |
| to study treatment (i.e., | safety, PK, immunogenicity, or other |
| predictive biomarkers), are | biomarker endpoints. |
| associated with progression | |
| to a more severe disease | |
| state (i.e., prognostic | |
| biomarkers), are associated | |
| with resistance to study | |
| treatment, are associated | |
| with susceptibility to | |
| developing adverse events | |
| (i.e., safety biomarkers), | |
| can provide evidence of | |
| study treatment activity | |
| (i.e., pharmacodynamic | |
| biomarkers), or can | |
| increase the knowledge and | |
| understanding of disease | |
| biology | |

ADA = anti-drug antibody; ASTCT = American Society for Transplantation and Cellular Therapy; CR = complete response; CRS = cytokine-release syndrome; DOR = duration of response; iRECIST = modified RECIST v1.1 for immune-based therapeutics; NCI CTCAE v5.0 = National Cancer Institute Common Terminology Criteria for Adverse Events, Version 5.0; ORR = objective response rate; OS = overall survival; PFS = progression-free survival; PK = pharmacokinetic; PR = partial response; RECIST v1.1 = Response Evaluation Criteria in Solid Tumors, Version 1.1.
Note:
Overall response at a single timepoint is assessed by the investigator using RECIST v1.1.

Two cohorts are enrolled in parallel in this study. Cohort 1 enrolls patients with resectable Stage III melanoma with measurable lymph node metastases according to Response Evaluation Criteria in Solid Tumors, Version 1.1 (RECIST v1.1) that can be biopsied, who have no history of in-transit metastases within the last 6 months, and who have not received systemic CIT for their disease, e.g., PD-1/PD-L1 and/or CTLA-4 blocking agents or other agents.

Cohort 2 enrolls patients with Stage IV melanoma who experienced disease progression during or after at least one but not more than two lines of treatment for metastatic disease. Up to two lines of checkpoint inhibition therapy (monotherapy or combination therapy) are allowed. Patients with BRAF-mutant disease may have received an additional line of targeted therapy (either before, intermittent with, or after the checkpoint inhibition therapy) or may have received targeted therapy and checkpoint inhibition therapy concurrently as one combination treatment.

### Treatment Assignment

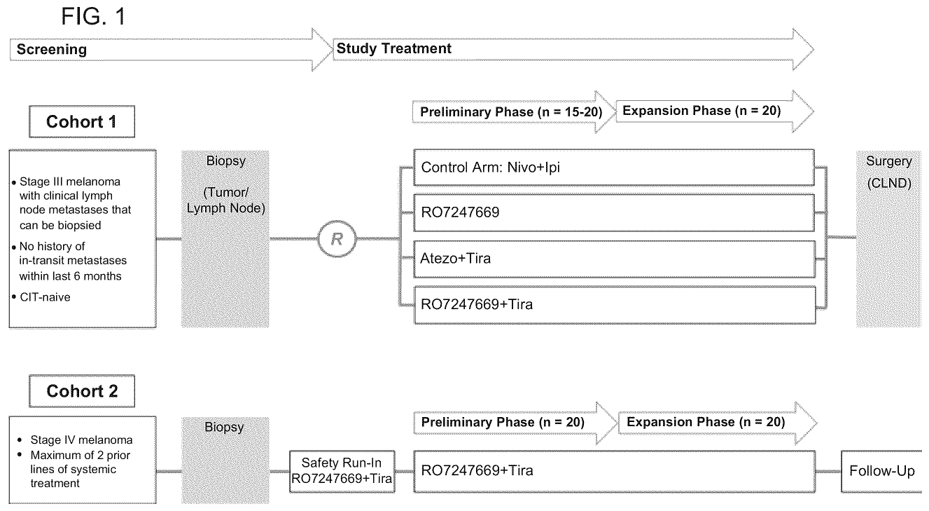

In Cohort 1, patients are randomly assigned to a control arm (nivolumab plus ipilimumab (Nivo+Ipi)) or an experimental arm consisting of RO7247669 (a bispecific antibody that binds to PD-1 and LAG3), atezolizumab in combination with tiragolumab (Atezo+Tira), or RO7247669 in combination with tiragolumab (RO7247669+Tira). Patients are stratified by geographic region (Australia vs. Rest of the World) and baseline LDH (≤ the upper limit of normal (ULN) vs. >ULN). Details on the treatment regimens are provided in Table 7 and FIG. 1.

In Cohort 2, patients are enrolled into an experimental arm consisting of RO7247669 in combination with tiragolumab (RO7247669+Tira). Enrollment begins with a 6-patient safety run-in phase.

Approximately 61-191 patients are enrolled during the study, including approximately 6 patients who are enrolled in the safety run-in phase of Cohort 2. Enrollment within the experimental arms takes place in two phases: a preliminary phase, followed by an expansion phase. Approximately 15-20 patients are enrolled in each treatment arm during the preliminary phase. If clinical activity (pathologic response in Cohort 1) is observed in an experimental arm during the preliminary phase, approximately 20 additional patients may be enrolled in that arm during the expansion phase.

The Sponsor may decide to delay or suspend enrollment within a given treatment arm. Experimental arms with insufficient clinical activity or unacceptable toxicity are not expanded. Additional patients may be enrolled to ensure balance among treatment arms with respect to demographic and baseline characteristics, including potential predictive biomarkers, in order to enable further subgroup analyses.

The randomization ratio depends on the number of experimental arms that are available (e.g., if an arm is added or enrollment in an arm is suspended, pending analysis of results from the preliminary phase), with the stipulation that the likelihood of being allocated to the control arm is no more than 35%. Randomization takes into account arm-specific exclusion criteria. Patients are ineligible for a specific arm if they meet any of the exclusion criteria outlined for that arm.

Details on the treatment regimens are provided in Table 7.

TABLE 7
Treatment Regimens

| | | Number of | | |
|---|---|---|---|---|
| | | Patients | | |
| | | (Sponsor | Number of Patients | |
| | | Assignment) ^{b} | (Random Assignment) ^{c} | |
| | Study | Safety Run- | Preliminary | Expansion |
| Cohort | Treatment ^{a} | in Phase | Phase | Phase ^{d} |
| 1 | Control arm: | N/A | Variable ^{c} | |
| | nivolumab + | | | |
| | ipilimumab | | | |
| 1 | RO7247669 | N/A | 20 ^{e} | 20 |
| 1 | Atezolizumab + | N/A | 20 ^{e} | 20 |
| | tiragolumab | | | |
| 1 | RO7247669 + | N/A | 20 ^{e} | 20 |
| | tiragolumab ^{f} | | | |
| 2 | RO7247669 + | ~6 | 20 | 20 |
| | tiragolumab | | | |

^{a} The Sponsor may decide to delay or suspend enrollment within a given treatment arm. Thus, all experimental arms may not open for enrollment at the same time.
^{b} During the safety run-in phase, patients are assigned to available treatment arms. The treatment assignment ratio depends on the number of experimental arms that are open for enrollment.
^{c} The randomization ratio depends on the number of experimental arms that are open for randomization (e.g., if an arm is added or randomization into an arm is suspended pending analysis of results from the preliminary phase), with the stipulation that the likelihood of being allocated to the control arm is no more than 35%.
^{d} If clinical activity is observed in an experimental arm during the preliminary phase, approximately 20 additional patients are enrolled in that arm during the expansion phase. Experimental arms with minimal clinical activity or unacceptable toxicity do not undergo expansion.
^{e} Enrollment is suspended in the Cohort 1 RO7247669, Atezo + Tira, and RO7247669 + Tira arms to allow for a safety evaluation in a minimum of 6 patients.
^{f} Enrollment in the RO7247669 + Tira arm opens in Cohort 1 after safety assessment of the treatment combination in Cohort 2.

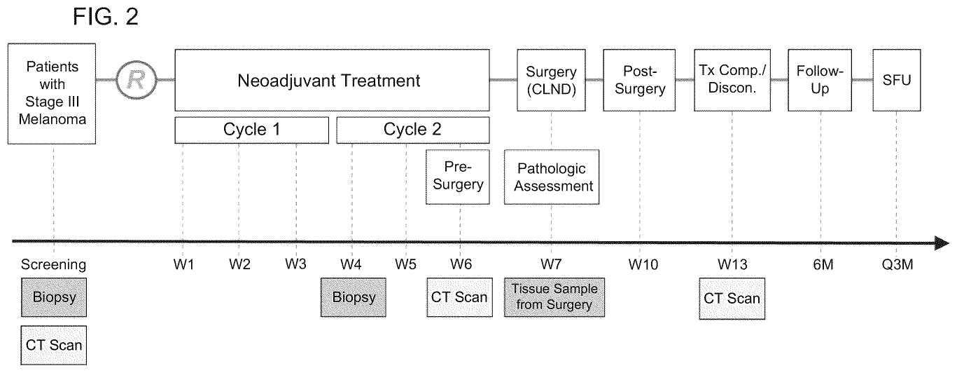

In Cohort 1, patients in the control arm and the experimental arms receive neoadjuvant treatment during a 6-week period. After completion of neoadjuvant treatment, or in case of discontinuation due to toxicity and in absence of disease progression, patients undergo surgery (completion lymph node dissection (CLND)) in Week 7. At the discretion of the investigator, outside this study, patients subsequently start either adjuvant therapy or observation commencing in Week 13 (FIG. 2).

Because of the possibility of an initial increase in the size of metastatic lymph nodes caused by immune-cell infiltration in the context of a T-cell response (termed pseudoprogression) with CITs, suspected clinical or radiographic progression per RECIST v1.1 may not be indicative of true disease progression. In the absence of unacceptable toxicity, patients who meet the criteria for disease progression per RECIST v1.1 while receiving treatment with a CIT drug are permitted to continue study treatment until surgery. Before discontinuation of study treatment and/or cancellation of surgery, progression is confirmed by biopsy or repeated radiographic assessment by an additional expert reviewer. All patients are expected to proceed with surgery, provided that there are no distant metastases and the surgeon considers the disease to be completely resectable.

In Cohort 2, patients continue to receive treatment until unacceptable toxicity or loss of clinical benefit as determined by the investigator after an integrated assessment of radiographic and biochemical data, local biopsy results (if available), and clinical status (e.g., symptomatic deterioration such as pain secondary to disease). Because of the possibility of an initial increase in tumor burden caused by immune-cell infiltration in the setting of a T-cell response (termed pseudoprogression) with atezolizumab and other CITs, radiographic progression per RECIST v1.1 may not be indicative of true disease progression. In the absence of unacceptable toxicity, patients who meet criteria for disease progression per RECIST v1.1 while receiving treatment with a CIT combination are permitted to continue treatment if they meet all of the following criteria:
Evidence of clinical benefit, as determined by the investigator following a review of all available data.
Absence of symptoms and signs (including laboratory values, such as new or worsening hypercalcemia) indicating unequivocal progression of disease.
Absence of decline in Eastern Cooperative Oncology Group (ECOG) Performance Status that can be attributed to disease progression.
Absence of tumor progression at critical anatomical sites (e.g., leptomeningeal disease) that cannot be managed by protocol-allowed medical interventions.

Patients eligible for treatment beyond progression are informed by the investigator that they may be foregoing other treatment options known to confer clinical benefit while continuing to receive the study treatment. Patients have the right to voluntarily withdraw from the study at any time for any reason. In addition, the investigator has the right to withdraw a patient from the study for medical conditions that the investigator or Sponsor determines may jeopardize the patient's safety if he/she continues in the study.

If at a subsequent tumor assessment, pseudoprogression is ruled out and progression of the disease is confirmed, the patient is discontinued from study treatment.

### Safety Evaluation Phase (Cohort 1)

To evaluate the toxicities of the experimental treatments in the neoadjuvant setting, enrollment is suspended after approximately 6 patients have been enrolled to allow for a safety evaluation. The safety evaluation is based on safety data from a minimum of 6 patients who have received at least one dose of treatment (i.e., one dose of each agent for a given combination) and who have completed safety follow-up assessments until surgery. Notably, timely conduct of surgery (CLND) is an indicator of treatment tolerability. During the 6-patient safety evaluation, or at any time following the safety evaluation, if ≥30% of patients experience one or more of the following events that is considered to be at least possibly related to study treatment, enrollment for that combination is put on hold while the Sponsor evaluates the benefit-risk profile of that treatment:
A treatment-related Grade ≥3 adverse event that does not improve (with or without treatment) to Grade 2 or better within 2 weeks.
A treatment-related adverse event causing >2-week delay in surgery.
A treatment-related serious adverse event.
A treatment-related adverse event that requires permanent discontinuation of study drug.
Death, except those that are incontrovertibly related to disease progression or extraneous causes such as accidents.

If no new safety signals are detected, enrollment resumes in that arm.

### Safety Run-In Phase (Cohort 2)

To assess the safety and tolerability of novel combinations that are tested clinically for the first time, an initial safety run-in phase is implemented in Cohort 2. Approximately 6 patients with metastatic disease are treated with the novel combination (i.e., RO7247669+Tira) and assessed for safety and tolerability for a minimum of 28 days.

A minimum of 6 patients in Cohort 2 must complete the initial safety run-in phase. If the RO7247669+Tira combination is determined to be tolerable, enrollment for the preliminary phase may be opened, and the RO7247669+Tira arm in Cohort 1 can be opened for enrollment. Patients in the safety run-in phase are enrolled and treated in a sequential manner, with at least one week between the first patient and the remaining patients.

The assessment is based on safety data from a minimum of 6 patients who have received at least one dose of treatment (i.e., one dose of each agent) and who have completed safety follow-up assessments for at least 28 days. If ≥30% of patients experience one or more of the following events that is considered to be at least possibly related to study treatment, enrollment for that combination is put on hold while the Sponsor evaluates the benefit-risk profile of that treatment:
A treatment-related Grade ≥3 adverse event that does not improve (with or without treatment) to Grade 2 or better within 2 weeks.
A treatment-related serious adverse event.
A treatment-related adverse event that requires permanent discontinuation of study drug.
Death, except those that are incontrovertibly related to disease progression or extraneous causes such as accidents.

If no new safety signals are detected, the combination is also initiated in Cohort 1.

### B. End of Study and Length of Study

The end of this study is defined as the date when the last patient completes the last visit, including survival follow-up visits conducted by telephone or in the clinic. The total length of the study, from screening of the first patient to the end of the study, is expected to be approximately 5 years.

### C. Rationale for Study Design

### Rationale for Patient Population

Cohort 1 enrolls patients with resectable Stage III melanoma with measurable lymph node metastases (according to RECIST v1.1) that can be biopsied, and who have no history of in-transit metastases within the last 6 months. Enrolled patients must not have received prior immunotherapy for their disease.

This same patient population is enrolled in the OpACIN and OpACIN-neo studies, including the PRADO extension cohort. These studies evaluated the neoadjuvant (and adjuvant) combination of nivolumab and ipilimumab in patients with resectable melanoma. Neoadjuvant therapy was found to have a statistically significant and clinically meaningful benefit as compared with adjuvant therapy (Rozeman et al. Lancet Oncol. 20:948-960, 2019; Blank et al. J Clin Oncol. 38: 15S, 2020; Rozeman et al. Nat Med. 27:256-263, 2021). In addition, the safety profile of the treatment was found to be tolerable in an optimized treatment schedule.

Despite the recently demonstrated benefit of checkpoint inhibition therapy, there is a continuing need for treatment regimens that are more efficacious (i.e., broader and deeper pathologic response in the surgical specimen) and better tolerated for patients with resectable melanoma. The multiple treatment options in this study are expected to stimulate the immune system through a variety of mechanisms. The aim is to extend the benefit of CIT beyond that of current checkpoint inhibition to a larger population with resectable melanoma.

Cohort 2 enrolls patients with Stage IV melanoma who experienced disease progression during or after at least one but not more than two lines of treatment for metastatic disease. Up to two lines of checkpoint inhibition therapy (monotherapy or combination therapy) are allowed. Patients with BRAF-mutant disease may have received an additional line of targeted therapy (either before, intermittent with, or after the checkpoint inhibition therapy) or may have received targeted therapy and checkpoint inhibition therapy concurrently as one combination treatment.

Novel combinations of compounds with a clinical and/or biological rationale for anti-melanoma activity that have not yet been tested clinically are investigated in Cohort 2. Importantly, for individual compounds considered for novel combinations, safety and tolerability have been already established in other studies, and a safe dose and schedule are available. The Cohort 2 safety run-in phase assesses the safety of the novel combination with regards to potential overlapping toxicities.

### D. Inclusion Criteria

### Shared Inclusion Criteria for Cohort 1 and Cohort 2

Patients must meet all of the following criteria to qualify for Cohort 1 and Cohort 2:
Age≥18 years at the time of signing Informed Consent Form.
Availability of a representative tumor specimen that is suitable for determination of PD-L1 and/or additional biomarker status via central testing.
   Baseline tumor tissue samples are collected from all patients (except patients in the Cohort 2 safety run-in phase) by biopsy of a metastatic lymph node (Cohort 1) or other metastatic lesion (Cohort 2) at screening.
   In addition, archival primary tumor tissue is submitted from all patients if available. In case no archival primary tissue is available (e.g., for patients with unknown primary tumor), enrollment is permitted. For archival tissue, a formalin-fixed, paraffin-embedded (FFPE) tumor specimen in a paraffin block (preferred) with sufficient size and tumor content representation, preferably including the invasive margin, or at least 16 slides containing unstained, freshly cut, serial sections must be submitted along with an associated pathology report. If only 10-15 slides are available, the patient is still eligible for the study.
Adequate hematologic and end-organ function, defined by the following laboratory test results, obtained within 14 days prior to initiation of study treatment: Absolute neutrophil count (ANC)≥1.5×10⁹/L (1500/μL); lymphocyte count≥0.5×10⁹ cells/L (500/μL) (borderline machine lymphocyte counts may be confirmed by a manual count); platelet count≥100×10⁹/L (100,000/μL); hemoglobin≥90 g/L (9 g/dL); AST, ALT, and ALP≤2.5×ULN (participants with documented liver metastases may have AST and ALT≤5×ULN; participants with documented liver or bone metastasis may have ALP≤5×ULN); total bilirubin≤1.5×ULN (patients with known Gilbert disease may have bilirubin level≤3×ULN); creatinine≤1.5×ULN or creatinine clearance≥30 mL/min (calculated using the Cockcroft-Gault formula); serum albumin≥25 g/L (2.5 g/dL). Patients not receiving therapeutic anticoagulation may have INR and aPTT≤1.5×ULN.
For patients receiving therapeutic anticoagulation: stable anticoagulant regimen (i.e., no new thrombosis, thromboembolic event, or bleeding episode within 3 months prior to study treatment start).
Negative HIV test at screening. Patients without a prior positive HIV test result undergo an HIV test at screening, unless not permitted per local regulations.
Negative hepatitis B surface antibody (HBsAb), and negative total hepatitis B core antibody (HBcAb) test at screening. If a patient has a negative hepatitis B surface antigen (HBsAg) test and a positive total HBcAb test at screening, an hepatitis B virus (HBV) DNA test must also be performed to rule out active HBV.
Negative hepatitis C virus (HCV) antibody test at screening, or positive HCV antibody test followed by a negative HCV RNA test at screening. The HCV RNA test is performed only for patients who have a positive HCV antibody test.
For women of childbearing potential: agreement to remain abstinent (refrain from heterosexual intercourse) or use contraceptive measures.
For men: agreement to remain abstinent (refrain from heterosexual intercourse) or use contraceptive measures, and agreement to refrain from donating sperm, as outlined for each specific treatment arm.

### Inclusion Criteria for Cohort 1

Patients must meet all of the following criteria to qualify for Cohort 1:
ECOG performance status (PS) of 0 or 1.
Histologically confirmed resectable Stage III melanoma (T: TO, Tx or T1-4; N: cN1-3, pN1b/2b/3b; M: M0 according to AJCC-8 (Gershenwald et al. C A Cancer J Clin. 67:472-492, 2017)) and no history of in-transit metastases within the last 6 months.
   Patients may present with primary melanoma with concurrent regional nodal metastasis, or a history of primary melanoma or unknown primary melanoma with clinically detected regional nodal recurrence, and may belong to any of the following groups: Primary cutaneous melanoma with concurrent clinically/radiologically apparent regional lymph node metastases; clinically/radiologically detected recurrent melanoma at the proximal regional lymph node(s) basin; or clinically/radiologically detected nodal melanoma (if single site) arising from an unknown primary.
Fit and planned for CLND (as assessed by surgeon prior to randomization according to local guidelines).
Measurable disease (at least one target lesion) according to RECIST v1.1. At least one macroscopic lymph node metastasis (measurable according to RECIST v1.1) to be biopsied.

### Inclusion Criteria for Cohort 2

Patients must meet all of the following criteria to qualify for Cohort 2:
ECOG PS of 0, 1, or 2.
Life expectancy≥3 months, as determined by the investigator.
Histologically confirmed Stage IV (metastatic) melanoma according to AJCC-8 (Gershenwald et al. C A Cancer J Clin. 67:472-492, 2017).
Disease progression during or following at least one but no more than two lines of treatment for metastatic disease. Up to two lines of checkpoint inhibition therapy (monotherapy or combination therapy) are allowed. Patients with BRAF-mutant disease may have received an additional line of targeted therapy (either before, intermittent with, or after the checkpoint inhibition therapy), or may have received targeted therapy and checkpoint inhibition therapy concurrently as one combination treatment.
Patients who relapse or systemically progress during or within 6 months of completion of adjuvant therapy for localized melanoma may be eligible.
Measurable disease (at least one target lesion) according to RECIST v1.1.

At least one metastasis (measurable according to RECIST v1.1).

### E. Exclusion Criteria

Patients are excluded from enrollment in specific arms if they meet any of the applicable criteria outlined in subsequent sections, as specified by treatment arm below:

### Exclusion Criteria for Cohort 1 and Cohort 2

Patients who meet any of the following criteria are excluded from study entry:
Mucosal and uveal melanoma.
   Acral lentiginous melanoma is excluded for Cohort 1.
   For Cohort 2, acral lentiginous melanoma is permitted; however, the proportion of patients should not exceed 20% of response-evaluable patients.
Treatment with investigational therapy within 28 days prior to initiation of study treatment.
Treatment with systemic immunostimulatory agents (including, but not limited to, IFN and IL-2) within 4 weeks or 5 drug-elimination half-lives (whichever is longer) prior to initiation of study treatment.
Prior allogeneic stem cell or solid organ transplantation.
Known immunodeficiency or conditions requiring treatment with systemic immunosuppressive medication (including, but not limited to, cyclophosphamide, azathioprine, methotrexate, thalidomide, and anti-tumor necrosis factor-α agents), or anticipation of need for systemic immunosuppressant medication during study treatment, with the following exceptions: patients on replacement doses of corticosteroids to manage hypopituitary or adrenal insufficiency are eligible for the study; patients who received acute, low-dose, systemic immunosuppressant medications, or a one-time pulse dose of systemic immunosuppressant medication (e.g., 48 hours of corticosteroids for a contrast allergy) are eligible for the study after discussion with Medical Monitor; patients who received mineralocorticoids (e.g., fludrocortisone), corticosteroids for chronic obstructive pulmonary disease or asthma, or low-dose corticosteroids for orthostatic hypotension or adrenal insufficiency are eligible for the study.
Treatment with a live, attenuated vaccine within 4 weeks prior to initiation of study treatment, or anticipation of need for such a vaccine during study treatment or within 5 months after the final dose of study treatment.
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barre syndrome, or multiple sclerosis, with the following exceptions:
   Patients with a history of autoimmune-related hypothyroidism who are on thyroid-replacement hormone are eligible for the study.
   Patients with controlled Type 1 diabetes mellitus who are on a stable insulin regimen are eligible for the study.
   Patients with eczema, psoriasis, lichen simplex chronicus, or vitiligo with dermatologic manifestations only (e.g., patients with psoriatic arthritis are excluded) are eligible for the study provided all of following conditions are met: rash must cover <10% of body surface area; disease is well controlled at baseline and requires only low-potency topical corticosteroids; there is no occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on screening chest computed tomography (CT) scan. Patients with a history of CIT-related pneumonitis Grade <2 may be eligible after discussion with the Medical Monitor.
History of malignancy other than malignant melanoma within 2 years prior to screening, with the exception of malignancies with a negligible risk of metastasis or death (e.g., 5-year OS rate>90%), such as adequately treated carcinoma in situ of the cervix, non-melanoma skin carcinoma, localized prostate cancer, ductal carcinoma in situ, or Stage I uterine cancer.
Active tuberculosis (TB).
Severe infection within 4 weeks prior to initiation of study treatment, including, but not limited to, hospitalization for complications of infection, bacteremia, or severe pneumonia, or any active infection that, in the opinion of the investigator, could impact patient safety.
Treatment with therapeutic or prophylactic oral or IV antibiotics within 2 weeks prior to initiation of study treatment.
Significant cardiovascular disease such as New York Heart Association cardiac disease (Class II or greater), myocardial infarction or cerebrovascular accident within 3 months prior to initiation of study treatment, unstable arrhythmia, or unstable angina.
Uncontrolled hypertension (defined as resting systolic blood pressure>150 mmHg and/or diastolic blood pressure>100 mmHg in two or more serial measurements).
Major surgical procedure, other than for diagnosis, within 4 weeks prior to initiation of study treatment, or anticipation of need for a major surgical procedure other than CLND, during the study.
   Placement of central venous access catheter (e.g., port or similar) is not considered a major surgical procedure and is therefore permitted.
Any other disease, metabolic dysfunction, physical examination finding, or clinical laboratory finding that contraindicates the use of an investigational drug, may affect the interpretation of the results, impair the ability of the patient to participate in the study, or may render the patient at high risk from treatment complications.
History of severe allergic reactions to chimeric or humanized antibodies or fusion proteins.
Known hypersensitivity to Chinese hamster ovary cell products or recombinant human antibodies.
Known allergy or hypersensitivity to any of the study drugs or their excipients.
Known intolerance to any of the drugs required for premedication (acetaminophen, ranitidine, diphenhydramine, and methylprednisolone).
Pregnancy or breastfeeding, or intention of becoming pregnant during the study.
   Women of childbearing potential must have a negative serum pregnancy test result within 14 days prior to initiation of study treatment.
Eligible only for the control arm.

### Exclusion Criteria for Cohort 1

Patients who meet any of the following criteria are excluded from Cohort 1:
Distantly metastasized melanoma.
History of in-transit metastases within the last 6 months.
Prior radiotherapy.
Prior immunotherapy, including anti-CTLA-4, anti-PD-1, and anti-PD-L1 therapeutic antibodies, and other systemic therapy for melanoma

### Exclusion Criteria for Cohort 2

Patients who meet any of the following criteria are excluded from Cohort 2:
Symptomatic, untreated, or progressing CNS metastases.
   Asymptomatic patients with treated CNS lesions are eligible, provided that all of the following criteria are met: Measurable disease, per RECIST v1.1, must be present outside the CNS; the patient has no history of intracranial hemorrhage or spinal cord hemorrhage; CNS metastases are stable for ≥4 weeks prior to initiation of study, or neurosurgical resection occurred ≥28 days prior to initiation of study treatment; the patient has no requirement for corticosteroids as therapy for CNS disease for at least 14 days prior to initiation of study treatment; anti-convulsant therapy at a stable dose is permitted.
Active or history of carcinomatous meningitis/leptomeningeal disease.
Uncontrolled tumor-related pain. Patients requiring pain medication must be on a stable regimen at screening. Symptomatic lesions (e.g., bone metastases or metastases causing nerve impingement) amenable to palliative radiotherapy should be treated prior to enrollment. Patients should be recovered from the effects of radiation. There is no required minimum recovery period. Asymptomatic metastatic lesions that would likely cause functional deficits or intractable pain with further growth (e.g., epidural metastasis that is not currently associated with spinal cord compression) should be considered for loco-regional therapy, if appropriate, prior to enrollment.
Uncontrolled pleural effusion, pericardial effusion, or ascites requiring recurrent drainage procedures (once monthly or more frequently). Patients with indwelling catheters (e.g., PLEURX®) are allowed.
Uncontrolled or symptomatic hypercalcemia (ionized calcium>1.5 mmol/L, calcium>12 mg/dl, or corrected calcium>ULN).
Any history of an immune-mediated Grade 4 adverse event attributed to prior CIT (other than endocrinopathy managed with replacement therapy or asymptomatic elevation of serum amylase or lipase) that resulted in permanent discontinuation of the prior immunotherapeutic agent.
All immune-mediated adverse events related to prior immunomodulatory therapy (other than endocrinopathy managed with replacement therapy or stable vitiligo) that have not resolved completely to baseline. Patients treated with corticosteroids for immune-mediated adverse events, except for corticosteroids replacement therapy for adrenal insufficiency (provided that the patient receives≤10 mg prednisone/day or equivalent), must not have related symptoms or signs for ≥4 weeks following discontinuation of corticosteroids.
Adverse events related to any prior radiotherapy, chemotherapy, targeted therapy, CPI therapy or surgical procedure must have resolved to Grade 1 or better, except alopecia (any grade), Grade 2 peripheral neuropathy, and hypothyroidism and/or hypopituitarism on a stable dosage of hormone replacement therapy (e.g., thyroxine, hydrocortisone, prednisolone, others).

### Exclusion Criteria for RO7247669-Containing Arms (Cohort 1 and Cohort 2)

Patients who meet any of the following criteria are excluded from the RO7247669-containing arm:
Prior treatment with an anti-LAG-3 agent.
History of myocarditis (regardless of etiology).
Left ventricular ejection fraction (LVEF)<50% assessed by either transthoracic echocardiogram (TTE) or multiple-gated acquisition (MUGA) scan (TTE preferred test) within 6 months prior to initiation of study treatment.
Troponin T (TnT) or troponin I (TnI)>institutional ULN. Patients with TnT or TnI levels between >1 and <2×ULN are eligible if repeat levels within 24 hours are ≤1×ULN. If repeat levels within 24 hours are between >1 and <2×ULN, patients need to undergo a cardiac evaluation and may be considered for treatment if there are no clinically significant findings.

### Exclusion Criteria for Tiragolumab-Containing Arms (Cohort 1 and Cohort 2)

Patients who meet any of the following criteria are excluded from the tiragolumab-containing arm:
Prior treatment with an anti-TIGIT agent.
Active Epstein-Barr virus (EBV) infection or known or suspected chronic active EBV infection at screening. Patients with a positive EBV viral capsid antigen (VCA) IgM test at screening are excluded from the tiragolumab-containing arms. An EBV PCR test should be performed as clinically indicated to screen for active infection or suspected chronic active infection. Patients with a positive EBV PCR test are excluded from the tiragolumab-containing arms.

### F. Study Treatment

The investigational medicinal products for this study are atezolizumab, tiragolumab, RO7247669, nivolumab, and ipilimumab.

### Control Arm (Nivolumab+Ipilimumab)

Patients in the nivolumab plus ipilimumab (Nivo+Ipi) control arm receive treatment for 2 cycles (6 weeks) as outlined in Table 8 until surgery, or until unacceptable toxicity or loss of clinical benefit, whichever occurs first. It is recommended that treatment be initiated no later than 7 days after randomization.

TABLE 8
Treatment Regimen for Nivolumab + Ipilimumab Arm

| | Dose, Route, and Regimen |
|---|---|
| Cycle Length | (drugs listed in order of administration) |
| 21 days | Nivolumab 3 mg/kg IV on Day 1 of each cycle |
| | Ipilimumab 1 mg/kg IV on Day 1 of each cycle |

Nivolumab is administered by IV infusion at a dose of 3 mg/kg on Day 1 of each 21-day cycle (Q3W). Ipilimumab is administered by IV infusion at a dose of 1 mg/kg on Day 1 of each 21-day cycle (Q3W).

### RO7247669 Arm

Patients in the RO7247669 arm receive treatment for 2 cycles (6 weeks) as outlined in Table 9 until surgery, or until unacceptable toxicity or loss of clinical benefit, whichever occurs first. It is recommended that treatment be initiated no later than 7 days after randomization.

TABLE 9
Treatment Regimen for RO7247669 Arm

| Cycle | Dose, Route, and Regimen |
|---|---|
| Length | (drugs listed in order of administration) |
| 21 days | RO7247669 2100 mg by IV infusion on Day 1 of each cycle |

RO7247669 is administered at a fixed dose of 2100 mg Q3W (2100 mg on Day 1 of each 21-day cycle). Administration of RO7247669 is performed in a monitored setting where there is immediate access to trained personnel and adequate equipment and medicine to manage potentially serious reactions. RO7247669 infusions are administered per the instructions outlined in Table 10.

TABLE 10
Administration of First and Second RO7247669 Infusions

| First Infusion | Second Infusion |
|---|---|
| No premedication is permitted prior | If the patient experienced an |
| to the first RO7247669 infusion. | IRR with the first infusion, |
| Vital signs (pulse rate, respiratory | premedication with antihista- |
| rate, blood pressure, pulse oximetry, | mines, antipyretics, and/or |
| and temperature) should be measured | analgesics may be administered |
| within 60 minutes prior to the | for subsequent doses at the |
| infusion. | discretion of the investigator. |
| RO7247669 should be infused over 60 | Vital signs should be measured |
| (±10) minutes. | within 60 minutes prior to the |
| After the infusion of RO7247669, | infusion. |
| the patient begins a 60-minute | RO77247669 should be infused |
| observation period. | over 30 (±10) minutes if the |
| If clinically indicated, vital signs | first infusion was tolerated |
| should be measured every 15 (±5) | without an IRR, followed by a |
| minutes during the infusion and at | 30-minute observation period. |
| 30 (±10) minutes after the infusion. | If the patient experienced an |
| Patients should be informed about | IRR with the previous infusion |
| the possibility of delayed post- | or if clinically indicated, |
| infusion symptoms and instructed | vital signs should be measured |
| to contact their study physician | every 15 (±5) minutes during |
| if they develop such symptoms. | the infusion and at 30 (±10) |
| | minutes after the infusion. |

IRR = infusion-related reaction.

For patients who experience a Grade 2 infusion-related reaction (IRR), premedication with paracetamol 500-1000 mg orally (PO) or IV and diphenhydramine 25-50 mg PO or IV (or an alternative histamine H_{1/2} antagonist at an adequate dose) is required prior to subsequent infusions. In case of Grade 3 or 4 IRRs related to study treatment, the patient should be permanently discontinued from the study treatment.

No dose modification for RO7247669 is allowed.

### Atezolizumab+Tiragolumab

Patients in the atezolizumab plus tiragolumab (Atezo+Tira) arm will receive treatment for 2 cycles (6 weeks) as outlined in Table 11 until surgery, or until unacceptable toxicity or loss of clinical benefit, whichever occurs first. It is recommended that treatment be initiated no later than 7 days after randomization.

TABLE 11
Treatment Regimen for Atezolizumab + Tiragolumab Arm

| Cycle | Dose, Route, and Regimen |
|---|---|
| Length | (drugs listed in order of administration) |
| 21 days | Atezolizumab 1200 mg IV on Day 1 of each cycle |
| | Tiragolumab 600 mg IV on Day 1 of each cycle |

Atezolizumab is administered at a fixed dose of 1200 mg every 3 weeks (Q3W) (1200 mg on Day 1 of each 21-day cycle). Administration of atezolizumab is performed in a monitored setting where there is immediate access to trained personnel and adequate equipment and medicine to manage potentially serious reactions. Atezolizumab infusions are administered per the instructions outlined in Table 12.

No dose modification for atezolizumab is allowed.

TABLE 12
Administration of First and Second Atezolizumab Infusions

| First Infusion | Second Infusion |
|---|---|
| No premedication is permitted prior | If the patient experienced an |
| to the atezolizumab infusion. | IRR with the first infusion, |
| Vital signs (pulse rate, respiratory | premedication with antihista- |
| rate, pulse oximetry, blood pressure, | mines, anti-pyretics, and/or |
| and temperature) should be measured | analgesics may be administered |
| within 60 minutes prior to the infusion. | for subsequent doses at the |
| Atezolizumab should be infused over | discretion of the investigator. |
| 60 (±15) minutes. | Vital signs should be measured |
| After the infusion of atezolizumab, | within 60 minutes prior to the |
| the patient begins a 30-minute | infusion. |
| observation period. | Atezolizumab should be infused |
| If clinically indicated, vital | over |
| signs should be measured every 15 | 30 (±10) minutes if the |
| (±5) minutes during the infusion and | previous infusion was |
| 30 (±10) minutes after the infusion. | tolerated without an IRR, or |
| Patients should be informed about | 60 (±15) minutes if the patient |
| the possibility of delayed post- | experienced an IRR with the |
| infusion symptoms and instructed | previous infusion. |
| to contact their study physician | If the patient experienced an |
| if they develop such symptoms. | IRR with the previous infusion |
| | or if clinically indicated, vital |
| | signs should be measured during |
| | the infusion and at 30 (±10) |
| | minutes after the infusion. |

IRR = infusion-related reaction.

Tiragolumab is administered at a fixed dose of 600 mg IV Q3W (600 mg on Day 1 of each 21-day cycle). Administration of tiragolumab is performed in a monitored setting where there is immediate access to trained personnel and adequate equipment and medicine to manage potentially serious reactions. Tiragolumab infusions are administered per the instructions outlined in Table 13.

TABLE 13
Administration of First and Subsequent Tiragolumab Infusions

| First Infusion | Second and Susequent Infusions |
|---|---|
| No premedication is permitted prior | If the patient experienced an |
| to the tiragolumab infusion. | IRR with the first infusion, |
| Vital signs (pulse rate, respiratory | premedication with antihista- |
| rate, pulse oximetry, blood pressure, | mines, antipyretics, and/or |
| and temperature) should be measured | analgesics may be administered |
| within 60 minutes prior to the infusion. | for subsequent doses at the |
| Tiragolumab should be infused over | discretion of the investigator. |
| 60 (±15) minutes. | Vital signs should be measured |
| After the infusion of tiragolumab, | within 60 minutes prior to the |
| the patient begins a 60-minute | infusion. |
| observation period. | Tiragolumab should be infused |
| Record vital signs every 15 (±5) | over 30 (±10) minutes if the |
| minutes during the infusion and | previous infusion was tolerated |
| at 30 (±10) minutes after the | without an IRR, or 60 (±15) |
| infusion. | minutes if the patient |
| Patients are informed about the | experienced an infusion-related |
| possibility of delayed post- | reaction with the previous |
| infusion symptoms and are | infusion. |
| instructed to contact their | Patients should be observed |
| study physician if they develop | for 30 minutes after completion |
| such symptoms. | of the tiragolumab infusion if |
| | the previous infusion was |
| | tolerated without an IRR, or |
| | for 60 minutes after completion |
| | of the tiragolumab infusion |
| | if the patient experienced |
| | an IRR with the previous |
| | infusion. |
| | If clinically indicated, vital |
| | signs should be recorded |
| | every 15 (±5) minutes |
| | during the infusion and at |
| | 30 (±10) minutes after the |
| | infusion. |

IRR = infusion-related reaction.

Atezolizumab and tiragolumab treatment may be temporarily suspended in patients experiencing toxicity considered to be related to study treatment. If corticosteroids are initiated for treatment of the toxicity, they must be tapered over ≥1 month to the equivalent of ≤10 mg/day oral prednisone before study treatment can be resumed, if warranted. In the neoadjuvant setting, the study treatment is limited to a pre-surgery window of 6 weeks. Treatment during this period should not be interrupted, unless a patient experiences toxicity. If toxicity meets criteria for interrupting/withholding atezolizumab and/or tiragolumab, atezolizumab and/or tiragolumab should be interrupted/withheld. After resolution of the toxicity, subsequent treatment cycles should only be considered if the benefit/risk profile is acceptable and if the surgery can be conducted within 2 weeks of the planned date. Otherwise, subsequent treatment cycles should be omitted to allow the patient to proceed directly to surgery without further delay.

On the basis of the available characterization of mechanism of action, tiragolumab may cause adverse events similar to, but independent of, atezolizumab. Tiragolumab may also exacerbate the frequency or severity of atezolizumab-related adverse events or may have non-overlapping toxicities with atezolizumab. Because these scenarios may not be distinguishable from each other in the clinical setting, adverse events should generally be attributed to both agents, and dose interruptions or treatment discontinuation in response to adverse events should be applied to both tiragolumab and atezolizumab. If atezolizumab is withheld or discontinued, tiragolumab should also be withheld or discontinued. If tiragolumab is withheld or discontinued, atezolizumab should also be withheld or discontinued.

### RO7247669+Tiragolumab (Cohorts 1 and 2)

Patients in the RO7247669 plus tiragolumab (RO7247669+Tira) arm receive treatment as outlined in Table 14.

Patients in Cohort 1 receive treatment for 2 cycles (6 weeks) until surgery, or until unacceptable toxicity or loss of clinical benefit, whichever occurs first.

Patients in Cohort 2 receive treatment until unacceptable toxicity or loss of clinical benefit as determined by the investigator after an integrated assessment of radiographic and biochemical data, local biopsy results (if available), and clinical status (e.g., symptomatic deterioration such as pain secondary to disease).

It is recommended that treatment be initiated no later than 7 days after randomization (Cohort 1) or enrollment (Cohort 2).

TABLE 14
Treatment Regimen for RO7247669 + Tiragolumab Arm

| | Dose, Route, and Regimen |
|---|---|
| Cycle Length | (drugs listed in order of administration) |
| 21 days | RO7247669 2100 mg IV on Day 1 of each cycle ^{a} |
| | Tiragolumab 600 mg IV on Day 1 of each cycle |

^{a} After the safety run-in has been completed, the Sponsor may decide to explore lower doses (e.g., 1200 mg and 600 mg).

RO7247669 is administered by IV infusion at a fixed dose of 2100 mg on Day 1 of each 21-day cycle. Tiragolumab is administered by IV infusion at a fixed dose of 600 mg on Day 1 of each 21-day cycle with a post-infusion observation period as described in Table 13.

Administration of RO7247669 is performed in a monitored setting where there is immediate access to trained personnel and adequate equipment and medicine to manage potentially serious reactions. RO7247669 infusions are administered per the instructions outlined in Table 15.

TABLE 15
Administration of First, Second, and
Subsequent RO7247669 Infusions

| First and Second Infusion | Subsequent Infusions |
|---|---|
| No premedication is permitted | If the patient experienced an |
| prior to the first RO7247669 | IRR with any previous infusion, |
| infusion. | premedication with antihistamines, |
| Vital signs (respiratory rate, | antipyretics, and/or analgesics |
| pulse rate, blood pressure, pulse | may be administered for subsequent |
| oximetry, and temperature) should | doses at the discretion of the |
| be measured within 60 minutes | investigator. |
| prior to the infusion. | Vital signs should be measured |
| For the first infusion, RO7247669 | within 60 minutes prior to the |
| should be infused over 60 (±10) | infusion. |
| minutes. | RO77247669 should be infused over |
| After the infusion of RO7247669, | 30 (±10) minutes if the previous |
| the patient begins a 60-minute | infusion was tolerated without an |
| observation period. | IRR. |
| For the second infusion, | If the patient tolerated the |
| RO7247669 should be infused | previous infusion of RO77247669 |
| over 30 (±10) minutes if | well without infusion-associated |
| the first infusion was | adverse events, the observation |
| tolerated without an IRR. | period may be reduced to 30 minutes. |
| If clinically indicated, vital | If the patient experienced an |
| signs should be measured every 15 | IRR with the previous infusion or |
| (±5) minutes during the infusion | if clinically indicated, vital |
| and at 30 (±10) minutes after the | signs should be measured every 15 |
| infusion. | (±5) minutes during the infusion |
| Patients should be informed about | and at 30 (±10) minutes after the |
| the possibility of delayed post- | infusion. |
| infusion symptoms and instructed | |
| to contact their study physician | |
| if they develop such symptoms. | |

IRR = infusion-related reaction.

For patients who experience a Grade ≥2 infusion-related reaction (IRR), premedication with paracetamol 500-1000 mg orally (PO) or IV and diphenhydramine 25-50 mg PO or IV (or an alternative histamine H_{1/2} antagonist at an adequate dose) is required prior to subsequent infusions. In case of Grade 3 or 4 IRRs related to study treatment, the patient should be permanently discontinued from the study treatment.

No dose modification for RO7247669 is allowed. However, based on emerging safety and efficacy data, the Sponsor may explore lower doses (e.g., 1200 mg and 600 mg). RO7247669 treatment may be interrupted for reasons other than toxicity (e.g., surgical procedures).

The investigator and the Medical Monitor will determine the acceptable length of treatment interruption.

Treatment with RO7247669 and tiragolumab may be temporarily suspended in patients experiencing toxicity considered to be related to study treatment. If corticosteroids are initiated for treatment of the toxicity, they must be tapered over >1 month to the equivalent of ≤10 mg/day oral prednisone before study treatment can be resumed, if warranted.

For Cohort 1, in the neoadjuvant setting the study treatment is limited to a pre-surgery window of 6 weeks. Treatment during this period should not be interrupted unless a patient experiences toxicity. If toxicity meets criteria for interrupting/withholding RO7247669 and tiragolumab, RO7247669 and tiragolumab should be interrupted/withheld. After resolution of the toxicity, subsequent treatment cycles should only be considered if the benefit/risk profile is acceptable and if the surgery can be conducted within 2 weeks of the planned date. Otherwise, subsequent treatment cycles should be omitted to allow the patient to proceed directly to surgery without further delay.

For Cohort 2, if RO7247669 and tiragolumab are withheld for 12 weeks or longer due to toxicity, the patient should be discontinued from RO7247669 and tiragolumab. However, RO7247669 and tiragolumab may be withheld for more than 12 weeks to allow for patients to taper off corticosteroids prior to resuming treatment. RO7247669 and tiragolumab may be resumed after being withheld for more than 12 weeks if the Medical Monitor agrees that the patient is likely to derive clinical benefit. RO7247669 and tiragolumab treatment may be suspended for reasons other than toxicity (e.g., surgical procedures). The acceptable length of the extended period of time must be agreed upon by the investigator and the Medical Monitor.

On the basis of the available characterization of mechanism-of-action, tiragolumab may cause adverse events similar to, but independent of, RO7247669. Tiragolumab may also exacerbate the frequency or severity of RO7247669-related adverse events or may have non-overlapping toxicities with RO7247669. Because these scenarios may not be distinguishable from each other in the clinical setting, adverse events should generally be attributed to both agents, and dose interruptions or treatment discontinuation in response to adverse events should be applied to both tiragolumab and RO7247669. If RO7247669 is withheld or discontinued, tiragolumab should also be withheld or discontinued. If tiragolumab is withheld or discontinued, RO7247669 should also be withheld or discontinued.

### G. Concomitant Therapy

Concomitant therapy consists of any medication (e.g., prescription drugs, over-the-counter drugs, vaccines, herbal or homeopathic remedies, nutritional supplements) used by a patient in addition to protocol-mandated study treatment from 7 days prior to initiation of study treatment to the treatment discontinuation visit.

In general, investigators should manage a patient's care (including preexisting conditions) with supportive therapies other than those defined as cautionary or prohibited therapies as clinically indicated, per local standard practice. Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β2-adrenergic agonists).

### Permitted Therapy for RO7247669, Atezo+Tira, and RO7247669+Tira Arms

Patients are permitted to use the following therapies during the study:
Oral contraceptives with a failure rate of <1% per year.
Hormone-replacement therapy.
Prophylactic or therapeutic anticoagulation therapy (such as warfarin at a stable dose or low-molecular-weight heparin).
Inactivated vaccinations (e.g., influenza).
Megestrol acetate administered as an appetite stimulant.
Mineralocorticoids (e.g., fludrocortisone)
Corticosteroids administered for chronic obstructive pulmonary disease (COPD) or asthma.
Low-dose corticosteroids administered for orthostatic hypotension or adrenocortical insufficiency.
Local therapy (e.g., surgery (other than complete lymph node dissection (CLND) and not melanoma specific).

For the RO7247669 arm, premedication with antihistamines, antipyretics, and/or analgesics may be administered for the second RO7247669 infusion only, at the discretion of the investigator.

For the atezolizumab plus tiragolumab arm, premedication with antihistamines, antipyretics, and/or analgesics may be administered for the second atezolizumab and tiragolumab infusions only, at the discretion of the investigator.

### Additional Permitted Therapy for RO7247669+Tira Arm in Cohort 2

Patients are permitted to use the following therapies during the study:
Palliative radiotherapy (e.g., treatment of known bony metastases or symptomatic relief of pain) as outlined: Palliative radiotherapy is permitted, provided it does not interfere with the assessment of tumor target lesions (e.g., the lesion to be irradiated must not be the only site of measurable disease). Treatment with tiragolumab may be continued during palliative radiotherapy. Treatment with RO7247669 may be continued during palliative radiotherapy with one exception: palliative radiotherapy is not permitted on days when RO7247669 is administered.
Local therapy (e.g., surgery, stereotactic radiosurgery, radiotherapy, radiofrequency ablation) as outlined: Patients experiencing a mixed response requiring local therapy for control of three or fewer lesions may still be eligible to continue study treatment after Medical Monitor approval has been obtained. Patients who receive local therapy directed at a target lesion will no longer be evaluable for radiographic response but will remain evaluable for progression.

Premedication with antihistamines, antipyretics, and/or analgesics may be administered for the second and subsequent RO7247669 and tiragolumab infusions only, at the discretion of the investigator.

### H. Assessments

All patients are closely monitored for adverse events throughout the study. Adverse events are graded according to the National Cancer Institute Common Terminology Criteria for Adverse Events, Version 5.0 (NCI CTCAE v5.0). Cytokine-release syndrome (CRS) severity is also graded according to the American Society for Transplantation and Cellular Therapy (ASTCT) CRS Consensus Grading Scale.

Patients in Cohort 1 receive neoadjuvant treatment for 2 cycles (6 weeks) and undergo surgery (CLND) in Week 7. All patients are expected to proceed with surgery, provided that there are no distant metastases and the surgeon considers the disease to be completely resectable. Pathologic response is assessed locally and by independent pathologic review.

Patients who discontinue treatment due to unacceptable toxicity and continue to have no evidence of metastatic disease are still eligible for surgery and proceed with CLND after the adverse event has resolved and re-staging confirms Stage III disease. If patients have confirmed disease progression, patient management and treatment selection are at the discretion of the treating physician. These patients remain in the study for follow-up.

Patients undergo radiological tumor assessments in Week 6 (from Day 1 of Cycle 1) prior to surgery (CLND). Response is assessed and determined by the investigator in accordance with RECIST v1.1, but confirmation by later imaging studies is not required.

Patients in Cohort 2 undergo tumor assessments every 9 weeks (from Day 1 of Cycle 1) for the first 54 weeks and then every 12 weeks thereafter. Response is assessed by the investigator using RECIST v1.1. Response per modified RECIST v1.1 for immune-based therapeutics (iRECIST) is determined programmatically by the Sponsor on the basis of investigator-assessed individual lesion data.

For Cohort 1 and Cohort 2, if clinical activity is demonstrated in an experimental arm, the Sponsor may request that tumor assessment scans for that arm be submitted for evaluation by an independent review facility.

### Tumor and Response Evaluations

All measurable and evaluable lesions should be assessed and documented at screening. Tumor assessments performed as standard of care prior to obtaining informed consent and within 14 days prior to randomization/enrollment do not have to be repeated at screening.

All measurable and/or evaluable lesions identified at baseline should be re-assessed at subsequent tumor evaluations for Cohorts 1 and 2. The same radiographic procedures used to assess disease sites at screening should be used for subsequent tumor assessments (e.g., the same contrast protocol for CT scans).

### Cohort 1 Tumor and Response Evaluations

Patients in Cohort 1 are assessed for pathologic and radiologic response to treatment. Patients undergo pathological tumor assessments at baseline, and after 6 weeks of treatment at surgery (CLND). The complete resection of Stage III lymph nodes (CLND) in Week 7 must be performed in compliance with the criteria for adequate surgical procedures for therapeutic lymph node dissection. CLND should be performed as planned if the patient is receiving corticosteroids or other anti-inflammatory drugs for the management of immune-mediated adverse events, provided these are being given at a stable or tapering dose and the severity of adverse events is Grade 2 or better. CLND may be delayed for up to 2 weeks if study treatment-related adverse events have not improved sufficiently at the time of planned surgery. Pathological response is determined by local and independent pathologic review according to INMC guidelines (Tetzlaff et al. Ann Oncol. 29:1861-1868, 2018).

Surgical complications are scored according to Clavien-Dindo classification. Complication rates for every grade are reported and scored for patients that underwent CLND.

Patients undergo radiographic tumor assessments at baseline, after 6 weeks of treatment before surgery (CLND), and at treatment completion/discontinuation at Week 13 according to RECIST v1.1.

Overall response at a single timepoint is assessed by the investigator using RECIST v1.1.

### Disease Follow-Up and Confirmation of Disease Progression or Recurrence

During the neoadjuvant treatment, diagnosis of disease progression should be confirmed by clinical, laboratory, radiological, and/or histological findings. After surgery, prior to commencing adjuvant treatment or observation, a tumor assessment is performed in Week 13 to conclude the neoadjuvant therapy-surgery intervention window.

Thereafter, outside the study during the adjuvant treatment/observation phase (i.e., commencing in Week 13), all patients must be followed to assess disease recurrence and survival as outlined for each arm.

Patients who complete the treatment period have their first survival follow-up visit 3 months after surgery. Patients who discontinue study drug prematurely have their first survival follow-up visit 3 months after the final dose of study treatment. The designation of disease recurrence, whether local, regional, or distant, can be made only when clinical, laboratory, radiological and/or histological findings confirm the diagnosis.

During the post-surgery period, disease status should be clinically evaluated and documented as per institutional guidelines (e.g., every 3 months for the first 2 years; every 6 months in the third year; once a year in the fourth and following years). In addition, liver function tests, bone scans, chest X-ray/diagnostic CT scan, liver imaging, and/or other radiographic modalities may be considered when clinically indicated to exclude metastatic disease.

The diagnosis of a progression or recurrence should be confirmed histologically whenever clinically possible. The earliest date of diagnosis of disease progression or recurrent disease should be used and recorded. This date should be based on objective clinical, radiological, histological, or cytological evidence. Recurrent disease includes local, regional, or distant recurrence.

The definitions of and procedures for confirming disease recurrence, death, and other noteworthy events on follow-up are provided below. Documentation of recurrence requires specification of all sites involved to establish the pattern of recurrence. The following criteria of treatment failure constitute the only acceptable evidence of disease recurrence:
Lung: Positive cytology or biopsy in the presence of a single new lesion or the appearance of multiple lesions consistent with metastatic disease.
Liver: Positive cytology or biopsy in the presence of a single new lesion or the appearance of multiple lesions consistent with metastatic disease.
Central nervous system: A positive brain CT or MRI scan or CSF cytology.
Cutaneous, subcutaneous, and lymph node recurrence: Positive cytology or biopsy in the presence of a single new lesion or the appearance of multiple lesions consistent with metastatic disease.
Bone and other organs: Positive cytology or biopsy in the presence of a single new lesion or the appearance of multiple lesions consistent with metastatic disease identified on two different radiologic studies (i.e., positive nuclear bone scan or PET scan and contrast GI series or ultrasound, X-ray or CT of abdomen for abdominal disease).

### Cohort 2 Tumor and Response Evaluations

Patients in Cohort 2 undergo tumor assessments every 9 (+1) weeks (from Day 1 of Cycle 1) for the first 54 weeks and then every 12 (+2) weeks thereafter, regardless of dose delays. The exception is patients who continue treatment after radiographic disease progression. Such patients undergo tumor assessments every 9 weeks until loss of clinical benefit as determined by the investigator. Thus, tumor assessments continue according to schedule in patients who discontinue treatment for reasons other than loss of clinical benefit, even if they start new, non-protocol-specified anti-cancer therapy. At the investigator's discretion, tumor assessments may be repeated at any time if progressive disease is suspected.

Brain metastases treated with radiotherapy or surgery are not considered measurable or evaluable but are documented at screening as a site of metastatic disease. Brain metastases identified at baseline that have been treated with radiotherapy or surgery are not considered to be measurable or evaluable unless there is suspected disease progression in the brain (i.e., the patient becomes symptomatic). Thus, subsequent head scans are not required unless clinically indicated.

To facilitate evaluation of response per iRECIST in Cohort 2, tumor assessments must be continued after disease progression per RECIST v1.1 for patients who receive treatment beyond progression. This includes continued measurement of target lesions, evaluation of non-target lesions (including monitoring for further worsening of any non-target lesions that have shown unequivocal progression), and evaluation of any newly identified lesions (including measurements, if lesions are measurable) at all subsequent assessments.

Overall response at a single timepoint is assessed by the investigator using RECIST v1.1.

### Biomarker Assessments

Baseline tumor tissue samples are collected from all patients (except patients in the Cohort 2 safety run-in phase) by biopsy of a metastatic lymph node (Cohort 1) or other metastatic lesion (Cohort 2) at screening. For patients in Cohort 1, on-treatment tissue samples are collected by biopsy on Day 1 of Cycle 2, and at surgery (CLND). For patients enrolled in Cohort 2, on-treatment tissue samples are collected by biopsy on Day 8 of Cycle 2.

Exploratory biomarker analyses are performed in an effort to understand the association of biomarkers with response to study drugs, taking into account efficacy and safety endpoints. Exploratory biomarker research may include, but is not limited to, analysis of genes or gene signatures associated with tumor immunobiology, PD-L1, lymphocyte subpopulations, T-cell receptor repertoire, or cytokines associated with T-cell activation. Research may involve DNA or RNA extraction, analysis of somatic mutations, and use of next-generation sequencing (NGS) (including whole exome sequencing (WES)). Research may involve extraction of DNA, cell-free DNA, or RNA; analysis of mutations, single nucleotide polymorphisms, and other genomic variants; and genomic profiling through use of NGS of a comprehensive panel of genes. DNA extracted from blood may be compared with DNA extracted from tissue to identify somatic variants by distinguishing germline variants from somatic variants.

NGS methods may include whole genome sequencing (WGS) or WES of tissue and blood samples. At participating sites, blood samples are collected for DNA extraction to enable WGS or WES to identify variants that are predictive of response to study drug, are associated with progression to a more severe disease state, are associated with acquired resistance to study drug, are associated with susceptibility to develop adverse events, can lead to improved adverse event monitoring or investigation, or can increase the knowledge and understanding of disease biology and drug safety. DNA extracted from blood may be compared with DNA extracted from tissue to identify somatic variants by distinguishing germline variants from somatic variants.

### I. Analysis

The final study analysis is based on patient data collected through study discontinuation. If not otherwise specified, efficacy analyses are based on the efficacy-evaluable population, defined as all patients who receive at least one dose of each drug for their assigned treatment regimen, and safety analyses are based on the safety-evaluable population, defined as all patients who receive any amount of study treatment.

Enrollment is summarized by region, country, and investigator by treatment arm. Patient disposition is summarized by treatment arm. Major protocol deviations, including major deviations with regard to the inclusion and exclusion criteria, are summarized by treatment arm.

For safety-evaluable patients, study drug administration data are tabulated or listed by treatment arm, and any dose modifications are flagged. Means and standard deviations are used to summarize the total dose and dose intensity for each study drug. Reasons for discontinuation of study drugs are tabulated.

Demographic and baseline characteristics (including age, sex, race/ethnicity, weight, malignancy duration, metastatic disease site (if applicable), and baseline ECOG PS) are summarized overall and by treatment arm.

### Determination of Sample Size

This study is not designed to make explicit power and type I error considerations for a hypothesis test. Instead, this study is designed to obtain preliminary efficacy, safety, and PK data on treatments or treatment combinations when administered to patients with melanoma. Cohort 1 consists of patients with resectable Stage III melanoma who have not received prior systemic therapy for their disease. Cohort 2 consists of patients with Stage IV melanoma who experienced disease progression during or after at least one but not more than two lines of treatment for metastatic disease.

In Cohort 1, approximately 55-145 patients are randomly allocated to the control and experimental arms during the study. In Cohort 2, approximately 6-46 patients are assigned to an experimental arm.

### Efficacy Analyses

### Primary Efficacy Endpoint in Cohort 1

The primary efficacy endpoint in Cohort 1 is pRR at time of surgery. pRR is assessed after completion of neoadjuvant treatment (Week 7) at time of CLND. The pRR is defined as the proportion of patients who achieve pCR (a complete absence of viable tumor in the treated tumor bed), pathologic near complete response (pnCR; <10% of viable tumor in the treated tumor bed); and pathologic partial response (pPR; <50% of the treated tumor bed is occupied by viable tumor cells) as determined by independent pathologic review. pRR is calculated for each arm along with 90% CIs. The difference in the pRR between the experimental arms and the control arm is also calculated along with 90% CIs. Confidence intervals are estimated by the exact method or the Wald method, depending on the sample size.

### Secondary Efficacy Endpoints in Cohort 1

The secondary efficacy endpoints in Cohort 1 are pRR at time of surgery as determined by local pathologic assessment, event-free survival (EFS), RFS, OS, and ORR prior to surgery. pRR is defined in Table 5.

EFS is defined as the time from randomization to any of the following events (whichever occurs first): disease progression that precludes surgery, as assessed by the investigator according to RECIST v1.1; local, regional, or distant disease recurrence; or death from any cause. Patients who have not experienced such events are censored at the time of their last post-tumor tumor assessment.

RFS is defined as the time from surgery to the first documented recurrence of disease or death from any cause. For patients who do not have documented recurrence of disease or death, RFS is censored at the day of the last tumor assessment.

OS is defined as the time from randomization to death from any cause. Patients who are still alive at the time of OS analysis are censored at the last date they were known to be alive.

The Kaplan-Meier method is used to estimate the median for RFS, EFS, and OS, 90% CIs are constructed using the Brookmeyer and Crowley method. The RFS, EFS, and OS rate at specific timepoints are also estimated using the Kaplan-Meier method, with 90% CIs calculated on the basis of Greenwood's estimate for the variance.

The ORR according to RECIST v1.1 is assessed after completion of neoadjuvant treatment (Week 7) and is defined as the proportion of patients with a CR or PR, as determined by the investigator according to RECIST v1.1. Patients with missing or no response assessments are classified as non-responders. Note that ORR is determined using unconfirmed pre-operative radiologic responses. Although RECIST v1.1 requires confirmatory imaging assessments to be completed at least 4 weeks after the initial response, due to the timing of CLND, these responses cannot be confirmed with subsequent imaging.

ORR is calculated for each arm, along with 90% CIs, using the Clopper-Pearson method. The difference in ORR between the experimental arms and the control arm is also calculated, along with 90% CIs. CIs are estimated by the exact method or the Wald method, depending on the sample size.

### Exploratory Efficacy Endpoints in Cohort 1

The exploratory efficacy endpoints in Cohort 1 are landmark EFS, landmark RFS, and landmark OS at specific timepoints (1, 2, 3, and 5 years).

Landmark EFS rates, landmark RFS rates, and landmark OS rates are estimated for each study arm using the Kaplan-Meier method, with 90% CIs calculated through use of Greenwood's formula.

### Primary Efficacy Endpoint in Cohort 2

The primary efficacy endpoint in Cohort 2 is ORR, as defined in Table 6. ORR is determined by the investigator according to RECIST v1.1. Patients with missing or no response assessments are classified as non-responders.

ORR, the proportion of patients with a complete or partial response, is calculated for each arm, along with 90% CIs (Clopper-Pearson method). CIs are estimated by the exact method or the Wald method, depending on the sample size.

### Secondary Efficacy Endpoints in Cohort 2

The secondary efficacy endpoints in Cohort 2 are PFS, OS, OS at specific timepoints (e.g., 6 months), duration of response (DOR), and disease control, as defined in Table 6. PFS, DOR, and disease control are determined by the investigator according to RECIST v1.1.

DOR is derived for efficacy-evaluable patients with a CR or PR.

For patients who do not have documented disease progression or death, PFS and DOR is censored at the day of the last tumor assessment.

Patients who are still alive at the time of OS analysis are censored at the last date they were known to be alive.

The Kaplan-Meier method is used to estimate the median for PFS, OS, and DOR, with 90% CIs constructed through use of the Brookmeyer and Crowley method. OS rate at specific timepoints is also estimated using the Kaplan-Meier method, with 90% CIs calculated on the basis of Greenwood's estimate for the variance.

Disease control rate (the proportion of patients with SD for ≥12 weeks), a PR, or a CR, will be calculated for each treatment arm, with 90% CIs estimated through use of Clopper-Pearson's exact method.

### Exploratory Efficacy Endpoints in Cohort 2

The exploratory efficacy endpoints are ORR, PFS, DOR, and disease control as determined by the investigator according to iRECIST.

ORR, PFS, DOR, and disease control are analyzed through use of the same methods described in the above sections, “Primary Efficacy Endpoint in Cohort 2” and “Secondary Efficacy Endpoints in Cohort 2.” DOR is derived for efficacy-evaluable patients with a complete or partial response.

### Safety Analyses

Verbatim adverse event terms are mapped to Medical Dictionary for Regulatory Activities thesaurus terms, and adverse event severity is graded according to NCI CTCAE v5.0 and also according to the ASTCT CRS Consensus Grading Scale for CRS.

Safety is assessed through summaries of adverse events, changes in laboratory test results, changes in vital signs and ECGs, and exposure to study drugs. Exposure to combination treatment and length of safety follow-up is summarized by treatment arm.

All verbatim adverse event terms are mapped to Medical Dictionary for Regulatory Activities thesaurus terms. Adverse event severity is graded according to NCI CTCAE v5.0, and severity of CRS will also be graded by the investigator according to the ASTCT Consensus Grading (Lee et al. Biol Blood Marrow Transplant. 25:625-638, 2019). All adverse events, serious adverse events, adverse events leading to death, adverse events of special interest, and adverse events leading to study treatment discontinuation that occur on or after the first dose of study treatment (i.e., treatment-emergent adverse events) are summarized by mapped term, appropriate thesaurus level, and severity grade. For events of varying severity, the highest grade is used in the summaries. Deaths and causes of death are summarized.

Relevant laboratory, vital sign (pulse rate, respiratory rate, blood pressure, pulse oximetry, and temperature), and ECG data will be displayed by time, with grades identified where appropriate. Additionally, a shift table of selected laboratory test results is used to summarize the baseline and maximum postbaseline severity grade. Changes in vital signs and ECGs are summarized.

Additionally, in Cohort 1, the incidence, nature of immune-related adverse events Grade ≥3 during the first 12 weeks, and the rate and duration of delayed surgery due to treatment-related adverse events will be summarized by treatment arm. CLND may be delayed for up to 2 weeks if study treatment-related adverse events have not improved sufficiently at the time of planned surgery.

Additionally, surgical complications are scored according to Clavien-Dindo classification. Complication rates for every grade are reported and scored for patients that underwent CLND.

### Immunogenicity Analyses

Immunogenicity may be assessed for atezolizumab and other study treatments as appropriate. The immunogenicity analyses include all patients with at least one anti-drug antibody (ADA) assessment. Patients are grouped according to treatment received or, if no treatment is received prior to study discontinuation, according to treatment assigned.

For atezolizumab, the numbers and proportions of ADA-positive patients and ADA-negative patients at baseline (baseline prevalence) and after drug administration (postbaseline incidence) are summarized by treatment group. When determining postbaseline incidence, patients are considered to be ADA positive if they are ADA negative or have missing data at baseline but develop an ADA response following study drug exposure (treatment-induced ADA response), or if they are ADA positive at baseline and the titer of one or more postbaseline samples is at least 0.60-titer unit greater than the titer of the baseline sample (treatment-enhanced ADA response). Patients are considered to be ADA negative if they are ADA negative or have missing data at baseline and all postbaseline samples are negative, or if they are ADA positive at baseline but do not have any postbaseline samples with a titer that is at least 0.60-titer unit greater than the titer of the baseline sample (treatment unaffected).

For other study treatments where ADA is tested, positivity is determined according to standard methods established in previous studies of that drug.

The relationship between ADA status and safety, efficacy, PK, and biomarker endpoints may be analyzed and reported via descriptive statistics.

### Interim Analyses

Given the exploratory nature of this study, it is anticipated that interim analyses are conducted during the study, with the earliest interim analysis taking place when at least one experimental arm has completed enrollment in the preliminary phase, and patients have completed their pathologic response assessment (Cohort 1), or when at least one experimental arm has completed enrollment in the preliminary phase and patients have been followed for a minimum of 9 weeks for the primary endpoint analysis (ORR) (Cohort 2). Further interim analyses may be conducted as deemed appropriate by the Sponsor. In Cohort 1, a posterior probability may be used to guide further enrollment based on the interim analysis of clinical activity in the experimental arm compared with the control arm. If the interim analysis suggests that the activity in an experimental arm is higher than that in the control arm, there may be further enrollment of 20 additional patients in the experimental arm (expansion phase).

In Cohort 2, a posterior probability may be used to guide further enrollment in a treatment arm based on an interim analysis of clinical activity in the experimental arm compared with a predefined ORR threshold, defined as an improvement over standard of care. For example, if available data suggest a standard of care ORR of 10%, and an ORR improvement of 10% is considered to be a clinical meaningful change, this would lead to an ORR threshold of 20% in the calculation of the posterior probability.

The ORR for standard of care treatment is based on emerging internal and external data for in-class immune-modulating investigational and other compounds for the patient population in Cohort 2 who have received at least two lines of prior treatment at the time of the analysis.

The Sponsor may make a decision to expand enrollment in an arm based on the totality of available data including, but not limited to, duration of the observed responses, PFS, and potentially early OS data. Safety and biomarker data (available at the time of making this decision) are also taken into consideration from the perspective of an adequate benefit-risk assessment.

### Example 2: Rationale for 600 mg Q3W Dose and Schedule

### A. Introduction

RO7247669 (PD1-LAG3) is a novel bispecific antibody being investigated for the treatment of inflamed solid tumor types. It aims to reinvigorate T-cells by blocking two co-inhibitory checkpoint receptors, PD-1 and LAG-3. RO7247669 incorporates monovalent high-affinity binding to PD-1 and monovalent high-affinity binding to LAG-3 (20-fold lower than that to PD-1), allowing avidity-mediated selectivity gain. LAG-3 is expressed at higher levels on regulatory T-cells than other T-cells, and its blockade by monospecific anti-LAG3 antibodies has been reported to detrimentally enhance their suppressive activity. However, Tregs express lower levels of PD-1 than dysfunctional T-cells and are, therefore, less likely to be targeted and “activated” in response to treatment with RO7247669, wherein binding to PD-1 serves also as a handle. LAG3 has recently been validated as a clinical target in first-line (1L) treatment of melanoma with the recent approval of the anti-LAG3 antibody relatlimab in combination with the anti-PD-1 antibody nivolumab.

The dose optimization and dose selection paradigms in oncology have been required to shift in recent years due to the move away from cytotoxic agents to cancer immunotherapy and molecular targeted agents. Despite the need for dose and schedule to be adequately characterized prior to registration trials, there is still a mindset that a higher dose provides higher efficacy.

The pharmacological effect of an agent that blocks an inhibitory checkpoint receptor is driven by engaging the receptor on immune cells. Saturation of target engagement (TE) can thus be used as a surrogate for pharmacology saturation, i.e., maximal effect on downstream signaling. Thus, after achieving saturation of the target receptor, additional drug is not expected to lead to additional pharmacological effect. The dose required for target saturation in the tumor can thus be proposed as a recommended phase II dose.

### B. Methods

i. Clinical Study

RO7247669 is currently being evaluated as a single agent in the Phase I Study NP41300. Study NP41300 is an open-label, multicenter, dose escalation, Phase I study to evaluate the safety/tolerability, pharmacokinetics (PK), pharmacodynamics, and preliminary anti-tumor activity of RO7247669 in patients with locally advanced and/or metastatic solid tumors. The study consists of a dose escalation arm having two schedules (Part A) and a tumor-specific expansion arm (Part B). Part A consists of Part A1 (dosing once every two weeks (Q2W)) and Part A2 (dosing once every three weeks (Q3W)) dose escalation design to determine the maximum tolerated dose (MTD) and/or recommended dose for expansion (RDE) of RO7247669 in patients with solid tumors. Part B consists of tumor-specific expansions of RO7247669 administered at the MTD and/or RDE and at other doses of interest for future development in a subset of patients with selected solid tumor indications. Adult patients with advanced and/or metastatic solid tumors were enrolled as per the protocol.

ii. Clinical Pharmacokinetics

A validated target-binding competent PK assay was used to determine RO7247669 serum concentrations. For the assessment of RO7247669 PK, serum samples were collected at regular prespecified time points in patients in the NP41300 study. These included both peak (within 30 minutes after end of infusion) and trough (within 24 hours before the next dose) samples and rich sampling at Cycle 1 and 5.

The population PK analysis was performed using a nonlinear mixed effects modeling approach. One- and two-compartment models were evaluated, followed by testing different residual error structures. The model was then refined by testing interindividual variability for each PK parameter, followed by inspection of the correlations among the random effect values to guide the development of a parsimonious omega structure. Model selection was based on the log-likelihood criterion, goodness-of-fit plots, and scientific plausibility.

iii. Tumor Receptor Occupancy Modeling

The pharmacological effect of PD1-LAG3 is driven by the engagement of PD1 and LAG3 on CD8+ T cells. Saturation of these receptors on the CD8 cells can thus be used as a surrogate for maximal effect on downstream signaling; additional PD1-LAG3 is not expected to lead to additional pharmacological effect.

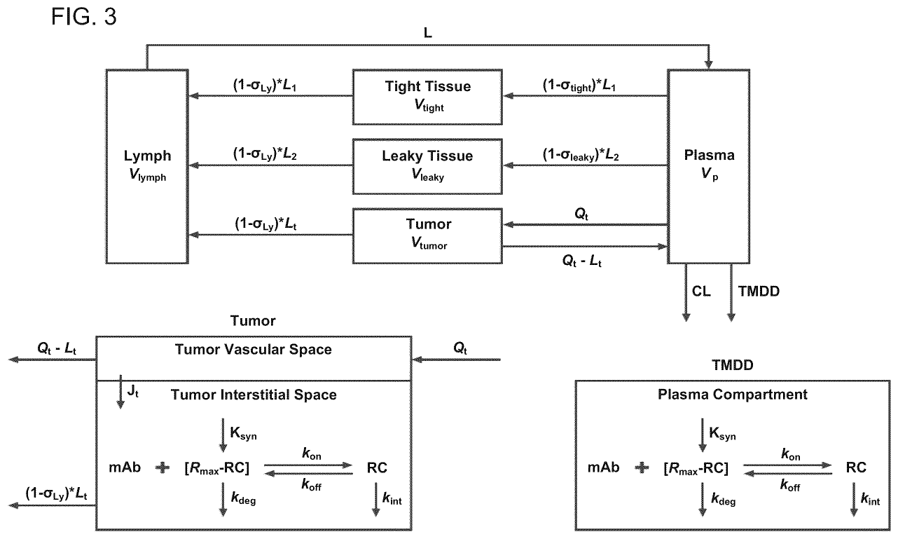

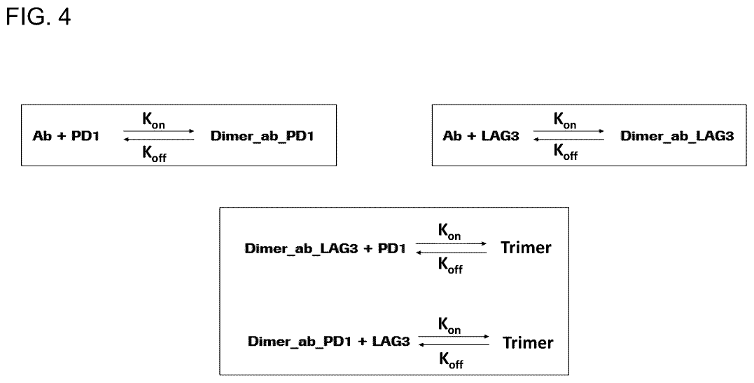

Tumor receptor occupancy modeling was performed. The goal was to characterize intratumoral PD1 and LAG3 engagement as a function of RO7247669 concentration and dose. For the purpose of estimating the dose required to saturate the target with PK data, a minimal physiologically-based pharmacokinetic (mPBPK) model was used. The model simplifies different tissues/organs into two distinct types of tissues sufficient to describe RO7247669 systemic concentration, and includes a tumor compartment to describe intratumoral RO7247669 concentration and PD1 and LAG3 engagement, providing a fit-for-purpose approach. The two tumor subcompartments, represented the vascular and interstitial space. PD1-LAG3 enters and leaves tumor vascular space based on tumor blood flow rate. Once inside the tumor, PD1-LAG3 can bind to PD-1 and/or LAG3 on tumor-associated immune cells, be eliminated via target-mediated drug disposition (TMDD), or be eliminated by convective flow. A tumor compartment was also added to the model to describe the tumor uptake of RO7247669. A simpler model (with only one target) has been previously described by Li et al., Clinical Pharmacology and Therapeutics, 110 (1): 200-209, 2021 to predict a recommended Phase 2 dose for pembrolizumab (FIG. 3). A randomized dose comparison study later confirmed the RP2D presented in Li et al. as the pivotal dose across tumor type. The additional LAG3 receptor added to the model for PD1-LAG3 is shown in FIG. 4. Population PK model parameters are shown in Table 16. Model parameters in addition to the pembrolizumab model reported by Li et al. are provided in Table 17.

TABLE 16
Population PK Model Parameters

| | | Relative | 95% | |
|---|---|---|---|---|
| | | Standard | Confidence | |
| Parameter | Estimate | Error | Intervals | Shrinkage |
| CL (L/h) | 0.0086 | 4.70 | 0.00785-0.00944 | — |
| V1 (L) | 3.11 | 2.08 | 2.98-3.24 | — |
| Q (L/h) | 0.0229 | 7.77 | 0.0194-0.0264 | — |
| V2 (L) | 1.98 | 7.64 | 1.68-2.27 | — |
| HT on V1 | 1.99 | 43.1 | 0.31-3.67 | — |
| WT on CL | 2.35 | 12.7 | 1.76-2.93 | — |
| HT on Q | 0.572 | 52.3 | −0.0146-1.16 | — |
| IIV-CL | 0.114 | 17.7 | 0.0748-0.154 | 11.2 |
| IIV-V1 | 0.0217 | 20.1 | 0.0131-0.0303 | 12.8 |
| IIV-Q | 0.214 | 31.6 | 0.0814-0.346 | 32.1 |
| IIV-V2 | 0.209 | 22.8 | 0.116-0.303 | 23.6 |
| Proportional Error | 0.148 | 4.61 | 0.134-0.161 | — |
| Additive Error | 0.712 | 35.6 | 0.214-1.21 | — |

CL: clearance; V1: central volume; Q: intercompartmental clearance; V2: peripheral volume; IIV: interindividual variability.

TABLE 17
Model parameters in addition to the pembrolizumab model

| Parameter | Value and Unit | |
|---|---|---|
| K_{D} LAG3 | 780 | pM |
| Koff LAG3 | 0.864 | day − 1 |
| Kon LAG3 | 1.104 | nM − 1 day − 1 |
| K_{D} PD1 | 250 | pM |
| Koff PD1 | 22.464 | day − 1 |
| Kon PD1 | 89.76 | nM − 1 day − 1 |
| CD8 blood PD1 receptors | 166.05 | pM |
| CD8 blood LAG3 receptors | 166.05 | pM |
| CD8 tumor PD1 receptors | 6.9743 | pM |
| CD8 tumor LAG3 receptors | 2.9059 | pM |
| Treg tumor PD1 receptors | 6.973 | pM |
| Treg tumor LAG3 receptors | 4.6495 | pM |

### C. Results

i. Clinical Efficacy and Safety

At the time of the clinical data cutoff (1 Mar. 2022), a total of 35 patients have been treated in Part A1 (Q2W) through six RO7247669 dose levels. In Part B, a total of 83 patients have been treated. Table 18 provides a summary of patients who received RO7247669 in NP41300.

The maximum tolerated dose (MTD) was not reached, and no dose-limiting toxicities (DLTs) were observed during dose escalation from 50 mg to 2100 mg Q2W. Greater than 90% occupancy of PD-1 and LAG3 receptors on peripheral CD8+ cells was observed following the first administration of 50 mg RO7247669 and saturation was observed at all doses to 2100 mg. In less than 20% of patients, formation of persistent ADAs against RO7247669 was observed. Reduction of exposure occurred in some ADA-positive patients receiving 300 mg or less. During the dose escalation phase, clinical responses were observed at doses of 600 mg and above.

Mean exposure increased with dose in a dose-proportional manner across the clinical dose range (50 to 2100 mg Q2W).

TABLE 18
Summary of patients who received RO7247669 in NP41300

| | |
|---|---|
| RO7247669 Dose and Schedule | Number of Patients |

Part A1

| | | |
|---|---|---|
| 50 | mg Q2W | 4 |
| 150 | mg Q2W | 4 |
| 300 | mg Q2W | 4 |
| 600 | mg Q2W | 4 |
| 1200 | mg Q2W | 6 |
| 2100 | mg Q2W | 13 |

Part B1 (melanoma)

| | | |
|---|---|---|
| 2100 | mg Q2W | 34 |

Part B2 (non-small cell lung cancer (NSCLC))

| | | |
|---|---|---|
| 2100 | mg Q2W | 19 |

Part B3 (esophageal cancer)

| | | |
|---|---|---|
| 2100 | mg Q2W | 8 |

Part B5

| | | |
|---|---|---|
| 600 | mg Q2W | 10 |

Part B6

| | | |
|---|---|---|
| 600 | mg Q3W | 12 |

As of 1 Mar. 2022, the disease control rate (DCR) in Part A of study NP41300 was 51.4% (18/35 patients) and the objective response rate (ORR) was 17.1% (6/35 patients). Among the patients dosed at the RDE of 2100 mg Q2W, 7 out of 13 patients (53.8%) had a best response of stable disease or better (DCR 53.8%) and the ORR was 30.8% (4/13 patients).

In addition to the confirmed partial responses (cPR) observed at the RDE, there were 2 cPRs at the 600 mg dose level (ORR 50.0%).

In Part B of the study, as of 1 Mar. 2022, the DCR was 48.3% (28/58) and the ORR was 5.2% (3/58) among patients of the study treated at the RDE of 2100 mq Q2W (Parts B1, B2 and B3). Within patients treated with 600 mg Q2W (Part B5), DCR was 40% (4/10), and ORR was 10% (1/10). Among patients treated with 600 mg Q3W, DCR was 28.6% (2/7) and ORR was 14.3% (1/7).

As of 1 Mar. 2022, DCR was 43.8% (14/32) and ORR was 6.3% (2/32) in Part B1, checkpoint inhibitor (CPI)-experienced melanoma patients. In Part B2, CPI-experienced NSCLC, 50% of the patients experienced clinical benefit (9/10 DCR), but none of them had a response. In Part B3, CPI-naïve esophageal squamous cell carcinoma (ESCC), DCR was 62.5% and ORR was 12.5% (1/8 patients). In the biomarker cohorts, DCR was 40% (44/10) and 28.6% (2/7), while ORR was 10% (1/10) and 14.3% (1 partial response (PR)/7) in Parts B5 and B6, respectively.

In Part A1 (Q2W dosing), the majority of patients (94.3%) reported at least one adverse event (AE). The most frequently reported AEs (occurring in at least 20% of patients) were anemia (37.1%), constipation (31.4%), dyspnea (28.6%), fatigue (25.7%), asthenia, decreased appetite (22.9% each), diarrhea, and pyrexia (20.0% each). Overall, 62.9% of patients reported treatment-related AEs. The incidence of serious adverse events (SAEs) was 25.7%, with 6 (17.1%) treatment-related SAEs. A total of 6 patients experienced SAEs that were considered to be related to study treatment by the investigator. The treatment-related SAEs include one AE of blood bilirubin increased in the 150 mg cohort, one AE of myositis in the 600 mg cohort, 2 AEs in the 1200 mg cohort (one AE of dyspnea and one of hyperthyroidism) and 2 AEs in the 2100 mg cohort (one AE of pleural effusion and one AE of anemia).

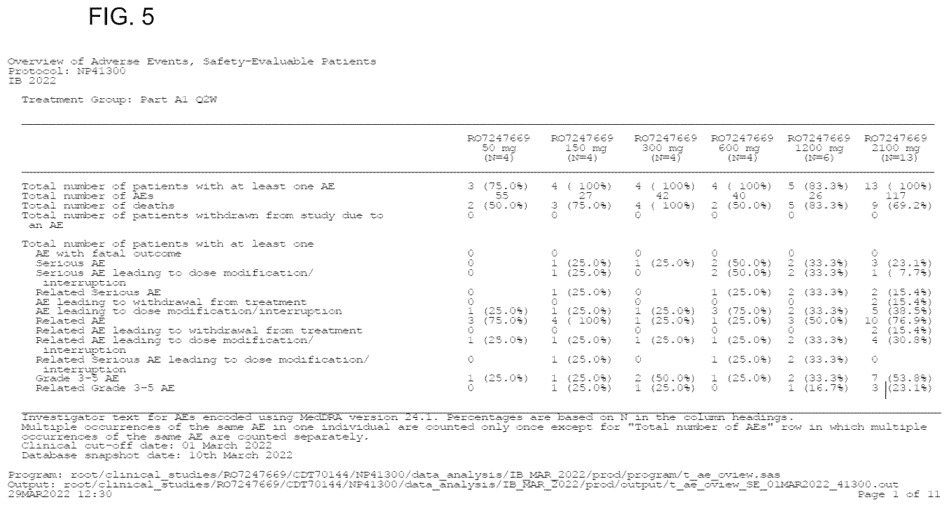

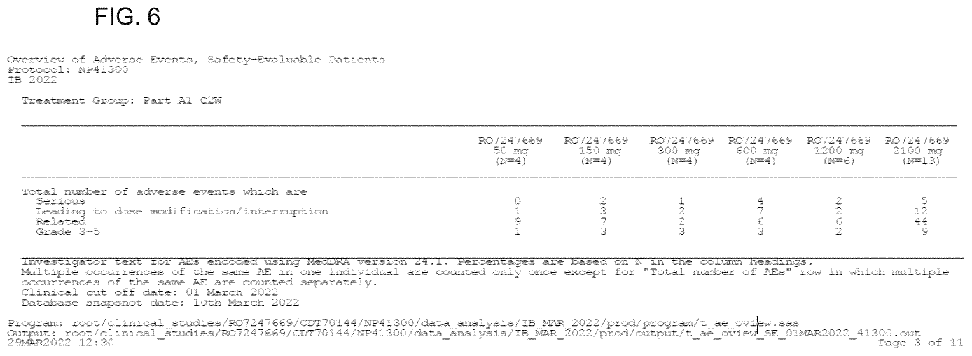

As of the clinical cutoff date of 1 Mar. 2022, preliminary safety data were available for 118 patients with solid tumors receiving RO7247669 monotherapy Q2W or Q3W in Part A1 and Part B. A total of 748 adverse events (AEs) were reported in 112 out of 118 patients (94.9%). Overall, 19 (54.3%) patients reported AEs of Grade 1 or 2 as maximum severity and 14 (40.0%) patients reported 21 Grade 3 AEs. No Grade 4-5 AEs were observed. Out of the 21 Grade 3 AEs, 6 AEs of Grade 3 were reported in 6 (17.1%) patients and were considered related to study treatment. Of these 6 related Grade 3 AEs, 4 AEs were considered serious. FIGS. 5 and 6 provide an overview of adverse events in safety evaluable patients in the dose escalation (Part A1, Q2W) portion of the study.

ii. Clinical Pharmacokinetics

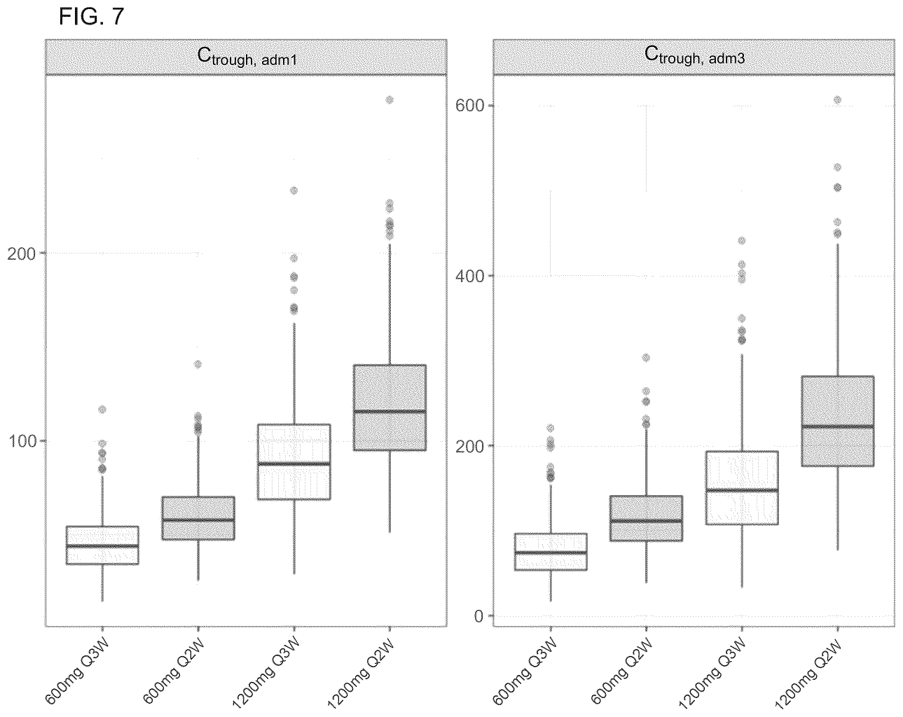

The PopPK model was used to simulate C_{trough} for 600 mg and 1200 mg for both Q2W and Q3W dosing regimens. As shown in FIG. 7, the trough concentrations after Q3W and Q2W of 600 mg and 1200 mg are predicted to overlap after the first and third administration.

A covariate analysis including tumor type has not yet been performed on the PD1-LAG3 PopPK model due to the limited dataset; however, analyses of nivolumab and pembrolizumab have reported minimal impact of tumor type on pharmacokinetics. In an analysis of nivolumab, the clearance (CL) was similar across tumor types, NSCLC, melanoma and RCC, indicating that PK is independent of tumor types. (Bajaj et al., CPT Pharmacometrics Syst Pharmacol, 6 (1): 49-57, 2017). In an analysis of pembrolizumab, the PopPK model included patient data from advanced melanoma, non-small cell lung cancer (NSCLC), and other solid tumor types. NSCLC patients had a 13.9% increase in clearance over other tumor types; however, this was not clinically relevant. (Ahamadi et al., CPT Pharmacometrics Syst Pharmacol, 6 (1): 58-66, 2017).

iii. Tumor Receptor Engagement Modeling

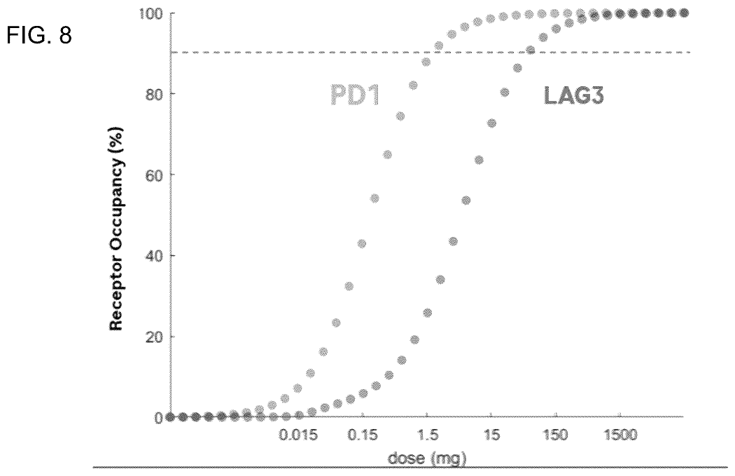

PD1 and LAG3 engagement was simulated for a wide range of doses of RO7247669, including 0.015 to 1500 mg administered Q3W (FIG. 8). The simulation shows that at 60 mg Q3W, PD1 and LAG3 are predicted to be saturated in the tumor, with 90% LAG3 receptor occupancy (RO) at 60 mg and 90% PD1 RO at 2.37 mg Q3W. From the population PK model, clearance was linear at this dose, suggesting that target-mediated drug disposition (TMDD) is saturated, which is consistent with the tumor receptor engagement model predictions.

To account for the fact that tumors are spatially heterogenous, and the prediction that RO7247669 will have a range of tumor penetrations, the model simulated for a range of vascularization, wherein poor vascularization was 25-fold lower than well-vascularized areas. In addition, a level of inter-subject variability in RO7247669 exposure is expected: therefore, it is predicted that 600 mg Q3W would ensure that the majority of patients would have at least a 90% LAG3 RO irrespective of tumor uptake levels.

### D. Discussion

A recommended RO7247669 dose and schedule of 600 mg Q3W was estimated using clinical and PK data from NP41003, the dose escalation study in solid tumors. A quantitative model based on published models and incorporating the RO7247669 PopPK model and data on the properties of the PD1 and LAG3 targets was used to simulate target engagement across a range of clinical doses at a Q3W regimen. Accounting for both intersubject and intrasubject variability in both exposure and vascularization of the tumor, 600 mg Q3W was predicted to saturate both PD1 and LAG3 receptors on CD8 cells in the tumor. The pharmacological effect of blocking inhibitory checkpoint receptors is driven by engaging the receptor on immune cells; saturation of target engagement can thus be used as a surrogate for pharmacology saturation, i.e., maximal effect on downstream signaling. Thus, after achieving saturation of the target receptor, additional drug is not expected to lead to additional pharmacological effect.

This conclusion was supported by the clinical data from NP41300, where responses were observed in the dose cohorts of 600 mg Q2W and above. In addition, 600 mg Q2W was well tolerated in the dose escalation cohort. The predicted C_{trough} for 600 mg Q2W and Q3W were overlapping, and therefore there is not expected to be a clinically relevant difference between the two schedules. The more patient-centric schedule of 600 mg Q3W was thus selected for further investigation.

### Example 3: A Randomized, Open-Label, Multicenter, Phase II Study of Multiple Doses of RO7247669 in Participants with Previously Untreated Unresectable or Metastatic Melanoma

### A. Study Design

Cancer remains a major cause of death worldwide despite several new agents providing survival benefits to patients. Many cancer indications have a poor prognosis and the management of most advanced solid tumors remains challenging because of the high rate of tumor recurrence or the development of distant metastases. Despite the effectiveness of currently available checkpoint inhibitor (CPI) therapies in various tumor types including melanoma, additional treatment options targeting immune checkpoints are needed because patients eventually progress after an initial response or fail to respond to the PD-1/L1 or cytotoxic T lymphocyte-associated antigen-4 (CTLA-4) checkpoint blockade.

Current standard of care for metastatic melanoma includes treatment with immune checkpoint inhibitors (CPIs), either alone or in combination, as well as treatment with targeted v-Raf murine sarcoma viral oncogene homolog B1 (BRAF) and mitogen-activated protein kinase (MEK) inhibitor therapies. Interleukin-2 (IL-2), oncolytic viruses, and interferon (IFN) therapy remain as options for a subset of patients. Overall, despite advances in therapeutic options, patients with metastatic melanoma continue to experience poor long-term clinical outcomes, reflecting the aggressive nature and complex etiology of the disease, and highlighting the continued unmet medical need.

RO7247669 (PD1-LAG3), an anti-programmed cell death protein 1 (PD-1)/lymphocyte activation gene 3 (LAG3) bispecific antibody (BsAb), was designed to target dysfunctional tumor antigen-specific T lymphocytes in order to establish or restore an effective anti-tumor immune response in patients with cancer with a high unmet medical need. By targeting both PD-1 and LAG3 on dysfunctional tumor-specific T lymphocytes, RO7247669 aims to restore an effective anti-tumor immune-response and provide survival benefit to more patients with cancer than currently available agents do. The combination blockage of LAG3 and PD-1 may have the potential to improve efficacy without adding significant toxicity compared to blocking PD-1 alone and be a therapeutic option for patients with melanoma.

Clinical proof-of-concept for the dual inhibition of PD-1 and LAG3 was recently provided by the combination of relatlimab and nivolumab in patients with previously untreated metastatic or unresectable melanoma (Tawbi et al., N Engl J Med, 386 (1): 24-34, 2022). The inhibition of both immune checkpoints provided a greater benefit with regard to progression-free survival (PFS) than inhibition of PD-1 alone.

The purpose of the study described in this Example, BP43963, is to assess the efficacy, safety, pharmacokinetics (PK), and pharmacodynamics of two dose levels of RO7247669 in participants with unresectable or metastatic melanoma to select the recommended dose for further development. The objectives and endpoints of the study are summarized in Table 19.

TABLE 19
Objectives and endpoints of BP43963 study

| | |
|---|---|
| Objectives | Endpoints |

Primary

| | |
|---|---|
| To evaluate the clinical | Progression-free survival (PFS) defined |
| activity of RO7247669 at | as the time from randomization to the |
| 600 mg and 1200 mg every | first occurrence of progression as |
| 3 weeks (Q3W). | determined by the Investigator according |
| | to Response Evaluation Criteria in |
| | Solid Tumors version 1.1 (RECIST v1.1) |
| | or death during the treatment period |
| | or within 60 days of the last tumor |
| | assessment after treatment discontin- |
| | uation from any cause, whichever |
| | occurs first. |

Secondary

| | |
|---|---|
| To evaluate the safety | Nature, frequency, and severity of |
| and tolerability of | adverse events (AEs) graded according |
| RO7247669 at 600 mg | to the National Cancer Institute (NCI) |
| and 1200 mg Q3W. | Common Terminology Criteria for |
| | Adverse Events (CTCAE) v5.0. |
| To evaluate the clinical | Objective response rate (ORR), defined |
| activity of RO7247669 | as the proportion of participants with |
| at 600 mg and 1200 mg | an objective response (i.e., complete |
| Q3W. | response (CR) or partial response |
| | (PR)), according to RECIST v1.1. |
| | Disease control rate (DCR), defined |
| | as ORR + stable disease rate (SDR). |
| | Duration of response (DoR) for |
| | participants with objective response, |
| | defined as the time from the first |
| | occurrence of a documented objective |
| | response to disease progression |
| | according to RECIST v1.1 or death |
| | from any cause, whichever occurs first. |
| To investigate the pharma- | Serum concentrations, PK profiles and |
| cokinetics (PK) of | parameters for RO7247669. |
| RO7247669 at 600 mg and | |
| 1200 mg Q3W. | |
| To evaluate the immune | Incidence and titer of RO7247669 anti- |
| response after administration | drug antibodies (ADAs) during the study |
| of RO7247669 at 600 mg and | relative to the prevalence of ADA at |
| 1200 mg Q3W. | baseline. |
| To evaluate potential | Relationship between ADA status and |
| effects of anti-drug | PK, safety, pharmacodynamics, and |
| antibodies (ADAs). | efficacy. |
| To assess treatment-induced | Changes from baseline in the phenotype |
| pharmacodynamic changes | and activation status of T-cell |
| (PD biomarkers) in | subsets in the peripheral blood |
| peripheral blood and tumor | (CD4/CD8 HLA-DR+/Ki67+). |
| microenvironment. | Changes from baseline in the tumor |
| | microenvironment (such as CD8 T-cell |
| | infiltration, proliferation (CD8+Ki67+)). |

Exploratory

| | |
|---|---|
| To evaluate the clinical | Overall survival (OS), defined as the |
| activity of RO7247669 at | time from randomization to death from |
| 600 mg and 1200mg Q3W | any cause. |
| To correlate anti-tumor | Compare anti-tumor activity with PK |
| activity of RO7247669 | parameters, safety events, and |
| with PK and exploratory | exploratory safety biomarkers |
| safety biomarkers | (including genetic and genomic |
| | analyses). |
| To explore potential | PD Biomarkers: Changes from baseline |
| pharmacodynamics (PD) | such as Treg profile, cytotoxic T |
| and predictive biomarkers | cells in the tumor microenvironment, |
| associated with the | and peripheral immune cell subsets |
| anti-tumor activity | such as Tregs, Tres cells and central/ |
| of RO7247669 | effector memory subsets. |
| | Predictive Biomarkers: Baseline profiles |
| | such as selected target expression |
| | (PD-L1, LAG3, etc.), (blood) tumor |
| | mutational burden ((b)TMB), tumor gene |
| | expression, circulating tumor DNA |
| | (ctDNA), peripheral blood immune-cell |
| | subsets and association with clinical |
| | outcome. |
| To evaluate the toler- | Participant-reported tolerability with |
| ability of RO7247669 | treatment-related symptoms through the |
| at 600 mg and 1200 mg | Patient-Reported Outcomes Common |
| Q3W | Terminology Criteria for Adverse Events |
| | (PROCTCAE) and European Organisation |
| | for Treatment and Research of Cancer |
| | (EORTC) treatment bother item. |

The primary objective is expressed in the estimand framework through the five attributes described in Table 20.

TABLE 20

| Attribute | Study Definitions |
|---|---|
| Population | Participants with previously untreated |
| | unresectable or metastatic melanoma. |
| Variable | Progression-free survival (PFS). |
| Treatments | Experimental: 600 mg Q3W RO7247669. |
| | Experimental: 1200 mg Q3W RO7247669. |
| Intercurrent events | Treatment discontinuation, handled via |
| and their handling | treatment policy (intercurrent event is |
| | ignored). |
| | New anti-cancer therapy, handled as |
| | hypothetical strategy (only TAs that occurred |
| | before starting a new anticancer therapy are |
| | considered). |
| | Symptomatic deterioration, handled as |
| | composite strategy (part of the definition |
| | of ORR, since considered as an event). |
| Summary measure | Kaplan-Meier estimate at 6 months. |

ORR: objective response rate; PFS: progression-free survival; Q3W: every 3 weeks; TA: tumor assessment.

i. Overall Design

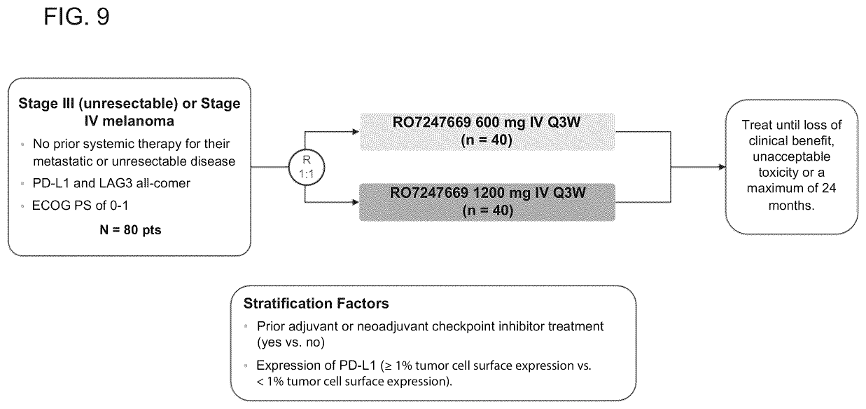

BP43963 is a Phase II, randomized, open-label, global, multicenter study designed to evaluate the safety and clinical activity of two different dose levels of RO7247669 in participants with unresectable or metastatic melanoma who have not received prior systemic therapy for their metastatic or unresectable disease. An overview of the study design is provided in FIG. 9.

The study enrolls approximately 80 participants aged ≥18 years with Eastern Cooperative Oncology Group (ECOG) Performance Status of 0 or 1. Participants are randomized in a 1:1 ratio to receive 600 mg every 3 weeks (Q3W) or 1200 mg Q3W RO7247669. Prior adjuvant or neoadjuvant CPI treatment (yes vs. no) and expression of PD-L1 (≥1% versus <1% expression based on immunohistochemistry (IHC) using antibody clone 22C3, SP263, or 28-8) are used as stratification factors.

Treatment may be continued as long as participants are experiencing clinical benefit, as assessed by the Investigator, in the absence of unacceptable toxicity or symptomatic deterioration attributed to disease progression after an integrated assessment of radiographic data, biopsy results (if available), and clinical status for a maximum of 24 months. Participants who meet the criteria for disease progression per RECIST v1.1 are permitted to continue study treatment if they meet all criteria for treatment beyond progression.

Participants undergo their first tumor assessments at 12 weeks (±1 week) relative to date of cycle 1, day 1 (C1D1). Subsequent tumor assessment occurs every 9 weeks (±1 week) up to 48 weeks and then every 12 weeks (±1 week) relative to date of C1D1, regardless of treatment delays.

Response is assessed according to RECIST v1.1. Objective response at a single time-point is determined by the Investigator according to RECIST v1.1. After study treatment discontinuation and disease progression per RECIST v1.1, survival follow-up information is collected by means of telephone calls, participant medical records, and/or clinic visits approximately every 3 months until death, loss to follow-up, study termination, or a maximum of 24 months after randomization,
whichever occurs first.

During the study, serum samples are collected to assess RO7247669 PK and to detect the presence of antibodies to RO7247669. Participant samples, including archival and fresh tumor tissue, serum, plasma, and blood samples, are also collected for biomarker assessments.

Safety assessments include the incidence, nature, and severity of adverse events (AEs), and other protocol-specified tests such as laboratory abnormalities that are deemed critical to the safety evaluation of the study.

### Length of Study and End of Study

The study duration per participant from screening until safety follow-up visit is up to approximately 25 months (not including long-term follow-up (LTFU)).

The duration in each period of the study for each participant is as follows:
Screening: Up to 28 days prior to randomization.
Treatment Period: Cycle 1 Day 1 (C1D1) up to a maximum of 24 months.
Survival follow-up: 90 (+1 week) days after last dose of study treatment; then every 3 months (+2 weeks) until death, loss to follow-up, study termination, or a maximum of 24 months after randomization.

The end of the study is defined as the date when the last participant last observation (LPLO) occurs. LPLO is expected to occur at the latest 24 months after the last participant is randomized.

### Participant Population

The participant population consists of female and male participants with unresectable or metastatic melanoma with no prior systemic anticancer therapy for unresectable or metastatic disease.

### Number of Participants

The planned number of participants randomized to study treatment is 80 such that approximately 80 participants will be evaluable for the analysis of the primary endpoint.

### Concomitant Medications

In general, no concomitant medication for the treatment of melanoma is permitted, with the exception of medications to treat adverse events (AEs), unless the rationale for exception is discussed and clearly documented.

### Background Information on Melanoma

Cancer of the skin is the most common of all cancers. Melanoma, which accounts for merely 1% of all skin cancers, is the most aggressive and dangerous form of skin cancer that develops from melanocytes. According to the American Cancer Society, approximately 100,000 new cases of invasive melanoma will be diagnosed in 2022 in the US. Although the outcome for promptly diagnosed superficial tumors is good, in the metastatic setting, melanoma continues to be one of the most deadly cancers, with a 5-year survival rate of 27% (Surveillance, Epidemiology, and End Results (SEER) 2021, available at the American Cancer Society web page). Prior to 2011, approved therapies for the treatment of metastatic melanoma were limited and included chemotherapy and immunotherapy (IL-2). Since then, new therapeutic options have been approved, which include drugs targeting the tyrosine kinase pathways, CPI therapies that target CTLA-4 and the PD-1 receptor, as well as a vaccine.

The anti-CTLA-4 antibody ipilimumab was the first CPI to show a significant improvement in clinical outcomes and was consequently approved for use in unresectable or metastatic melanoma (Hodi et al., N Engl J Med, 363:711-723, 2010). Subsequently, monoclonal antibodies that bind to the PD-1 receptor (pembrolizumab or nivolumab) yielded improved outcomes and reduced toxicity (Robert et al., N Engl J Med, 372:320-330, 2010; Robert et al., N Engl J Med, 372 (26): 2521-2532, 2010; Weber et al., Lancet Oncol, 16 (4): 375-384, 2015). Combined CTLA-4/PD-1 blockade resulted in higher response rates and longer OS compared to the respective single agent's activity. However, the combination therapy was accompanied by increased toxicity (Wolchok et al., Abstract 9506 ASCO Annual Meeting 2021).

Recent results from a Phase 2/3 study (RELATIVITY-047) in previously untreated metastatic or unresectable melanoma showed that combined blockade of PD-1 (via nivolumab) and LAG3 (via relatlimab) improves PFS compared to inhibition of PD-1 alone (Tawbi et al., N Engl J Med, 386 (1): 24-34, 2022). The PFS benefit observed for this combination appears similar compared to that of the combination of nivolumab and ipilimumab, however with a more favorable safety profile (Tawbi et al., N Engl J Med, 386 (1): 24-34, 2022, Wolchok et al., Abstract 9506 ASCO Annual Meeting 2021). At the same time, relatlimab and nivolumab in combination showed no new safety signals as compared to treatment with nivolumab alone.

Current standard of care for metastatic melanoma includes treatment with immune CPIs, either alone or in combination, as well as treatment with targeted BRAF and MEK inhibitor therapies. IL-2, oncolytic viruses, and IFN therapy remain as options for a subset of patients.

Overall, despite advances in therapeutic options, patients with metastatic melanoma continue to experience poor long-term clinical outcomes, reflecting the aggressive nature and complex etiology of the disease, and highlighting the continued unmet medical need.

### Background Information on RO7247669

RO7247669 is a novel, Fc-silent IgG1-based bispecific antibody in 1+1 format that incorporates monovalent binding to each of two CPIs: PD-1 and LAG3. RO7247669 is engineered to preferentially bind to T-cells in the tumor microenvironment that co-express both PD-1 and LAG3, or to a lesser extent either PD-1 or LAG3 alone. Preferential binding to T cells in the tumor microenvironment avoids the targeting of other LAG3 expressing cells such as regulatory T cells in the tumor and in the periphery (which do not express high levels of PD-1). Monovalent binding to LAG3 reduces internalization of the antibody (Ab) upon binding to the T-cell surface. An additional feature of the PD-1 BsAb is the engineered IgG1-based Fc-region that prevents binding to Fc gamma receptors by introduction of LALA P329G mutations. This avoids drug-shaving and thus tumor-associated macrophage resistance mechanisms which have been observed with IgG4-based antibodies such as pembrolizumab and nivolumab (Arlauckas et al., Sci Transl Med, 9 (389): eaal3604, 2017).

RO7247669 is currently being evaluated in four ongoing clinical studies:
1. A Phase I entry-into-human study in participants with advanced and/or metastatic solid tumors including a second-line melanoma expansion cohort (Study NP41300);
2. A Phase II study in second-line participants with advanced or metastatic esophageal squamous-cell carcinoma (Study BP42772);
3. A Phase Ib/II study in first-line participants with advanced hepatocellular carcinoma in combination with bevacizumab (Study GO42216); and
4. A Phase Ib/II study in neoadjuvant resectable Stage III and later lines Stage IV melanoma participants with or without tiragolumab (Study BO43328).

Across all studies, RO7247669 was well tolerated up to the maximum dose tested (2100 mg Q2W), and no specific safety concern associated with RO7247669 were identified. No dose-limiting toxicities (DLTs) were observed and no maximum tolerated dose (MTD) was identified during the dose escalation portion in Study NP41300.

Study BP42772 is a blinded study, and recruitment into Studies GO42216 and BO43328 only commenced recently. Relevant efficacy data are currently available from Study NP41300. As of the data cut-off date of 14 Jan. 2022, the disease control rate (DCR) in the dose-escalation of the study was 51% (18 of 35 evaluable participants) and the objective response rate (ORR) was 17% (6 of 35 participants). Responses were observed in both CPI naive and CPI-experienced participants across various tumor types, at 600 mg every 2 weeks (Q2W) and 2100 mg Q2W. While there were no responders at doses<600 mg (N=12), there were 7/23 responders at doses≥600 mg Q2W, indicating a dose-response relationship. At doses≥600 mg Q2W, no apparent relationship between exposure and best overall response (BOR) was observed. The mean Cₐᵥₑₛₛ (average concentration at steady state) of the patients with progressive disease was similar to the patients with partial response within the 600 and 2100 mg cohorts.

As of 14 Jan. 2022, 28 CPI-experienced uveal (9 of 28 participants) and non-uveal (19 of 28) PDL-1 and LAG3 all-comer melanoma participants have been evaluated for efficacy at 2100 mg Q2W in an expansion cohort of NP41300. Amongst the non-uveal melanoma participants, the ORR was 21% (4 of 19) and DCR was 58% (11 of 19).

Responses were observed in two participants who previously received pembrolizumab, one participant who received nivolumab, and one participant who received the combination treatment of nivolumab and ipilimumab. Enrollment is still ongoing.

### Benefit-Risk Assessment

Despite limited clinical experience with RO7247669, the clinical efficacy and safety profiles of anti-PD-1/PD-L1 monoclonal antibodies are well characterized and established. In recent years, three CPIs, namely pembrolizumab, nivolumab, and ipilimumab, have been approved by the Food and Drug Administration (FDA) and other Health Authorities for the treatment of unresectable or metastatic melanoma. The median overall survival (OS) for patients with advanced melanoma is reported to be

72.1 months for the combination of nivolumab and ipilimumab, 36.9 months for nivolumab monotherapy, and 19.9 months for ipilimumab monotherapy (Wolchok et al., Abstract 9506 ASCO Annual Meeting 2021).

Next-generation combination therapies could potentially lead to higher response rates, greater depth of response, longer or similar OS with clinically meaningful improved safety profiles as have been noted with the combination of anti-PD-1 and anti-CTLA-4 in advanced melanoma patients. Thus, compared to monospecific PD-1 directed antibodies, one might expect a better efficacy primarily coming from targeting both PD-1 and LAG3-mediated immune-resistance mechanisms.

As discussed above, clinical proof-of-concept for the dual inhibition of PD-1 and LAG3 was recently provided by the combination of relatlimab and nivolumab in patients with previously untreated metastatic or unresectable melanoma (Tawbi et al., N Engl J Med, 386 (1): 24-34, 2022). The inhibition of both immune checkpoints provided a greater benefit with regard to PFS than inhibition of PD-1 alone. At the same time, relatlimab and nivolumab in combination showed no new safety signals and had a significantly improved safety profile when retrospectively compared to nivolumab and ipilimumab combination (Tawbi et al., N Engl J Med, 386 (1): 24-34, 2022, Wolchok et al., Abstract 9506 ASCO Annual Meeting 2021).

As discussed above, RO7247669 was shown to be capable of inducing responses in patients with melanoma and other tumor types that have demonstrated resistance to CPI therapy including combination treatment with nivolumab and ipilimumab. In particular, the ORR of 21% in non-uveal melanoma participants observed in Study NP41300 compares well with the overserved response rate of 11.5% for the relatlimab and nivolumab combination in a comparable patient population (Ascierto et al., Ann Oncol, v611-v612, 2017).

RO7247669 has been tolerated at doses of up to 2100 mg Q2W; AEs have been manageable, and the safety profile is observed to be consistent across different solid tumor indications as well as with approved PD-1 directed antibodies. In summary, RO7247669 has the potential for increased benefit compared to nivolumab monotherapy, with a similar safety profile.

Taking into account the preliminary efficacy and manageable safety profiles, as well as the potential for improved outcomes due to their mechanisms of action of dual checkpoint inhibition, treatment with RO7247669 has therapeutic potential in solid tumors such as melanoma. Based on the considerations above, currently available data and the planned safety monitoring and management guidance, the proposed study treatment is considered to have an appropriate benefit/risk profile for the population included in this study.

### B. Rationale

### Rationale for Study Population

The study enrolls participants with unresectable or metastatic melanoma and who have not received prior systemic anti-cancer therapy for unresectable or metastatic disease. The clinical validation of a monospecific anti-LAG3 antibody in combination with a monospecific anti-PD-1 antibody recently occurred in this therapy setting (Tawbi et al., N Engl J Med, 386 (1): 24-34, 2022). Together with the emerging efficacy data from the ongoing CPI-experienced melanoma cohort in Study NP41300, it is expected that treatment with RO7247669 would provide clinical benefits in this indication.

First-line melanoma was also chosen because it allows for the testing of two different dose levels of monotherapy RO7247669, given that CPI treatment without chemotherapy is the current standard of care in this population. Consequently, it is possible to assess dose-dependent effects in the absence of potential confounding effects of combination partners.

The disease-specific eligibility criteria for this study are similar to those used in RELATIVITY-047. A benefit of treatment with relatlimab and nivolumab combination as compared to nivolumab alone was observed independent of tumor PD-L1 and LAG3 expression and regardless of patients' BRAF mutation status. For this reason, participants are eligible for this study independent of tumor PD-L1 and LAG3 expression and their BRAF mutation status. The inclusion of participants with BRAF-mutated melanoma is also considered justified since current guidelines recommend to consider treatment with immunotherapy prior to treatment with BRAF and MEK inhibitors, as it may provide very long-term disease control even after stopping the treatment (Keilholz et al., Ann Oncol, 31 (11): 1435-1448, 2020).

### Rationale for Primary Endpoint

While OS remains the gold standard for the demonstration of clinical benefit, measuring this endpoint requires larger patient numbers and longer follow-up time than other endpoints and may be confounded by subsequent therapies (Pazdur et al., Oncologist, 13 Suppl. 2:19-21, 2008). In contrast, PFS (including landmark PFS) is an early readout that allowed for a differentiation of treatment benefits in first-line melanoma in the past (Wolchok et al., Abstract 9506 ASCO Annual Meeting 2021). Consequently, PFS has been recognized as an acceptable regulatory endpoint. For example, dabrafenib and trametinib as monotherapy and in combination were approved by the FDA for treatment of unresectable or metastatic melanoma based on Phase 3 clinical trials where the primary endpoint was PFS (Flaherty et al., N Engl J Med, 367:107-114, 2012; Hauschild et al., Lancet, 380:358-365, 2012; Long et al., ASCO Annual Meeting Abstracts; 32:9011, 2014). PFS has also been the primary endpoint in the RELATIVITY-047 trial, and benefits were demonstrated already after 6 months of treatment (Tawbi et al., N Engl J Med, 386 (1): 24-34, 2022).

Compared to OS, PFS is not confounded by post-study treatment therapies. Thus, we consider PFS as the preferred option for selected patient population since treatment options that are clinically active (e.g., nivolumab, ipilimumab, vemurafenib, dabrafenib, and trametinib) are increasingly available in patients with unresectable or metastatic melanoma and their use after disease progression on the current study may confound an OS endpoint.

For these reasons, PFS at 6 months has been selected as the primary endpoint for efficacy.

### Rationale for Stratification Factors

In order to minimize the potential for imbalances across treatment arms, there are two stratification factors utilized in the trial: Prior adjuvant or neoadjuvant checkpoint inhibitor treatment (yes vs. no) and PD-L1 expression (≥1% tumor cell surface expression versus <1% tumor cell surface expression based on IHC using antibody clone 22C3, SP263, or 28-8).

Prior adjuvant or neoadjuvant CPI treatment (yes vs. no) was chosen since the use of CPI treatment has improved recurrence-free survival (RFS) after complete resection of high-risk Stage II/III melanoma. Adjuvant and neoadjuvant treatment with CPIs is or will become a part of the standard of care in the treatment of melanoma. For this reason, participants who completed adjuvant or neoadjuvant anti-PD-1 or anti-CTLA-4 therapy with at least 6 months between the last dose and date of recurrence are eligible for the study. Since the effect of prior adjuvant or neoadjuvant anti-PD-1 or anti-CTLA-4 therapy on responsiveness to anti-PD-1 and anti-LAG3 combination therapies in unresectable or metastatic disease is not yet known, participants in the current trial are stratified accordingly.

With regard to PD-L1 expression, previous clinical studies with nivolumab monotherapy have shown that patients with PD-L1 positive tumors may have higher response rates than those with indeterminate expression (Robert et al., N Engl J Med, 372:320-330, 2010). Similarly, median PFS for patients treated with the combination of relatlimab and nivolumab was reported to be 12.6 months among patients with PD-L1 expression of 1% or greater while for patients with PD-L1 expression of less than 1%, the median PFS with relatlimab and nivolumab combination was 6.4 months (Tawbi et al., N Engl J Med, 386 (1): 24-34, 2022).

### Rationale for Biomarker Assessments

The main hypothesis in the study is that additional checkpoint blockade via PD-1-LAG3 will re-invigorate exhausted T cells and lead to an increase in CD8 T-cell infiltration and proliferation in the tumor microenvironment. As such, treatment-induced biomarker exploration will focus on changes from baseline associated with PD-1-LAG3 in both the tumor microenvironment and peripheral blood. Serial blood sampling of peripheral blood is also done to assess kinetic changes in immune cellular profiles, profiles that will then be examined for association with treatment with RO7247669.

To explore predictive markers of response for RO7247669, the initial hypothesis focuses on association of response with baseline PD-L1, CD8, LAG3, and/or PD-1 expression. Other exploratory biomarkers include assessing baseline immune-cell subsets/effector gene signatures in the peripheral blood and tumor microenvironment or other soluble markers (such as blood tumor mutational burden, circulating tumor deoxyribonucleic acid (ctDNA), etc.). Additional biomarkers may be measured if initial data lead to a strong scientific rationale for these measurements.

### Justification for Dose

RO7247669 is administered at doses of 600 mg or 1200 mg Q3W (on Day 1 of each 21-day cycle).

As discussed in Example 2, in Study NP41300, RO7247669 was tolerated and no new safety concern associated with RO7247669 was identified. No DLT up to the highest dose of 2100 mg Q2W was observed, and no MTD was identified.
Anti-tumor activity, as measured by radiographic PRs, was observed at 600 mg and 2100 mg Q2W (Study NP41300).
The PK of RO7247669 were dose linear within the dose range tested in Study NP41300.
Further modelling of PD-1 and LAG3 target engagement in the tumor using a minimal physiological based pharmacokinetic model to describe intratumoral RO7247669 concentrations and estimation of target mediated drug disposition characterized by binding properties of RO7247669 was performed. It was estimated that 600 mg Q3W RO7247669 would result in >90% PD-1 and LAG3 target engagement at the site of the tumor throughout the treatment period in the majority of participants considering both the inter-participant variability of PK and the spatial heterogeneity of RO7247669 distribution within a tumor. A similar approach was used to confirm the recommended Phase II dose as the pivotal dose for pembrolizumab (Li et al., Clinical Pharmacology and Therapeutics, 110 (1): 200-209, 2021).
Tumor receptor occupancy simulations were performed for 600 mg Q2W and 600 mg Q3W and both regimens resulted in >90% PD-1 and LAG3 receptor occupancy through the treatment period, therefore the less frequent regimen of Q3W was selected to minimize treatment burden on the participant.
The immunogenicity of RO7247669 and the effects of anti-drug antibody (ADA) development on exposure and efficacy are not yet fully understood. However, there is evidence for an ADA-mediated impact on exposure observed at doses of ≤300 mg in Study NP41300. No ADAs were observed to date at 600 mg, which leaves uncertainty on a potential ADA-mediated impact on exposure at 600 mg.

There has been no observed impact on exposure in participants that developed ADAs at 1200 mg and 2100 mg to date: therefore, it is expected that 1200 mg would saturate circulating ADA and minimize impact on exposure.

Therefore, the dose and regimen of 600 mg and 1200 mg Q3W were selected to be administered to participants in this study.

### C. Inclusion and Exclusion Criteria

The participant population consists of participants diagnosed with unresectable or metastatic melanoma with no prior systemic anticancer therapy for unresectable or metastatic disease, who fulfill all of the given inclusion criteria and none of the exclusion criteria.

### Inclusion Criteria

Participants are eligible to be included in the study only if all of the following criteria apply:

### General

1. Able and willing to provide written informed consent and to comply with the study protocol according to International Council for Harmonisation (ICH) and local regulations.
2. Age≥18 years

### Type of Participants and Disease Characteristics

3. Participants must have histologically confirmed unresectable or metastatic melanoma, per the AJCC staging system (unresectable Stage III or Stage IV).
4. Radiologically measurable disease according to RECIST v1.1. Previously irradiated lesions should not be counted as target lesions unless clearly progressed after the radiotherapy.
5. ECOG Performance Status 0-1.
6. Participants must have known BRAF V600 mutation status.
7. Participants must have known PD-L1 status.
   PD-L1 expression is defined as the percent of tumor cells demonstrating plasma membrane PD-L1 staining of any intensity using an approved IHC assay using antibody clone 22C3, SP263, or 28-8.
   PD-L1 expression needs to be determined on a tissue sample obtained within 3 months prior to enrollment and with no intervening treatment between time of acquisition and PD-L1 testing.
8. Tumor tissue from an unresectable or metastatic site of disease must be provided for biomarker analyses.
9. Participants must have at least one non-target tumor lesion accessible to biopsy per clinical judgment of the treating physician and consent undergo mandatory on-treatment biopsy.

### Medical Conditions

10. Adequate cardiovascular function.

11. AEs from any prior radiotherapy, chemotherapy, or surgical procedure must have resolved to Grade ≤1, except alopecia (any grade), vitiligo, endocrinopathy managed with replacement therapy, and Grade 2 peripheral neuropathy.
12. Adequate hematological function.
13. Adequate liver function.
14. Adequate renal function.
15. Additional adequate laboratory parameters obtained:
Serum albumin≥25 g/L (2.5 g/dL)
For participants not receiving therapeutic anticoagulation: prothrombin time (PT) and activated partial thromboplastin time≤1.5×ULN
For participants receiving therapeutic anticoagulation: stable anticoagulant regimen.

### Contraception

16. Male and/or female participants:

The contraception and abstinence requirements are intended to prevent exposure of an embryo to the study treatment. The reliability of sexual abstinence for male and/or female enrollment eligibility needs to be evaluated in relation to the duration of the clinical study and the preferred and usual lifestyle of the participant. Periodic abstinence (e.g., calendar, ovulation, symptom-thermal, or post-ovulation methods) and withdrawal are not acceptable methods of contraception.
Female participants: A female participant is eligible to participate if she is not pregnant, not breastfeeding, and at least one of the following conditions applies:
   Not a woman of childbearing potential (WOCBP).
   WOCBP, who:
   Agree to remain abstinent (refrain from heterosexual intercourse) or use highly effective contraceptive methods that result in a failure rate of <1% per year during the treatment period and for at least 4 months.
   after the final dose of study drug. Women must refrain from donating eggs during this same period.
   Has a negative pregnancy test (blood) within the 7 days prior to randomization.
Male participants: During the treatment period and for at least 4 months after the final dose of study drug, agreement to:
   Remain abstinent (refrain from heterosexual intercourse) or use contraceptive measures such as a condom, with a WOCBP or pregnant female partner, to avoid exposing the embryo.
   Refrain from donating sperm.

### Exclusion Criteria

Participants are excluded from the study if any of the following criteria apply:

### General

1. Pregnancy, lactation, or breastfeeding.
2. Known hypersensitivity to any of the components of RO7247669, including but not limited to hypersensitivity to Chinese hamster ovary cell products or other recombinant human or humanized antibodies.

### Type of Participants and Disease Characteristics

3. Participants must not have ocular melanoma.

### Medical Conditions

4. Symptomatic central nervous system (CNS) metastases. Participants with previously treated brain metastases may participate provided they:
   Are stable (without evidence of progression by computed tomography (CT) or magnetic resonance imaging (MRI) for at least 28 days prior to randomization).
   Have no evidence of new or enlarging brain metastases for at least 28 days prior to randomization.
   Have not received treatment with systemic steroids for at least 28 days prior to randomization.
5. Spinal cord compression not definitively treated with surgery and/or radiation or without evidence that disease has been clinically stable for ≥14 days prior to randomization.
6. Active or history of carcinomatous meningitis/leptomeningeal disease.
7. Asymptomatic CNS primary tumors or metastases if they have requirement for steroids or enzyme-inducing anticonvulsants in the last 28 days prior to randomization.
8. Uncontrolled tumor-related pain. Participants requiring pain medication must be on a stable regimen at study entry.
9. Participants with an active second malignancy. Concurrent malignancy exceptions include: curatively treated carcinoma in situ of the cervix, good-prognosis ductal carcinoma in situ of the breast, basal- or squamous-cell skin cancer, or low-grade, early-stage localized prostate cancer and any previously treated early stage nonhematological malignancy that has been in remission for at least two years.
10. Evidence of significant, uncontrolled concomitant diseases that could affect compliance with the protocol or interpretation of results, including diabetes mellitus, history of relevant pulmonary disorders, known autoimmune diseases or immune deficiency, or other diseases with ongoing fibrosis (such as scleroderma, pulmonary fibrosis, emphysema, neurofibromatosis, palmar/plantar fibromatosis, etc.).
11. Encephalitis, meningitis, or uncontrolled seizures in the year prior to informed consent.
12. Significant cardiovascular/cerebrovascular disease within 6 months prior to randomization, including any of the following:
   Hypertensive crisis/encephalopathy.
   Unstable angina.
   Transient ischemic attack/stroke.
   Congestive heart failure.
   Serious cardiac arrhythmia requiring treatment (exceptions are atrial fibrillation, paroxysmal supraventricular tachycardia).
   History of thromboembolic events (such as myocardial infarction, stroke or pulmonary embolism)
13. Known active or uncontrolled bacterial, viral, fungal, mycobacterial (including but not limited to tuberculosis and typical mycobacterial disease), parasitic, or other infection (excluding fungal infections of nail beds), or any major episode of infection requiring treatment with IV antibiotics or hospitalization (relating to the completion of the course of antibiotics, except if for tumor fever) within 28 days prior to randomization.
14. Known clinically significant liver disease, including alcoholic hepatitis, cirrhosis, and inherited liver disease.
15. Major surgical procedure or significant traumatic injury (excluding biopsies) within 28 days prior to randomization, or anticipation of the need for major surgery during the course of the study.
16. Any other diseases, metabolic dysfunction, physical examination finding, or clinical laboratory finding giving reasonable suspicion of a disease or condition that contraindicates the use of an investigational drug or that may affect the interpretation of the results or render the participant at high risk from treatment complications.
17. Dementia or altered mental status that would prohibit informed consent.
18. Uncontrolled pleural effusion (with the exception of participants with indwelling catheters, e.g., PLEURX®), pericardial effusion, or ascites requiring recurrent drainage procedures (expected to occur once monthly or more frequently).
19. Active or history of autoimmune disease or immune deficiency with the following exceptions:
   Participants with a history of autoimmune-mediated hypothyroidism or endocrinopathy who are stable on thyroid-replacement hormone or appropriate replacement therapy are eligible for the study.
   Participants with controlled Type 1 diabetes mellitus who are on an insulin regimen are eligible for the study.
   Participants with eczema, psoriasis, lichen simplex chronicus, or vitiligo with dermatologic manifestations only (e.g., participants with psoriatic arthritis are excluded) are eligible for the study provided all of following conditions are met:
      Rash must cover <10% of body surface area.
      Disease is well controlled at baseline and requires only low-potency topical corticosteroids.
      No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
20. Positive human immunodeficiency virus (HIV) test at screening.
21. Positive hepatitis B surface antigen (HBsAg) or positive total hepatitis B core antibody (HBcAb) test at screening. Participants with a positive HBsAg or total HBcAb test followed by a negative hepatitis B virus (HBV) deoxyribonucleic acid (DNA) test at screening are eligible.
22. Positive hepatitis C virus (HCV) antibody test at screening. Participants with a positive HCV antibody test followed by a negative HCV ribonucleic acid (RNA) test at screening are eligible.

### Prior/Concomitant Therapy

23. Prior systemic anticancer therapy for unresectable or metastatic melanoma.
24. Prior anticancer therapy with any immunomodulatory agents including CPIs (such as anti-PD-L1/PD-1 and anti-CTLA-4). The following prior adjuvant or neoadjuvant melanoma therapies are allowed if all related AEs have either returned to baseline or stabilized:
Anti-PD-1 and/or anti-CTLA-4 based therapy with at least 6 months between the last dose and date of recurrence. Participants with recurrence during (neo) adjuvant treatment or within 6 months after completion are not eligible.
IFN therapy with the last dose at least 6 weeks prior to randomization.
25. Prior treatment with anti-LAG3 therapy.
26. Any history of life-threatening toxicity related to prior immune therapy (e.g., anti-CTLA-4 or anti-PD-1/PD-L1 treatment or any other antibody or drug specifically targeting T-cell co-stimulation or immune checkpoint pathways) except those that are unlikely to re-occur with standard countermeasures (e.g., hormone replacement after adrenal crisis).
27. Vaccination with live vaccines within 28 days prior to randomization, or anticipation that a live attenuated vaccine will be required during the study.
28. Treatment with therapeutic oral or IV antibiotics within 14 days prior to randomization.
29. Concurrent therapy with any other investigational drug (defined as treatment for which there is currently no regulatory authority-approved indication)<28 days or 5 half-lives of the drug (whichever is shorter) prior to randomization.
30. Treatment with immune-modulating and immune suppressive agents/medication<5 half-lives or 28 days (whichever is shorter) prior to randomization, with the following exceptions:
   The use of inhaled corticosteroids and mineralocorticoids (e.g., fludrocortisone) is allowed.
   Participants who have received acute and/or low-dose systemic immunosuppressive medications (e.g., limited use of dexamethasone for nausea or chronic use of ≤10 mg/day of prednisone or dose-equivalent corticosteroid) are allowed.
31. Regular immunosuppressive therapy (i.e., for organ transplantation, chronic rheumatologic disease).
32. Radiotherapy within the last 28 days before start of treatment with RO7247669 is not allowed, with the exception of limited palliative radiotherapy.
33. Prior treatment with adoptive cell therapies, such as chimeric antigen receptor T-cell (CAR-T) therapies.

### D. Study Treatments

Table 21 summarizes the treatments administered.

TABLE 21
Summary of treatments administered

| | |
|---|---|
| Study treatment name | RO7247669 |
| IMP and NIMP | IMP |
| Dose formulation | Liquid |
| Unit dose strength(s)/ | 300 mg/6 mL |
| dosage level(s) | |
| Dose | 600 mg or 1200 mg Q3W, for a maximum of |
| | 24 months |
| Route of administration | IV infusion |
| Packaging and labeling | Study treatment is provided in vials. |

IMP = investigational medicinal product;
NIMP = non-investigational medicinal product

RO7247669 is administered intravenously (IV). A 0.2 μm or 0.22 μm inline filter must be used with the infusion set during administration.

The initial dose of RO7247669 is delivered over 60±10 minutes (the infusion may be slowed or interrupted for participants who experience infusion-associated symptoms), followed by a 60-minute observation period. If the 60-minute infusion is tolerated without infusion-associated AEs, all subsequent infusions may be delivered over 30±10 minutes, followed by a 30-minute observation period.

### Premedication

No pre-medication is foreseen prior to the first administration of RO7247669.

Participants who experienced a Grade 2 infusion-related reaction (IRR) on a previous infusion should be pre-medicated for subsequent infusions. Pre-medication regimens for future cycles may be reduced or if the participants do not experience Grade 2 or higher IRR events with the current infusion.

### Permitted Therapy

Participants are permitted to use the following therapies during the study:
Oral contraceptives with a failure rate of <1% per year.
Hormone-replacement therapy.
Prophylactic or therapeutic anticoagulation therapy (such as warfarin at a stable dose or low-molecular-weight heparin).
Inactivated influenza vaccinations.
Megestrol acetate administered as an appetite stimulant.
Inhaled corticosteroids and mineralocorticoids (e.g., fludrocortisone).
Acute and/or low-dose systemic immunosuppressive medications (e.g., a one-time dose of dexamethasone for nausea or chronic use of ≤10 mg/day of prednisone or dose-equivalent corticosteroid).
Limited field palliative radiotherapy is allowed at any time during the study, except for days where RO7247669 is administered.

Concomitant use of herbal therapies is not recommended because their PK, safety profiles, and potential drug-drug interactions are generally unknown. However, herbal therapies not intended for the treatment of cancer may be used during the study.

### Prohibited Therapy

As a general rule, no concomitant medication is permitted, with the exception of medications to treat AEs.

Use of the following therapies is prohibited during the study (excluding survival follow up) and for at least 28 days or 5 half-lives of the drug, whichever is shorter, prior to randomization and during study treatment, unless otherwise specified below:
Investigational or unlicensed/unapproved agents.
Therapy intended for the treatment of cancer (including, but not limited to, chemotherapy, hormonal therapy, immunotherapy, surgical removal of target lesions and radiotherapy (with the exception of limited palliative radiotherapy), as well as herbal therapy or traditional Chinese medicines with anti-cancer activity in the label).
Chronic use of steroids (inhaled and topical steroids are permitted) at baseline of ≤10 mg of prednisone/day (or equivalent). Concurrent high doses of systemic corticosteroids.
Administration of a live, attenuated vaccine or anticipation that such a live attenuated vaccine will be required during the study or within 4 months after the administration of the final dose of the study drug.
Systemic immunostimulatory agents (including, but not limited to, IFNs and IL-2) because these agents could potentially increase the risk for autoimmune conditions when given in combination with CPIs.
Systemic immunosuppressive medications (including, but not limited to, cyclophosphamide, azathioprine, methotrexate, and thalidomide) because these agents could potentially alter the efficacy and safety of the study drug.
Adoptive cell therapies, such as CAR-T therapies.

### E. Efficacy Assessments

Participants undergo their first tumor assessments at 12 weeks (±1 week) relative to date of randomization. Subsequent tumor assessment occur every 9 weeks (±1 week) up to 48 weeks and then every 12 weeks (±1 week) relative to date of randomization, regardless of treatment delays. Tumor assessments are performed at the described interval regardless of treatment delays until radiographic disease progression per RECIST v1.1, initiation of a new anti-cancer therapy, withdrawal of consent, death, study termination, or a maximum of 24 months after randomization whichever occurs first. Thus, tumor assessments are to continue according to schedule in participants who discontinue treatment for reasons other than disease progression or loss of clinical benefit. For participants who continue treatment after progressive disease per RECIST v1.1, tumor assessments continue according to schedule until study treatment is discontinued.

### F. Eastern Cooperative Oncology Group Performance Status

ECOG Performance Status is assessed at screening, before each study treatment administration, and at the discontinuation visit. It is recommended, where possible, that a participant's performance status is assessed by the same person throughout the study.

### G. Infusion-Related Reactions

Administration of therapeutic antibodies may cause IRRs characterized by symptoms such as fever, chills, dizziness, hypertension, hypotension, dyspnea, restlessness, sweating, flushing, rash, tachycardia, tachypnea, headache, tumor pain, nausea, and/or vomiting. Respiratory and cardiac symptoms such as bronchospasm, larynx, and throat irritation, wheezing, laryngeal edema, and atrial fibrillation may also occur. Such reactions typically occur during or shortly after an infusion or within 24 hours after study treatment infusion, predominantly at the first infusion. The incidence and severity typically decrease with subsequent infusions.

Participants may also develop immunoglobulin (Ig) E-mediated hypersensitivity reactions. IRRs may be indistinguishable from an anaphylactic reaction; however, in case of IgE-mediated hypersensitivity, symptoms typically occur after previous exposure and very rarely with the first infusion. In case of confirmed IgE-mediated hypersensitivity reaction, treatment should be permanently discontinued. Recommendations for infusion-related reaction prevention and management are provided in Table 22.

TABLE 22
Recommendations for infusion-related
reaction prevention and management

| Infusion-related | |
|---|---|
| reactions^{a} | Guidance |
| Grades 1-2 | Slow infusion to ≤50% or interrupt infusion |
| | Give supportive treatment^{b} |
| | Upon symptom resolution may resume infusion (if |
| | interrupted) at 50% starting rate. The infusion |
| | must remain at the lower rate resulting in symptom |
| | resolution for the remainder of the infusion. |
| | For Grade 2 IRRs, subsequent cycles of RO7247669 |
| | should be administered with pre-medication |
| | including acetaminophen/paracetamol and an |
| | antihistamine, such as diphenhydramine. |
| | Notes: |
| | For Grade 2 wheezing or urticaria, the participant |
| | must also be pre-medicated prior to subsequent |
| | doses (as described above). |
| | If symptoms recur with the same or greater severity |
| | following the slower or interrupted infusion, the |
| | infusion must be stopped immediately. No further |
| | RO7247669 will be administered for the cycle. |
| Grades 3-4 | Discontinue infusion |
| | Give supportive treatment^{b} |
| | Permanently discontinue study treatment |

IRR = infusion-related reaction; NCI CTCAE = National Cancer Institute Common Terminology Criteria for Adverse Events.
^{a}Refer to the NCI-CTCAE, v5.0 scale for the grading of symptoms.
^{b}Supportive treatment: Participants should be treated with acetaminophen/paracetamol and an antihistamine, such as diphenhydramine, if they have not been administered in the last 4 hours.
Intravenous fluids (e.g., normal saline) may be administered as clinically indicated. For bronchospasm, urticaria, or dyspnea, antihistamines, oxygen, corticosteroids (e.g., 100 mg IV prednisolone or equivalent), and/or bronchodilators may be administered per institutional practice.

### H. Statistical Considerations

### Sample Size Determination

The planned number of enrolled participants is 80, randomized in a 1:1 ratio to receive RO7247669 Q3W at a dose of either 600 mg or 1200 mg. For the evaluation of the primary endpoint, participants are followed until at least 6 months after last participant in, ensuring there is no administrative censoring when calculating the 6-months PFS. The study aims to qualitatively characterize the two dose levels and appreciate any apparent differences between the doses. However, the study is not adequately powered to detect all clinically meaningful differences.

Some sample size considerations can be done around the 6-months PFS endpoint, assuming that no censoring occurs before the 6 months. Without censoring, the 6 months PFS will coincide with the proportion of participants who had not experienced any progression or death within the first 6 months, which can be described through a binomial distribution.

Table 23 shows the power for several possible true underlying differences in 6-months PFS assuming the proportion of participants who had not experienced any progression or death at 6 months of the least efficacious arm is around 43% (the observed 6-months PFS for the nivolumab arm in the RELATIVITY-047 trial).

The table shows that there is 70% power for detecting a difference of 20% in 6 months PFS (assuming a 20% 2-sided alpha), while it drops to 53% in presence of a 15% difference.

TABLE 23
Power for several possible true 6 months PFS
rates (assuming no censoring before 6 months)

| 6 Months PFS Rate for | 6 Months PFS for Most | |
|---|---|---|
| Least Efficacious Arm | Efficacious Arm | Power |
| 43% | 53% | 36% |
| 43% | 58% | 53% |
| 43% | 63% | 70% |

PFS = progression-free survival

### Sets for Analyses

For purposes of analysis, populations are defined as presented in Table 24.

TABLE 24
Analysis populations

| Population | Description |
|---|---|
| Intent-to-treat | All randomized participants are included in the |
| | intent-to-treat population. |
| Safety | All participants randomized to study treatment and |
| | who received any amount of the study treatment, |
| | whether prematurely withdrawn from the study or not, |
| | are included in the safety analysis. |
| Pharmacokinetic | All participants who have received at least one dose |
| (PK) | of study treatment and who have data from at least |
| | one postdose sample are included in the PK analysis |
| | population. Participants are excluded from the PK |
| | analysis population if they significantly violate |
| | the inclusion or exclusion criteria, deviate |
| | significantly from the protocol, or if data are |
| | unavailable or incomplete which may influence the |
| | PK analysis. |
| Immunogenicity | Participants who had at least one predose or at |
| | least one postdose ADA assessment are included and |
| | analyzed according to the treatment they actually |
| | received or were allocated to receive. The |
| | relationship between ADA status and safety, efficacy, |
| | PK, and biomarker endpoints is analyzed and reported |
| | descriptively via subgroup analyses. |

### Demographics and Baseline Characteristics

Study enrollment, study drug administration, reasons for study drug discontinuation, and reasons for discontinuation from the study are summarized by treatment arm. Demographic (including age, sex, and self-reported race/ethnicity) and baseline disease characteristics (e.g., ECOG Performance Status) are summarized overall and by treatment arm. Descriptive statistics (mean, median, standard deviation, and range) are presented for continuous data, and frequencies and percentages are presented for categorical data, as appropriate.

Baseline measurements are the last available data obtained prior to the participant receiving the study treatment.

### Efficacy Analyses

The primary and secondary efficacy analyses include all participants in the intent-to-treat (ITT) population, with participants grouped according to the dose level assigned at randomization. Efficacy statistical analysis methods are shown in Table 25.

TABLE 25
Efficacy statistical analysis methods

| Endpoint | Statistical Analysis Methods |
|---|---|
| Primary | PFS based on RECIST v1.1 |
| | PFS defined as the time from randomization to the first |
| | occurrence of progression as determined by the Investigator |
| | according to RECIST v1.1 or death during the treatment |
| | period or within on study (i.e., within 60 days of the |
| | last study treatment tumor assessment) after treatment |
| | discontinuation, whichever occurs first. Time to disease |
| | progression is censored at the time of the last tumor |
| | assessment for participants who have not experienced |
| | disease progression at the time of analysis and who |
| | have not died during the treatment period or within 60 |
| | days of the last tumor assessment after treatment |
| | discontinuation. For participants with no postbaseline |
| | tumor assessment the time to disease progression will |
| | be censored at the date of randomization. |
| | The requirement that participants without a radiographic |
| | disease progression per RECIST v1.1 are still followed |
| | for the tumor assessments until progression or when the |
| | participant starts a new anti-cancer therapy (whatever |
| | occurs first) means that: |
| | Intercurrent events of treatment discontinuations in |
| | absence of disease progression per RECIST 1.1 do not |
| | per se censor the PFS observation, since the participant |
| | will still undergo tumor assessments (treatment policy |
| | strategy). |
| | Intercurrent events of new anticancer therapy imply |
| | instead that the PFS observation is censored at the last |
| | available tumor assessment before the new anticancer |
| | treatment starts, therefore assuming that participants |
| | who experience this intercurrent event have the same |
| | risk as those who did not (hypothetical strategy). |
| | PFS is compared between treatment arms with use of the |
| | stratified log-rank test. |
| | The HR and its 95% CI for PFS are estimated using a |
| | stratified Cox proportional-hazards model. Kaplan-Meier |
| | methodology is used to estimate the median PFS for |
| | each treatment arm, and the Kaplan-Meier curves are |
| | constructed. In addition, 6 month PFS is estimated with |
| | its relative confidence intervals. |
| Secondary | ORR based on RECIST v1.1 |
| | ORR is defined as the proportion of participants who |
| | have achieved an objective response, characterized by |
| | a CR or PR according to RECIST v1.1. Objective response |
| | is evaluated by treatment arm, and participants without |
| | post-baseline overall response assessments are counted |
| | as non-responders. |
| | An estimate of ORR for each treatment arm as well as |
| | the difference between the arms is computed along with |
| | its 95% CI. The Mantel-Haenszel test is used at the |
| | two-sided significance level of 10%, stratified by the |
| | protocol-defined stratification factors. |
| | DCR based on RECIST v1.1 |
| | DCR is defined as ORR + stable disease rate (SDR). |
| | DoR based on RECIST v1.1 |
| | DoR is defined as the time from the first occurrence |
| | of a documented objective response to disease progression |
| | according to RECIST v1.1 or death from any cause, |
| | whichever occurs first. |
| | The analysis of DoR includes only participants who |
| | achieved an objective response to study treatment. DoR |
| | is estimated using the Kaplan-Meier methodology. As |
| | the determination of DoR is based on a non-randomized |
| | subset of participants, no formal hypothesis testing |
| | is performed. |
| Exploratory | OS is defined as the time from randomization to death |
| | from any cause. Data for participants who are alive at |
| | the time of the data cut-off is censored at the last date |
| | they were known to be alive. Data from participants |
| | without post-baseline information is censored at the |
| | date of randomization. |
| | Kaplan-Meier methods are used to estimate median OS for |
| | each treatment arm and the 95% CIs for median OS are |
| | computed using the Brookmeyer and Crowley method. The |
| | stratified Cox proportional hazard model is used to |
| | estimate the hazard ratio (i.e., the magnitude of the |
| | treatment effect) and the corresponding 95% confidence |
| | interval, stratified by the protocol-defined |
| | stratification factors. |

CI = confidence interval; CR = complete response; DCR = disease control rate; DoR = duration of response; EORTC = European Organisation for Research and Treatment of Cancer; HR = hazard ratio; ORR = objective response rate; OS = overall survival; PFS = progression-free survival; PR = partial response; RECIST = response evaluation criteria in solid tumors.

### Example 4: A Phase Ib/II, Open-Label, Multicenter, Randomized Umbrella Study Evaluating the Efficacy and Safety of Multiple Immunotherapy-Based Treatment Combinations in Patients with Advanced Liver Cancers (MORPHEUS-Liver)

### A. Study Design

GO42216 is a Phase Ib/II, open-label, multicenter, randomized umbrella study that evaluates the efficacy, safety, and pharmacokinetics of immunotherapy-based treatment combinations in patients with advanced liver cancers. The study is designed with the flexibility to open new treatment arms as new treatments become available, close existing treatment arms that demonstrate minimal clinical activity or unacceptable toxicity, modify the patient population (e.g., with regard to prior anti-cancer treatment or biomarker status), or introduce additional cohorts of patients with other types of advanced primary liver cancer (e.g., intrahepatic cholangiocarcinoma (iCCA)).

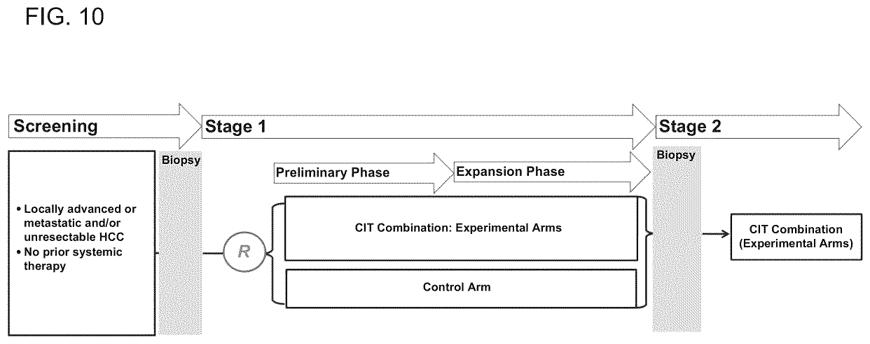

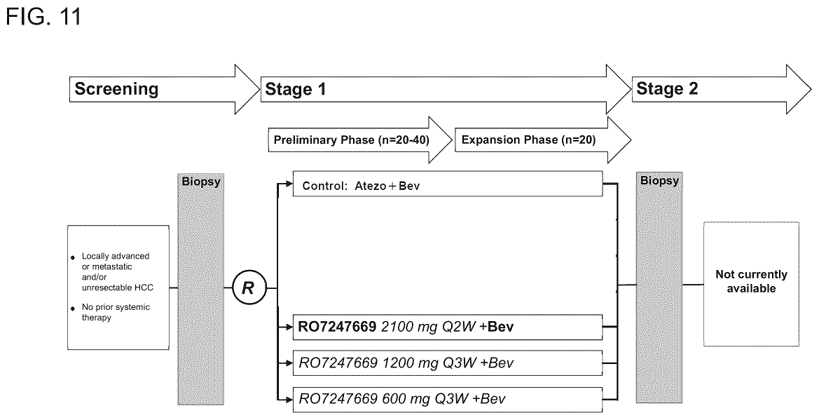

Cohort 1 enrolls patients with locally advanced or metastatic hepatocellular carcinoma (HCC) who have not received prior systemic therapy for their disease (FIGS. 10 and 11). Eligible patients are initially randomly assigned to one of several treatment arms (Stage 1). Patients who experience loss of clinical benefit or unacceptable toxicity during Stage 1 may be eligible to receive treatment with a different treatment combination (Stage 2).

### Stage 1

During Stage 1, patients are randomly assigned to a control arm (atezolizumab plus bevacizumab (Atezo+bev)) or an experimental arm consisting of bevacizumab in combination with RO7247669 (RO7247669+bev). Specific objectives and corresponding endpoints for the study are outlined in Table 26.

TABLE 26
Efficacy statistical analysis methods

| | Corresponding Endpoint |
|---|---|
| Primary Efficacy Objective | |
| To evaluate the efficacy of | Objective response rate (ORR), |
| immunotherapy-based | defined as the proportion of |
| treatment combinations | patients with a complete response |
| during Stage 1. | or partial response on two |
| | consecutive occasions ≥4 weeks |
| | apart during Stage 1, as |
| | determined by the investigator |
| | according to RECIST v1.1. |
| Secondary Efficacy Objective | |
| To evaluate the efficacy of | Progression-free survival (PFS) |
| immunotherapy-based | after randomization, defined as |
| treatment combinations | the time from randomization to the |
| during Stage 1. | first occurrence of disease |
| | progression or death from any cause |
| | (whichever occurs first) in Stage 1, |
| | as determined according to RECIST |
| | v1.1. |
| | Overall survival (OS) after |
| | randomization, defined as the time from |
| | randomization to death from any cause. |
| | OS at specific timepoints (e.g., |
| | 6 months). |
| | Duration of response (DOR), defined |
| | as the time from the first occurrence |
| | of a documented objective response to |
| | disease progression or death from any |
| | cause (whichever occurs first) in Stage |
| | 1, as determined according to RECIST |
| | v1.1. |
| | Disease control, defined as stable |
| | disease for ≥12 weeks or a complete |
| | or partial response, as determined |
| | according to RECIST v1.1. |
| Exploratory Efficacy | |
| Objective | |
| To evaluate the efficacy of | ORR, PFS, DOR, and disease control, |
| immunotherapy-based treatment | as determined by the investigator |
| combinations during Stage 1. | according to HCC mRECIST. |
| Safety Objective | |
| To evaluate the safety of | Incidence, nature, and severity of |
| immunotherapy-based treatment | adverse events and laboratory |
| combinations during Stage 1. | abnormalities, with severity |
| | determined according to NCI |
| | CTCAE v5.0. |
| | Change from baseline in vital |
| | signs and ECG parameters. |
| | Change from baseline in targeted |
| | clinical laboratory test results. |
| Exploratory Pharmacokinetic | |
| Objectives | |
| To characterize the PK profile | Plasma or serum concentrations of |
| of drugs that are administered | each drug (as appropriate) at |
| as part of an immunotherapy- | specified timepoints. |
| based treatment combination | |
| during Stage 1. | |
| To evaluate potential | Relationship between plasma or |
| relationships between drug | serum concentrations or PK |
| exposure during Stage 1 and the | parameters for each drug (as |
| efficacy and safety of | appropriate on the basis of |
| immunotherapy-based | available data) and efficacy |
| treatment combinations. | endpoints. |
| | Relationship between plasma or |
| | serum concentrations or PK |
| | parameters for each drug (as |
| | appropriate on the basis of |
| | available data) and safety |
| | endpoints. |
| Exploratory Immunogenicity | |
| Objectives | |
| To evaluate the immune | For drugs for which anti-drug antibody |
| response to drugs that are | (ADA) formation is measured: presence |
| administered as part of an | of ADAs during the study relative to |
| immunotherapy-based treatment | the presence of ADAs at baseline. |
| combination during Stage 1. | |
| To evaluate potential effects | For drugs for which ADA formation is |
| of ADAs during Stage 1. | measured: relationship between ADA |
| | status and efficacy, safety, or PK |
| | endpoints. |
| Exploratory Biomarker | |
| Objective | |
| To identify biomarkers during | Relationship between biomarkers in |
| Stage 1 that are predictive of | blood and tumor tissue and efficacy, |
| response to study treatment | safety, PK, immunogenicity, or other |
| (i.e., predictive biomarkers), | biomarker endpoints. |
| are associated with progression | |
| to a more severe disease state | |
| (i.e., prognostic biomarkers), are | |
| associated with resistance to | |
| study treatment, are associated | |
| with susceptibility to developing | |
| adverse events (i.e., safety | |
| biomarkers), can provide | |
| evidence of study treatment | |
| activity (i.e., pharmacodynamic | |
| biomarkers), or can increase the | |
| knowledge and understanding of | |
| disease biology. | |

ADA = anti-drug antibody; DOR = duration of response; HCC = hepatocellular carcinoma; HCC mRECIST = HCC-specific modified RECIST; NCI CTCAE v5.0 = National Cancer Institute Common Terminology Criteria for Adverse Events, Version 5.0; ORR = objective response rate; OS = overall survival; PFS = progression-free survival; PK = pharmacokinetic; RECIST v1.1 = Response Evaluation Criteria in Solid Tumors, Version 1.1.
Note:
Overall response at a single timepoint is assessed by the investigator using RECIST v1.1 and HCC mRECIST.

Approximately 170-320 patients are enrolled during Stage 1. Enrollment within the experimental arms takes place in two phases: a preliminary phase followed by an expansion phase. For most arms, approximately 20 patients are enrolled during the preliminary phase. If clinical activity is observed in an experimental arm during the preliminary phase, approximately 20 additional patients may be enrolled in that arm during the expansion phase. In some arms, randomization is suspended to allow for a safety evaluation in a minimum of 6 patients. Experimental arms with minimal clinical activity or unacceptable toxicity do not undergo expansion. Additional patients may be enrolled to ensure balance among treatment arms with respect to demographic and baseline characteristics, including potential predictive biomarkers, to enable further subgroup analyses.

New experimental arms may be added during the course of the study by amending the protocol. Patients in Stage 1 are randomly assigned to treatment arms, and the randomization ratio depends on the number of experimental arms that are open for enrollment (e.g., if an arm is added or enrollment in an arm is suspended pending analysis of results from the preliminary phase), with the stipulation that the likelihood of being allocated to the control arm will be no more than 35%. Randomization takes into account arm-specific exclusion criteria. Patients are ineligible for a specific arm if they meet any of the exclusion criteria outlined for that arm. Details on treatment assignment and randomization are provided in Table 27.

TABLE 27
Stage 1 treatment regimens

| | | Number of Patients | |
|---|---|---|---|
| | | Preliminary | Expansion |
| Arm Name | Study Treatment | Phase | Phase |
| Control | Atezolizumab plus | 20 | 20 |
| (Atezo + Bev) | bevacizumab | | |
| RO7247669 2100 | RO7247669 2100 mg | 20^{a} | 20 |
| mg Q2W + Bev | Q2W plus bevacizumab | | |
| RO7247669 1200 | RO7247669 1200 mg | 20 | 20 |
| mg Q3W + Bev | Q3W plus bevacizumab | | |
| RO7247669 600 | RO7247669 600 mg | 20 | 20 |
| mg Q3W + Bev | Q3W plus bevacizumab | | |

Atezo = atezolizumab; Bev = bevacizumab; Q2W = every 2 weeks; Q3W = every 3 weeks.
^{a}Enrollment is suspended in the RO7247669 2100 mg Q2W + Bev arm to allow for a safety evaluation in a minimum of 6 patients.

Patients in the control and experimental arms continue to receive treatment until unacceptable toxicity or loss of clinical benefit as determined after an integrated assessment of radiographic and biochemical data, local biopsy results (if available), and clinical status (e.g., symptomatic deterioration such as pain secondary to disease). Because of the possibility of an initial increase in tumor burden caused by immune cell infiltration in the setting of a T-cell response (termed pseudoprogression) with cancer immunotherapies (CITs), radiographic progression per Response Evaluation Criteria in Solid Tumors, Version 1.1 (RECIST v1.1) may not be indicative of true disease progression. In the absence of unacceptable toxicity, patients who meet criteria for disease progression per RECIST v1.1 while receiving treatment with a CIT combination will be permitted to continue treatment if they meet all of the following criteria:
Evidence of clinical benefit, as determined following a review of all available data.
Absence of symptoms and signs (including laboratory values, such as new or worsening hypercalcemia) indicating unequivocal progression of disease.
Absence of decline in Eastern Cooperative Oncology Group (ECOG) Performance Status that can be attributed to disease progression.
Absence of tumor progression at critical anatomical sites (e.g., leptomeningeal disease) that cannot be managed by protocol-allowed medical interventions.

### Safety Evaluation Phase

To account for potential overlapping toxicities in the RO7247669 2100 mg every 2 weeks (Q2W)+Bev arm, enrollment is suspended after approximately 6 patients have been enrolled to allow for a safety evaluation. The safety evaluation is based on safety data from a minimum of 6 patients who receive at least one dose of treatment (i.e., one dose of each agent for a given combination) and completed safety follow-up assessments during at least one full treatment cycle. If a combination is determined to be sufficiently safe, enrollment is resumed in that arm. The decision not to resume enrollment is based on study treatment-related Grade ≥3 events that are unmanageable and lead to discontinuation of all study drugs in at least one-third of patients.

### Stage 2

Patients in a control or experimental arm who experience loss of clinical benefit (as described above) during Stage 1 are given the option of receiving a different treatment combination during Stage 2 provided they meet eligibility criteria and a Stage 2 arm is open for enrollment. Patients who experience unacceptable toxicity during Stage 1 may be eligible to receive treatment during Stage 2.

Stage 2 treatment must begin within 3 months after the patient has experienced loss of clinical benefit or unacceptable toxicity in Stage 1 and continues until unacceptable toxicity or loss of clinical benefit. However, it is recommended that patients begin Stage 2 treatment as soon as possible. No Stage 2 treatment regimens are currently available.

### Assessments and Monitoring

All patients are closely monitored for adverse events throughout the study, and adverse events are graded according to the National Cancer Institute Common Terminology Criteria for Adverse Events, Version 5.0 (NCI CTCAE v5.0). The severity of cytokine release syndrome (CRS) is also graded according to the American Society for Transplantation and Cellular Therapy CRS Consensus Grading Scale.

Patients undergo tumor assessments every 6 weeks (from Day 1 of Cycle 1) during the first 48 weeks and then every 6 or 12 weeks thereafter. Response is assessed using RECIST v1.1 and HCC-specific modified RECIST (HCC mRECIST).

Baseline tumor tissue samples are collected from all patients, preferably by means of a biopsy performed at study entry. These samples are utilized for biomarker research.

To characterize the pharmacokinetic (PK) properties and/or immunogenicity of the therapeutic agents, blood samples are taken at various timepoints before and during study treatment administration.

On the basis of a review of real time safety data and available PK data, treatment regimens may be modified as deemed appropriate.

### Target Population

### Inclusion Criteria for Stage 1

Patients must meet all of the following criteria to qualify for Stage 1:
Age≥18 years.
ECOG Performance Status of 0 or 1 within 7 days prior to randomization.
Locally advanced or metastatic and/or unresectable HCC with diagnosis confirmed by histology/cytology or clinically by American Association for the Study of Liver Diseases (AASLD) criteria in cirrhotic patients. For cirrhotic patients with no histological confirmation of diagnosis, clinical confirmation is required per AASLD criteria.
Child-Pugh class A within 7 days prior to randomization.
Disease that is not amenable to curative surgical and/or locoregional therapies. Patients with progressive disease after surgical and/or locoregional therapies are eligible.
No prior systemic treatment (including systemic investigational agents) for HCC. Prior treatment with herbal therapies, including traditional Chinese medicines, with anti-cancer activity noted in the label are allowed, provided that these medications are discontinued prior to randomization.
Life expectancy≥3 months.
Availability of a representative tumor specimen that is suitable for determination of PD-L1 and/or additional biomarker status via central testing. Baseline tumor tissue samples are collected from all patients, preferably by means of a biopsy performed at study entry.

### Inclusion Criteria for Stage 1 and Stage 2

Patients must meet all of the following criteria to qualify for Stage 1 and to qualify for Stage 2:
Ability to comply with the study protocol.
Measurable disease (at least one target lesion) according to RECIST v1.1. Patients who received prior locoregional therapy (e.g., radiofrequency ablation, percutaneous ethanol or acetic acid injection, cryoablation, high-intensity focused ultrasound, transarterial chemoembolization, transarterial embolization, etc.) are eligible provided the target lesion(s) have not been previously treated with locoregional therapy or the target lesion(s) within the field of local therapy have subsequently progressed in accordance with RECIST v1.1.
Adequate hematologic and end-organ function, defined by the following laboratory test results, obtained within 7 days prior to initiation of study treatment:
   ANC≥1.5×10⁹/L (≥1500/μL) without granulocyte colony-stimulating factor support.
   Lymphocyte count≥0.5×10⁹/L (≥500/μL).
   Platelet count≥75×10 g/L (≥75,000/μL) without transfusion.
   Hemoglobin≥90 g/L (≥9.0 g/dL) without transfusion.
   Patients must not have required transfusion during screening or within 2 weeks prior to screening to meet this criterion.
   AST, ALT, and ALP≤5×upper limit of normal (ULN).
   Bilirubin≤3×ULN.
   Creatinine≤1.5×ULN or creatinine clearance≥50 mL/min (calculated using the Cockcroft-Gault formula).
   Albumin≥28 g/L (≥2.8 g/dL) without transfusion.
   For patients not receiving anticoagulation: INR or aPTT≤1.5×ULN.
Documented virology status of hepatitis, as confirmed by screening tests for hepatitis B virus (HBV) and hepatitis C virus (HCV). For patients with active HBV: HBV DNA<500 IU/mL during screening, initiation of anti-HBV treatment at least 14 days prior to randomization and willingness to continue anti-HBV treatment during the study (per local standard of care; e.g., entecavir). Patients with HCV, either with resolved infection (as evidenced by detectable antibody) or chronic infection (as evidenced by detectable HCV RNA), are eligible.
Negative HIV test at screening.
For women of childbearing potential: agreement to remain abstinent (refrain from heterosexual intercourse) or use contraception.
For men: agreement to remain abstinent (refrain from heterosexual intercourse) or use contraception, and agreement to refrain from donating sperm.

### Inclusion Criteria for Stage 2

Patients must meet all of the following criteria to qualify for Stage 2:
ECOG Performance Status of 0, 1, or 2.
Ability to initiate Stage 2 treatment within 3 months after experiencing unacceptable toxicity not related to atezolizumab or RO7247669 or loss of clinical benefit while receiving Stage 1 treatment.
Availability of a tumor specimen from a biopsy performed upon discontinuation of Stage 1 (if clinically feasible).

### Exclusion Criteria

Patients are excluded from enrollment in specific arms during Stage 1 or enrollment during Stage 2 if they meet any of the criteria outlined below.

### Exclusion Criteria for Stage 1.

Patients who meet any of the following criteria will be excluded from Stage 1:
Prior treatment with CD137 agonists or immune checkpoint blockade therapies, including anti-CTLA-4, anti-PD-1, and anti-PD-L1 therapeutic antibodies.
Treatment with investigational therapy within 28 days prior to initiation of study treatment.
Treatment with locoregional therapy to liver (e.g., radiofrequency ablation, percutaneous ethanol or acetic acid injection, cryoablation, high-intensity focused ultrasound, transarterial chemoembolization, transarterial embolization, etc.) within 28 days prior to initiation of study treatment, or non-recovery from side effects of any such procedure.
Untreated or incompletely treated esophageal and/or gastric varices with bleeding or that are at high risk for bleeding.
Patients must undergo an esophagogastroduodenoscopy (EGD), and all size of varices (small to large) must be assessed and treated per local standard of care prior to enrollment. Patients who have undergone an EGD within 6 months of prior to initiation of study treatment do not need to repeat the procedure.
A prior bleeding event due to esophageal and/or gastric varices within 6 months prior to initiation of study treatment.
Adverse events from prior anti-cancer therapy that have not resolved to Grade 1 or better, with the exception of alopecia of any grade.
Inadequately controlled hypertension, defined as systolic blood pressure (BP)>150 mmHg and/or diastolic BP>100 mmHg (average of at least three readings at two or more sessions). Anti-hypertensive therapy to achieve these parameters is allowed.
History of hypertensive crisis or hypertensive encephalopathy.
Significant vascular disease (e.g., aortic aneurysm requiring surgical repair or recent peripheral arterial thrombosis) within 6 months prior to initiation of study treatment.
History of hemoptysis (≥2.5 mL of bright red blood per episode) within 1 month prior to initiation of study treatment.
Evidence of bleeding diathesis or significant coagulopathy (in the absence of therapeutic anticoagulation).
Current or recent (≤10 days prior to initiation of study treatment) use of aspirin (>325 mg/day) or treatment with clopidogrel, dipyramidole, ticlopidine, or cilostazol.
Current or recent (≤10 days prior to initiation of study treatment) use of full-dose oral or parenteral anticoagulants or thrombolytic agents for therapeutic (as opposed to prophylactic) purpose. Prophylactic anticoagulation for the patency of venous access devices is allowed provided the activity of the agent results in an INR<1.5×ULN and aPTT is within normal limits within 14 days prior to initiation of study treatment. For prophylactic use of anticoagulants or thrombolytic therapies, the approved dose as
described in the local label may be used.
Core biopsy or other minor surgical procedure, excluding placement of a vascular access device, within 3 days prior to initiation of study treatment.
History of abdominal or tracheoesophageal fistula, gastrointestinal (GI) perforation, or intra-abdominal abscess within 6 months prior to initiation of study treatment.
History of intestinal obstruction and/or clinical signs or symptoms of GI obstruction, including subocclusive or occlusive syndrome related to the underlying disease, or requirement for routine parenteral hydration, parenteral nutrition, or tube feeding prior to initiation of study treatment. Patients with signs or symptoms of subocclusive or occlusive syndrome or with intestinal obstruction at the time of initial diagnosis may be enrolled if they had received definitive (surgical) treatment for symptom resolution.
Evidence of abdominal free air that is not explained by paracentesis or recent surgical procedure.
Serious, non-healing or dehiscing wound, active ulcer, or untreated bone fracture.
Grade ≥2 proteinuria, as demonstrated by ≥2+ protein on dipstick urinalysis and ≥1.0 g of protein in a 24-hour urine collection. All patients with ≥2+ protein on dipstick urinalysis at screening must undergo a 24-hour urine collection for protein. Patients with <2+ protein on dipstick urinalysis are eligible for the study.
Metastatic disease that involves major airways or blood vessels, or centrally located mediastinal tumor masses (<30 mm from the carina) of large volume. Patients with vascular invasion of the portal or hepatic veins may be enrolled.
History of intra-abdominal inflammatory process within 6 months prior to initiation of study treatment, including, but not limited to, peptic ulcer disease, diverticulitis, or colitis.
Radiotherapy within 28 days or abdominal/pelvic radiotherapy within 60 days prior to initiation of study treatment with the exception of palliative radiotherapy to bone lesions within 7 days prior to initiation of study treatment.
Major surgical procedure, open biopsy, or significant traumatic injury within 28 days prior to initiation of study treatment; or abdominal surgery, abdominal interventions or significant abdominal traumatic injury within 60 days prior to initiation of study treatment; or anticipation of need for major surgical procedure during the course of the study or non-recovery from side effects of any such procedure.
Chronic daily treatment with a non-steroidal anti-inflammatory drug (NSAID). The occasional use of NSAIDs for the symptomatic relief of medical conditions such as headache or fever is allowed.

### Exclusion Criteria for Stage 1 and Stage 2

Patients who meet any of the following criteria are excluded from Stage 1 and from Stage 2:
Known fibrolamellar HCC, sarcomatoid HCC, or mixed cholangiocarcinoma and HCC.
History of hepatic encephalopathy.
Moderate or severe ascites.
Co-infection with HBV and HCV. Patients with a history of HCV infection but who are negative for HCV RNA by polymerase chain reaction (PCR) are considered non-infected with HCV.
Symptomatic, untreated, or actively progressing central nervous system (CNS) metastases. Asymptomatic patients with treated CNS lesions are eligible, provided that all of the following criteria are met:
Measurable disease, per RECIST v1.1, must be present outside the CNS.
The patient has no history of intracranial hemorrhage or spinal cord hemorrhage.
The patient has not undergone stereotactic radiotherapy within 7 days prior to initiation of study treatment, whole-brain radiotherapy within 14 days prior to initiation of study treatment, or neurosurgical resection within 28 days prior to initiation of study treatment.
The patient has no ongoing requirement for corticosteroids as therapy for CNS disease. Anticonvulsant therapy at a stable dose is permitted.
Metastases are limited to the cerebellum or the supratentorial region (i.e., no metastases to the midbrain, pons, medulla, or spinal cord).
There is no evidence of interim progression between completion of CNS-directed therapy and initiation of study treatment. Asymptomatic patients with CNS metastases newly detected at screening are eligible for the study after receiving radiotherapy or surgery, with no need to repeat the screening brain scan.
History of leptomeningeal disease.
Uncontrolled tumor-related pain. Patients requiring pain medication must be on a stable regimen at study entry. Symptomatic lesions (e.g., bone metastases or metastases causing nerve impingement) amenable to palliative radiotherapy should be treated prior to enrollment. Patients should be recovered from the effects of radiation. There is no required minimum recovery period. Asymptomatic metastatic lesions that would likely cause functional deficits or intractable pain with further growth (e.g., epidural metastasis that is not currently associated with spinal cord compression) should be considered for locoregional therapy if appropriate prior to enrollment.
Uncontrolled pleural effusion, pericardial effusion, or ascites requiring recurrent drainage procedures (once monthly or more frequently). Patients with indwelling catheters (e.g., PLEURX®) are allowed.
Uncontrolled or symptomatic hypercalcemia (ionized calcium>1.5 mmol/L, calcium>12 mg/dl, or corrected calcium>ULN).
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barre syndrome, or multiple sclerosis, with the following exceptions:
Patients with a history of autoimmune-related hypothyroidism who are on thyroid-replacement hormone are eligible for the study.
Patients with controlled Type 1 diabetes mellitus who are on an insulin regimen are eligible for the study.
Patients with eczema, psoriasis, lichen simplex chronicus, or vitiligo with dermatologic manifestations only (e.g., patients with psoriatic arthritis are excluded) are eligible for the study provided all of following conditions are met:
   Rash must cover <10% of body surface area.
   Disease is well controlled at baseline and requires only low-potency topical corticosteroids.
   No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on screening chest computed tomography scan. History of radiation pneumonitis in the radiation field (fibrosis) is permitted.
Active tuberculosis (TB), as documented by a positive purified protein derivative (PPD) skin test or TB blood test and confirmed by a positive chest X-ray within 3 months prior to initiation of study treatment. Patients with a positive PPD skin test or TB blood test followed by a negative chest X-ray may be eligible for the study.
Significant cardiovascular disease (such as New York Heart Association Class II or greater cardiac disease, myocardial infarction, or cerebrovascular accident) within 3 months prior to initiation of study treatment, unstable arrhythmia, or unstable angina.
Major surgical procedure, other than for diagnosis, within 4 weeks prior to initiation of study treatment, or anticipation of need for a major surgical procedure during the study.
History of malignancy other than HCC within 5 years prior to screening, with the exception of malignancies with a negligible risk of metastasis or death (e.g., 5-year OS rate>90%), such as adequately treated carcinoma in situ of the cervix, non-melanoma skin carcinoma, localized prostate cancer, ductal carcinoma in situ, or Stage I uterine cancer.
Severe infection within 4 weeks prior to initiation of study treatment, including, but not limited to, hospitalization for complications of infection, bacteremia, or severe pneumonia, or any active infection that could impact patient safety.
Treatment with therapeutic oral or IV antibiotics within 2 weeks prior to initiation of study treatment. Patients receiving prophylactic antibiotics (e.g., to prevent a urinary tract infection or chronic obstructive pulmonary disease exacerbation) are eligible for the study.
Prior allogeneic stem cell or solid organ transplantation.
Any other disease, metabolic dysfunction, physical examination finding, or clinical laboratory finding that contraindicates the use of an investigational drug, may affect the interpretation of the results, or may render the patient at high risk from treatment complications.
Pregnancy or breastfeeding, or intending to become pregnant during the study.
Treatment with a live, attenuated vaccine within 4 weeks prior to initiation of study treatment.
History of severe allergic anaphylactic reactions to chimeric or humanized antibodies or fusion proteins.
Known hypersensitivity to Chinese hamster ovary cell products or recombinant human antibodies.
Known allergy or hypersensitivity to any of the study drugs or any of their excipients.
Treatment with systemic immunostimulatory agents (including, but not limited to, interferon and interleukin 2) within 4 weeks or 5 drug-elimination half-lives (whichever is longer) prior to initiation of study treatment.
Treatment with systemic immunosuppressive medication (including, but not limited to, corticosteroids, cyclophosphamide, azathioprine, methotrexate, thalidomide, and anti-tumor necrosis factor agents) within 2 weeks prior to initiation of study treatment, or anticipation of need for systemic immunosuppressive medication during study treatment, with the following exceptions:
Patients who received acute, low-dose systemic immunosuppressant medication or a one-time pulse dose of systemic immunosuppressant medication (e.g., 48 hours of corticosteroids for a contrast allergy) are eligible.
Patients who received mineralocorticoids (e.g., fludrocortisone), corticosteroids for chronic obstructive pulmonary disease or asthma, or low-dose corticosteroids for orthostatic hypotension or adrenal insufficiency are eligible for the study.
Grade ≥3 hemorrhage or bleeding event within 8 weeks prior to initiation of study treatment.
Patients entering Stage 2: immunotherapy-related adverse events that have not resolved to Grade 1 or better or to baseline at the time of consent with the following exception: patients with ongoing endocrine events that are adequately managed with supplemental therapy are eligible.

### Exclusion Criteria for RO7247669-Containing Arm

Patients who meet any of the following criteria are excluded from the RO7247669-containing arm during Stage 1:
Prior treatment with an anti-lymphocyte activation gene-3 (LAG-3) agent.
Left ventricular ejection fraction (LVEF)<50% assessed by either transthoracic echocardiogram (TTE) or multiple-gated acquisition (MUGA) scan (TTE preferred test) within 6 months from first study drug administration.
Troponin T (TnT) or troponin I (TnI)>institutional ULN. Patients with TnT or TnI levels between >1 and <2×ULN are eligible if repeat levels within 24 hours are ≤1×ULN. If repeat levels within 24 hours are between >1 and <2×ULN, patients may undergo a cardiac evaluation and be considered for treatment.

### End of Study and Length of Study

The end of this study is defined as the date when the last patient completes the last visit, including survival follow-up visits conducted by telephone or in the clinic. The total length of the study, from screening of the first patient to the end of the study, is expected to be approximately 3-5 years.

### Rationale for RO7247669 Dose and Schedule

RO7247669 is administered at a fixed dose of 600 mg Q3W (600 mg on Day 1 of each 21-day cycle), 2100 mg Q2W (2100 mg on Days 1 and 15 of each 28-day cycle), or 1200 mg Q3W (1200 mg on Day 1 of each 21-day cycle) to identify the optimized dose of RO7247669, when administered in combination with bevacizumab, for the treatment of patients with HCC. A fixed dosing regimen of 600 mg Q3W was selected on the basis of available clinical pharmacokinetic, efficacy, and safety data from Study NP41300. During the dose-escalation Part A phase of the study, RO7247669 was well tolerated in the patients and no specific safety concern associated with RO7247669 was identified. No DLT up to the highest dose of 2100 mg every 2 weeks (Q2W) was observed, and no MTD was identified. Anti-tumor activity, as measured by radiographic PRs, was observed starting at a dose of 600 mg Q2W. The pharmacokinetics of RO7247669 were dose linear within the dose range tested in Study NP41300. Further modelling of intratumoral PD-1 and LAG3 target engagement using estimated target properties was performed. It was estimated that a dose and schedule of 600 mg Q3W for RO7247669 would result in >90% PD-1 and LAG3 target engagement at the site of the tumor throughout the treatment period. In addition, RO7247669 was well tolerated, up to the highest dose of 100 mg/kg, in both Good Laboratory Practice toxicology and dose range-finding monkey toxicology studies. Toxicology findings were consistent with reported findings in cynomolgus monkey studies with marketed CPIs.

### Rationale for Bevacizumab Dose and Schedule

In the Q3W treatment arms, bevacizumab is administered at a dose of 15 mg/kg Q3W (15 mg/kg on Day 1 of each 21-day cycle), which is an approved dosage for bevacizumab.

In the Q2W treatment arm, bevacizumab is administered at a dose of 10 mg/kg Q2W (10 mg/kg on Days 1 and 15 of each 28-day cycle), which is an approved dosage for bevacizumab.

### Control Arm (Atezo+Bev)

Patients in the atezolizumab plus bevacizumab (Atezo+bev) arm receive treatment as outlined in Table 28 until unacceptable toxicity or loss of clinical benefit as determined after an integrated assessment of radiographic and biochemical data, local biopsy results (if available), and clinical status (e.g., symptomatic deterioration such as pain secondary to disease). It is recommended that treatment be initiated no later than 7 days after randomization; however, the first dose of study treatment should not occur within 3 days after a core biopsy or other surgical procedure.

TABLE 28
Treatment regimen for atezo + bev arm

| | Dose, Route, and Regimen |
|---|---|
| Cycle Length | (drugs listed in order of administration) |
| 21 days | Atezolizumab 1200 mg by IV infusion on Day 1 |
| | Bevacizumab 15 mg/kg by IV infusion on Day 1 ^{a} |

Atezo + Bev = atezolizumab plus bevacizumab.
^{a} On Day 1 of each cycle, bevacizumab is administered at least 5 minutes after completion of the atezolizumab infusion.

### RO7247669 2100 mg Q2W+bev

Patients in the RO7247669 2100 Q2W+bev arm will receive treatment as outlined in Table 29, until unacceptable toxicity or loss of clinical benefit as determined after an integrated assessment of radiographic and biochemical data, local biopsy results (if available), and clinical status (e.g., symptomatic deterioration such as pain secondary to disease). It is recommended that treatment be initiated no later than 7 days after randomization; however, the first dose of study treatment should not occur within 3 days after a core biopsy or other surgical procedure.

TABLE 29
Treatment regimen for RO7247669 2100 mg Q2W + bev arm

| Cycle | Dose, Route, and Regimen |
|---|---|
| Length | (drugs listed in order of administration) |
| 28 days | RO7247669 2100 mg by IV infusion on Days 1 and 15 |
| | Bevacizumab 10 mg/kg by IV infusion on Days 1 and 15^{a} |

Q2W = every 2 weeks.
RO7247669 + Bev = RO7247669 plus bevacizumab.
^{a}On Day 1 of Cycle 1, bevacizumab is administered at least 90 minutes after completion of the RO7247669 infusion. If the first infusion is tolerated without an infusion-related reaction (IRR), bevacizumab is administered at least 60 minutes after the RO7247669 infusion. If the second infusion is tolerated without an IRR, bevacizumab is administered at least 30 minutes after all subsequent RO7247669 infusions.

### RO7247669 1200 mg Q3W+bev

Patients in the RO7247669 1200 Q3W+bev arm receive treatment as outlined in Table 30, until unacceptable toxicity or loss of clinical benefit as determined after an integrated assessment of radiographic and biochemical data, local biopsy results (if available), and clinical status (e.g., symptomatic deterioration such as pain secondary to disease). It is recommended that treatment be initiated no later than 7 days after randomization; however, the first dose of study treatment should not occur within 3 days after a core biopsy or other surgical procedure.

TABLE 30
Treatment regimen for RO7247669 1200 mg Q3W + bev arm

| | Dose, Route, and Regimen |
|---|---|
| Cycle Length | (drugs listed in order of administration) |
| 21 days | RO7247669 1200 mg by IV infusion on Day 1 |
| | Bevacizumab 15 mg/kg by IV infusion on Day 1^{a} |

Q3W = every 3 weeks.
RO7247669 + Bev = RO7247669 plus bevacizumab.
^{a} On Day 1 of Cycle 1, bevacizumab is administered at least 90 minutes after completion of the RO7247669 infusion. If the first infusion is tolerated without an infusion-related reaction (IRR), bevacizumab is administered at least 60 minutes after the RO7247669 infusion. If the second infusion is tolerated without an IRR, bevacizumab is administered at least 30 minutes after all subsequent RO7247669 infusions.

### RO7247669 600 mg Q3W+bev

Patients in the RO7247669 600 Q3W+bev arm receive treatment as outlined in Table 31, until unacceptable toxicity or loss of clinical benefit as determined after an integrated assessment of radiographic and biochemical data, local biopsy results (if available), and clinical status (e.g., symptomatic deterioration such as pain secondary to disease). It is recommended that treatment be initiated no later than 7 days after randomization; however, the first dose of study treatment should not occur within 3 days after a core biopsy or other surgical procedure.

TABLE 31
Treatment regimen for RO7247669 600 mg Q3W + bev arm

| | Dose, Route, and Regimen |
|---|---|
| Cycle Length | (drugs listed in order of administration) |
| 21 days | RO7247669 600 mg by IV infusion on Day 1 |
| | Bevacizumab 15 mg/kg by IV infusion on Day 1^{a} |

Q3W = every 3 weeks.
RO7247669 + bev = RO7247669 plus bevacizumab.
^{a} On Day 1 of Cycle 1, bevacizumab is administered at least 90 minutes after completion of the RO7247669 infusion. If the first infusion is tolerated without an infusion-related reaction (IRR), bevacizumab is administered at least 60 minutes after the RO7247669 infusion. If the second infusion is tolerated without an IRR, bevacizumab is administered at least 30 minutes after all subsequent RO7247669 infusions.

### B. Statistical Methods

### Primary Analysis

The primary efficacy endpoint is objective response rate (ORR) during Stage 1, as defined in Table 26. ORR is determined according to RECIST v1.1. Patients with missing or no response assessments are classified as non-responders. ORR, the proportion of patients with a complete or partial response, is calculated for each arm, along with 95% CIs (Clopper-Pearson method). The difference in ORR between the experimental arms and the control arm is also calculated, along with 95% CIs, using normal approximation of the binomial distribution.

### Determination of Sample Size

The study is designed to obtain preliminary efficacy, safety, and PK data on immunotherapy-based treatment combinations when administered to patients with advanced liver cancers. Cohort 1 consists of patients with locally advanced or metastatic HCC who have not received prior systemic therapy for their disease. Approximately 170-320 patients are randomly allocated to the control and experimental arms during the study.

### Interim Analyses

It is anticipated that interim analyses will be conducted during the study, with the earliest (Stage 1) interim analysis taking place when at least one experimental arm has completed enrollment in the preliminary phase and patients have been followed for a minimum of 6 weeks. A posterior probability may be used to guide further enrollment in a treatment arm based on an interim analysis of clinical activity in the experimental arm compared with the control arm. If the interim analysis suggests that the activity in an experimental arm is higher than that in the control arm, there may be further enrollment of 20 additional patients in the experimental arm.

An interim analysis is also conducted after approximately 15 patients have been enrolled in a Stage 2 treatment arm and followed for a minimum of 6 weeks. If no clinical activity is observed in a Stage 2 treatment arm, further enrollment in that arm is stopped.

### C. Background and Rationale

### Rationale for Patient Population

The study enrolls patients with locally advanced or metastatic HCC who have not received prior systemic therapy for their disease, regardless of PD-L1 expression or HCC etiology. This broad population is similar to the one that enrolled in two studies evaluating the combination of atezolizumab plus bevacizumab in patients with HCC: Study GO30140, the initial Phase Ib study, and Study YO40245, the randomized Phase III study that demonstrated a statistically significant and clinically meaning benefit for the combination compared with sorafenib.

Advanced HCC is an incurable disease with a high unmet medical need. Sorafenib is currently approved for the first-line treatment of patients with advanced HCC and is regarded as the standard of care in this disease setting. However, the prognosis remains poor, with a median OS of 6.5-10.7 months, and treatment is associated with significant toxicities. Despite the recently demonstrated clinical benefit of atezolizumab plus bevacizumab, there is a continuing need for more efficacious, better tolerated treatment combination regimens for patients with locally advanced or metastatic HCC.

Patients who enroll in the study are also required to have adequate liver function, defined as Child-Pugh Class A. Patients with Child-Pugh Class B or Class C liver function have increased risk of death due to underlying cirrhosis, which could potentially confound the appropriate evaluation of treatment-related anti-tumor efficacy; these patients are thus excluded from the study. The proposed study population is the recommended patient population in the initial study of a new agent or combination of agents for HCC, as noted by an expert panel convened by the American Association for the Study of Liver Diseases (AASLD) (Llovet et al., N Engl J Med, 359:378-380, 2008).

### Rationale for Immunotherapy-Based Treatment Beyond Initial Radiographic Progression

In studies of immunotherapeutic agents, complete response, partial response, and stable disease have each been shown to occur after radiographic evidence of an apparent increase in tumor burden. This initial increase in tumor burden caused by immune cell infiltration in the setting of a T-cell response has been termed pseudo-progression (Hales et al., Ann Oncol, 21:1944-1951, 2010). Evidence of tumor growth followed by a response has been observed in several tumor types. In addition, in some responding patients with radiographic evidence of progression, biopsies of new lesions or areas of new growth in existing lesions revealed immune cells and no viable cancer cells. Because of the potential for a response after pseudoprogression, the study allows patients randomly allocated to immunotherapy-based treatment arms to continue combination treatment after apparent radiographic progression per RECIST v1.1, provided the benefit-risk ratio is judged to be favorable.

### Rationale for the Use of HCC-Specific Modified RECIST

For advanced HCC, RECIST was found to be a poor correlate for the clinical benefit demonstrated in the SHARP trial and other clinical studies for sorafenib (Llovet et al., N Engl J Med, 359:378-380, 2008; Liu et al., Clin Cancer Res, 20:1623-1631, 2014; Takada et al., BMC Res Notes, 8:609, 2015). Similar findings were seen after locoregional therapies for HCC such as radiofrequency ablation and chemoembolization. These therapies reduce the vascularity of the tumor, producing necrosis without necessarily causing a change in overall tumor size. To provide a common framework for the design of clinical trials in HCC, the AASLD has proposed modified criteria (HCC mRECIST), which quantify only the viable portions of the tumor to provide an improved endpoint for assessment (Llovet et al., J Natl Cancer Inst, 100:698-711, 2008; Lencioni and Llovet, Semin Liver Des, 30:52-60, 2010; Lencioni et al., J Hepatol, 66:1166-1172, 2017).

Considering the anti-angiogenic mode of action of bevacizumab, exploratory efficacy endpoints include analyses based on HCC mRECIST. These analyses allow for evaluation of HCC mRECIST as an improved measure of efficacy relative to standard RECIST v1.1 in patients with locally advanced or metastatic HCC.

### Background on Liver Cancer

Liver cancer is the fifth most common cancer and the second most frequent cause of cancer-related death globally, with 854,000 new cases and 810,000 deaths per year. Hepatocellular carcinoma (HCC) is the most prevalent form of primary liver cancer and represents approximately 90% of all primary hepatic malignancies. Less prevalent primary liver cancers include intrahepatic cholangiocarcinoma (iCCA), angiosarcoma, and hepatoblastoma. Upon diagnosis, most patients with primary liver cancer present with advanced disease, a stage when treatment with curative therapies is not recommended. The WHO estimates that more than 1 million people will die from liver cancer in 2030, highlighting a significant global public health issue (Villanueva, N Engl J Med, 380:1450-1462, 2019).

### Background on Hepatocellular Carcinoma

The majority of HCCs occur in patients with underlying liver disease, mostly as a result of hepatitis B virus (HBV) or hepatitis C virus (HCV) infection or alcohol abuse. HBV infection accounts for the majority of HCC cases worldwide; however, in Western countries and Japan, HCV is the main cause of HCC (Villanueva, N Engl J Med, 380:1450-1462, 2019). Universal HBV vaccination and wide implementation of direct-acting antiviral agents against HCV are likely to change the etiologic landscape of HCC. However, the incidence of non-alcoholic fatty liver disease (NAFLD), which is a risk factor for HCC, is increasing worldwide, and NAFLD will soon become a leading cause of liver cancer in Western countries (Villanueva, N Engl J Med, 380:1450-1462, 2019).

HCC is a highly lethal disease with the highest mortality-to-incidence rate ratio (0.98) of any solid tumor (Kamangar et al., J Clin Oncol, 24:2137-2150, 2006). Up to 80% of patients first presenting with HCC have advanced unresectable or metastatic disease because of the late appearance of symptoms. It is a medically complex and difficult-to-treat disease as the majority of patients with HCC have underlying cirrhosis requiring management of both the malignancy and the cirrhosis. In the United States, the 5-year overall survival (OS) rate of patients with HCC is 17% and falls substantially to only 3% if present with distant metastasis (Siegel et al., CA Cancer J Clin, 66:7-30, 2016). In China, the 5-year OS rate of patients with HCC is 10.1% (Chen et al., CA Cancer J Clin, 66:115-132, 2016).

### First-Line Treatment for Advanced Hepatocellular Carcinoma

Sorafenib is an oral multikinase inhibitor that is currently considered the global standard of care for the first-line treatment of patients with advanced HCC. The efficacy of sorafenib has been demonstrated in two large multicenter, randomized, double-blind, placebo-controlled Phase III trials: the Sorafenib HCC Assessment Randomized Protocol (SHARP) trial and a trial conducted in the Asia-Pacific region. Both studies demonstrated a survival benefit of sorafenib versus placebo. In the SHARP trial, median OS was 10.7 months with sorafenib versus 7.9 months with placebo; in the Asia-Pacific trial, median OS was 6.5 months versus 4.2 months. A benefit in median time-to-radiographic progression was also demonstrated: 5.5 months versus 2.8 months in the SHARP trial and 2.8 months versus 1.4 months in the Asia-Pacific trial. The objective response rate (per Response Evaluation Criteria in Solid Tumors, Version 1.0 (RECIST v1.0)) was 2.3% (7 of 299 patients) in the SHARP trial and 3.3% (5 of 150 patients) in the Asia-Pacific trial. The numerically shorter OS and duration of benefit in the Asia-Pacific trial may be attributed to the fact that patients had more advanced disease at the time of recruitment, and potentially also to regional differences in etiology and supportive care (Llovet et al., N Engl J Med, 359:378-380, 2008; Cheng et al., Eur J Cancer, 48:1452-1465, 2009; Cheng et al., Lancet Oncol, 10:25-34, 2012).

Despite the survival benefit reported from these two Phase III studies, the overall benefit-risk ratio of sorafenib is modest because of the known toxicity. Adverse events commonly reported across both sorafenib studies included hand-foot skin reaction, diarrhea, hypertension, weight loss, fatigue, anorexia, alopecia, nausea, and rash/desquamation. Since the approval of sorafenib, there have been a number of negative Phase III studies in head-to-head comparison with sorafenib, including sunitinib, brivanib, and linifanib (Cheng et al., J Clin Oncol, 31:4067-4075, 2013; Johnson et al., J Clin Oncol, 31:3517-3524, 2013; Cainap et al., J Clin Oncol, 33:172-179, 2015). While first-line treatment with lenvatinib, a multi-targeted receptor tyrosine kinase inhibitor, was shown to be non-inferior to sorafenib for OS, a clinically meaningful difference in OS compared with standard of care has, until recently, remained elusive (Kudo et al. 2018).

Study YO40245 (IMbrave150) is a randomized Phase III study evaluating atezolizumab plus bevacizumab versus sorafenib as first-line treatment in patients with advanced or metastatic HCC. This study is the first to demonstrate a statistically significant and clinically meaningful improvement in OS and progression-free survival (PFS) for a novel treatment combination in a head-to-head comparison with sorafenib. At the time of the primary analysis, the risk of death was reduced by 42% for the atezolizumab plus bevacizumab arm compared with the sorafenib arm (stratified hazard ratio (HR)=0.58 (95% CI: 0.42 to 0.79); p=0.0006; median OS, not estimable (NE) vs. 13.24 months). Independent Review Facility-assessed PFS per RECIST v1.1 also demonstrated a statistically significant and clinically meaningful improvement favoring the combination treatment (stratified HR=0.59 (95% CI: 0.47 to 0.76]; p<0.0001; median PFS, 6.83 vs. 4.27 months). Overall, the atezolizumab plus bevacizumab combination in HCC was generally well tolerated with manageable toxicities and the safety profile was consistent with the known risks of the individual study treatments and with the underlying disease (Finn et al., N Engl J Med, 382:1894-1905, 2020).

### Study Rationale

Cancer immunotherapy (CIT) has demonstrated clear clinical efficacy, with significant survival benefits observed across multiple advanced malignancies. Currently, the prevailing CIT approach is to circumvent immune evasion mechanisms and reinvigorate anti-tumor responses by targeting T-cell inhibitory factors such as PD-L1/PD-1. While these targets have resulted in remarkable clinical therapeutic success for various cancer indications, ongoing research indicates that a series of stepwise events is necessary for the generation of a continuous anti-tumor immune response (Chen and Mellman, Immunity, 39:1-10, 2013). Each event is critical for an effective response, and each is also susceptible to several tumor immune-evasion mechanisms. Thus, the need to identify and circumvent the various factors that account for the absence of an effective anti-cancer immune response will be critical for propagating cancer immunity and advancing the field of CIT, most likely through combined targeted therapy regimens.

The present Phase Ib/II umbrella study is designed to accelerate the development of CIT combinations by identifying early signals and establishing proof-of-concept clinical data in patients with advanced liver cancers.

The study assesses the importance of simultaneously targeting multiple mechanisms of immune escape through immune cell priming and activation, tumor infiltration, and/or recognition of tumor cells for elimination. To improve the confidence of clinical signal detection in the experimental arms, the study includes a control arm. Moreover, patients who experience disease progression with the initial treatment regimen (Stage 1) may be eligible to continue treatment with a different treatment regimen (Stage 2), which may advance the scientific understanding of immune escape mechanisms in patients who fail to respond to, or experience disease progression during, treatment with a CIT regimen.

The study initially enrolls patients with locally advanced or metastatic HCC who have not received prior systemic therapy for their disease. Despite the recently demonstrated clinical benefit of atezolizumab plus bevacizumab, there remains a high unmet medical need for patients with unresectable locally advanced or metastatic HCC, requiring further evaluation of novel, more efficacious treatment combinations.

The target and proposed mechanism-of-action classification for each experimental investigational medicinal product (IMP) is summarized in Table 32.

TABLE 32
Target and proposed mechanism-of-action classification
for experimental investigational medicinal products

| Experimental | | |
|---|---|---|
| IMP | Target | Proposed mechanism-of-action classification |
| Atezolizumab | PD-L1 | Checkpoint inhibitor |
| Bevacizumab | VEGF | Angiogenesis inhibitor; recruitment of |
| | | T cells to the tumor microenvironment ^{a} |
| RO7247669 | PD-1, | Bispecific, dual immune checkpoint inhibitor |
| | LAG-3 | |

IMP = investigational medicinal product; LAG-3 = lymphocyte activation gene-3; NK = natural killer; VEGF = vascular endothelial growth factor.
^{a} Wallin et al., Nat Commun, 7: 12624, 2016.

### D. Assessment of Safety

Safety assessments consist of monitoring and recording adverse events, including serious adverse events and adverse events of special interest, performing protocol-specified safety laboratory assessments, measuring protocol-specified vital signs, and conducting other protocol-specified tests that are deemed critical to the safety evaluation of the study.

According to the ICH guideline for Good Clinical Practice, an adverse event is any untoward medical occurrence in a clinical investigation subject administered a pharmaceutical product, regardless of causal attribution. An adverse event can therefore be any of the following:
Any unfavorable and unintended sign (including an abnormal laboratory finding), symptom, or disease temporally associated with the use of a medicinal product, whether or not considered related to the medicinal product.
Any new disease or exacerbation of an existing disease (a worsening in the character, frequency, or severity of a known condition).
Recurrence of an intermittent medical condition (e.g., headache) not present at baseline.
Any deterioration in a laboratory value or other clinical test (e.g., ECG, X-ray) that is associated with symptoms or leads to a change in study treatment or concomitant treatment or discontinuation from study treatment.
Adverse events that are related to a protocol-mandated intervention, including those that occur prior to assignment of study treatment (e.g., screening invasive procedures such as biopsies).

### E. Statistical Considerations and Analysis Plan

The final study analysis is based on patient data collected through study discontinuation. If not otherwise specified, efficacy analyses are based on the efficacy-evaluable population, defined as all patients who receive at least one dose of each drug for their assigned treatment regimen, and safety analyses are based on the safety-evaluable population, defined as all patients who receive any amount of study treatment.

The analysis results are summarized by the treatment regimen that patients actually received, as well as by stage (Stage 1 or Stage 2). Data are described and summarized as warranted by sample size. Continuous variables are summarized through use of means, standard deviations, medians, and ranges. Categorical variables are summarized through use of counts and percentages. Listings are used in place of tables in the event of small sample sizes.

New baseline values are established for the Stage 2 efficacy and safety analyses.

### Primary Efficacy Endpoint

The primary efficacy endpoint is ORR during Stage 1, as defined above. ORR is determined according to RECIST v1.1. Patients with missing or no response assessments will be classified as non-responders.

ORR, the proportion of patients with a complete or partial response, is calculated for each arm, along with 95% CIs (Clopper-Pearson method). The difference in ORR between the experimental arms and the control arm is also calculated, along with 95% CIs, using normal approximation of the binomial distribution.

### Secondary Efficacy Endpoints

The secondary efficacy endpoints are PFS, OS, OS at specific timepoints (e.g., 6 months), duration of response (DOR), and disease control during Stage 1, as defined above. PFS, DOR, and disease control are determined according to RECIST v1.1.

DOR is derived for efficacy-evaluable patients with a complete or partial response. For patients who do not have documented disease progression or death in a study stage, PFS and DOR are censored at the day of the last tumor assessment. Patients who are still alive at the time of OS analysis are censored at the last date they were known to be alive.

The Kaplan-Meier method is used to estimate the median for PFS, OS, and DOR, with 95% CIs constructed through use of the Brookmeyer and Crowley method. The OS rate at specific timepoints is also estimated using the Kaplan-Meier method, with 95% CIs calculated on the basis of Greenwood's estimate for the variance. Disease control rate (the proportion of patients with stable disease for ≥12 weeks), a partial response, or a complete response, is calculated for each treatment arm, with 95% CIs estimated through use of Clopper-Pearson's exact method.

### Exploratory Efficacy Endpoints

The exploratory efficacy endpoints are ORR, PFS, DOR, and disease control during Stage 1, as determined according to HCC mRECIST; ORR, PFS, DOR, and disease control during Stage 2, as determined according to RECIST v1.1 and HCC mRECIST. ORR, PFS, DOR, and disease control are analyzed through use of the same methods described above. DOR is derived for efficacy-evaluable patients with a complete or partial response.

### F. Study Details Specific to RO7247669+Bev Arms

### Background on RO7247669

RO7247669 is a novel, fragment crystallizable (Fc)-silent IgG1-based bispecific antibody (bsAb) in the 1+1 format that incorporates monovalent binding to two immune checkpoint proteins: PD-1 and lymphocyte activation gene-3 (LAG-3). RO7247669 is designed to target dysfunctional tumor antigen-specific T lymphocytes (expressing PD-1 and LAG-3) to establish or re-establish an effective anti-tumor

immune response in cancer patients, which may result in improved therapeutic responses over currently available therapies. In addition, RO7247669 is engineered to prevent binding to Fc-gamma receptors, thus potentially avoiding tumor-associated macrophage resistance mechanisms, which have been observed with IgG4-based anti-PD-1 antibodies, such as pembrolizumab and nivolumab (Arlauckas et al., Sci Transl Med, 9:389, 2017).

Clinical evaluation of RO7247669 is ongoing in a first-in-human, dose-finding study (NP41300) as a single agent in patients with and without prior checkpoint inhibitor (CPI) exposure.

### Background on Bevacizumab

Bevacizumab is a recombinant humanized monoclonal antibody that recognizes all isoforms of vascular endothelial growth factor (VEGF). It may exert a direct anti-angiogenic effect by binding to and clearing VEGF from the tumor microenvironment. Additional anti-tumor activity may be on tumor vasculature, interstitial pressure, and blood vessel permeability, providing for enhanced chemotherapy delivery to tumor cells (Jain, Nat Med, 7:987-989, 2001).

Bevacizumab is approved for the first-line and second-line treatment of metastatic colorectal cancer (mCRC), first-line treatment of advanced non-small cell lung cancer (NSCLC), metastatic breast cancer, advanced renal cell carcinoma (RCC), and ovarian cancer, and treatment of recurrent glioblastoma. Bevacizumab is currently being tested in combination with atezolizumab in Phase I-III clinical trials. Bevacizumab has been generally well tolerated, and adverse events have been manageable.

### Rationale for RO7247669 600 mg Q3W+Bev Arm

### The PD-1/PD-L1 Pathway

Encouraging clinical data in the field of tumor immunotherapy have demonstrated that therapies focused on enhancing T-cell responses against cancer can result in a significant survival benefit in patients with advanced malignancies (Hodi et al., N Engl J Med, 363:711-723, 2010; Kantoff et al., N Engl J Med, 363:411-422, 2010; Chen et al., Clin Cancer Res, 18:6580-6587, 2012).

The PD-1/PD-L1 pathway serves as an immune checkpoint to temporarily dampen immune responses in states of chronic antigen stimulation, such as chronic infection or cancer. PD-1 is an inhibitory receptor that is expressed on activated and exhausted T cells, including tumor infiltrating CD8+ T cells that recognize mutated tumor antigens (neo-antigens). Binding of PD-L1 to PD-1 inhibits T-cell proliferation, activation, cytokine production, and cytolytic activity, leading to a functionally inactivated and exhausted T-cell state (Butte et al., Immunity, 27:111-122. 2007; Yang et al., J Immunol, 187:1113-1119, 2011).

Therapeutic targeting of the PD-1/PD-L1 pathway to enhance anti-tumor T-cell responses has been clinically validated across multiple solid tumors, as both a single agent and in combination with chemotherapy and other targeted agents. Cancer immunotherapy (CIT) agents, particularly immune CPIs, have made a significant impact on the treatment of patients with advanced malignancies in recent years. However, despite the remarkable clinical efficacy of these therapies, it has become clear that they are not sufficiently active as monotherapy for many patients. To date, treatment with single-agent CPIs that target the PD-1/PD-L1 pathway has shown minimal activity in the treatment of patients with hepatocellular carcinoma (HCC).

### The LAG-3 Pathway

LAG-3 is an immune checkpoint protein involved in the regulation of anti-tumor immunity and chronic infection. LAG-3 is expressed on activated T cells, B cells, natural killer (NK) cells, and a subset of tolerogenic plasmacytoid dendritic cells (DCs), and constitutively on T-regulatory cells (Huard et al., Immunogenetics, 39:213-217, 1994). Structurally similar to CD4, LAG-3 is a member of the Ig superfamily and binds to major histocompatibility complex class II (MHC-II). The interaction of LAG-3 with MHC-II inhibits T-cell proliferation, activation, cytolytic function, and proinflammatory cytokine production

(Goldberg and Drake, Curr Top Microbiol Immunol, 344:269-278, 2011). The effect of LAG-3 expression on T-regulatory cells is controversial. An early report concluded that LAG-3 promotes T-regulatory cell-mediated immune suppression (Camisaschi et al., J Immunol, 184:6545-6551, 2010). However, a more recent report from the same authors describes LAG-3 to limit T-regulatory cell mediated immune suppression (Zhang et al., Sci Immunol, 2eaah4569, 2017).

Expression of LAG-3 has been reported across various tumor types, including breast cancer, ovarian cancer, NSCLC, melanoma, RCC, prostate cancer, and HCC, and is associated with poor prognosis (Matsuzaki et al., Proc Natl Acad Sci USA, 107:7875-7800, 2010; Baitsch et al., J Clin Invest, 121:2350-2360, 2011; Thommen et al., Cancer Immunol Res, 31344-55, 2015; He et al., Cancer Sci, 107:1193-1197, 2016; Norstrom et al., Oncotarget, 7:23581-23593, 2016). Clinical evaluation of anti-LAG-3 agents, given as a single agent and in combination with other CPIs, is ongoing in several early-phase studies in patients with advance solid tumors (Long et al., Genes Cancer, 9 (5-6): 176-189, 2018). Preliminary data demonstrate that anti-LAG-3 therapy was well tolerated as a single agent and in combination with anti-PD-1 therapies, and the safety profiles were consistent with those of other CPIs (Ascierto et al., Ann Onco, 28 (Suppl 5): c611-612, 2017; Hong et al., J Clin Oncol, 36:3012, 2018; Stratton et al., Society for Immunotherapy of Cancer (SITC) Abstract P325 2018).

Emerging clinical data highlight a role for the LAG-3 pathway in the pathogenesis of HCC. The expression of LAG-3 is significantly elevated on tumor-infiltrating lymphocytes (TILs) of hepatitis B virus-infected patients with HCC compared with peripheral blood lymphocytes, and this is associated with a severe functional T-cell defect at the tumor site (Li et al., Immunol Lett, 150:1116-1122, 2013). In patients with HCC who have not received prior systemic therapy for their disease, a similar elevation in LAG-3 expression on TILs was reported, and no relationship was identified between LAG-3 expression and any disease-related characteristics, including viral status (Yarchoan et al., Clin Cancer Res, 23:7333-7339, 2017).

LAG-3 expression has also recently been identified as a prognostic factor (in addition to vascular invasion, tumor size, and cirrhosis) for poor survival outcomes in patients with HCC (Guo et al., J Transl Med, 18:306, 2020).

Taken together, therapeutic targeting of LAG-3 may represent an attractive strategy for the treatment of patients with HCC.

### The VEGF Pathway

VEGF-A is a pro-angiogenic molecule produced by endothelium, tumors, and tumor-associated macrophages. In addition to promoting tumor angiogenesis, there is increasing evidence that the VEGF pathway also plays an important role in cancer immune evasion by exerting and maintaining an immunosuppressive tumor microenvironment through several mechanisms.

VEGF-A inhibits the maturation of dendritic cells (DCs), promotes the expression of inhibitory immune checkpoint molecules on intratumoral CD8+ T-cells, and induces Fas ligand (FasL) expression on endothelial cells, which acquired the ability to kill effector CD8+ T cells, but not T-regulatory cells (Gabrilovich et al., Nat Med, 2:1096-1103, 1996; Huang et al., Blood, 110:624-631, 2007; Motz et al., Nat Med, 20:607-615, 2014; Voron et al., J Exp Med, 212:139-148, 2015). Experiments with activated endothelial cells also suggest that VEGF may reduce lymphocyte adhesion to vessel walls in the tumor microenvironment, thus contributing to decreased immune cell recruitment to the tumor site (Bouzin et al., J Immunol, 178:1505-1511, 2007). In addition, VEGF recruits macrophages into the tumor microenvironment that have a M2 polarization state, which is typically involved in wound healing. These M2 tumor-associated macrophages ultimately help to establish and to maintain an immunosuppressive microenvironment (Chen and Mellman, Immunity, 39:1-10, 2013).

Targeting the VEGF pathway with agents such as bevacizumab has been clinically validated with demonstrated anti-tumor activity in patients with several advance malignancies. However, when administered as a single agent to patients with HCC, bevacizumab has demonstrated only minimal activity (Siegel et al., J Clin Oncol, 26:2992-2998, 2008; Boige et al., Oncologist, 17:1063-1072, 2012).

Combination Treatment with an Anti-PD-1/LAG-3 Bispecific Antibody and an Anti-VEGF Agent

Durable clinical benefit is limited to a minority of patients treated with single-agent PD-L1/PD-1 inhibitors. Therapies targeting the mechanisms of resistance to anti-PD-L1/PD-1 therapies are needed to improve outcomes in patients with solid tumor cancers. A strong scientific rationale and emerging clinical data suggest that combined PD-1, LAG-3, and VEGF inhibition may be clinically beneficial in a number of tumor types.

Anti-VEGF agents promote the normalization of tumor vasculature, thereby increasing access of therapeutic agents (Jain, Nat Med, 7:987-989, 2001). In addition, bevacizumab can restore and/or maintain the antigen-presentation capacity of DCs, leading to enhanced T-cell infiltration in tumors (Oelkrug and Ramage, Clin Exp Immunol, 178:1-8, 2014; Wallin et al., Nat Commun, 7:12624, 2016). Administration of anti-VEGF-A has been shown to attenuate tumor endothelial FasL expression and produce a significant increase in the influx of tumor-rejecting CD8+ T cells, leading to tumor growth suppression (Motz et al., Nat Med, 20:607-615, 2014). Anti-VEGF therapies can also reduce the frequency of myeloid-derived suppressor cells and decrease production of suppressive cytokines (Roland et al., PLOS One, 4: e7669, 2009). In addition, VEGF-A was shown to induce the expression of PD-1, and other inhibitory immune checkpoint proteins, including TIM-3 and LAG-3 on CD8+ T cells, which could be reversed by VEGF inhibition (Voron et al., J Exp Med, 212:139-148, 2015).

The immunomodulatory effects of bevacizumab are anticipated to increase CD8+ T-cell recruitment and relieve intratumoral immunosuppression, thereby boosting the effects of immunotherapy. Indeed, clinical data have demonstrated a beneficial effect of anti-angiogenesis and immunomodulation within the context of PD-L1/PD-1 pathway blockade. Early-phase studies have demonstrated that combination treatment of the anti-PD-1 therapies nivolumab and pembrolizumab with different anti-angiogenesis agents can elicit responses in patients with metastatic urothelial carcinoma, RCC, ovarian, and endometrial cancers (Apolo et al., J Clin Oncol, 35 (Suppl. 6): 293, 2017; Lee et al., Ann Oncol, 28 (Suppl. 5): v295-v329, 2017; Makker et al., J Clin Oncol, 35 (Suppl 15): 5598, 2017; Liu et al., JAMA Oncol, 5:1731-1738, 2019; Dudek et al., J Clin Oncol, 38:1138-1145, 2020). The activity of combination treatment with the anti-PD-L1 therapy atezolizumab and bevacizumab has also been demonstrated in multiple, large, randomized Phase III clinical studies in patients with NSCLC, RCC, and HCC (Socinski et al., N Engl J Med, 378:2288-2301, 2018; Rini et al., Lancet, 393:2404-2415, 2019; Finn et al., N Engl J Med, 382:1894-1905, 2020). Resistance to PD-L1/PD-1 blockade may result in the expression of multiple co-inhibitory immune checkpoints on the surface of effector T cells. LAG-3 is frequently co-expressed with PD-1 on tumor-infiltrating lymphocytes (TILs), and dual blockade of PD-1 and LAG-3 has been shown to enhance CD8+ T-cell effector function and potentiate anti-tumor immunity in nonclinical models. Blockade of these two receptors in mice with colon, fibrosarcoma, or ovarian tumors resulted in tumor remission in approximately 80% of animals compared with 10% to 40% with either as a single agent (Woo et al., Cancer Res, 72:917-927, 2012; Huang et al., Oncotarget, 6:27359-27377, 2015). TILs from patients with ovarian cancer showed that antigen-specific CD8+ T cells co-expressing PD-1 and LAG-3 exhibited greater impairment in their ability to respond to cognate antigen stimulation compared with CD8+ T cells that expressed one checkpoint molecule (Matsuzaki et al., Proc Natl Acad Sci USA, 107:7875-7880, 2010). In patients with NSCLC, overexpression of LAG-3 on TILs was correlated with PD-1/PD-L1 expression and was linked to higher risk of recurrence and poor survival outcomes (He et al., J Thorac Oncol, 12:814-823, 2017). In clinical samples from patients with untreated HCC, the majority showed co-staining for LAG-3 and PD-1 with higher expression among TILs compared with expression in background liver tissue (Yarchoan et al., Clin Cancer Res, 23:7333-7339, 2017). Based on the above-described data, it is hypothesized that simultaneous targeting of the PD-1 and LAG-3 pathways with the bsAb RO7247669, in combination with the immunomodulatory effects of bevacizumab, may augment the anti-tumor immune response, resulting in an improved and more durable clinical benefit in patients with HCC.

### OTHER EMBODIMENTS

Although the foregoing invention has been described in some detail by way of illustration and example for purposes of clarity of understanding, the descriptions and examples should not be construed as limiting the scope of the invention.

1. A method for treating a subject having a cancer, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3, wherein the method comprises administering to the subject the bispecific antibody at a fixed dose of 600 mg every three weeks.

2. The method of claim 1, wherein the cancer is:
(a) a solid tumor;
(b) a locally advanced or metastatic cancer; or
(c) a skin cancer, a liver cancer, a lung cancer, a renal cancer, a bladder cancer, a breast cancer, or an esophageal cancer.

3-4. (canceled)

5. The method of claim 2, wherein the cancer is:
(a) a skin cancer, and wherein the skin cancer is a melanoma;
(b) a liver cancer, and wherein the liver cancer is a hepatocellular carcinoma (HCC);
(c) a lung cancer, and wherein the lung cancer is a non-small cell lung cancer (NSCLC);
(d) a renal cancer, and wherein the renal cancer is a renal cell carcinoma (RCC);
(e) a bladder cancer, and wherein the bladder cancer is a metastatic urothelial carcinoma (mUC);
(f) a breast cancer, and wherein the breast cancer is a triple-negative breast cancer (TNBC); or
(g) an esophageal cancer, and wherein the esophageal cancer is an esophageal squamous cell carcinoma (ESCC).

6. The method of claim 2, wherein the cancer is a skin cancer, and wherein the skin cancer is a previously untreated unresectable or metastatic melanoma.

7. The method of claim 5, wherein the cancer is a melanoma, and wherein the melanoma is:
(a) a Stage III melanoma with measurable lymph node metastases;
(b) an unresectable Stage III melanoma;
(c) a Stage IV melanoma; or
(d) not a mucosal melanoma or a uveal melanoma.

8-13. (canceled)

14. A method for treating a subject having a melanoma, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3, wherein the method comprises administering to the subject the bispecific antibody at a fixed dose of 600 mg every three weeks, and wherein the melanoma is:
(a) an unresectable Stage III melanoma; or
(b) a Stage IV melanoma.

15. (canceled)

16. A method for treating a subject having a liver cancer, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3, wherein the method comprises administering to the subject the bispecific antibody at a fixed dose of 600 mg every three weeks.

17-22. (canceled)

23. The method of claim 1, wherein the method further comprises administering to the subject bevacizumab at a dose of about 15 mg/kg every three weeks.

24-25. (canceled)

26. The method of claim 1, wherein the subject has not previously been treated:
(a) for metastatic or unresectable disease;
(b) with an anti-cancer therapy comprising an immunomodulatory agent; or
(c) with an anti-LAG3 therapy.

27-28. (canceled)

29. The method of claim 1, wherein the bispecific antibody targeting PD-1 and LAG3 comprises:
(a) a first antigen-binding domain that specifically binds to PD-1 comprising:
(1) a VH domain comprising:
(i) an HVR-H1 sequence comprising the amino acid sequence of SEQ ID NO: 25;
(ii) an HVR-H2 sequence comprising the amino acid sequence GGR; and
(iii) an HVR-H3 sequence comprising an amino acid sequence of SEQ ID NO: 26; and
(2) a VL domain comprising:
(i) an HVR-L1 sequence comprising the amino acid sequence of SEQ ID NO: 27;
(ii) an HVR-L2 sequence comprising the amino acid sequence RSS; and
(iii) an HVR-L3 sequence comprising the amino acid sequence of SEQ ID NO: 28; and
(b) a second antigen-binding domain that specifically binds to LAG3 comprising:
(1) a VH domain comprising:
(i) an HVR-H1 sequence comprising the amino acid sequence of SEQ ID NO: 31:
(ii) an HVR-H2 sequence comprising the amino acid sequence of SEQ ID NO: 32; and
(iii) an HVR-H3 sequence comprising an amino acid sequence of SEQ ID NO: 33; and
(2) a VL domain comprising:
(i) an HVR-L1 sequence comprising the amino acid sequence of SEQ ID NO: 34:
(ii) an HVR-L2 sequence comprising the amino acid sequence of SEQ ID NO: 35; and
(iii) an HVR-L3 sequence comprising the amino acid sequence of SEQ ID NO: 36.

30. (canceled)

31. The method of claim 29, wherein the first antigen-binding domain comprises a VH domain comprising the amino acid sequence of SEQ ID NO: 29 and a VL domain comprising the amino acid sequence of SEQ ID NO: 30, and the second antigen-binding domain comprises a VH domain comprising the amino acid sequence of SEQ ID NO: 37 and a VL domain comprising the amino acid sequence of SEQ ID NO: 38.

32. The method of claim 29, wherein the bispecific antibody is a full-length antibody.

33. The method of claim 32, wherein the bispecific antibody targeting PD-1 and LAG3 comprises a Fc domain:
(a) that is an IgG;
(b) comprising one or more amino acid substitutions that reduce binding to an Fc receptor; or
(c) comprising a modification promoting the association of the first and second subunit of the Fc domain.

34. (canceled)

35. The method of claim 33, wherein the bispecific antibody targeting PD-1 and LAG3 comprises:
(a) an Fc domain of human IgG1 subclass with the amino acid mutations L234A, L235A, and P329G (numbering according to Kabat EU index); or
(b) an Fc domain comprising a modification promoting the association of the first and second subunit of the Fc domain, wherein the first subunit of the Fc domain comprises the amino acid substitutions S354C and T366W (numbering according to Kabat EU index) and the second subunit of the Fc domain comprises the amino acid substitutions Y349C, T366S, and Y407V (numbering according to Kabat EU index).

36. (canceled)

37. The method of claim 29, wherein the bispecific antibody targeting PD-1 and LAG3 comprises an Fc domain, a first Fab fragment comprising the first antigen-binding domain, and a second Fab fragment comprising the second antigen-binding domain.

38. The method of claim 37, wherein in one of the Fab fragments of the bispecific antibody targeting PD-1 and LAG3 the variable domains VL and VH are replaced by each other so that the VH domain is part of the light chain and the VL domain is part of the heavy chain.

39. The method of claim 37, wherein:
(a) in the constant domain CL of one of the Fab fragments the amino acid at position 124 is substituted independently by lysine (K), arginine (R), or histidine (H) (numbering according to Kabat EU Index);
(b) in the constant domain CH1 the amino acids at positions 147 and 213 are substituted independently by glutamic acid (E) or aspartic acid (D) (numbering according to Kabat EU index);
(c) in the constant domain CL of the second Fab fragment the amino acid at position 124 is substituted independently by lysine (K), arginine (R), or histidine (H) (numbering according to Kabat EU Index); or
(d) in the constant domain CH1 the amino acids at positions 147 and 213 are substituted independently by glutamic acid (E) or aspartic acid (D) (numbering according to Kabat EU index).

40. The method of claim 29, wherein the bispecific antibody comprises a first heavy chain comprising an amino acid sequence with at least 95% sequence identity to the sequence of SEQ ID NO: 39, a first light chain comprising an amino acid sequence with at least 95% sequence identity to the sequence of SEQ ID NO: 40, a second heavy chain comprising an amino acid sequence with at least 95% sequence identity to the sequence of SEQ ID NO: 41, and a second light chain comprising an amino acid sequence with at least 95% sequence identity to the sequence of SEQ ID NO: 42.

41. The method of claim 40, wherein the bispecific antibody comprises a first heavy chain comprising the amino acid sequence of SEQ ID NO: 39, a first light chain comprising the amino acid sequence of SEQ ID NO: 40, a second heavy chain comprising the amino acid sequence of SEQ ID NO: 41, and a second light chain comprising the amino acid sequence of SEQ ID NO: 42.

42. (canceled)

43. The method of claim 1, wherein the subject is a human.

44-129. (canceled)


SEQUENCE LISTING (71 sequences)

SEQ ID NO: 1 - protein, 7 aa, synthetic construct
SNSAAWN

SEQ ID NO: 2 - protein, 18 aa, synthetic construct
KTYYRFKWYSDYAVSVKG

SEQ ID NO: 3 - protein, 14 aa, synthetic construct
ESTTYDLLAGPFDY

SEQ ID NO: 4 - protein, 16 aa, synthetic construct
SSQTVLYSSNNKKYLA

SEQ ID NO: 5 - protein, 7 aa, synthetic construct
WASTRES

SEQ ID NO: 6 - protein, 9 aa, synthetic construct
QQYYSTPFT

SEQ ID NO: 7 - protein, 23 aa, synthetic construct
DIVMTQSPDSLAVSLGERATINC

SEQ ID NO: 8 - protein, 15 aa, synthetic construct
WYQQKPGQPPNLLIY

SEQ ID NO: 9 - protein, 32 aa, synthetic construct
GVPDRFSGSGSGTDFTLTISSLQAEDVAVYYC

SEQ ID NO: 10 - protein, 10 aa, synthetic construct
FGPGTKVEIK

SEQ ID NO: 11 - protein, 30 aa, synthetic construct
  VARIANT 1: note = E or Q
XVQLQQSGPGLVKPSQTLSLTCAISGDSVS

SEQ ID NO: 12 - protein, 14 aa, synthetic construct
WIRQSPSRGLEWLG

SEQ ID NO: 13 - protein, 32 aa, synthetic construct
RITINPDTSKNQFSLQLNSVTPEDTAVFYCTR

SEQ ID NO: 14 - protein, 11 aa, synthetic construct
WGQGTLVTVSS

SEQ ID NO: 15 - protein, 30 aa, synthetic construct
EVQLQQSGPGLVKPSQTLSLTCAISGDSVS

SEQ ID NO: 16 - protein, 30 aa, synthetic construct
QVQLQQSGPGLVKPSQTLSLTCAISGDSVS

SEQ ID NO: 17 - protein, 126 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 18 - protein, 126 aa, synthetic construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 19 - protein, 113 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 20 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 21 - protein, 223 aa, Homo sapiens [sequence omitted]

SEQ ID NO: 22 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 23 - protein, 456 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 220 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 7 aa, synthetic construct
GFSFSSY

SEQ ID NO: 26 - protein, 9 aa, synthetic construct
TGRVYFALD

SEQ ID NO: 27 - protein, 11 aa, synthetic construct
SESVDTSDNSF

SEQ ID NO: 28 - protein, 6 aa, synthetic construct
NYDVPW

SEQ ID NO: 29 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 30 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 31 - protein, 5 aa, synthetic construct
DYTMN

SEQ ID NO: 32 - protein, 17 aa, synthetic construct
VISWDGGGTYYTDSVKG

SEQ ID NO: 33 - protein, 12 aa, synthetic construct
GLTDTTLYGSDY

SEQ ID NO: 34 - protein, 11 aa, synthetic construct
RASQSISSYLN

SEQ ID NO: 35 - protein, 7 aa, synthetic construct
AASTLQS

SEQ ID NO: 36 - protein, 9 aa, synthetic construct
QQTYSSPLT

SEQ ID NO: 37 - protein, 121 aa, synthetic construct
EVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSS

SEQ ID NO: 38 - protein, 107 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIK

SEQ ID NO: 39 - protein, 443 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 227 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 451 aa, synthetic construct
EVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 42 - protein, 214 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 44 - protein, 497 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 45 - protein, 422 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 46 - protein, 5 aa, synthetic construct
GGGGS

SEQ ID NO: 47 - protein, 10 aa, synthetic construct
GGGGSGGGGS

SEQ ID NO: 48 - protein, 10 aa, synthetic construct
SGGGGSGGGG

SEQ ID NO: 49 - protein, 14 aa, synthetic construct
GGGGSGGGGSGGGG

SEQ ID NO: 50 - protein, 10 aa, synthetic construct
GSPGSSSSGS

SEQ ID NO: 51 - protein, 15 aa, synthetic construct
GGGGSGGGGSGGGGS

SEQ ID NO: 52 - protein, 20 aa, synthetic construct
GGGGSGGGGSGGGGSGGGGS

SEQ ID NO: 53 - protein, 8 aa, synthetic construct
GSGSGSGS

SEQ ID NO: 54 - protein, 8 aa, synthetic construct
GSGSGNGS

SEQ ID NO: 55 - protein, 8 aa, synthetic construct
GGSGSGSG

SEQ ID NO: 56 - protein, 6 aa, synthetic construct
GGSGSG

SEQ ID NO: 57 - protein, 4 aa, synthetic construct
GGSG

SEQ ID NO: 58 - protein, 8 aa, synthetic construct
GGSGNGSG

SEQ ID NO: 59 - protein, 8 aa, synthetic construct
GGNGSGSG

SEQ ID NO: 60 - protein, 6 aa, synthetic construct
GGNGSG

SEQ ID NO: 61 - protein, 461 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 62 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 10 aa, synthetic construct
GFTFSDSWIH

SEQ ID NO: 65 - protein, 18 aa, synthetic construct
AWISPYGGSTYYADSVKG

SEQ ID NO: 66 - protein, 9 aa, synthetic construct
RHWPGGFDY

SEQ ID NO: 67 - protein, 11 aa, synthetic construct
RASQDVSTAVA

SEQ ID NO: 68 - protein, 7 aa, synthetic construct
SASFLYS

SEQ ID NO: 69 - protein, 9 aa, synthetic construct
QQYLYHPAT

SEQ ID NO: 70 - protein, 118 aa, synthetic construct [sequence omitted]

SEQ ID NO: 71 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR